SEQUENCE LISTING (23 sequences)

SEQ ID NO: 1 - DNA, 18 bp, synthetic construct
  misc_feature 1..18: note = Description of Artificial Sequence: Synthetic primer
  source 1..18: mol_type = other DNA
aacacaagctttcggcag

SEQ ID NO: 2 - DNA, 22 bp, synthetic construct
  misc_feature 1..22: note = Description of Artificial Sequence: Synthetic primer
  source 1..22: mol_type = other DNA
gaaatttggatctttgtcatcc

SEQ ID NO: 3 - DNA, 41 bp, synthetic construct
  misc_feature 1..41: note = Description of Artificial Sequence: Synthetic primer
  source 1..41: mol_type = other DNA
tgcggccaatgtttgtaatcagccaaggaaattttggggac

SEQ ID NO: 4 - DNA, 39 bp, synthetic construct
  misc_feature 1..39: note = Description of Artificial Sequence: Synthetic primer
  source 1..39: mol_type = other DNA
cgcattggcatggaagtcactttgatggcacctgtgtag

SEQ ID NO: 5 - DNA, 19 bp, synthetic construct
  misc_feature 1..19: note = Description of Artificial Sequence: Synthetic primer
  source 1..19: mol_type = other DNA
ttccttgtctgattagttc

SEQ ID NO: 6 - DNA, 18 bp, synthetic construct
  misc_feature 1..18: note = Description of Artificial Sequence: Synthetic primer
  source 1..18: mol_type = other DNA
accttcgggaacgtggtt

SEQ ID NO: 7 - DNA, 19 bp, synthetic construct
  misc_feature 1..19: note = Description of Artificial Sequence: Synthetic primer
  source 1..19: mol_type = other DNA
tccagttacgctggagtct

SEQ ID NO: 8 - DNA, 18 bp, synthetic construct
  misc_feature 1..18: note = Description of Artificial Sequence: Synthetic primer
  source 1..18: mol_type = other DNA
aagatcacgcgccatcag

SEQ ID NO: 9 - DNA, 41 bp, synthetic construct
  misc_feature 1..41: note = Description of Artificial Sequence: Synthetic primer
  source 1..41: mol_type = other DNA
gctcgcaggtccaaatctgcactgaatgatatgcggcctcg

SEQ ID NO: 10 - DNA, 39 bp, synthetic construct
  misc_feature 1..39: note = Description of Artificial Sequence: Synthetic primer
  source 1..39: mol_type = other DNA
ctctgcgcggacttgtggagcagggacatcccagagact

SEQ ID NO: 11 - DNA, 18 bp, synthetic construct
  misc_feature 1..18: note = Description of Artificial Sequence: Synthetic primer
  source 1..18: mol_type = other DNA
caccgccatgctgaagtc

SEQ ID NO: 12 - DNA, 19 bp, synthetic construct
  misc_feature 1..19: note = Description of Artificial Sequence: Synthetic primer
  source 1..19: mol_type = other DNA
cagccgctcaccgtgagtt

SEQ ID NO: 13 - DNA, 19 bp, synthetic construct
  misc_feature 1..19: note = Description of Artificial Sequence: Synthetic primer
  source 1..19: mol_type = other DNA
tggctactaccgaagagct

SEQ ID NO: 14 - DNA, 20 bp, synthetic construct
  misc_feature 1..20: note = Description of Artificial Sequence: Synthetic primer
  source 1..20: mol_type = other DNA
tgcagcattgttagcaggat

SEQ ID NO: 15 - DNA, 41 bp, synthetic construct
  misc_feature 1..41: note = Description of Artificial Sequence: Synthetic primer
  source 1..41: mol_type = other DNA
tctggcccagttcctaggtagtccagacgaattcgtggtgg

SEQ ID NO: 16 - DNA, 40 bp, synthetic construct
  misc_feature 1..40: note = Description of Artificial Sequence: Synthetic primer
  source 1..40: mol_type = other DNA
agacggcatcatatgggttgcacgggtgccaatgtgatct

SEQ ID NO: 17 - DNA, 25 bp, synthetic construct
  misc_feature 1..25: note = Description of Artificial Sequence: Synthetic primer
  source 1..25: mol_type = other DNA
ggactgagatctttcattttaccgt

SEQ ID NO: 18 - DNA, 21 bp, synthetic construct
  misc_feature 1..21: note = Description of Artificial Sequence: Synthetic primer
  source 1..21: mol_type = other DNA
actgagggagccttgaataca

SEQ ID NO: 19 - DNA, 30 bp, synthetic construct
  misc_feature 1..30: note = Description of Artificial Sequence: Synthetic probe
  source 1..30: mol_type = other DNA
agcggcaccttcgggaacgtggttgccgct

SEQ ID NO: 20 - DNA, 33 bp, synthetic construct
  misc_feature 1..33: note = Description of Artificial Sequence: Synthetic probe
  source 1..33: mol_type = other DNA
agcggctcagccgctcaccgtgagttagccgct

SEQ ID NO: 21 - DNA, 35 bp, synthetic construct
  misc_feature 1..35: note = Description of Artificial Sequence: Synthetic probe
  source 1..35: mol_type = other DNA
agcggctactgagggagccttgaatacaagccgct

SEQ ID NO: 22-23 - skipped (2 entries)

(19) United States
(12) Patent Application Publication
(10) Pub. No.: US 2023/0279512 A1
(43) Pub. Date: Sep. 7, 2023

(54) METHODS AND SYSTEMS FOR DROPLET MANIPULATION

(71) Applicant: Volta Labs, Inc., Cambridge, MA (US)

(72) Inventors: Liam MASTERS, Cambridge, MA (US); Udayan UMAPATHI, Somerville, MA (US); Spurti AKKI, Cambridge, MA (US); Magali SOUMILLON, Cambridge, MA (US); William LANGFORD, Cambridge, MA (US); Leonid KRASNOBAEV, Cambridge, MA (US)

(21) Appl. No.: 18/061,333

(22) Filed: Dec. 2, 2022

Related U.S. Application Data
(63) Continuation of application No. 17/680,173, filed on Feb. 24, 2022, now abandoned.
Application No. 17/680,173 is a continuation of application No. PCT/US20/48241, filed on Aug. 27, 2020.
(60) Provisional application No. 62/892,495, filed on Aug. 27, 2019.
(60) Provisional application No. 62/980,013, filed on Feb. 21, 2020.
(60) Provisional application No. 63/005,097, filed on Apr. 3, 2020.
(60) Provisional application No. 63/009,376, filed on Apr. 13, 2020.

(51) Int. Cl. C12Q 1/70; B01L 3/00; C12Q 1/6844; G01N 33/569

(52) U.S. Cl. CPC C12Q 1/701; B01L 3/502761; C12Q 1/6844; G01N 33/56983; B01L 2300/0645; B01L 2300/0636; B01L 2200/0652; B01L 2400/0427; G01N 2469/20

(57) ABSTRACT

Described herein are systems and methods for processing at least one biological sample. The systems and methods may process the biological sample, or plurality thereof, using at least one droplet. The droplet, or plurality thereof, may be manipulated using the systems and methods described herein.

### CROSS-REFERENCE

This application is a continuation of U.S. application Ser. No. 17/680,173, filed Feb. 24, 2022, which is a continuation of International Application No. PCT/US2020/048241, filed Aug. 27, 2020, which claims benefit of U.S. Provisional Application No. 63/009,376 filed Apr. 13, 2020, U.S. Provisional Application No. 63/005,097 filed Apr. 3, 2020, U.S. Provisional Application No. 62/980,013 filed Feb. 21, 2020, and U.S. Provisional Application No. 62/892,495 filed Aug. 27, 2019, which are herein incorporated by reference in their entirety.

### SEQUENCE LISTING

The instant application contains a Sequence Listing which has been submitted electronically in XML format and is hereby incorporated by reference in its entirety. Said XML copy, created on May 1, 2023 is named 56252-703.304 Sequence Listing.xml and is 28,715 bytes in size

### BACKGROUND

Biological samples may be processed for various applications. For example, a deoxyribonucleic acid (DNA) molecule or a ribonucleic acid (RNA) molecule may be processed (e.g., sequenced) to identify genetic variants, which may be useful to identify a disease, such as cancer. Such biological samples may be processed in partitions, such as droplets. Sequences of DNA or RNA may be determined by sequence identification, such as nucleic acid sequencing.

Droplets containing biological samples may be manipulated by using electrowetting, which may employ electric fields from electrodes to move a droplet adjacent to a surface.

### SUMMARY

In an aspect the present disclosure provides a method for processing a plurality of biological samples, comprising (i) receiving, adjacent to an array, a plurality of droplets comprising the plurality of biological samples, and (ii) using at least the array to process the plurality of biological samples in the plurality of droplets or derivatives thereof at a coefficient of variation (CV) of at least one parameter of the plurality of droplets or derivatives thereof, or the array, of less than 20% at cross-talk between the plurality of droplets at less than 5%, thereby processing the plurality of biological samples.

In another aspect, the present disclosure provides a method for customizing an array system for processing a plurality of biological samples, comprising (i) receiving a request for a configured array system from a user, which request comprises one or more specifications, and (ii) using the one or more specifications to configure the array system to yield the configured array system, which configured array system is configured to receive a plurality of droplets comprising the plurality of biological samples and process the plurality of droplets or derivatives thereof at a coefficient of variation (CV) of at least one parameter of the plurality of droplets or derivatives thereof, or the array, of less than 20% at cross-talk between the plurality of droplets at less than 5%.

In another aspect, the present disclosure provides a method for processing a biological sample, comprising providing, adjacent to an open array, a droplet comprising the biological sample, and using the open array to process the biological sample in the droplet or derivative thereof, wherein, during processing, a position of the droplet varies by at most 5% over a time period of at least 10 seconds.

In another aspect, the present disclosure provides a method for processing a biological sample, comprising (i) receiving, adjacent to an array, a droplet comprising the biological sample, and (ii) using at least the array to process the biological sample in the plurality of droplets or derivatives thereof at a coefficient of variation (CV) of at least one parameter of the droplet or derivative thereof, or the array, of less than 20% at cross-talk between the droplet at less than 5%.

In some embodiments, the at least one parameter comprises one or more members selected from the group consisting of droplet size, droplet volume, droplet position, droplet speed, droplet wetting, droplet temperature, droplet pH, beads in droplets, number of cells in droplets, droplet color, concentration of chemical material, concentration of biological substance, or any combination thereof. In some embodiments, the configuration of the array is selected from the group consisting of an open configuration with an electrode array, open configuration with no electrode array, open configuration with non-coplanar set of electrodes, two plates with an array of electrodes on one plate and no electrodes on the other plate, two plates with non-coplanar set of electrodes on one plate and no electrodes on the other plate, two plates with an array of electrodes on one plate and single electrode on the other plate, two plates with an non-coplanar set of electrodes on one plate and single electrode on other plate, two plates with electrodes arrays on both plates, two plates with non-coplanar set of electrodes on both plates, or any combination thereof.

In some embodiments, the plurality of biological samples is processed by combining a force field with an electric field. In some embodiments, the force field is generated by fluid flow, vibration, or a combination thereof, on the array. In some embodiments, the force field is selected from the group consisting of acoustic waves, vibrations, air pressure, light field, magnetic field, gravitational field, centrifugal force, hydrodynamic forces, electrophoretic forces, dielectrowetting force, and capillary forces. In some embodiments, the plurality of biological samples is processed with no more than four pipetting operations. In some embodiments, the plurality of biological samples is processed with no more than three pipetting operations. In some embodiments, the plurality of biological samples is processed with no more than two pipetting operations. In some embodiments, the plurality of biological samples is processed with no more than one pipetting operation. In some embodiments, the array comprises a plurality of sensors, and wherein the plurality of sensors measure signals from the plurality of droplets or derivatives thereof before, during, or subsequent to the processing the plurality of biological samples. In some embodiments, the plurality of sensors comprises an impedance sensor, a pH sensor, a temperature sensor, an optical sensor, a camera, a current measurement sensor, an electronic sensor for biomolecular detection, an x-ray sensor, biological materials as sensors, cells as sensors, tissues as sensors, chemical materials as sensors, electrochemical sensors, electrochemilumine scent sensors, piezoelectric sensors, nucleic acids as sensors, proteins as sensors, nanoparticle sensors, small molecule sensors, or any combination thereof.

In some embodiments, the plurality of sensors further comprises a feedback loop to regulate one or more parameters of the array while processing the plurality of biological samples. In some embodiments, the plurality of sensors and the feedback loop are used to discover, optimize, or a combination thereof, reaction conditions autonomously. In some embodiments, the at least one sensor of the plurality of sensors measures location, droplet volume, presence of biological material, activity of biological material, droplet velocity, kinematics, droplet radius, droplet shape, droplet height, color, surface area, contact angle, reaction state, emittance, absorbance, or any combination thereof. In some embodiments, the measurement of the at least one sensor of the plurality of sensors is used to further process at least one droplet, biological sample, or a combination thereof of the plurality of droplets, the plurality of biological samples, or a combination thereof.

In some embodiments, the further processing comprises giving a command to actuate inputs, outputs, or a combination thereof adjacent to or on, or a combination thereof, the array in real time. In some embodiments, the command provides instructions to correct an error of the array. In some embodiments, the error is an error in location, droplet volume, presence of biological material, activity of biological material, droplet velocity, droplet kinetics, droplet radius, droplet shape, droplet height, color, surface area, contact angle, reaction state, emittance, absorbance, or any combination thereof. In some embodiments, the array comprises a plurality of elements comprising: heaters, coolers, magnetic field generators, electroporation units, light sources, radiation sources, nucleic acids sequencers, biological protein channels, solid state nanopores, protein sequencers, acoustic transducers, microelectromechanical system (MEMS) transducers, capillary tubes as liquid dispensers holes for dispensing or transferring liquids using gravity, electrodes in a hole to dispense or transfer liquids using electric field, holes for optical inspection, holes for liquids to interact through membranes, or any combination thereof.

In some embodiments, the array interfaces with a liquid handling unit, which the liquid handling unit directs the plurality of droplets adjacent to the array. In some embodiments, the liquid handling unit is selected from the group consisting of robotic liquid handling systems, acoustic liquid dispensers, syringe pumps, inkjet nozzles, microfluidic devices, needles, diaphragm based pump dispensers, piezoelectric pumps, or any combination thereof. In some embodiments, the array is coupled to at least one reagent or sample storage unit, or a combination thereof. In some embodiments, the array further comprises at least one multi-well plate, tubes, bottles, reservoirs, inkjet cartridges, plates, petri dishes, or any combination thereof. In some embodiments, the tubes are selected from Eppendorf tubes or falcon tubes. In some embodiments, the plurality of wells of the at least one multi-well plate is thermally conductive, electronically conductive, or a combination thereof. In some embodiments, a reagent or sample of the at least one reagent or sample storage unit, or a combination thereof, is manipulated in or out of the well by an electric field, a magnetic field, an acoustic wave, heat, vibration, or a combination thereof.

In some embodiments, the array comprises a coating. In some embodiments, the coating is a hydrophobic coating. In some embodiments, the coating is a hydrophilic coating. In some embodiments, the coating comprises both hydrophobic and hydrophilic coatings. In some embodiments, the coating is cleaned by washing. In some embodiments, the coating reduces evaporation. In some embodiments, the evaporation is reduced by 50% to 100%. In some embodiments, the coating reduces biofouling. In some embodiments, the biofouling is reduced by 10% to 100%. In some embodiments, the coating is resistant to biofouling. In some embodiments, the coating is antibiofouling. In some embodiments, the CV is less than 15%. In some embodiments, the CV is less than 10%. In some embodiments, the CV is less than 5%. In some embodiments, the CV is less than 1%.

In some embodiments, the processing the plurality of biological samples comprises a nucleic acid, protein, cell, salt, buffer, or enzyme, wherein the droplet comprises one or more reagents for nucleic acid isolation, cell isolation, protein isolation, nucleic acid purification, peptide purification, isolation or purification of a biopolymer, immunoprecipitation, in vitro diagnostics, exosome isolation, cell activation, cell expansion, nucleic acid synthesis, protein synthesis, peptide synthesis, enzyme synthesis, chemical synthesis, cell culture, cell lysis, creation of synthetic cell, nucleic acid amplification, nucleic acid manipulation, cell manipulation, nucleic acid detection, protein detection, gene editing or isolation of a specific biomolecule, and wherein the droplet is manipulated by the reagents to perform the nucleic acid isolation, cell isolation, protein isolation, nucleic acid purification, peptide purification, isolation or purification of a biopolymer, immunoprecipitation, in vitro diagnostics, exosome isolation, cell activation, cell expansion, nucleic acid synthesis, protein synthesis, peptide synthesis, enzyme synthesis, chemical synthesis, cell culture, cell lysis, creation of synthetic cell, nucleic acid amplification, nucleic acid manipulation, cell manipulation, nucleic acid detection, protein detection, gene editing or isolation of a specific biomolecule.

In some embodiments, the processing the plurality of biological samples comprises nucleic acid sequencing. In some embodiments, the nucleic acid sequencing comprises polymerase chain reaction (PCR). In some embodiments, the PCR comprises highly multiplexed PCR, quantitative PCR, droplet digital PCR, reverse transcriptase PCR, or any combination thereof. In some embodiments, the processing the plurality of biological samples comprises sample preparation for genomic sequencing. In some embodiments, the processing the plurality of biological samples comprises combinatorial assembly of genes. In some embodiments, the combinatorial assembly of genes comprises Gibson Assembly, restriction enzyme cloning, gBlocks fragments assembly (IDT), BioBricks assembly, NEBuilder HiFi DNA assembly, Golden Gate assembly, site-directed mutagenesis, sequence and ligase independent cloning (SLIC), circular polymerase extension cloning (CPEC), and seamless ligation cloning extract (SLiCE), topoisomerase mediated ligation, homologous recombination, Gateway cloning, GeneArt gene synthesis, or any combination thereof.

In some embodiments, the processing the plurality of biological samples comprises extracting ribosomes, mitochondria, endoplasmic reticulum, golgi apparatus, lysosomes, peroxisomes, centrioles, or any combination thereof. In some embodiments, the ribosomes, mitochondria, endoplasmic reticulum, golgi apparatus, lysosomes, peroxisomes, centrioles, or any combination thereof remain intact. In some embodiments, the processing the plurality of biological samples comprises cell-free protein expression. In some embodiments, the processing the plurality of biological samples comprises preparation for plasmid DNA extraction. In some embodiments, the processing the plurality of biological samples comprises extraction of nucleic acids from cells. In some embodiments, the processing further comprises extracting long strands of nucleic acid, wherein the long strands of nucleic acid remain intact.

In some embodiments, the long strands of nucleic acid are at least 10 base pairs. In some embodiments, the long strands of nucleic acid are at least 100 base pairs. In some embodiments, the long strands of nucleic acid are at least 1000 base pairs. In some embodiments, the long strands of nucleic acid are at least 10,000 base pairs. In some embodiments, the long strands of nucleic acid are at least 100,000 base pairs. In some embodiments, the long strands of nucleic acid are at least 1,000,000 base pairs. In some embodiments, the processing the plurality of biological samples comprises sample preparation for mass spectrometry. In some embodiments, the processing the plurality of biological samples comprises sample extraction and library preparation for nucleic acid sequencing. In some embodiments, the nucleic acid sequencing is selected from the group consisting of sequencing by synthesis, pyrosequencing, sequencing by hybridization, sequencing by ligation, sequencing by detection of ions released during polymerization of DNA, Sanger sequencing, and single-molecule sequencing. In some embodiments, the single molecule sequencing is nanopore sequencing. In some embodiments, the single molecule sequencing is single molecule real time (SMRT) sequencing.

In some embodiments, the processing the plurality of biological samples comprises DNA synthesis using oligonucleotide synthesis, enzymatic synthesis, or any combination thereof. In some embodiments, the processing the plurality of biological samples comprises DNA data storage, random-access of stored DNA, DNA data retrieval through DNA sequencing, or any combination thereof. In some embodiments, the processing the plurality of biological samples comprises nucleic acid extraction and sample preparation integrated directly into a sequencer. In some embodiments, the processing the plurality of biological samples comprises protein extraction and sample preparation directly integrated to a mass spectrometer. In some embodiments, the mass spectrometer further comprises a matrix-assisted laser desorption ionization mass spectrometer. In some embodiments, the array ionizes, transfers, or a combination thereof a chemical or biological sample directly into an inlet of a mass spectrometer. In some embodiments, the ionization is electrospray. In some embodiments, the transfer is pipetting.

In some embodiments, the processing the plurality of biological samples comprises CRISPR genome editing. In some embodiments, the CRISPR genome editing comprises Cas9 protein, crRNA, tracrRNA, or any combination thereof. In some embodiments, a repair DNA template is used during the CRISPR genome editing process. In some embodiments, the processing the plurality of biological samples comprises transcription activator-like effector nucleases (TALENs) genome editing. In some embodiments, the processing the plurality of biological samples comprises Zinc Fingers Nuclease gene editing.

In some embodiments, the processing the plurality of biological samples comprises at least one high-throughput process. In some embodiments, the processing the plurality of biological samples comprises screening of a plurality of chemical compounds against a plurality of cells. In some embodiments, the chemical compounds are antibacterial. In some embodiments, the cells are prokaryotic cells. In some embodiments, the prokaryotic cells are bacterial cells. In some embodiments, the cells are eukaryotic cells. In some embodiments, the eukaryotic cells are animal cells. In some embodiments, the eukaryotic cells are mammalian cells. In some embodiments, the eukaryotic cells are plant cells. In some embodiments, the eukaryotic cells are fungi cells. In some embodiments, the chemical compounds are screened for biological activity. In some embodiments, the screening further comprises determining biological activity using sensors of the array. In some embodiments, the screening further comprises isolating of at least one chemical compound. In some embodiments, the processing the plurality of biological samples comprises culturing cells, thereby producing cultured cells. In some embodiments, the cultured cells are in at least one discrete droplet. In some embodiments, the cultured cells are in at least one discrete physical compartment. In some embodiments, interactions between the cultured cells or between cultured cells and at least one biological sample are determined.

In some embodiments, the cultured cells are assayed on the array, or the plurality thereof. In some embodiments, the cultured cells are isolated from the culture, thereby producing isolated cells. In some embodiments, the isolated cells are transferred to an external container. In some embodiments, the external container is a Society for Biomolecular Screening (SBS) format plate. In some embodiments, the isolated cells are prepared for nucleic acid sequencing. In some embodiments, the isolated cells are prepared for protein analysis. In some embodiments, the isolated cells are prepared for metabolomic analysis. In some embodiments, the array comprises a plurality of lyophilized reagents, dry reagents, stored beads, or any combination thereof. In some embodiments, the plurality of lyophilized reagents, dry reagents, stored beads, or any combination thereof are reconstituted. In some embodiments, at least one droplet, or derivative thereof, is used to reconstitute the lyophilized reagents, dry reagents, beads, or any combination thereof. In some embodiments, the lyophilized reagents comprise molecular barcodes. In some embodiments, the lyophilized reagents comprise oligonucleotides. In some embodiments, the lyophilized reagents comprise primers. In some embodiments, the lyophilized reagents comprise DNA sequences for hybridization. In some embodiments, the lyophilized reagents comprise enzymes. In some embodiments, the beads comprise molecular barcodes. In some embodiments, the beads comprise oligonucleotides, nucleic acids, antibodies, PCR primers, ligands or any combinations thereof.

In some embodiments, the method further comprises at least one reagent, wherein the at least one reagent is prefabricated into a component of the array. In some embodiments, the array stores a plurality of reagents as a solid, liquid, gas, or any combination thereof. In some embodiments, the array condenses, sublimes, thaws, evaporates, or any combination thereof, the plurality of reagents. In some embodiments, the array dispenses a plurality of liquids. In some embodiments, the array mixes a plurality of liquids. In some embodiments, the processing of the plurality of biological samples is automated. In some embodiments, the array is reusable, thereby producing a reusable array. In some embodiments, the array further comprises a replaceable surface. In some embodiments, the array further comprises a replaceable film. In some embodiments, the array comprises a replaceable cartridge. In some embodiments, the replaceable cartridge is a film. In some embodiments, a vacuum is used to attach the film to the array. In some embodiments, the replaceable cartridge may be coupled to the array using an adhesive. In some embodiments, the adhesive is selected from the group consisting of silicone, acrylic, epoxy, pressure sensitive adhesives, thermal glue, or any combination thereof. In some embodiments, the reusable array is washed, thereby producing a washed array. In some embodiments, the washed array is washed entirely. In some embodiments, the washed array is washed partially.

In some embodiments, the array is disposable. In some embodiments, a volume of biomolecules of the array is manipulated as a mixture. In some embodiments, the volume of biomolecules comprises a plurality of nucleic acids, protein sequences, or a combination thereof. In some embodiments, the plurality of nucleic acid, protein sequences, or a combination thereof are manipulated by modulation of local surface charge without physical contact on the mixture by another component of the array. In some embodiments, the mixture is within a droplet. In some embodiments, the droplet comprises a volume from 1 pl to 10 ml. In some embodiments, the mixture comprises a protein with DNA ligase activity. In some embodiments, the mixture comprises a protein with DNA transposase activity. In some embodiments, the volume of biomolecules of the assay is manipulated with lateral geospatial movement of the mixture of at least 1 mm. In some embodiments, the array comprises reagents for conducting a Strand Displacement Amplification reaction, a self-sustained sequence replication and amplification reaction, a Q3 replicase amplification reaction, or any combination thereof.

In some embodiments, the array comprises reagents, including a DNA ligase, a nuclease, a restriction endonuclease, or any combination thereof. In some embodiments, the array comprises reagents for the preparation of an amplified nucleic acid product. In some embodiments, the plurality of biological samples is derived from an animal In some embodiments, the animal has or is suspected of having a disease. In some embodiments, the animal is a mammalian subject. In some embodiments, the plurality of biological samples is derived from a plant. In some embodiments, the plurality of biological samples is derived from a prokaryote. In some embodiments, the array is a component in the manufacture of a kit or system for the diagnosis or prognosis of a disease. In some embodiments, the array includes a protein with nucleic acid cleavage activity. In some embodiments, the array includes a biomolecule with RNA cleavage activity. In some embodiments, an interchangeable set of reagents is introduced by at least one solid phase support. In some embodiments, the solid phase support is a paper strip. In some embodiments, the solid phase support is a microbead. In some embodiments, the solid phase support is a pillar. In some embodiments, the solid phase support is a strip of microwells. In some embodiments, an interchangeable set of reagents is introduced by at least one secondary support. In some embodiments, the secondary support is a strip of microwells. In some embodiments, the secondary support is a bead.

In some embodiments, the array contains a template independent polymerase. In some embodiments, the template independent polymerase is a terminal deoxynucleotidyl transferase (TdT). In some embodiments, the array includes an enzyme that limits nucleic acid polymerization. In some embodiments, the enzyme that limits nucleic acid polymerization is an apyrase. In some embodiments, the array has sensors to detect a presence of at least one terminal ‘C’ tail in a nucleic acid molecule. In some embodiments, the at least one terminal ‘C’ tail is isolated. In some embodiments, the plurality of biological samples of the array is stored by drying. In some embodiments, the plurality of biological samples of the array is retrieved by rehydration. In some embodiments, the plurality of biological samples is deposited onto the plurality of arrays in Society for Biomolecular Screening (SBS) format or on any random location of the plurality of arrays, thereby producing at least one deposited biological sample. In some embodiments, the plurality of biological samples is deposited using commercial acoustic liquid handlers in preparation for manipulating samples on the chip. In some embodiments, the acoustic liquid handlers are Echo. In some embodiments, the at least one deposited biological sample is used for cell free synthesis. In some embodiments, the at least one deposited biological sample is used for combinatorially assembling large DNA constructs.

In some embodiments, the processing the plurality of biological samples comprises at least one of the following assays, or any combination thereof: digital PCR, isothermal amplification of nucleic acids, antibody mediated detection, enzyme linked immunoassay (ELISA), oxidation- or reduction-based electrochemical detection, colorimetric assay, fluorometric assay, and micronucleus assay. In some embodiments, the processing the plurality of biological samples comprises isothermal amplification of at least one selected nucleic acid, comprising: (a) providing at least one sample that comprises at least one nucleic acid by merging droplets containing a plurality of reagents effective to permit at least one isothermal amplification reaction of the sample without mechanical manipulation and (b) conducting at least one isothermal amplification reaction to amplify the nucleic acid. In some embodiments, the processing the plurality of biological samples comprises a device to detect a polymerase chain reaction (PCR) product on at least one aqueous droplet, wherein the device: (a) creates at least one droplet containing a plurality of nucleic acid and protein molecules on an electrowetting array, (b) performs the PCR reaction while the aqueous droplets are present on the array, and (c) interrogates the droplet with a detector.

In some embodiments, the device to detect a PCR product further comprises a plurality of fluorescent reporter molecules. In some embodiments, the plurality of fluorescent reporter molecules is separated by at least one enzyme from at least one quencher molecule during the PCR reaction. In some embodiments, the at least one enzyme comprises a polymerase. In some embodiments, the nucleic acid is detected by a sensor. In some embodiments, the sensor detects a radiolabel. In some embodiments, the sensor detects a fluorescent label. In some embodiments the sensor detects a chromophore. In some embodiments the sensor detects a redox label. In some embodiments, the sensor is a p-n-type diffusion diode. In some embodiments, the nucleic acid is detected by a smartphone. In some embodiments, the processing the plurality of biological samples includes binding at least one biomolecule on the array. In some embodiments, the at least one biomolecule is immobilized on a surface. In some embodiments, the at least one biomolecule is immobilized on a diffusible matrix. In some embodiments, the at least one biomolecule is immobilized on a diffusible bead. In some embodiments, a location of the biomolecule is identified by a coding scheme.

In some embodiments, the coding scheme is based on a moiety to which it is immobilized. In some embodiments, the array induces an interaction of a plurality of biomolecules from two or more non-continuous liquid volumes without mechanical manipulations. In some embodiments, the array prepares an amplified nucleic acid product. In some embodiments, the array conducts a diagnostic test on a nucleic acid sample. In some embodiments, the array conducts a diagnostic or prognostic test on a biological sample. In some embodiments, the plurality of biological samples is suspected of containing a nucleic acid biomarker. In some embodiments, the array comprises a gas source that contacts and is absorbed by at least one droplet. In some embodiments, the at least one droplet is manipulated on the device. In some embodiments, the plurality of biological samples includes reagents for conducting a Strand Displacement Amplification reaction, a self-sustained sequence replication, an amplification reaction, a Q3 replicase amplification reaction, or any combination thereof. In some embodiments, the array receives at least one instruction from a remote computer to process the array of biological samples. In some embodiments, the array is preprogrammed to perform the process on the array of biological samples.

In some embodiments, the array receives information related to a DNA sequence. In some embodiments, the DNA sequence triggers an automated process. In some embodiments, the automated process includes conversion of the DNA sequence into at least one constituent oligonucleotide sequence. In some embodiments, the at least one constituent oligonucleotide sequence is assembled, error corrected, reassembled into DNA amplicons, or any combination thereof. In some embodiments, the DNA amplicons direct production of RNA, proteins, biological particles, or any combination thereof. In some embodiments, the biological particles are derived from a virus. In some embodiments, the array produces at least one peptide or antibody from a DNA template. In some embodiments, the array partitions at least one droplet into a plurality of droplets comprising using electrowetting force, dielectrowetting (DEW) force, dielectrophoretic (DEP) effect, acoustic force, hydrophobic knife, or any combination thereof, thereby producing at least one partitioned droplet. In some embodiments, the partitioning dispenses reagents. In some embodiments, the partitioning dispenses samples. In some embodiments, the at least one partitioned droplet is mixed to execute a reaction. In some embodiments, the at least one partitioned droplet is analyzed using the sensors. In some embodiments, the at least one partitioned droplet is mixed with at least one target droplet to maintain a constant volume on the at least one target droplet.

In some embodiments, the array processes a multiphase fluid. In some embodiments, the array uses dielectrophoretic forces (DEP) for cell sorting, cell separation, manipulating at least one bead, or any combination thereof. In some embodiments, the sorting or separation is used for pre-concentrating at least one cell in raw clinical samples. In some embodiments, the biological samples are deposited on a plurality of arrays. In some embodiments, the plurality of arrays comprises at least two arrays. In some embodiments, an array of the at least two arrays is adjacent to another array of the at least two arrays. In some embodiments, the array of the at least two arrays is horizontally adjacent to another array of the at least two arrays. In some embodiments, the array of the at least two arrays is vertically adjacent to another array of the at least two arrays. In some embodiments, the array of the at least two arrays comprises a surface. In some embodiments, the surface comprises at least one EWOD array, at least one DEW array, at least one DEP array, at least one microfluidic array, glass, plastic, or any combination thereof.

In some embodiments, the plurality of arrays comprises at least one channel, at least one hole, or any combination thereof. In some embodiments, the at least one channel traverses between at least one surface. In some embodiments, gas, liquid, solid, or any combination thereof is transferred by the at least one hole. In some embodiments, the gas, liquid, solid, or any combination thereof is transferred in or out of the plurality of arrays. In some embodiments, the gas, liquid, solid, or any combination thereof is transferred between at least two surfaces of the plurality of arrays. In some embodiments, at least two droplets of the plurality of droplets are separated by at least one permeable membrane. In some embodiments, at least a portion of the components of the at least two droplets of the plurality of droplets are exchanged from one droplet of the at least two droplets of the plurality of droplets to another droplet of the at least two droplets of the plurality of droplets by the at least one permeable membrane. In some embodiments, the at least one permeable membrane is permanently or temporarily attached to the array.

In another aspect, the present disclosure provides a system for processing a plurality of biological samples, comprising (i) receiving, adjacent to an array, a plurality of droplets comprising the plurality of biological samples, and (ii) using at least the array to process the plurality of biological samples in the plurality of droplets or derivatives thereof at a coefficient of variation (CV) of at least one parameter of the plurality of droplets or derivatives thereof, or the array, of less than 20% at cross-talk between the plurality of droplets at less than 5%, thereby processing the plurality of biological samples.

In another aspect, the present disclosure provides a system for biological sample processing, comprising: a housing configured to contain a plurality of arrays, wherein an array of the plurality of arrays is configured to (i) receive, adjacent to the array, a plurality of droplets comprising the plurality of biological samples, and (ii) use at least the array to process the plurality of biological samples in the plurality of droplets or derivatives thereof at a coefficient of variation (CV) of at least one parameter of the plurality of droplets or derivatives thereof, or the array, of less than 20% at cross-talk between the plurality of droplets at less than 5%.

In some embodiments, the plurality of arrays is removable from the housing. In some embodiments, the housing is configured to couple to a nucleic acid sequencing platform. In some embodiments, the housing is a nucleic acid sequencing platform. In some embodiments, an environment of the array is controlled by the housing, thereby producing a controlled environment. In some embodiments, ambient humidity, droplet coating, temperature, pressure, droplet size, lighting condition, or any combination thereof are maintained by the controlled environment. In some embodiments, the housing comprises an enclosure. In some embodiments, the enclosure comprises a cover, a seal, a chamber, an immiscible high vapor-pressure fluid, a film, or any combination thereof.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

In another aspect, the present disclosure provides a method for processing a droplet, comprising: providing said droplet on an array, wherein said droplet comprises one or more detectable labels, wherein a detectable label of said one or more detectable labels corresponds to a physical property of said droplet; using one or more light sources to illuminate said droplet on said array, wherein upon illumination by said one or more light sources, said detectable label generates a signal; using a detector to detect said signal; using said signal detected in to determine said physical property of said droplet; and manipulating said droplet if said physical property determined does not meet a threshold.

In some embodiments, said physical property is selected from the group consisting of droplet size, droplet volume, droplet position, droplet speed, droplet wetting, droplet temperature, droplet pH, beads in droplets, number of cells in droplets, droplet color, concentration of chemical material, concentration of biological substance, or any combination thereof. In some embodiments, said droplet comprises a plurality of detectable labels corresponding to different physical properties of said droplet, wherein said plurality of detectable labels comprises said detectable label.

In some embodiments, said detector comprises at least one camera. In some embodiments, manipulating said droplet comprises computer processing said physical property and a threshold value or value range. In some embodiment, said signal is detected from said droplet at multiple time points on said array. In some embodiments, wherein said detector comprises one or more optical filters, and wherein said one or more optical filters is used to detect said signal. In some embodiments, the method further comprises altering at least a subset of said one or more optical filters to detect an additional signal from said droplet.

In some embodiments, said parameter is droplet volume, and wherein said volume is determined to be below a threshold volume, and wherein said droplet is contacted with one or more replenishing droplets. In some embodiments, said replenishing droplets are contacted with said droplet through electrowetting motion. In some embodiments, said replenishing droplets replenish about 1% to about 50% of a volume of said droplet.

In some embodiments, said parameter is employed in generating a machine learning model for determining said parameter for one or more additional droplets to be introduced to said array.

In some embodiments, the method further comprises a step of heating one or more fluids surrounding said droplet to reduce evaporation of said droplet. In some embodiments, said heating is performed by the actuation of a heater displaced below said array, the heating of a plate displaced above said array, the heating of one or more sidewalls contacting said array, or a combination thereof. In some embodiments, said one or more fluids comprises water, and the method further comprises contacting an area displaced on said array with said one or more fluids.

In some embodiments, a relative humidity of about 50% to about 100% is maintained in said area. In some embodiments, contacting said area with said one or more fluids comprises introducing one or more sacrificial droplets to said prior to or subsequent to introducing a droplet comprising a sample for analysis. In some embodiments, said contacting said area with said one or more fluids comprises displacing a water reservoir within said area. In some embodiments, the method further comprises encapsulating said area in a chamber. In some embodiments, the method further comprises uniformly heating said chamber.

In some embodiments, said plate displaced above said array comprises electrodes. In some embodiments, an electrode of said electrodes is individually encapsulated. In some embodiments, said electrodes are transparent. In some embodiments, said plate displaced above said array is transparent. In some embodiments, said sidewalls comprise a conductor, a circuit board, or both. In some embodiments, said circuit board comprises serpentine traces. In some embodiments, said array, said plate displaced above said array, said sidewalls, or a combination thereof further comprise a resistive film heater, thermal insulation, a temperature sensor, or any combination thereof. In some embodiments, said temperature senor is coupled to a side of said array, wherein said side is opposite of a side comprising said droplet. In some embodiments, said heater displaced below said array comprises an electrode. In some embodiments, said electrode is individually encapsulated. In some embodiments, said volume of said droplet is maintained within at least about 30%, 20%, 10%, 5%, 1%, 0.1%, or 0.01% of an original droplet volume.

Another aspect of the present disclosure provides a method of synthesizing a polynucleotide on an array, comprising providing a droplet on said array, wherein said droplet comprises a nucleic acid molecule, and using said nucleic acid molecule to synthesize said polynucleotide, wherein said has a volume from about 1 picoliter to about 2 microliters, and wherein during said synthesis, a volume of said droplet changes by at most 50%.

In some embodiments, during said synthesis, said volume changes by at most 10%. In some embodiments, during said synthesis, said volume changes by at most 1%. In some embodiments, said polynucleotide is synthesized at least in part by ligating an additional nucleic acid molecule to said nucleic acid molecule. In some embodiments, said polynucleotide is synthesized at least in part by hybridizing an additional nucleic acid molecule to said nucleic acid molecule. In some embodiments, said additional nucleic acid molecule is comprised in an additional droplet. In some embodiments, the method further comprises contacting said droplet with said additional droplet. In some embodiments, said biomolecule is synthesized at in least in part by addition of a single nucleotide to a 3′-overhang or 3′ blunt end or 3′ recessed end of a nucleic acid molecule.

Another aspect of the present disclosure provides system for manipulating a droplet, comprising: an array configured to support said droplet; a plurality of magnets; a shield disposed between said array and said plurality of magnets, wherein said shield comprises one or more cutouts and wherein said one or more cutouts are aligned to said plurality of magnets; and a controller coupled to said plurality of magnets, wherein said controller is configured to direct actuation of a magnet of said plurality of magnets to manipulate said droplet on said array.

In some embodiments, the system further comprises a stage supporting said plurality of magnets wherein said coupled to an actuator, wherein said actuator is configured to move said stage upon a linear axis. In some embodiments, said plurality of magnets comprises ferromagnet flux focusers, ferromagnetic back iron, or a combination thereof. In some embodiments, said shield comprises ferromagnetic material.

In some embodiments, said plurality of magnets comprises one or more rotary switchable magnets. In some embodiments, said one or more rotary switchable magnets is configured to rotate a magnet of said plurality of magnets.

Another aspect of the present disclosure provides a method for manipulating a droplet with a magnetic field, comprising: disposing said droplet on an array, wherein said array comprises a shield, wherein said shield comprises one or more cutouts; displacing said array within a proximity of a plurality of magnets, wherein a magnet of said plurality of magnets contacts said one or more cutouts; actuating at least one magnet of said plurality of magnets to manipulate said droplet on said array with said magnetic field.

In some embodiments, displacing said array comprises actuating a stage configured to support said plurality of magnets. In some embodiments, said plurality of magnets comprises ferromagnet flux focusers, ferromagnetic back iron, or a combination thereof. In some embodiments, said shield comprises ferromagnetic material. In some embodiments, said plurality of magnets comprises one or more rotary switchable magnets. In some embodiments, said one or more rotary switchable magnets is configured to rotate a magnet of said plurality of magnets.

Another aspect of the present invention provides a system for processing one or more droplets, comprising: a holder configured to support a cartridge, said cartridge comprising an array that is configured to process one or more droplets, wherein said array does not comprise an overlying electrowetting electrode; and a computer processor configured to direct processing of said one or more droplets when said cartridge is supported.

In some embodiments, the system further comprises a plurality of electrodes. In some embodiments, said plurality of electrodes is in electrical communication with said cartridge. In some embodiments, said cartridge further comprises a dielectric. In some embodiments, said dielectric is adjacent to said array. In some embodiments, said cartridge further comprises a plurality of electrodes. In some embodiments, said plurality of electrodes is adjacent to said array. In some embodiments, said cartridge further comprises an additional plurality of electrodes. In some embodiments, said plurality of electrodes and said additional plurality of electrodes are non-coplanar.

In some embodiments, said array comprises a polymeric film. In some embodiments, said array comprises a liquid layer. In some embodiments, said liquid layer forms a liquid to liquid interface with said one or more droplets. In some embodiments, said cartridge comprises a frame configured to maintain or generate a tension of a surface of said array. In some embodiments, said frame generates vacuum pressure on said surface of said array. In some embodiments, said frame comprises a fluid dispensing unit. In some embodiments, said frame is configured to replenish said liquid layer. In some embodiments, said cartridge further comprises one or more additional arrays. In some embodiments, said cartridge is removable from said holder.

In some embodiments, said array is in communication with said device by fine-pitched elastomeric connectors, board-to-board connectors, pogo-pins, or any combination thereof. In some embodiments, said device further comprises a module configured to house said array. In some embodiments, said module comprises one or more electrical connectors, wherein said electrical connectors are in communication with said processor and said plurality of electrodes coupled to said processer.

In some embodiments, said module comprises a cover, wherein said cover is configured to contact said array with said electrical connectors. In some embodiments, said cover is transparent.

In some embodiments, said electrical connectors comprise fine-pitched elastomeric connectors, board-to-board connectors, pogo-pins, spring connectors, conductive paste, or any combination thereof. In some embodiments, said module is configured to house one or more additional arrays.

In some embodiments, the system further comprises a projector configured to emit a light onto one or more tiles of said array. In some embodiments, said light comprises positional information specific to a location on said array. In some embodiments, the system further comprises one or more scanning mirrors or galvanometers configured to direct said light onto said array.

In one aspect, the present disclosure provides device for processing a sample, comprising: an array comprising a surface configured to support said droplet, wherein said array comprises an electrode that is configured to subject droplet to motion adjacent said surface; and a manipulation feature disposed on or adjacent to said surface, wherein said manipulation feature is configured to split said droplet.

In some embodiments, said manipulation feature comprises a microstructure disposed on said surface. In some embodiments, said microstructure comprises a hydrophobic material. In some embodiments, said manipulation feature comprises a hydrophilic region displaced on said surface. In some embodiments, said hydrophilic region is configured to bind to a hydrophobic article comprised in said droplet. In some embodiments, said manipulation feature is conformed specific to a volume of said fraction droplet. In some embodiments, said manipulation feature is non-coplanar with said surface. In some embodiments, said droplet comprises a volume between 1 femtometer and 1 milliliter.

Another aspect of the present disclosure provides a method of synthesizing a polynucleotide on an array, comprising: providing a droplet on said array, wherein said droplet comprises a nucleic acid molecule; and using said nucleic acid molecule to synthesize said polynucleotide, wherein during said synthesis, said droplet or a derivative thereof has a volume from about 1 femtoliter to about 2 microliters; and a volume of said droplet or said derivative thereof changes by at most 50%.

In some embodiments, during said synthesis, said volume changes by at most 10%. In some embodiments, during said synthesis, said volume changes by at most 1%. In some embodiments, said polynucleotide is synthesized at least in part by ligating an additional nucleic acid molecule to said nucleic acid molecule. In some embodiments, said polynucleotide is synthesized at least in part by hybridizing an additional nucleic acid molecule to said nucleic acid molecule. In some embodiments, said additional nucleic acid molecule is comprised in an additional droplet or a derivative thereof.

In some embodiments, the method further comprises detecting said volume of said droplet or said derivative thereof with a detector. In some embodiments, said detector comprises at least one camera. In some embodiments, said volume is detected from said droplet or said derivative thereof at multiple time points on said array. In some embodiments, the method further comprises manipulating said droplet or derivative thereof if said volume does not meet a threshold. In some embodiments, said threshold comprises a value range. In some embodiments, said manipulating comprises contacting said droplet or derivative thereof with a replenishing droplet. In some embodiments, said replenishing droplet does not comprise a biological sample.

Another aspect of the present disclosure provides a method for processing a plurality of biological samples, comprising receiving, adjacent to an array, a plurality of droplets comprising said plurality of biological samples, and using at least said array to process said plurality of biological samples in said plurality of droplets or derivatives thereof at a coefficient of variation (CV) of at least one parameter of said plurality of droplets or derivatives thereof, or said array, of less than 20% at cross-talk between said plurality of droplets at less than 5%, thereby processing said plurality of biological samples.

In some embodiments, said at least one parameter comprises one or more members selected from the group consisting of droplet size, droplet volume, droplet position, droplet speed, droplet wetting, droplet temperature, droplet pH, beads in droplets, number of cells in droplets, droplet color, concentration of chemical material, concentration of biological substance, or any combination thereof.

In some embodiments, said plurality of biological samples is processed by combining a force field with an electric field. In some embodiments, said force field is generated by fluid flow, vibration, or a combination thereof, on said array. In some embodiments, said force field is selected from the group consisting of acoustic waves, vibrations, air pressure, light field, magnetic field, gravitational field, centrifugal force, hydrodynamic forces, electrophoretic forces, dielectrowetting force, and capillary forces.

In some embodiments, said array comprises a plurality of sensors, and wherein said plurality of sensors measure signals from said plurality of droplets or derivatives thereof before, during, or subsequent to said processing said plurality of biological samples. In some embodiments, said plurality of sensors comprises an impedance sensor, a pH sensor, a temperature sensor, an optical sensor, a camera, a current measurement sensor, an electronic sensor for biomolecular detection, an x-ray sensor, electrochemical sensors, electrochemiluminescent sensors, piezoelectric sensors, or any combination thereof. In some embodiments, the method further comprises using said plurality of sensors in a feedback loop to regulate one or more parameters of said array while processing said plurality of biological samples. In some embodiments, the method further comprises using said plurality of sensors and said feedback loop to discover, optimize, or a combination thereof reaction conditions autonomously. In some embodiments, at least one sensor of said plurality of sensors measures location, droplet volume, presence of biological material, activity of biological material, droplet velocity, kinematics, droplet radius, droplet shape, droplet height, color, surface area, contact angle, reaction state, emittance, absorbance, or any combination thereof. In some embodiments, a measurement of said at least one sensor of said plurality of sensors is used to further process at least one droplet, biological sample, or a combination thereof of said plurality of droplets, said plurality of biological samples, or a combination thereof.

In some embodiments, further processing comprises giving a command to actuate inputs, outputs, or a combination thereof adjacent to or on, or a combination thereof, said array in real time. In some embodiments, said command provides instructions to correct an error of said array. In some embodiments, said error is an error in location, droplet volume, presence of biological material, activity of biological material, droplet velocity, droplet kinetics, droplet radius, droplet shape, droplet height, color, surface area, contact angle, reaction state, emittance, absorbance, or any combination thereof. In some embodiments, said array interfaces with a liquid handling unit, which said liquid handling unit directs said plurality of droplets adjacent to said array. In some embodiments, said liquid handling unit is selected from the group consisting of robotic liquid handling systems, acoustic liquid dispensers, syringe pumps, inkjet nozzles, microfluidic devices, needles, microdiaphragm based pump dispensers, piezoelectric pumps, or any combination thereof. In some embodiments, said array is coupled to at least one reagent or sample storage unit, or a combination thereof. In some embodiments, said liquid handling unit further comprises at least one multi-well plate, tubes, bottles, reservoirs, inkjet cartridges, plates, petri dishes, or any combination thereof. In some embodiments, said tubes are selected from Eppendorf tubes or falcon tubes. In some embodiments, said plurality of wells of said at least one multi-well plate is thermally conductive, electronically conductive, or a combination thereof.

In some embodiments, a reagent or sample of said at least one reagent or sample storage unit, or a combination thereof, is manipulated in or out of said well by an electric field, a magnetic field, an acoustic wave, heat, vibration, or a combination thereof. In some embodiments, said array comprises a coating. In some embodiments, said coating is a hydrophilic coating. In some embodiments, said coating comprises both hydrophobic and hydrophilic coatings. In some embodiments, said coating reduces evaporation. In some embodiments, said evaporation is reduced by 50% to 100%. In some embodiments, said coating reduces biofouling.

In some embodiments, said processing said plurality of biological samples comprises screening of a plurality of chemical compounds against a plurality of cells. In some embodiments, said processing said plurality of biological samples comprises culturing cells, thereby producing cultured cells. In some embodiments, said array further comprises at least one reagent, wherein said at least one reagent is prefabricated into a component of said array.

In some embodiments, wherein said array is reusable, thereby producing a reusable array. In some embodiments, said array further comprises a replaceable surface. In some embodiments, said array further comprises a replaceable cartridge. In some embodiments, said replaceable cartridge is a film. In some embodiments, a vacuum is used to attach said film to said array.

In some embodiments, said replaceable cartridge is coupled to said array using an adhesive. In some embodiments, said adhesive is selected from the group consisting of silicone, acrylic, epoxy, pressure sensitive adhesives, thermal glue, or any combination thereof. In some embodiments, said reusable array is washable.

In some embodiments, a volume of biomolecules of said array is manipulated as a mixture within a droplet, wherein said volume of biomolecules of said assay is manipulated with lateral geospatial movement of said mixture of at least 1 mm. In some embodiments, an interchangeable set of reagents is introduced by at least one solid phase support. In some embodiments, said solid phase support is a paper strip. In some embodiments, said solid phase support is a microbead. In some embodiments, said solid phase support is a pillar structure. In some embodiments, said solid phase support is a strip of microwells. In some embodiments, an interchangeable set of reagents is introduced by at least one secondary support. In some embodiments, said secondary support is a strip of microwells. In some embodiments, said secondary support is a bead.

In some embodiments, said array has sensors to detect a presence of at least one terminal ‘C’ tail in a nucleic acid molecule. In some embodiments, said at least one terminal ‘C’ tail is isolated. In some embodiments, said processing said plurality of biological samples comprises isothermal amplification of at least one selected nucleic acid, comprising: providing at least one sample that comprises at least one nucleic acid by merging droplets containing a plurality of reagents effective to permit at least one isothermal amplification reaction of said sample without mechanical manipulation; conducting at least one isothermal amplification reaction to amplify said nucleic acid.

In some embodiments, said processing said plurality of biological samples comprises a device to detect a polymerase chain reaction (PCR) product on at least one aqueous droplet, wherein the device: creates at least one droplet containing a plurality of nucleic acid and protein molecules on an electrowetting array; performs said PCR reaction while said aqueous droplets are present on said array; interrogates said droplet with a detector.

In some embodiments, said processing said plurality of biological samples includes binding at least one biomolecule on said array. In some embodiments, said array comprises a gas source that contacts and is absorbed by at least one droplet. In some embodiments, said array partitions at least one droplet into a plurality of droplets comprising using: electrowetting force, dielectrowetting force, dielectrophoretic (DEP) effect, acoustic force, hydrophobic knife, or any combination thereof, thereby producing at least one partitioned droplet. In some embodiments, said partitioning dispenses reagents. In some embodiments, said partitioning dispenses samples. In some embodiments, said at least one partitioned droplet is mixed to execute a reaction. In some embodiments, said at least one partitioned droplet is analyzed using said sensors In some embodiments, said at least one partitioned droplet is mixed with at least one target droplet to maintain a constant volume on said at least one target droplet. In some embodiments, said array processes a multiphase fluid. In some embodiments, said array uses dielectrophoretic forces (DEP) for cell sorting, cell separation, manipulating at least one bead, or any combination thereof.

In some embodiments, said biological samples is deposited on a plurality of arrays. In some embodiments, said plurality of arrays comprises at least two arrays. In some embodiments, an array of said at least two arrays is adjacent to another array of said at least two arrays. In some embodiments, said array of said at least two arrays is horizontally adjacent to another array of said at least two arrays. In some embodiments, said array of said at least two arrays is vertically adjacent to another array of said at least two arrays. In some embodiments, said plurality of arrays comprises at least one channel, at least one hole, or any combination thereof. In some embodiments, said at least one channel traverse between at least one surface. In some embodiments, gas. liquid, solid, or any combination thereof is transferred by said at least one hole.

Another aspect of the present disclosure provides a system for biological sample processing, comprising: a housing configured to contain a plurality of arrays, wherein an array of said plurality of arrays is configured to receive, adjacent to said array, a plurality of droplets comprising said plurality of biological samples, and use at least said array to process said plurality of biological samples in said plurality of droplets or derivatives thereof at a coefficient of variation (CV) of at least one parameter of said plurality of droplets or derivatives thereof, or said array, of less than 20% at cross-talk between said plurality of droplets at less than 5%.

In some embodiments, said plurality of arrays is removable from said housing. In some embodiments, said housing is configured to couple to a nucleic acid sequencing platform. In some embodiments, said housing is a nucleic acid sequencing platform. In some embodiments, an environment of said array is controlled by said housing, thereby producing a controlled environment. In some embodiments, ambient humidity, droplet coating, temperature, pressure, droplet size, lighting condition, or any combination thereof are maintained by said controlled environment. In some embodiments, said housing comprises an enclosure. In some embodiments, said enclosure comprises a cover, a seal, a chamber, an immiscible high vapor-pressure fluid, a film, or any combination thereof. In some embodiments, said enclosure comprises an immiscible high vapor-pressure fluid.

One aspect of the present disclosure provides a method for customizing an array system for processing a plurality of biological samples, comprising receiving a request for a configured array system from a user, which request comprises one or more specifications, and using said one or more specifications to configure said array system to yield said configured array system, which configured array system is configured to receive a plurality of droplets comprising said plurality of biological samples and process said plurality of droplets or derivatives thereof at a coefficient of variation (CV) of at least one parameter of said plurality of droplets or derivatives thereof, or said array, of less than 20% at cross-talk between said plurality of droplets at less than 5%.

Another aspect of the present disclosure provides a system for processing one or more droplets, comprising: an array, wherein said array comprises an open configuration with an electrode array, open configuration with no electrode array, open configuration with non-coplanar set of electrodes, two plates with an array of electrodes on one plate and no electrodes on the other plate, two plates with non-coplanar set of electrodes on one plate and no electrodes on the other plate, two plates with an array of electrodes on one plate and single electrode on the other plate, two plates with an non-coplanar set of electrodes on one plate and single electrode on other plate, two plates with electrodes arrays on both plates, two plates with non-coplanar set of electrodes on both plates, or any combination thereof, and wherein said array does not comprise a filler liquid adjacent to said array; one or more liquid handling units, wherein said one or more liquid handling units direct said one or more droplets adjacent to said array.

In some embodiments, said one or more liquid handling units comprise robotic liquid handling systems, acoustic liquid dispensers, syringe pumps, inkjet nozzles, microfluidic devices, needles, microdiaphragm based pump dispensers, piezoelectric pumps, piezo acoustic devices, or any combination thereof. In some embodiments, said array is coupled to at least one reagent or sample storage unit, or a combination thereof. In some embodiments, the system further comprises one or more sensors, wherein said one or more sensors are configured to detect a signal generated from said droplet on said array, said array, an area adjacent to said array or said droplet, or any combination thereof. In some embodiments, said one or more sensors comprise an impedance sensor, a pH sensor, a temperature sensor, an optical sensor, a humidity sensor, a camera, a current measurement sensor, an electronic sensor for biomolecular detection, an x-ray sensor, electrochemical sensors, electrochemiluminescent sensors, piezoelectric sensors, or any combination thereof.

In some embodiments, the system further comprises a computer processor configured to process a signal detected by said one or more sensors and a threshold value or value range, wherein said threshold value or value range is specific to said signal. In some embodiments, the system further comprises a feedback loop, wherein said feedback loop comprises communication between said array, said one or more liquid handling units, said one or more sensors, said computer processor, or any combination thereof. In some embodiments, said feedback loop is configured to discover, optimize, or both, reaction conditions on said array autonomously.

In some embodiments, said plurality of arrays comprises at least two arrays. In some embodiments, an array of said at least two arrays is adjacent to another array of said at least two arrays. In some embodiments, said array of said at least two arrays is horizontally adjacent to another array of said at least two arrays. In some embodiments, said array of said at least two arrays is vertically adjacent to another array of said at least two arrays.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A shows a top view of the plan view of droplets on an electrowetting surface.

FIG. 1B shows a side sectional view of the plan view of droplets on an electrowetting surface.

FIG. 2A depicts a side sectional view of a droplet with less contact on the liquid-on-liquid electrowetting (LLEW) surface of FIG. 1A.

FIG. 2B depicts a side sectional view of a droplet with more contact on the liquid-on-liquid electrowetting (LLEW) surface of FIG. 1A.

FIG. 3A depicts a side sectional view of a droplet on the LLEW surface of FIG. 1A. wherein a droplet is being manipulated by an electrical force.

FIG. 3B represents the movement of the droplet represented in FIG. 3A, resulting from a change in electrical current of adjacent electrodes.

FIG. 3C represents the movement of the droplet represented in FIG. 3B, resulting from a change in electrical current of adjacent electrodes, to a final position on the electrowetting surface.

FIG. 4A depicts a side sectional view of a droplet on the LLEW surface of FIG. 1A. wherein two droplets are being manipulated by an electrical force.

FIG. 4B represents the merging of the two droplets of FIG. 4A using a change in electrical force.

FIG. 4C represents the complete merging of the two droplets of FIG. 4B.

FIG. 5A depicts a side sectional view of a droplet on the electrowetting surface of FIG. 1A wherein a droplet has less contact with the electrowetting surface.

FIG. 5B depicts a side sectional view of a droplet on the electrowetting surface of FIG. 1A wherein a droplet has more contact with the electrowetting surface. The amount of contact of the droplets is controlled with an electric field.

FIGS. 6A-6C depict side sectional views of a droplet containing a biological sample on the electrowetting surface of FIG. 1A, wherein the surface is in an open configuration. Additionally, FIGS. 6A-6C show various placements of reference electrodes in relation to actuation electrode on an electrowetting array.

FIG. 7A shows a cross section of a circuit board at low magnification.

FIG. 7B shows a cross section of a circuit board at high magnification.

FIGS. 8A-8F show side section views for printed circuit boards with various steps in application of dielectric coating and steps in the process of planarization.

FIGS. 9A-9D represent various manufacturing processes of microstructures on dielectric towards achieving a slippery surface.

FIGS. 10A-10I depict motion of a droplet on an array of electrodes.

FIG. 11A represents a perspective view of a laboratory apparatus.

FIG. 11B represents a top view of example processing station of the system described herein wherein an electrowetting device may incorporate one or more mixing station(s) (1120).

FIG. 11C represents a top view of example processing station of the system described herein wherein an electrowetting chip may include one or more temperature control station(s) (1128).

FIG. 11D represents a top view of example processing station of the system described herein wherein a magnetic bead wash station (1134) may contain samples with nucleic acids, proteins, cells, buffers, magnetic beads, wash buffers, elution buffers, and other liquids (1136) on an electrode grid.

FIG. 11E represents a top view of example processing station of the system described herein wherein an electrowetting chip may include one or more nucleic acid delivery station(s) (1140).

FIG. 11F represents a perspective view of example processing station of the system described herein wherein sample (1156) may be on an open surface with single plate electrowetting device (100).

FIG. 11G represents a perspective view of example processing station of the system described herein wherein sample (1156) may be sandwiched between two plates (100, 1160).

FIG. 11H represents a perspective view of example processing station of the system described herein wherein the optical detection may be performed on any M×N format.

FIG. 11I represents a side section view of processing stations of example electrowetting devices described herein wherein droplets may be loaded onto the electrowetting surface through acoustic droplet ejection.

FIG. 11J represents a side section view of processing stations of example electrowetting devices described herein wherein an electrowetting device may include one or more stations (1180) designed to load biological samples, chemical reagents, and liquids (1182) through a microdiaphragm pump (1184) based dispenser onto an electrowetting chip.

FIG. 12A represents an exploded view of two configurations of example electrowetting devices described herein.

FIG. 12B represents side section views of example an electrowetting array for optoelectrowetting described herein.

FIG. 12C represents side section views of example an electrowetting array for photoelectrowetting described herein.

FIG. 13 represents a cartoon of computer systems used for arrays described herein.

FIG. 14 illustrates an array for manipulating liquids that may use electrowetting, dielectrowetting or dielectrophoresis for dispensing liquid and droplet creation.

FIG. 15 depicts an array for a computer-vison system to monitor a droplet.

FIG. 16 depicts an array for a computer-vison system comprising an optical filter.

FIGS. 17A-17B depict example computer-vison configurations to monitor a droplet.

FIG. 18 depicts an array for a computer-vison system that can monitor the properties of a droplet (e.g., change in volume).

FIG. 19 depicts an array design for visualization or optical inspection of the array.

FIG. 20 illustrates an example of a chamber to reduce droplet evaporation.

FIG. 21 illustrates an example of a chamber to reduce droplet evaporation.

FIG. 22A illustrates an open plate example of cloaking to reduce droplet evaporation.

FIG. 22B illustrates a closed plate example of cloaking to reduce droplet evaporation.

FIG. 23 illustrates an example of submersion to reduce droplet evaporation.

FIG. 24 illustrates an example of a membrane covering a droplet surface to reduce droplet evaporation.

FIG. 25A illustrates a side view of an example of a seal to reduce droplet evaporation.

FIG. 25B illustrates a top view of an example of a seal to reduce droplet evaporation.

FIGS. 26A-26F depict configurations to control liquid evaporation.

FIG. 27 shows an example array that can actively control temperature.

FIGS. 28A-28B depict internal components for an array comprising temperature elements (e.g., heating, cooling, and temperature sensing elements).

FIGS. 29A-29F depict configurations to replenish liquid evaporation.

FIGS. 30A-30B depict configurations of magnets to introduce magnetic fields onto an array that can control droplet motion on a surface.

FIG. 31A illustrates an example configuration of electro-permanent magnets to control the magnetic field of an array.

FIG. 31B illustrates an example configuration of rotary switchable magnets to control the magnetic field of an array.

FIG. 32 depicts an array design (top and side views) for reference electrode design and placement.

FIG. 33 depicts an array design for mesh or individual wire reference electrode design and placement. As shown, the reference electrode array may be non-coplanar with the actuation electrode array.

FIG. 34 illustrates an example of a position of a reference electrode, or a set thereof, of an array.

FIG. 35A represents an electrically conductive dielectric layer as an example system and method for reference electrode placement on an electrode array.

FIG. 35B represents a liquid coating that functions as a reference electrode as an example system and method for reference electrode placement on an electrode array.

FIG. 35C represents electrically conductive ionized particles as an example system and method for reference electrode placement on an electrode array.

FIG. 36 depicts an example of electrowetting-on-dielectric (EWOD)-enabled magnetic bead washing.

FIG. 37A represents an example of a design and the components of an open disposable cartridge.

FIG. 37B represents an example of a design and the components of a closed disposable cartridge.

FIGS. 38A-38B depict an array tile constructed separately from the electronics.

FIG. 39 shows an example technique to process large volumes of liquid on an array.

FIG. 40 depicts example circuitry for array multiplexing.

FIG. 41 shows a reconfigurable bay with reconfigurable trays for array multiplexing.

FIG. 42 depicts an example of bonding films or cartridges.

FIG. 43 depicts an example configuration for stacking dielectric and slippery layers of an array.

FIG. 44 depicts an example of the configuration of a frame that can contain polymer film layers.

FIG. 45 depicts an example of the configuration of an array that comprises polymer film layers under tension.

FIGS. 46A-46B depict configurations to apply a polymer film layer with rollers, dispensers, or a combination thereof.

FIG. 47A illustrates an example of a design (47A) for single cell isolation, cellular barcoding, and cell tracking.

FIGS. 47B-47C illustrate an example of methods for single cell isolation, cellular barcoding, and cell tracking.

FIG. 48A illustrates an example of horizontal multi-story chip design.

FIG. 48B illustrates an example of horizontal multi-story chip design with holes and channels.

FIG. 48C illustrates an example of vertical multi-story chip design.

FIGS. 49A-49B illustrates example designs for polymer printing. The same system can be used for polymer-based data storage.

FIG. 49C illustrates an example workflow for polymer printing. The same system can be used for polymer-based data storage.

FIG. 50A illustrates example of open systems of membranes for separating droplets.

FIG. 50B illustrates example of closed systems of membranes for separating droplets.

FIG. 51 depicts a configuration of an array for capacitive sensing.

FIG. 52A shows an example of a droplet on an open array undergoing electroporation.

FIG. 52B shows a side view of the open array depicted in FIG. 52A.

FIG. 53A shows an example of an array that can perform electroporation with an electric field.

FIGS. 53B-53C show side views of a two-plate system described herein.

FIG. 54 represents an example design of capacitive sensors for droplet sensing and droplet visualization.

FIG. 55 shows an example configuration for an array that can be used for performing the polymerase chain reaction (PCR) and quantitative PCR (qPCR).

FIGS. 56A-56B depicts arrays that are capable of controlling microliter-, nanoliter- or picoliter-sized droplets on an open surface.

FIG. 57 shows an example configuration for an optical-based detection array.

FIG. 58 depicts an example of a next-generation sequencing library preparation platform.

FIG. 59 represents evaporation time as a function of droplet volume for example systems described herein.

FIG. 60 depicts an example Next-Generation Sequencing chip and array set-up.

FIG. 61 depicts an example factory scale box comprising a plurality of arrays described herein.

FIG. 62 depicts an example configuration for library preparation for next-generation sequencing preparation.

FIG. 63 shows an example configuration for next generation sequencing (NGS) library preparation using an array described herein.

FIG. 64A depicts data for the yield of DNA isolated using systems and methods described herein.

FIG. 64B depicts data for the size of DNA isolated using systems and methods described herein.

FIG. 65 depicts data for the distribution of the size of DNA isolated using systems and methods described herein.

FIGS. 66A-66B show example workflows to afford the synthesis of DNA.

FIG. 66C shows a schematic diagram for a single reaction site that performs step by step addition of nucleotides to synthesize a long molecule of DNA.

FIG. 67 depicts an array tile described herein.

FIG. 68 shows the library size distribution for on-chip vs. off-chip experiments of a NGS library preparation using systems and methods described herein.

FIG. 69 depicts the quality for sequencing libraries for on-chip vs. off-chip experiments of a NGS library preparation using systems and methods described herein.

FIG. 70 depicts the level of duplicates for sequencing libraries for on-chip vs. off-chip experiments of a NGS library preparation using systems and methods described herein.

FIGS. 71A-71B depict the level of adapter contamination for experiments of a NGS library preparation using systems and methods described herein.

FIG. 72 depicts the level coverage across the human genome for experiments of a NGS library preparation using systems and methods described herein.

FIG. 73 depicts the single nucleotide polymorphism (SNP) sensitivity for experiments of a NGS library preparation using systems and methods described herein.

FIG. 74 depicts an example schematic NGS workflow using systems and methods described herein. The example workflow comprises manipulating (e.g., lysing cells, digesting protein, and DNA clean-up) biological samples on an array described herein.

FIG. 75 exemplifies an embodiment of the module and cover described herein.

FIG. 76 exemplifies an embodiment of the module and projector described herein.

FIG. 77 depicts an embodiment of a splitting process during library quantification using systems and methods described herein.

FIG. 78 depicts an embodiment of a reverse transcription loop-mediated isothermal amplification (RT-LAMP) process carried out on an array described herein.

FIG. 79 depicts an embodiment of detecting viral RNA using LAMP dye and a fluorescent camera on an array described herein.

FIG. 80 depicts an embodiment of results of analyzation by gel electrophoresis after RT-LAMP amplification described herein.

FIGS. 81A-81B depict an embodiment of linking an antibody or antigen to a surface of an array described herein.

FIG. 82 depicts an embodiment of detecting viral RNA antibodies on an array described herein.

FIG. 83A-83F depicts an embodiment of detecting viral RNA antibodies on an array described herein.

FIG. 84 illustrates a workflow for DNA assembly to gene amplification and/or protein expression process according to some embodiments described herein.

FIG. 85 depicts an embodiment of a Gibson DNA assembly method described herein.

FIG. 86 depicts disposition of droplets on an array for consecutive DNA assembly, purification, and amplification according to some embodiments described herein.

FIG. 87 shows results of analyzation by gel electrophoresis after PCR amplification of a synthetic GFP gene carried out on an array described herein.

FIG. 88 illustrates a Golden Gate assembly method according to some embodiments described herein.

FIG. 89 illustrates implementation of an electrophoresis device on an array according to some embodiments described herein.

FIG. 90 depicts a method of DNA cloning as described herein.

FIG. 91 depicts an electrowetting (EWOD) array comprising a delimited-surface coated with agarose according to some embodiments described herein.

FIG. 92 illustrates a method of rolling circle amplification according to some embodiments described herein.

FIG. 93 depicts a cell screening process according to some embodiments described herein.

FIG. 94 depicts a shearing module implemented on an array according to some embodiments described herein.

FIG. 95 depicts a droplet splitting mechanism according to some embodiments described herein.

FIG. 96 depicts a droplet splitting mechanism according to some embodiments described herein.

FIG. 97 depicts a droplet aliquoting mechanism according to some embodiments described herein.

FIG. 98 depicts a droplet aliquoting mechanism according to some embodiments described herein.

FIG. 99 depicts a waste disposal mechanism according to some embodiments described herein.

FIGS. 100A-100B depict an array according to some embodiments described herein.

FIG. 101 depicts an array according to some embodiments described herein.

FIGS. 102A-102B depict an array according to some embodiments described herein.

FIGS. 103A-103B depict an array according to some embodiments described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “slide angle”, as used herein, generally refers to the angle from horizontal at which a droplet of a given size begins to move under the force of gravity. For example, a surface that holds a 5 microliter (id) droplet at 4° but allows it to slide at 5° may be said to have a 5 μl slide angle of 5°. For various applications, 5 μl slide angles of less than or equal to 70°, 60 0, 50 0, 40°, 30°, 25°, 20°, 15°, 10°, 5°, 3°, 2°, 1° or less may be used. The smaller the slide angle, the more slippery the surface, and generally the lower the voltage required to move droplets across the surface.

The term “contact angle hysteresis”, as used herein, generally refers to the observed differences between advancing and receding contact angles. For example, in a surface with lower surface adhesion, as a liquid droplet moves across the surface, the contact angle between the leading edge and the surface vs. the trailing edge and the surface can be close the same. However, in a surface with higher adhesion, the difference between the leading and trailing contract angles can become larger. Low surface roughness, high surface hydrophobicity, and low surface energy can result in less difference in this angle. Contact angle hysteresis (that is, the difference between leading and trailing contact angles) of less than or equal to 70°, 60°, 50°, 40°, 30°, 25°, 20°, 15°, 10°, 7°, 5°, 3°, 2° or less may be used.

The term “droplet”, as used herein, generally refers to a discrete or finite volume of a fluid (e.g., a liquid). A droplet may be generated by one phase separated from another phase by an interface. The droplet may be a first phase phase-separated from another phase. The droplet me include a single phase or multiple phases (e.g., an aqueous phase containing a polymer). The droplet may be a liquid phase disposed adjacent to a surface and in contact with a separate phase (e.g., gas phase, such as air).

The term “biological sample,” as used herein, generally refers to a biological material. Such biological material may display bioactivity or be bioactive. Such biological material may be, or may include, a deoxyribonucleic acid (DNA) molecule, a ribonucleic acid (RNA) molecule, a polypeptide (e.g., protein), or any combination thereof. A biological sample (or sample) may be a tissue sample, such as a biopsy, core biopsy, needle aspirate, or fine needle aspirate. The sample may be a fluid sample, such as a blood sample, urine sample, stool sample, or saliva sample. The sample may be a skin sample. The sample may be a cheek swab. The sample may be a plasma or serum sample. The sample may be a plant derived sample, water sample or soil sample. The sample may be extraterrestrial. The extraterrestrial sample may contain biological material. The sample may be a cell-free (or cell free) sample. A cell-free sample may include extracellular polynucleotides. Extracellular polynucleotides may be isolated from a bodily sample that may be selected from a group consisting of blood, plasma, serum, urine, saliva, mucosal excretions, sputum, stool and tears. The sample may include a eukaryotic cell or a plurality thereof. The sample may include a prokaryotic cell or a plurality thereof. The sample may include a virus. The sample may include a compound derived from an organism. The sample may be from a plant. The sample may be from an animal. The sample may be from an animal suspected of having or carrying a disease. The sample may be from a mammal.

The term “% glycerol,” as used herein, generally refers to the viscosity of a solution as compared to a glycerol in water solution wherein the amount of glycerol in water (by volume) is determined by the value of the percentage. For example, a solution described herein with a viscosity of about “30% glycerol” expresses that the viscosity of the solution is the equivalent of a glycerol in water solution comprising about 30% glycerol.

The term “subject,” as used herein, generally refers to an animal, such as a mammal (e g, human) or avian (e.g., bird), or other organism, such as a plant. The subject can be a vertebrate, a mammal, a rodent (e.g., a mouse), a primate, a simian or a human Animals may include, but are not limited to, farm animals, sport animals, and pets. A subject can be a healthy or asymptomatic individual, an individual that has or is suspected of having a disease (e.g., cancer) or a pre-disposition to the disease, an individual that needs therapy or suspected of needing therapy, or any combination thereof. A subject can be a patient

The term “coefficient of variation,” as used herein, generally refers to repeatability and precision. This may be given by Equation 1, where s is the standard deviation of the responsivities of the different materials and x is the mean responsivity of all materials.

$$C V = \frac{s}{x} \times 100 \qquad Equation 1$$

The term “cross-talk,” as used herein, generally refers to contamination of a droplet. Cross-talk may refer to a percentage of a droplet, a biological sample, or a combination thereof acquired from another droplet. If p₁ represents the material of interest in the droplet and p₂ is the total material from other droplets present in the droplet of interest, the cross-talk may be given by Equation 2.

$$C T = \frac{{p}_{2}}{{p}_{1} + {p}_{2}} \qquad Equation 2$$

### Electrowetting Devices and Systems

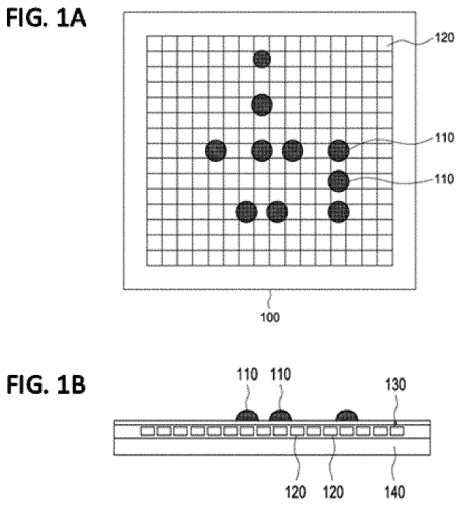

Referring to FIGS. 1A-1B, an electrowetting device may be used to move individual droplets of water (or other aqueous, polar, or conducting solution) from place to place. The surface tension and wetting properties of water may be altered by electric field strength using the electrowetting effect. The electrowetting effect may arise from the change in solid-liquid contact angle due to an applied potential difference between the solid and the liquid. Differences in wetting surface tension that may vary over the width of the droplet, and corresponding change in contact angle, may provide motive force to cause the droplets to move, without moving parts or physical contact. The electrowetting device (100) may include a grid of electrodes (120) with a dielectric layer (130) with appropriate electrical and surface priorities overlaying electrodes (120), all laid on a rigid insulating substrate (140).

The surface of the electrode grid may be prepared so that it has low adhesion with water. This may allow water droplets (110) to be moved along the surface by small forces generated by gradients in electric field and surface tension across the width of the droplet. A surface with low adhesion may reduce the trail left behind from a droplet. A smaller trail may reduce droplet cross contamination, and may reduce sample loss during droplet movement. Low adhesion to surface may also allow for low actuation voltage for droplet motion and repeatable behavior of droplet motion. There are several ways to measure low adhesion between a surface and a droplet including slide angle and contact angle hysteresis, such as, for example, using a contact angle goniometer or a charge-coupled device (CCD) camera.

There may be several ways to achieve low surface adhesion; for example, mechanically polishing, chemically etching, or a combination thereof until smooth within a few nanometers, applying coating to fill surface irregularities, applying liquids to fill surface irregularities, chemically modifying the surface to create desirable surface properties (hydrophobic, hydrophilic, resistance to biofouling, varying with electric field strength, etc.).

Liquid-On-Liquid Electrowetting (LLEW) for Electrowetting

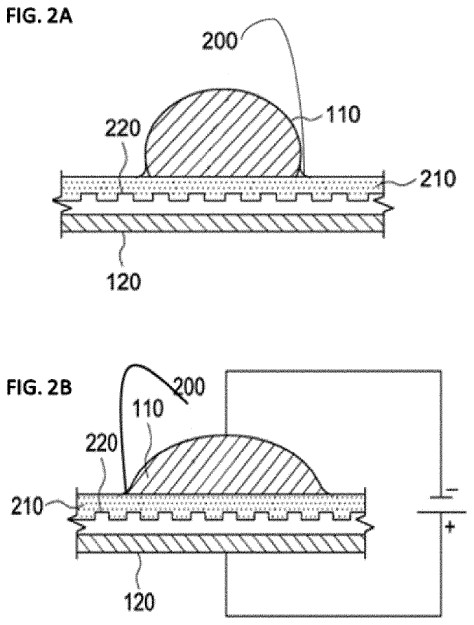

Referring to FIGS. 2A and 2B, an electrowetting mechanism called “liquid-on-liquid-electrowetting” (LLEW) takes advantage of an electrowetting phenomenon that occurs at a liquid-liquid-gas interface (200). A droplet (110) riding on the surface of a layer of a low surface energy liquid (210) (such as oil) and substantially surrounded by gas (such as air, nitrogen, argon, etc.) creates a liquid-liquid-gas interface at the contact line (200). The oil (210) may be stabilized in place on the solid substrate by a textured surface (220) of the solid substrate, and the conductive layer of metal electrodes (120) may be embedded in the body of this solid. Referring to FIG. 2B, when an electric potential is applied across the height of droplet (110), the liquid-liquid-gas interface (200) may cause droplet (110) to wet the oil (210) and spread across the surface while still riding on the oil (210).

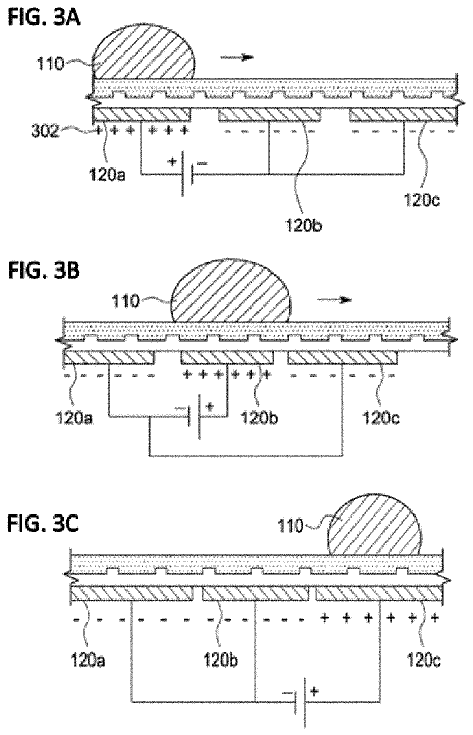

Referring to FIGS. 3A, 3B, and 3C, the liquid-on-liquid electrowetting technique may be used to manipulate droplets (110) that may contain biological and chemical samples. In FIG. 3A, droplet (110) may be in motion from left to right, and has just been attracted onto the left-most of three electrodes (120a) by a positive voltage (302) on that leftmost electrode (120a), with consequent addition of electric field at the liquid-liquid surface and enhanced wetting. In FIG. 3B, the voltage is withdrawn from the leftmost electrode (120a) and applied to the center electrode (120b). Because of the enhanced wetting over the center electrode (120b), the droplet may be attracted to the center position in FIG. 3B. In FIG. 3C, the voltage is withdrawn from the left and center electrodes (120a and 120b, respectively) and applied to the right electrode (120c), and the enhanced wetting over the right electrode (120c) has attracted the droplet to the right.

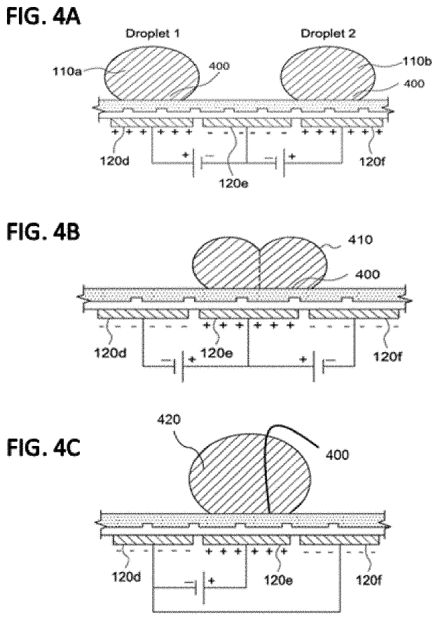

Referring to FIGS. 4A, 4B, and 4C, differential wetting may be used to merge two droplets (e.g., 110a and 110b) on a LLEW surface (400) over an electrode array (e.g., 120d, 120e, and 120f). In FIG. 4A, two droplets have been attracted to the leftmost and rightmost electrodes (120d and 120f respectively). In FIG. 4B, the voltage is removed from the left and right electrodes (120d and 120f, respectively) and applied to the center electrode (120e). The two droplets may be attracted from left and right to center and begin to merge (410). In FIG. 4C, merging of the two droplets is complete (420).

Referring to FIGS. 3A, 3B, 3C, 4A, 4B, and 4C, such a microfluidic selective wetting device may be capable of performing, for example, microfluidic droplet actuation such as droplet transport, droplet merging, droplet mixing, droplet splitting, droplet dispensing, droplet shape change, or a combination thereof. This LLEW droplet actuation may then be used for a microfluidic device to automate biological experiments such as liquid assays, in devices for medical diagnostics and in many lab-on-a-chip applications.

Electrowetting on a Dielectric (EWOD) for Droplet Manipulation

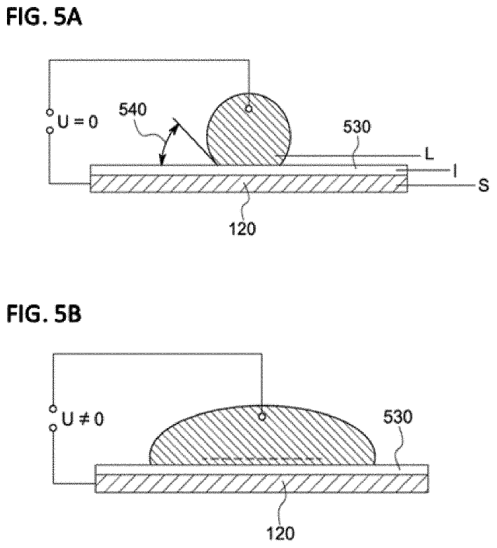

Referring to FIGS. 5A and 5B, Electrowetting on Dielectric (EWOD) is a phenomenon in which the wettability of an aqueous, polar, or conducting liquid (L) may be modulated through an electric field across a dielectric film (530) between the droplet and conducting electrode (120, S). Adding or subtracting charge from electrode (120) may change the wettability of an insulating dielectric layer (530, I), and that wettability change is reflected in a change to contact angle (540) of the droplet. The contact angle change may in turn cause the droplet to change shape, to move, to split into smaller droplets, or to merge with another droplet. As represented by Equation 4, the contact angle (540) is a function of the applied voltage.

The wetting behavior (wetting or wettability) of a liquid on a solid surface refers to how well a liquid spreads on the solid surface. The wettability of a droplet on a solid surface surrounded by a gas (e.g., air) is governed by interfacial tension between the solid, liquid, and gas medium. For an immobile droplet, the wettability may be measured in terms of the contact angle (540) with the solid surface, which may be governed by Young's equation (equation 3):

γ_{SL}=γ_{SG}+γ_{LG} cos(θₑ) Equation 3

where Y_{SL} is the solid-liquid surface tension, Y_{LG} is the liquid air surface tension, Y_{SG} the solid-gas surface tension and θₑ is the contact angle under equilibrium.

Gabriel Lippman observed that the capillary level of mercury in an electrolyte changes when a voltage is applied. This phenomenon (electro-capillarity) may be then described through Lippmann-Young's equation (Equation 4):

$$cos ( {θ}_{u} ) = cos ( {θ}_{0} ) + \frac{c {U}^{2}}{2 {γ}_{LG}} \qquad Equation 4$$

θ₀ is the contact angle when the electric field is zero (i.e. no voltage applied) and θᵤ is the contact angle when a voltage U is applied, and c is the capacitance per unit area between the electrode and the droplet.

Manufacturing Methods for Electrowetting Arrays

An electrowetting device may be used for transporting and mixing liquids that may contain biological liquids may consist of an array of electrodes (120) on an insulating substrate (140), a thin layer of dielectric (130) and, if necessary, a final slippery (low surface energy) coating. Sometimes the dielectric layer itself may provide sufficient hydrophobic and slippery behavior with or without additional chemical or topographical modification.

The electrode grid (120) on an insulating substrate (140) may be fabricated using some combination of one or more of the following methods—printed circuit board manufacturing (PCB manufacturing), CMOS, or HV CMOS or other semiconductor fabrication methods, manufactured using thin-film transistor (TFT), active matrix, or passive matrix backplane technology, or any other method that is capable of laying conductive circuits on an insulating substrate. To isolate the liquid during motion and mixing, the surface of the electrode array may be covered with a dielectric with one of the many methods described below.

The PCB and surface electrodes may be fabricated using thin-film-transistor (TFT), active matrix or passive matrix backplane technology.

The chemistry and texture of the top surface of the dielectric interacting with a droplet may govern the voltages required for successful and repeated motion of droplets. As a result of the chemical makeup and physical texture, a droplet on an electrowetting device may experience two phenomena when in motion: droplet pinning and contact angle hysteresis. Droplet pinning phenomenon may be when a droplet gets stuck to any local surface defects when it is being moved. Contact angle hysteresis may be the difference in the advancing and the receding contact angle for a droplet in motion. As a result of droplet pinning and high contact angle hysteresis, droplets on an electrowetting surface may require significantly high voltage. The chemical makeup of the surface, the texture and slipperiness of the surface and smoothness of the surface also may result in droplets leaving a trail behind as it is being moved. This trail may be as simple as just one molecule or as significant as over 99 percent of the droplet.

To reduce pinning, contact angle hysteresis and trail left behind by a droplet, the dielectric covering the electrode array may be smoothed and then chemically modified to create a surface with low surface energy. Surface energy may be the energy associated with the intermolecular forces at the interface between two media. A droplet interacting with a low surface energy surface may be repelled by the surface and considered hydrophobic. The dielectric layer itself may provide a sufficiently slippery surface for droplet motion.

The following section describes various materials that may be used in manufacturing an electrowetting device: substrate for laying conductive material, conductive materials for electrodes and interconnects, dielectric material, methods for depositing dielectric materials, achieving smooth surface on the dielectric and hydrophobic coating materials to provide slippery surface for droplet motion.

Substrates for Electrowetting

An electrowetting microfluidic device may be formed by creating a slippery (in the sense of low surface energy) surface directly on the electrode array (120). Electrode arrays may consist of conductive plates that charge electrically to actuate the droplets. Electrodes in an array may be arranged in an arbitrary layout, for example a rectangular grid, or a collection of discrete paths. The electrodes themselves may be made of one or more conductive metals (including gold, silver, copper, nickel, aluminum, platinum, titanium), one or more conductive oxides (including indium tin oxide, aluminum doped zinc oxide), one or more conductive organic compounds (including PEDOT and polyacetylene), one or more semiconductors (including, silicon dioxide), or any combination thereof. The substrates for laying out the electrode array may be any insulating materials of any thickness and any rigidity.

The electrode arrays may be fabricated on standard rigid and flexible printed circuit board substrates. The substrate for the PCB may be FR4 (glass-epoxy), FR2 (glass-epoxy), Rogers material (hydrocarbon-ceramic), or insulated metal substrate (IMS), polyimide film (example commercial brands include Kapton, Pyralux), polyethylene terapthalate (PET), ceramic or other commercially available substrates of thickness from 1 μm to 10,000 μm. Thicknesses from 500 μm to 2000 μm may be utilized in some embodiments.

The electrode arrays may also be made of conductive elements, semiconductive elements, or any combination thereof which may be fabricated with active matrix technologies and passive matrix technologies such as thin film transistor (TFT) technology. The electrode arrays may also be made of arrays of pixels fabricated with traditional CMOS or HV-CMOS fabrication techniques.

The electrode arrays may also be fabricated with transparent conductive materials such as indium tin oxide (ITO), aluminum doped zinc oxide (AZO), fluorine doped tin oxide (FTO) deposited on sheets of glass, polyethylene terapthalate (PET) and any other insulating substrates.

The electrode arrays may also be fabricated with metal deposited on glass, polyethylene terapthalate (PET) and any other insulating substrates.

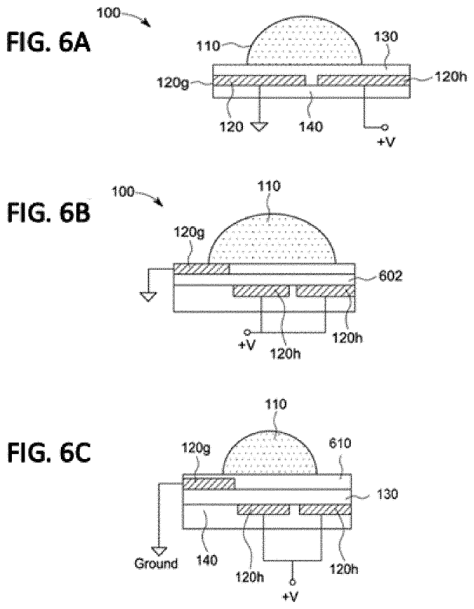

Referring to FIG. 6A, in some cases, the electrowetting microfluidic device (100) may be composed of coplanar electrodes (e.g., electrodes on same layer, 120g and 120h) with no second plate, and the droplet (110) may ride on an open surface above the plane of the electrodes. In this configuration the reference electrodes (e.g., ground signal, 120g) and actuation electrodes (120h) may be on the same plane, laid on a printed circuit board substrate (140), with a thin insulator above the electrodes (130). Droplets may ride on this insulator layer, and are not sandwiched between two plates. In some embodiments, the reference electrode (120g) may be of a different geometry compared to the actuation electrode. In some embodiments, dielectric elements or layers are placed so that the droplets (110) may not come into contact with electrodes (120) of differing polarity, so that the droplets may be exposed to electric fields, not electric current.

Referring to FIG. 6B, in some embodiments, the electrowetting microfluidic device may be composed of two layers of electrodes (one for reference electrode (120g) and one for actuation electrodes (120h)), one atop the other within the substrate (140) (as opposed to a sandwich of electrodes with the droplet between plates). Here, a droplet (110) may ride on an open surface and may sit above both layers of electrodes. The two layers of electrodes (120g and 120h) may be spaced apart by a thin layer (602) of insulator (e.g., from 10 nm to 30 μm). In some embodiments, the layer with reference electrode (120g) may be closer to the droplet. The reference electrode (120g) on the topmost layer may be directly in contact with a droplet. The reference electrode layer may be less than 500 nm in thickness and may be coated with hydrophobic materials. The second layer with reference electrode may be a single continuous trace of any arbitrary shape.

Referring to FIG. 6C, the layers from top down may be arranged as a hydrophobic layer (610), a layer with electrodes (120g) (e.g., reference or ground), a dielectric layer (130), a layer of actuation electrodes (120h), and the insulting circuit board substrate (140). The droplets (110) may ride on the top open surface hydrophobic layer (610). Because the electrodes (120) may be metallic the dielectric layer (130) may separate the two non-coplanar electrode arrays.

In constructing the electrowetting microfluidic device (100), many layers of laminations (from 1 to 50 layers) may be used to isolate multiple layers of electrical interconnect routing (from 2 to 50 layers). One of the outermost layers of lamination may contain electrode pads (120) for actuating droplets and may contain reference electrodes. The interconnects may connect the electrical pads to high voltages for actuation and for capacitive sensing. The actuation voltage may be from 1V to 350V. This actuation voltage may be an AC signal or DC signal.

Creatine Smooth Dielectric Surface on the Electrode Arras,

In order to isolate the droplet electrically from the electrode array, a layer of dielectric (130) may be applied on the top surface of the electrode array (120). The top surface of this dielectric layer (130) may be formed with a top surface that offers little to no resistance to droplet motion, so that droplets may be moved with low actuation voltages (less than 100V DC, less than 80V, less than 50V, less than 40V, less than 30V, less than 20V, less than 15V, less than 10V, less than 8V, or less, depending on the degree of smoothness, slipperiness, hydrophobicity, or any combination thereof). To achieve a low resistance slippery surface, the dielectric surface may have a smooth surface topography and may be hydrophobic or otherwise offer low adherence to the droplet. A chemical treatment may also be applied directly to the dielectric surface.

A smooth topography surface is typically characterized by its roughness value. By experimentation, it has been found that the voltages required to effect droplet motion may vary as the surface becomes smoother. The smoothness may be less than 2 μm, 1 μm, 500 nm, or less.

A smooth dielectric surface above the electrode arrays may be formed by some combination of techniques such as:
1. A two-step process where the surface defects may be patched to achieve a relatively smooth surface and then it may be covered with a dielectric material. Patching the defects may be done with a photoresist, epoxy or potting compound. The second layer of dielectric may either be the same material or a polymer film.
2. A second method may be to deposit excess photoresist or epoxy on the electrode array and then polish the excess material down to required thickness and surface roughness.
3. A third method may be to stretch and bond a thin polymer film on to the surface.

To prevent the droplet from adhering to the smoothed dielectric surface 130, the surface may be further modified to make it slippery by one or more of the following methods:
1. Modifying the surface chemistry
2. Modifying the surface topography
3. Applying a slippery liquid coating, which is called liquid-on-liquid electrowetting (LLEW).

The following section describes in details various methods to modify the rough non-slippery surface of electrode array into a smooth slippery surface.

Smoothing with Photoresist/Epoxy/Potting Compound

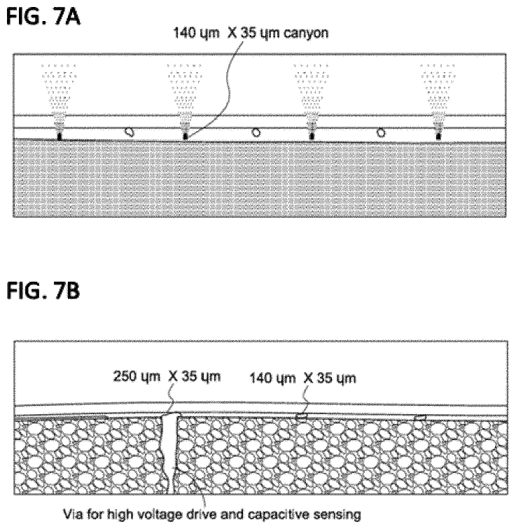

Referring to FIGS. 7A and 7B, printed circuit boards (PCBs) manufactured by typical processes may have surface roughness in the form of: canyons (gaps) between electrodes, holes for establishing connection between multiple layers (also known as vias), holes to solder through-hole components and any other imperfections from manufacturing errors, and the like. Typical dimensions of surface imperfections may be in the range of 30 μm to 300 μm, and may be as small as 1 μm, varying based on the fabrication process.

Several methods may be used singly or in conjunction to reduce these surface imperfections, to achieve a planar surface of roughness value less than 1 μm, more or less, which in turn, may provide desirable wetting properties and behavior, at lower voltages.

A smooth surface may be achieved by flowing photoresist, epoxy, potting compound or liquid polymers between canyons. A photoresist of interest may flow between canyons of size less than 10 μm in any dimension and may have a dynamic viscosity less than 8500 centipoise. Commercially available SU-8 photoresist is a good example of this. A suitable liquid polymer for this purpose may be liquid polyimide, for example.

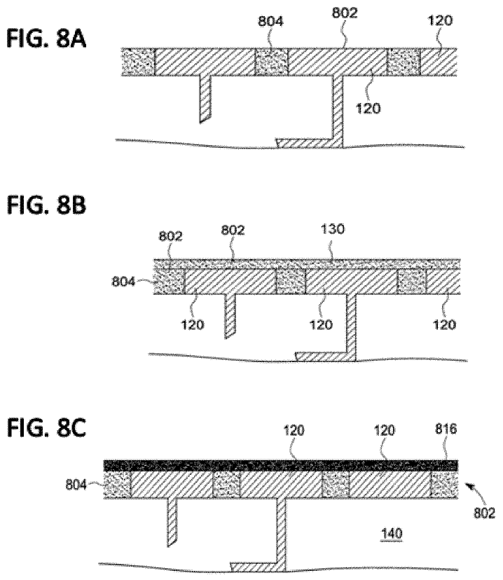

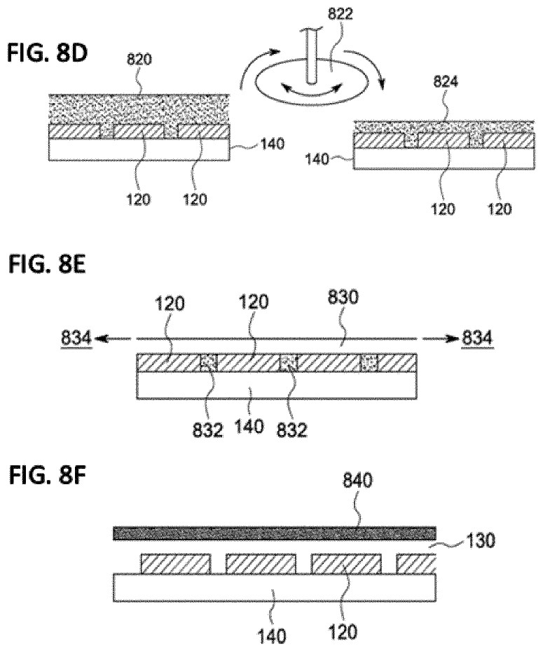

Referring to FIG. 8A, to fill canyons between electrodes (120), an approximately planarized surface (802) of an electrode array may be achieved by applying a coating (804) of photoresist, epoxy, potting compound, liquid polymer, or another dielectric. The material may have gap-filling properties that allows it to flow into small gaps (for example, 100 μm (width)×35 μm (height)), and to fill larger gaps. The coating may then be cured to achieve a surface of roughness value in the desirable range, which may be 1 μm more or less. The metal electrode surface may be exposed or covered with the coating.

Creating a Dielectric on the Smooth Photoresist/Epoxy/Potting Compound

Once the surface imperfections may be patched up by flowing a photoresist or epoxy or potting compound (804), the topmost surface of the electrode array may be planarized (802). Referring to FIG. 8B, the substantially planar surface may have metal electrodes (120) that may have an additional dielectric coating (130) surrounding it to isolate a droplet from a charged electrode, while allowing the electric field to propagate to where the droplet may still be influenced by the electric field. The thickness of this coating (130) may range from 10 nm to 30 μm. The dielectric layer (130) may be formed as a thin film by various deposition thin films via various coating methods, by bonding a polymer film as described next or by any other thin film deposition techniques.

Deposit Thin Film Coatings as Dielectric

Referring to FIG. 8B, the top planarized surface (802, exposed metal electrode (120) and photoresist (804) of FIG. 8A) may be coated with an additional layer of the same photoresist (or epoxy or potting compound) material, or a different material with different dielectric, bonding, and smoothing properties to create the dielectric layer (130) that may electrically isolate droplets from the electrodes. The photoresist may be applied by spin coating, spray coating, chemical vapor deposition, drop coating, dip coating, and the like.

The planarized surface (802) may also be coated with thin film (130) of dielectric by some form of chemical vapor deposition. This kind of deposition may result in the film following the topography of the coated surface. An example class of materials commercially available for vapor deposition is called conformal coating materials and may be well suited for scalable manufacturing. Conformal coating materials include, for example, Parylene conformal coating, epoxy conformal coating, polyurethane conformal coating, acrylic conformal coating, and fluorocarbon conformal coating. Other coating materials that may be used with vapor deposition include, for example, silicon dioxide, silicon nitride, hafnium oxide, tantalum pentoxide, titanium dioxide, or any combination thereof.

Bond Polymer Films to Form Top-Most Dielectric

Referring to FIG. 8C, the top planarized surface (802, metal electrode (120) and photoresist (804)) may be covered with an additional layer of polymer film (816) to isolate the droplet from the electrodes. The film (816) may be stretched to eliminate wrinkles, and ensure additional smoothness. The polymer film may be held on the electrode array by heat bonding or by vacuum suction or by electrostatically sucking it down or simply by mechanical holding it in place.

Using Excess Photoresist and Polishing to a Smooth Dielectric Surface

Referring to FIG. 8D, a smooth dielectric surface may be achieved by coating the electrode array with a photoresist or other curable dielectric materials (820) and then polishing (822) the topmost surface to achieve a smooth surface (824). The photoresist/dielectric material may be coated using techniques such as, for example, spin-coating, spray coating, vapor deposition or dip coating.

This process may comprise coating the electrode array (120) with a curable dielectric (820) to a thickness significantly higher than the height of the electrode. For example, if the electrode measures 35 μm in height, the dielectric coating thickness above the top surface of the electrode may be at least 70 μm. The dielectric may be polished (822) with a fine abrasive and a chemical slurry using a polishing pad typically larger than the electrode grid array. The polishing process may be continued until the dielectric above the electrode is of desirable thickness (from less than 500 nm to 15 μm or more) above the electrode. The polishing step may also smooth the surface to a surface roughness of roughness value less than 0.5 μm, 1 μm, and more preferably to smoother than 500 nm, or 200 nm, 100 nm, or less. After polishing, a follow-up with a hydrophobic coating may be desirable. The thin smooth surface with or without hydrophobic coating may provide sufficient electrowetting forces to move droplets at lower voltages.

Polymer Film as a Smooth Dielectric Surface

Referring to FIG. 8E, in some cases, a thin polymer film (830) (1 μm to 20 μm) may be used to form a smooth dielectric surface directly above the electrode array. In some embodiments, pre-processing may not be required to patch some of the canyons with a photoresist, epoxy or potting compound—these cavities (832) may be left filled with air. Instead, the film may be applied directly to the unmodified electrode surface. In these cases, the film may first be stretched (834) to remove any wrinkles and may then be bonded to the surface of the electrodes. Polymers films of low surface free energy may be used for such use. Many fluorinated polymers such as, for example, PTFE (polytetrafluoroethylene), ETFE (ethylene tetrafluoroethylene), FEP (fluorinated ethylene propylene), PFA (perfluouroalkoxy alkane), and other fluoropolymers with low surface energy may be suitable for electrowetting. Polydimethylsiloxane (PDMS) is another material with low surface energy that may be used as dielectric for electrowetting. These low surface energy polymer films may have an additional layer of hydrophobic material to reduce the surface energy further for low adhesion and good electrowetting droplet motion. Films made from polymers with slightly higher surface free energy such as, for example, polypropylene, polyimide, Mylar, polyvinylidene fluoride (PVDF) may also be suitable for electrowetting, however they may require an additional hydrophobic material coating or surface modification to aid droplet motion.

Creating a Final Slippery Surface Finish

A surface of an electrowetting microfluidic device may be further treated to reduce or eliminate adherence of the liquid droplet to the top surface. This additional treatment may permit a droplet to be repeatedly moved from one location to another by lowering actuation voltages. To turn the smooth dielectric surface into a slippery, low-adherence surface for a droplet, the surface of the dielectric material may be turned into a hydrophobic surface via chemical modification or surface topography modification. Alternatively, this slippery surface may be created by creating a thin layer of lubricating liquid on the smooth dielectric or directly on the electrode array. The hydrophobic coating material may be such that a 1 μl droplet on a surface tilted at angle of 3° or more slides away. The following section will describe these methods in detail.

Modifying Solid Dielectric to Achieve Desired Surface Energy

In some embodiments, the smooth dielectric surface may not have sufficiently low surface energy to allow for droplet motion induced by electrowetting. To reduce the surface energy further, the dielectric surface may be modified chemically or topographically.

In some embodiments, the smooth dielectric surface may have too low surface energy to allow for droplet motion induced by electrowetting. To increase the surface energy, the dielectric surface may be modified chemically or topographically.

Surface Chemistry Modification (Functionalization)

Referring to FIG. 8F, the surface energy may be reduced by chemical modification, for example, by coating over the electrodes (120), dielectric (130), or any combination thereof with hydrophobic or low-surface energy materials (840) such as, for example, fluorocarbon based polymers (fluoropolymers), polyethylenes, polypropylenes, or other hydrophobic surface coatings.

The surface coating may be applied by one or more methods, including spin coating, dip coating, spray coating, drop coating, chemical vapor deposition, or other methods.

In some cases, it may be desirable to choose a conformal coating that may act as both a dielectric (to insulate the droplets from the charge of the electrical pads while allowing the electric field to propagate) and as a hydrophobic or hydrophilic or both coating (to reduce adhesion and allow smooth droplet motion).

Surface Topography Modification

To induce hydrophobicity on the surface of the dielectric, its topography may be modified at a microscopic level. Such modifications may include patterning the surface to create microscopic pillar (micropillars) or deposition of microspheres.

Creating Micropillars

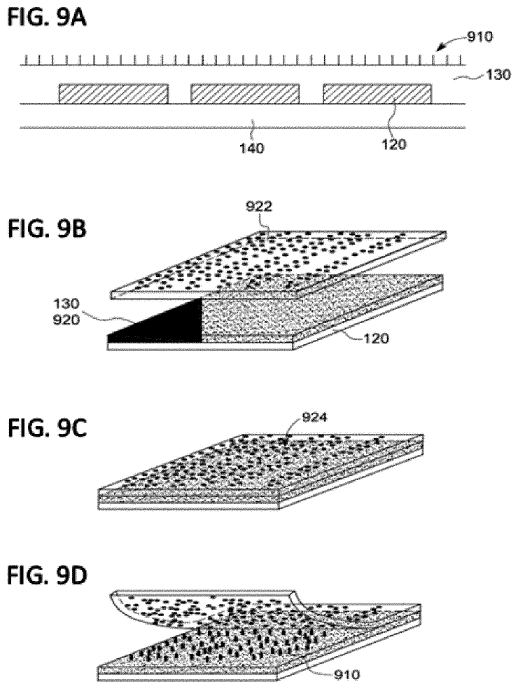

Referring to FIG. 9A, micropillar structures (910) may be created on a film of dielectric layer (130). This topmost layer over the electrode array may act as hydrophobic surface.

Referring to FIGS. 9B, 9C, and 9D, micropillar structures may be created by first heat bonding polymer films (920, 130) of, for example, polypropylene, polytetrafluoroethylene (PTFE), Mylar, Ethylene tetrafluoroethylene (ETFE), fluorinated ethylene propylene (FEP), perfluoroalkoxy alkane (PFA), other fluoro-carbon based polymers, or other low-surface energy polymers, as a dielectric over the electrodes. The polymer surface may be pressed against a micropillar template (922), such as, for example, a polycarbonate membrane that has holes of dimension 1 μm to 5 μm (or other porous membrane as templates). Referring to FIG. 9C, with heat and pressure (924), the polycarbonate micropillar template may impress itself into the dielectric film. Referring to FIG. 9D, when the polycarbonate membrane is peeled away, it may leave microscopic pillar like structures (910).

In another alternative, micropillar structures (910) may be created with, for example, polydimethylsiloxane (PDMS) elastomer on the planarized electrode array (after the electrode array is planarized). In this method the PDMS elastomer may be cast as a thin film through a spin coating method. The polycarbonate membrane may then be pressed against the PDMS surface. The PDMS membrane may be cured to solidify. The polycarbonate membrane may then be dissolved.

In another alternative, a polymer (e.g., ETFE, PTFE, FEP, PFA, PP, Mylar, and PVDC) or elastomer (e.g., PDMS, Silicone) may be bonded to the electrode array, and then it may be etched with laser to create micropillars.

In another alternative, a photoresist material may be deposited on to the electrode array, and then it may be etched with laser to create micropillars. The photoresist may also be patterned and etched using photolithography techniques.

Microspheres

Referring to FIG. 9E, an alternate method for modifying topography to achieve a slippery or low-adherence surface may be to deposit microspheres (930) of particle size from less than 200 nm to 2 μm or more. The microspheres may be tightly packed to make the surface hydrophobic. A good candidate for such microsphere particles may be silica beads, for example. In order to make the surface slippery, these microspheres may be covered by organofunctional alkoxysilane molecules. Alternately, fluorocarbon-based microspheres (PTFE, ETFE) may be deposited and may not need additional coating.

Slippery Liquid Coating and Liquid-On-Liquid Electrowetting (LLEW)

Droplets on Thin Film Liquid Layer in LLEW

In LLEW, a droplet may ride on a thin film of lubricating, low surface energy oil. The thin film of oil may be formed on a low surface energy textured solid surface. The textured solid and the lubricating oil may be selected such that the lubricating oil wets the solid entirely, and remains non-interacting with the liquid of the droplet. Once the bulk of the textured solid is filled with oil, a thin layer of oil may be formed just above the oil-filled body. The self-leveling nature of the oil layer on the top may hide any non-uniformity in the topography of the underlying surface. Thus, a surface of an electrode array with very high roughness (tens of micrometers) may be translated to a nearly-molecular-level smooth surface with a thin film of lubricating oil.

This molecular-level smooth surface may offer very little friction to droplet motion, and droplets may experience little to no droplet pinning Droplets on such a smooth surface may have very small contact angle hysteresis (as low as 2°). The resulting low contact angle hysteresis and absence of droplet pinning may lead to very low actuation voltage (from 1V to 100V) with robust droplet manipulation.

Oil in the bulk of the solid may be trapped within irregularities or pores that make up the texture of the solid. As opposed to a layer of oil on a smooth textureless surface, oil in a textured solid may have sufficient affinity for and molecular interaction with the solid's surface to reduce the influence of gravity. The trapping of the oil within the texture may allow the surface to retain its oil layer and its characteristics when inclined or upside down. Since the oil does may not leave the surface of the solid, the droplet being moved may ride on the lubricating oil and it may interact with the surface of the lubricating oil and not with the underlying textured solid. As a result, the droplet may leave little to no trail on the underlying solid. If the oil is immiscible with the droplet, a droplet may move on the liquid film layer without any contamination between two consecutive droplets crossing paths.

The textured solid may be made of regular or irregular micro-textures. Examples include:
A solid with regularly spaced microscopic pillar structures, with micron-scale spacing.
A solid with regularly spaced voids; the voids may be of any arbitrary shape.
A random matrix of fibers.
A solid with irregularly spaced microscopic pillar structures, with micron-scale spacing.
A solid with randomly spaced voids; the voids may be of any arbitrary shape.
A porous material such as porous Teflon, porous polycarbonate, porous polypropylene, porous paper and porous fabric may be used as irregular or regular micro-textured solid.

The lubricating oil may be any low-energy oil such as silicone oil, DuPont Krytox oil, Fluorinert FC-70 or other oil. The lubricating oil may be selected such that the oil is immiscible with the liquid droplets. A lubricant that is immiscible with the droplet solvent may improve the ability of the droplet to ride over the lubricant or oil with less diffusion of contents from the droplet into the oil and vice-versa. The viscosity of the lubricating oil may affect droplet mobility during electrowetting; with lower viscosity promoting higher mobility. Suitable lubricating oils may be non-volatile and immiscible with the riding droplet of interest. If the droplet contains biological constructs, a biocompatible oil may be desirable. In a LLEW device with on-chip heating elements for incubation and for thermocycling (for example, for polymerase chain reaction), the oil may be selected to withstand heating and high temperatures. An oil with sufficiently high dielectric constant may reduce actuation voltage that induces droplet motion.

Creating Textured Solid for LLEW

In LLEW, the oil-filled textured solid may act as an electrical barrier between the electrode array and liquid droplet and may also provide the slippery surface for droplet motion. There are a number of different ways in which textured dielectric surface may be created on an electrode array.

A textured solid surface may be formed on an electrode array by binding a polymer or other dielectric material as a film. The film itself may be textured before bonding to the electrode array. Alternatively, a non-textured film may be bonded on to the electrode array, and then textured either by laser etching, chemical etching or photolithography techniques, for example.

Alternatively, a layer of photosensitive material such as a photoresist (SU-8) may be coated onto the electrode array. The photoresist may be patterned by chemical etching, laser etching or any other photolithography techniques.

Alternatively, textured solids may be created by coating very thin layers of elastomeric material such as PDMS onto the electrode array and then using soft lithography techniques to selectively create pores. Following the creation of a thin elastomeric layer, the surface of the PDMS may also be laser etched to create textures.

Alternatively, textured solids may be created as follows—
Applying a conformal coating or liquid photoimageable (LPI) solder mask or dry film photoimageable solder mask
Etching the surface of this coating with a laser or by physical stamping.
Growing a mesh of polymer substance directly on the electrode array.
Growing one molecule at a time to achieve the required structure.

Applying Lubricating Oil onto the Textured Solid

The textured solid layer may be filled with lubricating oil by spin-coating, spraying, dip-coating, brushing, drop coating, or by dispensing from a reservoir.

The lubricating oil may be kept from flowing out of the LLEW chip by creating physical or chemical barriers at the periphery of the device.

### Unique Properties of Liquid-On-Liquid Electrowetting (LLEW)

The LLEW array has two unique properties that are desirable for biological sample manipulation. The electrowetting actuation voltage may be lowered significantly because a LLEW array has such a smooth surface. Additionally, the LLEW surface architecture may reduce cross-contamination between samples by lowering the trail droplets leave behind as well as improving cleaning mechanism.

Low Actuation Voltage

A nearly molecular level smoothness of oil surface on an LLEW electrode array may reduce or eliminate droplet pinning A droplet made of an aqueous solution riding on the oil surface may experience little to no drag from the surface and hence have a small difference between the advancing and receding angle. The elimination of these two phenomena may result in low actuation voltage. Droplets may be actuated at voltages as low as 1V.

In a LLEW device, a droplet riding on a thin layer of oil may never physically come in contact with the solid dielectric substrate below the oil. This may reduce or eliminate the amount of material left behind and hence cross-contamination between samples that go over the same spot.

Cleaning by Washing an LLEW Device Surface

When a LLEW device is contaminated with a solid particle such as dust, a droplet may be maneuvered over the contaminant to remove the contaminant from the liquid film surface as a part of a cleaning routine. This cleaning routine may be further extended to clean the entire surface of electrowetting device. For example, a cleaning routine may be used between two biological experiments on a LLEW microfluidic chip to reduce cross contamination. In some cases, when a droplet stays at a location for a long period of time, a few molecules may diffuse from the droplet into the oil below. Any residue left behind by a droplet through diffusion may also be cleaned with similar washing routines.

As droplets are transported on a LLEW device, the droplets may carry and deplete the oil film from the surface. The oil on the surfaces may be replenished by injecting oil from an external reservoir; for example, from an inkjet cartridge, syringe pump or other dispensing mechanisms.

The lubricating oil surface may be washed away entirely and replaced with a fresh layer of oil to prevent cross contamination between two consecutive experiments.

### Applications of Electrowetting

Arbitrarily-Large Open Face

Droplets may be manipulated on an open surface, without sandwiching them between the electrode array and a cover plate (either a neutral glass, or an upper electrode array, or simply just a large ground electrode). A cover plate above the droplet may be used that does not physically make contact with the droplet.

Electrode arrays and electrowetting on an open surface and arbitrarily large area may allow for actuation of droplets of volumes between 1 nanoliter from 1 milliliter (6 orders of magnitude apart). This implementation shows multi-scale fluid manipulation digitally on a single device.

Two-dimensional arrays (grids) of electrodes of arbitrarily-large size may be prepared for electrowetting droplet actuation. Two-dimensional arrays may allow for multiple paths for droplets compared to prescribed one-dimensional tracks. These grids may be leveraged to avoid cross-contamination between droplets of two different compositions. For example, a two-dimensional grid may allow for multiple droplets actuated in parallel. Droplets carrying different solutes may be run on separate parallel tracks to reduce contamination. Multiple distinct biological experiments may be run in parallel.

Droplet Motion, Merging and Splitting

A droplet may be moved, merged, split, or any combination thereof on an open surface electrowetting device. The same principles apply to two plate configuration (droplet sandwiched).

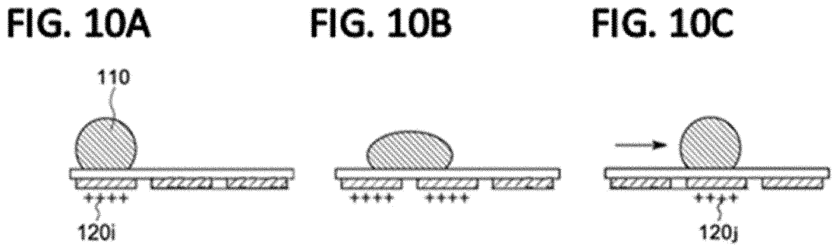

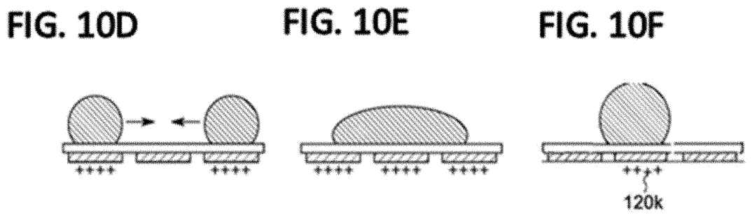

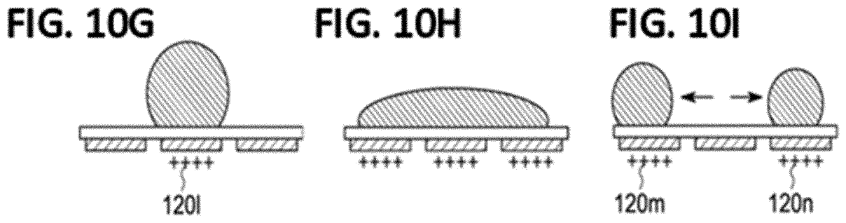

FIGS. 10A, 10B, and 10C show motion of a droplet (110) on an array of electrodes (120). In FIG. 10A, applying a voltage to an electrode (120i) may make the overlying surface hydrophilic and a droplet can then wet it. When voltage is applied on two neighboring electrodes, the droplet may spread across both actuated electrodes, as seen in FIG. 10B. When voltage is removed from electrode (120i) and applied to another adjacent electrode (120j), the surface returns to original hydrophobic state and the droplet may be pushed out, as shown in FIG. 10C. By sequentially controlling the voltage applied to an electrode grid, a droplet's position on a surface may be precisely controlled.

Referring to FIGS. 10D, 10E, and 10F, two droplets may be merged. When two droplets are pulled towards the same electrode 120k, they may naturally merge due to surface tension. This principle may be applied to merge a number of droplets to create a larger volume droplet spreading across multiple electrodes.

Referring to FIGS. 10G, 10(h), and 10I, a droplet may be split into two smaller ones through a sequence of voltages, applied across multiple electrodes (at least three electrodes). In FIG. 10G, a single large droplet is consolidated above a single electrode (1201). In FIG. 10H, an equal voltage is applied to three adjacent electrodes simultaneously, and this may cause the single droplet to spread across the three adjacent electrodes. In FIG. 10I, turning off the center electrode (1201) may force the droplet to move out to the two outer electrodes (120m and 120n). Due to the equal potential on both of the two outer electrodes, the droplet may then split into two smaller droplets.

Lab in a Box (Desktop Digital Wetlab)

Any combination of the manufacturing methods described so far may be used for the application described in this section.

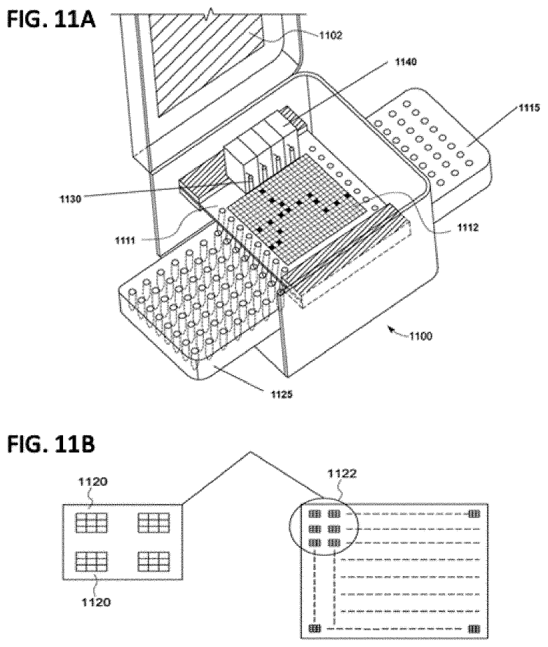

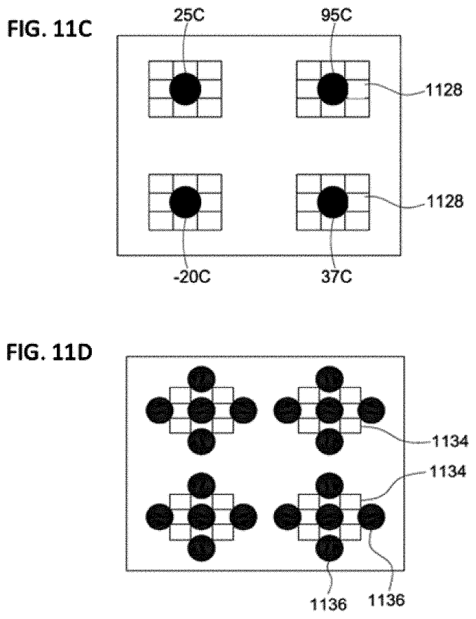

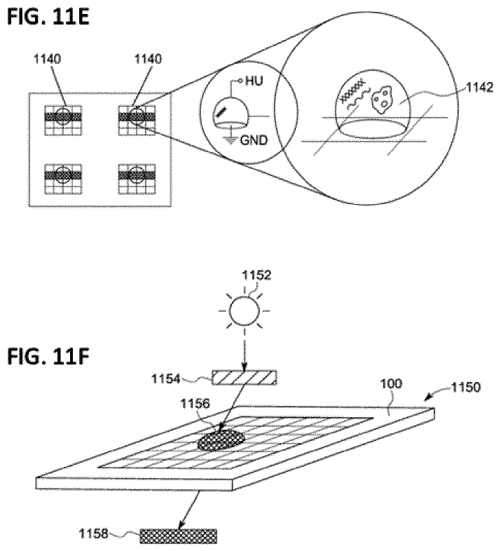

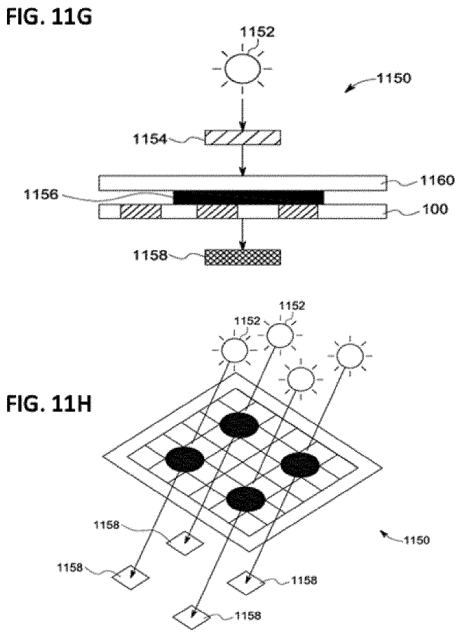

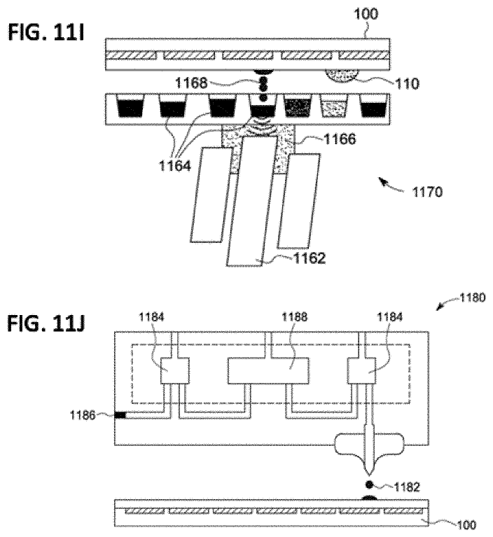

FIG. 11A shows a digital microfluidic based “desktop digital wetlab” (1100). This device may provide a general-purpose machine that may automate a large variety of biological protocols/assays/tests. The box may have a lid that can be opened and closed. The lid may have a clear window (1102) to view the motion of droplets on the electrode array, which may be formed as a digital microfluidic chip. The box may house a digital microfluidic chip (1111) capable of moving, merging, splitting droplets, in which the droplets may carry biological reagents. The microfluidic chip may also have one or more heaters or chillers that may be able to heat droplets from as high as 150° Celsius or more or cool the droplets from as low as −20° Celsius or less.

Droplets may be dispensed onto the chip through one or more “liquid dispenser(s)” (1130). Each liquid dispenser may be, for example, an electro-fluidic pump, syringe pump, simple tube, robotic pipettor, inkjet nozzle, acoustic ejection device, or other pressure or non-pressure driven device. Droplets may be fed in to the liquid dispenser from a reservoir labeled “reagent cartridge” (1140). The “lab-in-a-box” may have up to a several hundred reagent cartridges interfacing directly with the microfluidic chip.

Droplets may be moved from the digital microfluidic chip on to micro plates (e.g., 1115 and 1125). Microplates may be plates with wells that can hold samples. Microplates may have anywhere from one to a million wells on a single plate. Multiple microplates may interface with the chip in the box. To dispense droplets from the microfluidic chip to the microplate, electrowetting chips with various geometries may be used. In some cases, the dispensing chip may be in the form of a cone resembling a pipette tip. In another embodiment, the dispensing aperture may be a cylinder. In another embodiment, the dispensing apparatus may be two parallel plates with a gap in between. In another embodiment, the dispensing apparatus may be a single open surface with at least one droplet moving on the open surface. The dispensing mechanism may also use a number of other mechanisms such as, for example, electrofluidic pumps, syringe pump, tubes, capillaries, paper, wicks or even simple holes in the chip.

The “lab-in-a-box” may be climate controlled to regulate the internal temperature, humidity, lighting conditions, droplet size, pressure, droplet coating, oxygen concentration, or any combination thereof. The inside of the box may be at vacuum. The inside of the box may be purged with a combination of a variety of gasses. The gasses may include air, argon, nitrogen, or carbon dioxide.

The digital microfluidic chip (1111) at the center of the box may be removed, washed and replaced.

The digital microfluidic chip (1112) at the center of the box may be disposable.

The digital microfluidic device may include sensors to perform various assays, for example optical spectroscopy, or sonic transducers.

The digital microfluidic device may include a magnetic bead-based separation unit for DNA size selection, DNA purification, protein purification, plasmid extraction and any other biological workflow that uses magnetic beads. The device may perform a number of simultaneous magnetic bead-based operations—from one to a million on a single chip.

The box may be equipped with multiple cameras looking at the chip from the top, sides and bottom. The cameras may be used to locate droplets on the chip, to measure volumes of droplets, to measuring mixing, and to analyze reactions in progress. Information from these sensors may be provided as feedback to computers that control the electrical flow to the electrodes, so that the droplets may be accurately controlled to achieve high throughput rates with accurate drop positioning, mixing, etc. Information from these sensors may be provided to a machine learning algorithm or neural network.

The lab-in-a-box may be used to perform microplate operations such as plate stamping, serial dilution, plate replicate and plate rearray.

The lab-in-a-box may include equipment for PCR amplification and DNA assembly (Gibson Assembly, Golden Gate Assembly), molecular cloning, DNA library preparation, RNA library preparation DNA sequencing, single cell sorting, cell incubation, cell culture, cell assay, cell lysing, DNA extraction, protein extraction, RNA extraction, RNA and cell-free protein expression.

Processing Stations

An electrowetting chip (with or without a lab-in-a-box enclosure) may include one or more stations for various functions.

Mixing and Partitioning Stations

Referring to FIG. 11B, an electrowetting device may incorporate one or more mixing stations (1120). On the left is a 2×2 collection of electrowetting-based mixing stations that may be operated in parallel. A single mixing station (1120) may have a 3×3 grid of actuation electrodes. Each mixing station (1120) may be used to mix biological samples, chemical reagents, and liquids. For example, droplets of two reagents may be brought together at a mixing station, and then mixed by running the merged droplet around the outer eight electrodes of the 3×3 grid, or running through other patterns designed to mix the two original droplets. The center-to-center spacing between each mixing station may be 9 mm, equivalent to the spacing of a standard 96-well plate.

The mixing stations (1120) may be extended to have a number of different configurations. Each single mixer may be comprised of any number of actuation electrodes in an A×B pattern. Additionally, the spacing between mixers is arbitrary and may be altered to fit the application (such as other SDS plates). A parallel mixing station may also have any number of individual mixers in an M×N pattern (1122). Parallel mixing stations may have any configuration of top plate including but not limited to an open face, a closed plate, or a closed plate with liquid entry holes.

The mixing stations (1120) may be used as partitioning stations. Partitioning stations may use the electrowetting force to partition one droplet into a plurality of droplets. In addition to the electrowetting force, other methods can be used to partition droplets, including dielectrowetting forces, dielectrophoretic effects, acoustic forces, hydrophobic knives, or any combination thereof. Partitioning may be used for a variety of purposes, such as dispensing reagents or samples. Partitioned droplets may then be mixed with other droplets to execute a reaction in the other droplets. The partitioned droplets may be analyzed by the same sensors and methods as non-partitioned droplets.

Partitioned droplets may be mixed with target droplets to maintain a constant volume of at least one target droplet, where the at least one target droplet has lost volume (for example due to evaporation, being partitioned itself, etc.). The instruction to mix the droplets may come from an attached device such as a computer or smartphone.

Temperature Control Station

Referring to FIG. 11C, an electrowetting chip may include one or more temperature control station(s) (1128). Each station (1128) may integrate one or more functions to be applied to liquid samples such as mixing, heating (for example, to temperatures up to and including 150° Celsius), cooling (for example, down to and including −20° Celsius), compensating for fluid loss due to evaporation as well as homogenizing temperature of a sample. Heating or cooling may be accomplished by metal traces, foil heaters, Peltier elements external to the substrate, or a combination thereof. In some cases, the individualized heating elements may permit each station to be controlled to a separate temperature, for example, −20° C., 25° C., 37° C., and 95° C., depending on the heat transfer power of each element and the heat conduction levels between stations.

A parallel temperature control station may be configured in any of the same configurations as a parallel mixing station.

The heater may have a maximum temperature less than or equal to about 150° C., 125° C., 100° C., 75° C., 50° C., 25° C., or less. The heater may be thermoelectric, resistive, or heated by a heat transfer medium (e.g., a recirculated hot water loop). The cooler may have a minimum temperature greater than or equal to about −50° C., −25° C., −10° C., −5° C., 0° C., 10° C., or more. The cooler may be thermoelectric, evaporative, or cooled by a heat transfer medium (e.g., a water chiller).

The temperature control stations as described herein may configured to precisely control and manipulate the temperature applied to the liquid sample. In some embodiments, the temperature control stations are configured to heat/cool the liquid samples by about 0.1° C. to about 1° C. In some embodiments, the temperature control stations are configured to heat/cool the liquid samples by about 0.1° C. to about 0.2° C., about 0.1° C. to about 0.3° C., about 0.1° C. to about 0.4° C., about 0.1° C. to about 0.5° C., about 0.1° C. to about 0.6° C., about 0.1° C. to about 0.7° C., about 0.1° C. to about 0.8° C., about 0.1° C. to about 0.9° C., about 0.1° C. to about 1° C., about 0.2° C. to about 0.3° C., about 0.2° C. to about 0.4° C., about 0.2° C. to about 0.5° C., about 0.2° C. to about 0.6° C., about 0.2° C. to about 0.7° C., about 0.2° C. to about 0.8° C., about 0.2° C. to about 0.9° C., about 0.2° C. to about 1° C., about 0.3° C. to about 0.4° C., about 0.3° C. to about 0.5° C., about 0.3° C. to about 0.6° C., about 0.3° C. to about 0.7° C., about 0.3° C. to about 0.8° C., about 0.3° C. to about 0.9° C., about 0.3° C. to about 1° C., about 0.4° C. to about 0.5° C., about 0.4° C. to about 0.6° C., about 0.4° C. to about 0.7° C., about 0.4° C. to about 0.8° C., about 0.4° C. to about 0.9° C., about 0.4° C. to about 1° C., about 0.5° C. to about 0.6° C., about 0.5° C. to about 0.7° C., about 0.5° C. to about 0.8° C., about 0.5° C. to about 0.9° C., about 0.5° C. to about 1° C., about 0.6° C. to about 0.7° C., about 0.6° C. to about 0.8° C., about 0.6° C. to about 0.9° C., about 0.6° C. to about 1° C., about 0.7° C. to about 0.8° C., about 0.7° C. to about 0.9° C., about 0.7° C. to about 1° C., about 0.8° C. to about 0.9° C., about 0.8° C. to about 1° C., or about 0.9° C. to about 1° C. In some embodiments, the temperature control stations may be configured to heat/cool the liquid samples by about 0.1° C., about 0.2° C., about 0.3° C., about 0.4° C., about 0.5° C., about 0.6° C., about 0.7° C., about 0.8° C., about 0.9° C., or about 1° C. In some embodiments, the temperature control stations may be configured to heat/cool the liquid samples by at least about 0.1° C., about 0.2° C., about 0.3° C., about 0.4° C., about 0.5° C., about 0.6° C., about 0.7° C., about 0.8° C., or about 0.9° C. In some embodiments, the temperature control stations may be configured to heat/cool the liquid samples by at most about 0.2° C., about 0.3° C., about 0.4° C., about 0.5° C., about 0.6° C., about 0.7° C., about 0.8° C., about 0.9° C., or about 1° C. In some embodiments, the temperature control stations may be configured to heat/cool the liquid samples by about 0.5° C. In some embodiments, the temperature control stations are configured to heat/cool to the liquid samples to maintain the temperature of the liquid samples within about 0.1° C. to about 1° C. of a target temperature.

Magnetic Bead Station

Referring to FIG. 11D, a magnetic bead wash station (1134) may contain samples with nucleic acids, proteins, cells, buffers, magnetic beads, wash buffers, elution buffers, and other liquids (1136) on an electrode grid. The station may be configured to mix samples and reagents, apply heating or other processes, in sequential order to perform such actions as nucleic acid isolation, cell isolation, protein isolation, peptide purification, isolation or purification of biopolymers, immunoprecipitation, in vitro diagnostics, exosome isolation, cell activation, cell expansion, isolation, or any combination thereof of a specific biomolecule. In addition to mixing and heating of liquids, each magnetic bead station may have the ability to locally turn on and turn off a strong and varying magnetic field, which in turn may cause magnetic beads to move, for example, to the bottom of the electrowetting chip. Each magnetic bead station may also have the ability to remove excess supernatant liquids and wash liquids through electrowetting forces or through other forces.

In some cases, the sample may be on an open surface with single plate electrowetting device. In some cases, the samples may be sandwiched between two plates. Multiple magnetic bead stations may be configured to be operated in parallel, as described above for parallel mixing stations.

Nucleic Acid Delivery Station

Referring to FIG. 11E, an electrowetting chip may include one or more nucleic acid delivery stations (1140). Each nucleic acid delivery station may be designed to insert genetic material (1142), other nucleic acids and biologics into cells through various insertion methods. This insertion may be performed by applying a strong electric field, applying a strong magnetic field, applying ultrasonic waves, applying laser beams, or other techniques. One or more nucleic acid delivery station may be configured as a singleton on an electrowetting device, or multiple nucleic acid delivery stations may be provided to operate in parallel.

Optical Inspection Station

Referring to FIGS. 11F, 11G, and FIG. 11H, one or more optical inspection stations (1150) that use optical detection and assay methods may be provided on an electrowetting device (100). A light source (1152) (e.g., broad spectrum light, single frequency, etc.) may be passed through optics (1154) to condition the light (which may include, for example, filters, diffraction gratings, mirrors, etc.) and illuminate a sample (1156) sitting on an electrowetting device. An optical detector (1158), which may be placed on the same or other side of the electrowetting device, may be configured to detect the spectrum of light passing through the sample for analysis. The optical inspection may be used for measuring, for example, concentration of nucleic acids, measuring quality of nucleic acids, measuring density of cells, measuring extent of mixing between two liquids, measuring volume of sample, measuring fluorescence of sample, measuring absorbance of sample, quantification of proteins, colorimetric assays, optical assays, or any combination thereof.

As shown in FIG. 11F, sample (1156) may be on an open surface with single plate electrowetting device (100). As shown in FIG. 11G, sample (1156) may be sandwiched between two plates (100, 1160). In some embodiments, the electrowetting chip and the electrodes may be transparent. In some embodiments, there may be a hole in the electrode on which the sample is located, to allow passing of light from the source through the sample to the optical detector, or to introduce samples, reagents, or reactants.

Referring to FIG. 11H, the optical detection may be performed on samples arranged in 2×2 sample format or 96 well plate format for optical detection or any M×N format to measure, for example, a million samples. The samples and corresponding measurement units may be arranged in any regular and irregular format.

Liquid Handling Station

Referring to FIGS. 111 and 11J, an electrowetting device may include one or more stations (1170, 1180) for loading biological samples, chemical reagents and liquids from a source well, plate, or reservoir onto an electrowetting chip (100).

In FIG. 11I, droplets may be loaded onto the electrowetting surface through acoustic droplet ejection. The source plate may hold liquids in wells (1164) and may be coupled with a piezoelectric transducer (1162) via an acoustic coupling fluid (1166). Acoustic energy from a piezoelectric acoustic transducer (1162) may be focused on to the sample in the well (1164). Note in FIG. 11I, electrowetting chip (100) is on top, and is inverted. Droplet (110) may adhere to electrowetting chip (100) because of the additional wetting force induced by the voltage, which contributes to the droplet-sorting function of apparatus (1170). A droplet (1168) ejected from a well (1164) by acoustic energy may adhere to the upper electrowetting device (100) or may be incorporated into a droplet that has been moved to the acoustic injection station.

Referring to FIG. 11J, an electrowetting device may include one or more stations (1180) designed to load biological samples, chemical reagents and liquids (1182) through a microdiaphragm pump (1184) based dispenser onto an electrowetting chip.

Either the acoustic droplet ejection technique of FIG. 11I or a microdiaphragm pump (1184) may be used to dispense fluid droplets of picoliter, nanoliter, or microliter volumes. An electrowetting device (100) placed above (FIG. 11I) the source plate captures the droplets (1168) ejected from the well plate and holds the droplets through electrowetting force. In this manner, samples containing, for example, biological reagents, chemical reagents, or a combination thereof may be dispensed onto an electrowetting chip. In some embodiments, (FIG. 11J), the electrowetting plate (100) is on the bottom and the acoustic droplet ejection transducer (1162 of FIG. 11I) or microdiaphragm pump (1184) is on the top. An input valve (1186) and larger microdiaphragm pump (1188) may be used to meter fluid flow into microdiaphragm pumps (1184). In this method the dispenser may be used to put samples on to an electrowetting chip on any arbitrary location.

In some cases, the electrowetting chip may be in an open plate configuration (no second plate) and droplets may be loaded directly onto the chip. In some cases, the electrowetting chip may have a second plate that sandwiches the droplet between an electrode array and a ground electrode. In some cases, the second plate (cover plate with or without ground) may have holes to allow the droplets in transit. In some cases, the droplets may be first loaded on an open plate and then a second plate may be added. In some cases, the liquids loaded onto the electrowetting chip is in preparation to execute a workflow when the chip is located inside of an acoustic liquid handler. In some cases, the liquids loaded onto the electrowetting chip is in preparation to execute a workflow when the chip is located external to the acoustic liquid handler or microdiaphragm pump. In some cases, the liquids are loaded onto the electrowetting chip when a workflow is being executed. In some cases, the acoustic droplet injector or microdiaphragm pump may be mounted on a locatable carriage (somewhat like a 3D printer nozzle) capable of motion over the electrowetting device, so that droplets may be injected at a specific point over the electrowetting device.

In some cases, both the source and destination may be electrowetting chips. In this scenario, the chips may be organized with their electrode arrays facing each other. In some cases, droplets may be transferred between the top and bottom electrowetting chips, back and forth between top using acoustic fields or electric fields and differential wetting affinities. Here, there may be acoustic transducers and coupling fluids on both sides of the chips. In some cases, samples on an electrowetting chip may be a source and the destination may be a well plate. Here samples may be transferred from the electrowetting chip on to a well plate using acoustic droplet ejection.

The spacing between the wells in a well plate and hence the format in which the liquids are loaded on to (and transferred away from) the electrowetting chip may be in standard well plate form or any other SDS well plate format or any arbitrary formats. The number of wells in the plate may be any arbitrary number in the range from one to a million.

The electrowetting chips loaded with samples from an acoustic droplet ejection device or microdiaphragm pump device may be combined with one or more of the functionalities of mixing station, incubation station, magnetic bead station, nucleic acid delivery station, optical inspection station, other functionalities, or any combination thereof.

### Alternative Implementations

Droplet on Open Surface (Single Plate Configuration) or Sandwiched Between Two Plates (Two Plate Configuration)

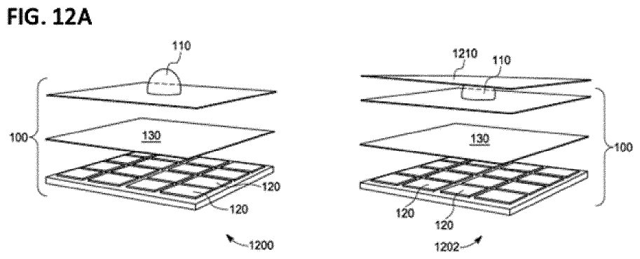

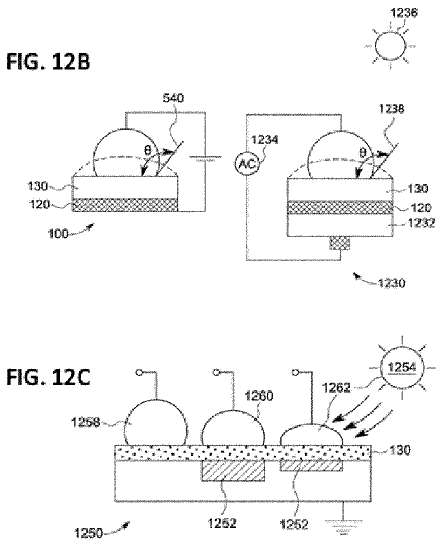

Referring to FIG. 12A, for electrowetting droplet manipulation, a droplet may either be placed on an open surface (single plate) (1200) or sandwiched between two plates (double plate) (1202). In the double plate configuration (1202), a droplet may be sandwiched between two plates (100, 1210), typically separated by 100 μm-500 μm. The two-plate configuration has electrodes (120) for providing actuation voltages on one side while the other side 1210 may provide a reference electrode (e.g., a common ground signal). A droplet's constant contact to the reference electrode in a two-plate configuration provides stronger force from the electric field on the droplet and hence robust control over droplets. In the two plate configuration (1202) droplets may be split at a lower actuation voltage. In the single plate configuration (1200) the actuation electrodes and the reference electrode are on the same side.

Two-plate electrowetting systems may be improved by the surface treatments described above. In two-plate systems, a droplet is sandwiched between plates separated by a small distance. The space between the plates may be filled with another fluid or just air. Smoothing the liquid-facing surfaces of the two plates to 2 μm, 1 μm, or 500 nm, using the techniques described above, may allow two-plate systems to operate at lower voltages, with reduced droplet pinning, reduced leave-behind tracks, reduced cross-contamination, and reduced sample loss.

Optoelectrowetting and Photoelectrowetting

Referring to FIGS. 12B and 12C, applying electric potential directly to an array of electrodes is one way of actuating droplets using electrowetting; however, there are alternate electrowetting mechanisms that differ from this conventional electrowetting mechanism. Two notable mechanisms, both of which use light for actuating the droplets, are described herein-optoelectrowetting and photoelectrowetting. The general principles for manufacturing the electrowetting arrays, creating a smooth surface and slippery surface described above are applicable not only to conventional electrowetting described earlier, but is also applicable to optoelectrowetting, photoelectrowetting and other forms of electrowetting.

A liquid film may be laid on a grid of photoconductors, to yield “liquid-on-liquid optoelectrowetting.” Instead of having a grid of electrodes under the lubricating liquid layer, the grid may be formed of light active photoconductor, either in a grid of pads, or as a single photoconductive circuit. Light shone on the photoconductor may form patterns and provide electrowetting effect. The textured solid and oil may be chosen to be sufficiently transparent to light so that the underlying surface is exposed to light to create differential wetting.

Optoelectrowetting

Referring to FIG. 12B, the optoelectrowetting mechanism (1230) may use a photoconductor (1232) underneath the conventional electrowetting circuit (100, left side), with an AC power source (1234) attached. Under normal (dark) conditions, the majority of the system's impedance lies in the photoconducting region (1232), and therefore the majority of the voltage drop may occur here. However, when light (1236) is shone on the system, carrier generation and recombination causes the conductivity of the photoconductor (1232) to spike and the voltage drop across the photoconductor (1232) reduces. As a result, a voltage drop occurs across the insulating layer (130), changing the contact angle, (540) vs. (1238), as a function of the voltage.

Photoelectrowetting

Referring to FIG. 12C, photoelectrowetting (1250) is a modification of the wetting properties of a surface (typically a hydrophobic surface) using incident light. Whereas ordinary electrowetting is observed in a droplet sitting on a dielectric coated conductor (liquid/insulator/conductor stack 110/130/120), photoelectrowetting may be observed by replacing the conductor (120) with a semiconductor (1252) (liquid/insulator/semiconductor stack).

Incident light (1254) above the band gap of semiconductor (1252) creates photo-induced carriers via electron-hole pair generation in the depletion region of the underlyingsemiconductor (1252). This leads to a modification of the capacitance of the insulator/semiconductor stack (130/1252), resulting in a modification of the contact angle of a liquid droplet resting on the surface of the stack. The figure illustrates the principle of the photoelectrowetting effect. At zero bias (0V) the conducting droplet (1258) has a large contact angle (left image) if the insulator is hydrophobic. As the bias is increased (positive for a p-type semiconductor, negative for an n-type semiconductor) the droplet (1260) spreads out—i.e. the contact angle decreases (middle image). In the presence of light (1254) (having an energy superior to the band gap of the semiconductor 1252) the droplet (1262) spreads out more due to the reduction of the thickness of the space charge region at the insulator/semiconductor interface (130/1252).

### Computer Systems

Various processes described herein may be implemented by appropriately programmed general purpose computers, special purpose computers, and computing devices. Typically, a processor (e.g., one or more microprocessors, one or more microcontrollers, one or more digital signal processors) will receive instructions (e.g., from a memory or like device), and execute those instructions, thereby performing one or more processes defined by those instructions. Instructions may be embodied in one or more computer programs, one or more 10 scripts, or in other forms. The processing may be performed on one or more microprocessors, central processing units (CPUs), computing devices, microcontrollers, digital signal processors, or like devices or any combination thereof. Programs that implement the processing and the data operated on, may be stored and transmitted using a variety of media. In some cases, hardwired circuitry or custom hardware may be used in place of, or in combination with, some or all 15 of the software instructions that can implement the processes. Algorithms other than those described may be used.

Programs and data may be stored in various media appropriate to the purpose, or a combination of heterogenous media that may be read and/or written by a computer, a processor or a like device. The media may include non-volatile media, volatile media, optical or magnetic 20 media, dynamic random access memory (DRAM), static ram, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge or other memory technologies. Transmission media include coaxial cables, copper wire and fiber optics, including 25 the wires that comprise a system bus coupled to the processor.

Databases may be implemented using database management systems or ad hoc memory organization schemes. Alternative database structures to those described may be readily employed. Databases may be stored locally or remotely from a device which accesses data in such a database.

In some cases, the processing may be performed in a network environment including a computer that is in communication (e.g., via a communications network) with one or more devices. The computer may communicate with the devices directly or indirectly, via any wired or wireless medium (e.g. the Internet, LAN, WAN or Ethernet, Token Ring, a telephone line, a cable line, a radio channel, an optical communications line, commercial on-line service providers, bulletin board systems, a satellite communications link, or a combination thereof). Each of the devices may themselves comprise computers or other computing devices, such as those based on the Intel® Pentium® or Centrino™ processor, that are adapted to communicate with the computer. Any number and type of devices may be in communication with the computer.

A server computer or centralized authority may or may not be necessary or desirable. In various cases, the network may or may not include a central authority device. Various processing functions may be performed on a central authority server, one of several distributed servers, or other distributed devices

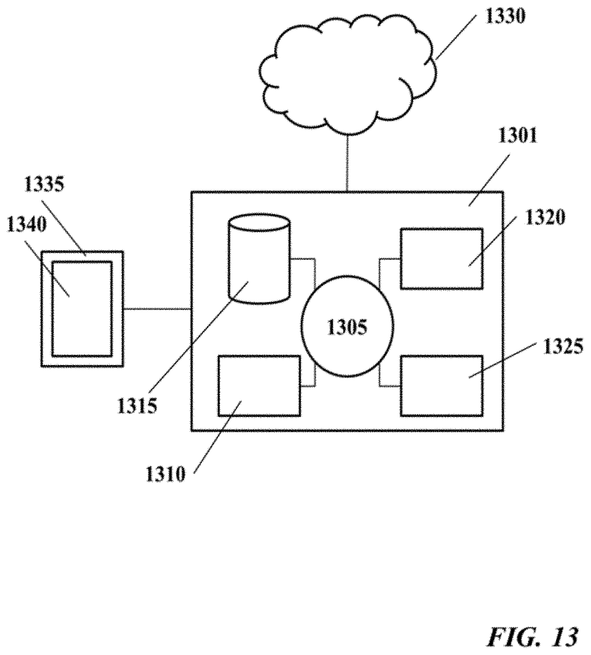

The present disclosure provides computer systems that are programmed to implement methods of the disclosure. FIG. 13 shows a computer system 1301 that is programmed or otherwise configured to manipulate a droplet, or a plurality thereof, on a system described herein. The computer system 1301 can regulate various aspects of sample manipulation of the present disclosure, such as, for example, droplet size, droplet volume, droplet position, droplet speed, droplet wetting, droplet temperature, droplet pH, beads in droplets, number of cells in droplets, droplet color, concentration of chemical material, concentration of biological substance, or any combination thereof. The computer system 1101 can be an electronic device of a user or a computer system that is remotely located with respect to the electronic device. The electronic device can be a mobile electronic device.

The computer system 1301 includes a central processing unit (CPU, also “processor” and “computer processor” herein) 1305, which can be a single core or multi core processor, or a plurality of processors for parallel processing. The computer system 1301 also includes memory or memory location 1310 (e.g., random-access memory, read-only memory, flash memory), electronic storage unit 1315 (e.g., hard disk), communication interface 1320 (e.g., network adapter) for communicating with one or more other systems, and peripheral devices 1325, such as cache, other memory, data storage, electronic display adapters, or any combination thereof. The memory 1310, storage unit 1315, interface 1320 and peripheral devices 1325 are in communication with the CPU 1305 through a communication bus (solid lines), such as a motherboard. The storage unit 1315 can be a data storage unit (or data repository) for storing data. The computer system 1301 can be operatively coupled to a computer network (“network”) 1330 with the aid of the communication interface 1320. The network 1330 can be the Internet, an internet, extranet, or any combination thereof, or an intranet, extranet, or any combination thereof that is in communication with the Internet. The network 1330 in some cases is a telecommunication, data network, or any combination thereof. The network 1330 can include one or more computer servers, which can enable distributed computing, such as cloud computing. The network 1330, in some cases with the aid of the computer system 1301, can implement a peer-to-peer network, which may enable devices coupled to the computer system 1301 to behave as a client or a server.

The CPU 1305 can execute a sequence of machine-readable instructions, which can be embodied in a program or software. The instructions may be stored in a memory location, such as the memory 1310. The instructions can be directed to the CPU 1305, which can subsequently program or otherwise configure the CPU 1305 to implement methods of the present disclosure. Examples of operations performed by the CPU 1305 can include fetch, decode, execute, and writeback.

The CPU 1305 can be part of a circuit, such as an integrated circuit. One or more other components of the system 1101 can be included in the circuit. In some cases, the circuit is an application specific integrated circuit (ASIC).

The storage unit 1315 can store files, such as drivers, libraries and saved programs. The storage unit 1315 can store user data, e.g., user preferences and user programs. The computer system 1301 in some cases can include one or more additional data storage units that are external to the computer system 1301, such as located on a remote server that is in communication with the computer system 1301 through an intranet or the Internet.

The computer system 1301 can communicate with one or more remote computer systems through the network 1330. For instance, the computer system 1301 can communicate with a remote computer system of a user (e.g., mobile electronic device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1301 via the network 1330.

Methods as described herein can be implemented by way of machine (e.g., computer processor) executable code stored on an electronic storage location of the computer system 1301, such as, for example, on the memory 1310 or electronic storage unit 1315. The machine executable or machine readable code can be provided in the form of software. During use, the code can be executed by the processor 1305. In some cases, the code can be retrieved from the storage unit 1315 and stored on the memory 1310 for ready access by the processor 1305. In some situations, the electronic storage unit 1315 can be precluded, and machine-executable instructions are stored on memory 1310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1301, can be embodied in programming Various aspects of the technology may be thought of as “products” or “articles of manufacture” typically in the form of machine (or processor) executable code, associated data, or any combination thereof that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code, data, or any combination thereof. Many of these forms of computer readable media may be involved in carrying one or more sequences of one or more instructions to a processor for execution.

The computer system 1301 can include or be in communication with an electronic display 1335 that comprises a user interface (UI) 1340 for providing, for example, information related to droplet manipulation, sample manipulation, or a combination thereof. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, provide additional liquid to a droplet, replace evaporated solvent of a droplet, map out a path for a droplet, or any combination thereof.

Video, input, and control of the system may be accessed through a web-based software application. User inputs through software may include, for example, droplet motion, droplet sizes, and images of the array, and user inputs may be recorded and stored in a cloud-based computing system. Stored user inputs may be accessed and retrieved in subsets or in entirety to inform machine-learning based algorithms. Droplet movement patterns may be recorded and analyzed for use in training navigation algorithms. Trained algorithms may be used for automation of droplet movement. Spatial fluid properties may be recorded and analyzed for use in training protocol optimization and generation algorithms. Trained algorithms may be used for optimizing biological and droplet movement protocols or in the generation of new biological and droplet movement protocols. Biological quality control techniques (e.g., amplification-based quantification methods, fluorescence-based, absorbance-based quantification, surface plasmon resonance methods, and capillary-electrophoretic methods to analyze nucleic acid fragment size) may be used to analyze the effectiveness of the workflows performed on the array. The data from these techniques may then be used as an input into machine learning algorithms to improve output. The process may be automated so that the system can iteratively improve the output.

### Dispensing Fluids and Droplet Creation

Various processes described herein may be implemented by dispensing liquids and creating droplets. Liquids may be dispensed individually or in combination to introduce liquids to an array. Introduced liquids may form a droplet, or a plurality thereof, on the array. A liquid handling system, robotic arm, acoustic dispenser, inkjet, or any combination thereof may be used to dispense fluids directly on an array or to a reservoir of the array. These dispensers may use channels, such as, for example, tubes, nozzles, pipettes, or any combination thereof as well as holes in the array.

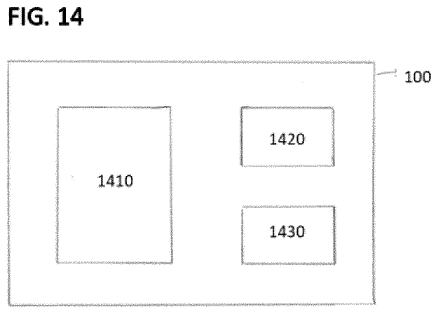

The array (100) may have regions that perform electrowetting on dielectric (EWOD, 1410), dielectrowetting (DEW, 1420), dielectrophoresis (DEP, 1430), or a combination thereof (FIG. 14). Liquids may be stored on the array in reservoirs. Droplets may be dispensed from the reservoirs on the array using DEP, DEW, EWOD, or any combination thereof, and subsequently actuated by EWOD. EWOD actuation of droplets can be used to move droplets of reagents to desired reactions. Alternatively, EWOD may be used to compensate for evaporative losses in existing droplets of the array. Additionally, a droplet may be split into two, three, four, five, six, or more droplets using EWOD, DEW, DEP, or any combination thereof.

### Alternative Droplet Actuation Mechanisms

Splitting a Droplet Using Hydrophobic “Slicer”

For a droplet on an array device it might be possible to slice a large droplet into one or more smaller droplets using a thin (sharp) hydrophobic structure (“slicer”). For this, the array device can be in any configuration described here (open or closed, with any arbitrary electrode configuration). The thin hydrophobic structure can be positioned above, below or on the sides of the droplet of interest.

Slicing the droplets this way is one mechanism to split a droplet into two equal droplets or aliquot a known amount of liquid from a larger droplet precisely. For this slicing/splitting mechanism to work, a droplet on the array device is shuttled using electromotive forces described herein (for example using electrowetting) and then dragged across the thin hydrophobic structure. The dragging action is done such that the thin structure cuts through the droplet vertically, horizontally or at an angle. This cutting action results in two droplets of equal or unequal volume. This method of slicing/cutting/splitting droplets is akin to how cheese grating is done.

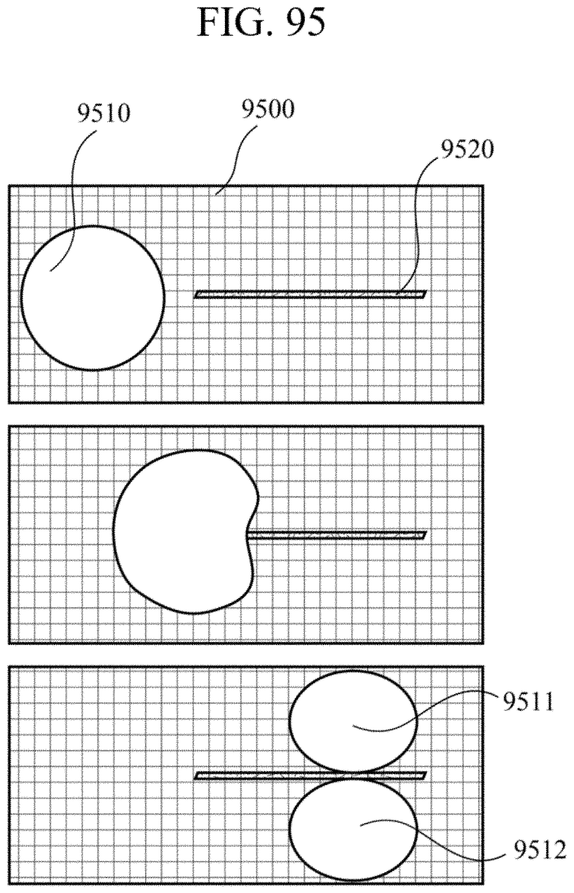

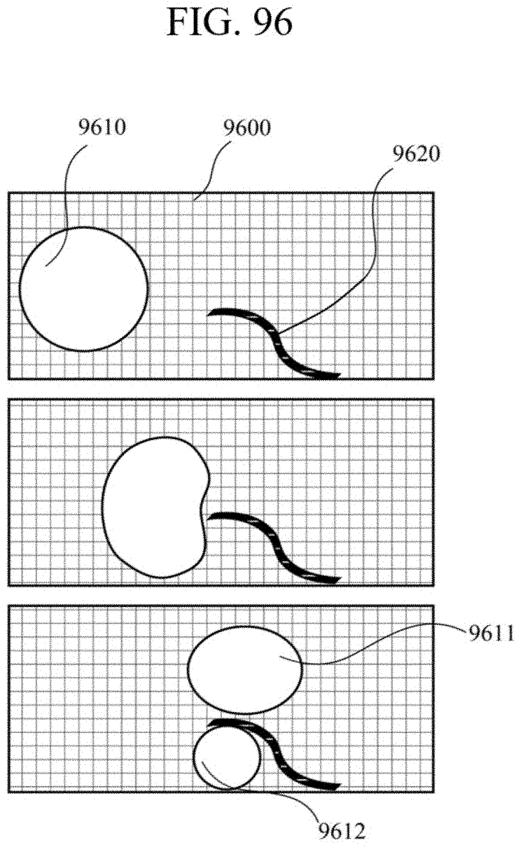

By carefully tuning how the droplet moves in relation to the “slicer”, it may be possible to regulate the volume of the droplets generated. It may also be possible to regulate the volume of the droplets generated by changing the size and shape of the thin hydrophobic “slicer”. A representative hydrophobic droplet “slicer” is shown in FIG. 95 (top view). As shown in FIG. 95, a droplet 9510 is cut into separted into two portions 9511, 9512 as it is dragged along a hydrophobic slicer/knife 9520. This technique may be used to cut/split a droplet in an array device 9500 with open configuration (droplets not making contact with top plate) or in closed configuration (droplets sandwiched between two plates). The hydrophobic knife could be attached to the any surface of the array device or a separate structure not part of the array device (for example a lid on the array device). In FIG. 95, the Slicer may be attached to a transparent top plate or to the top surface of the array device itself. In FIG. 96, the slicer 9620 may be attached to the side wall of the array device 9600 to split a droplet 9610 into two portions 9611, 9612. In some embodiments, the hydrophobic slicer may split a droplet into two approximately equal halves, as depicted in FIG. 95. In some embodiments, the slicer may split a droplet into two unequal portions, as depicted in FIG. 96. In some embodiments, the larger portion 9611 of the droplet 9610 continues to be processed on array 9600, while the smaller portion 9612 is disposed of.

Splitting by Binding Part of the Droplet to a Hydrophilic Picker

For a droplet on an array device it might be possible to aliquot a small amount of liquid from a large droplet using one or more tiny hydrophilic spots patterned on a largely hydrophobic surface (referred to as “picker”). For this, the array device can be in any configuration described here (open or closed, with any arbitrary electrode configuration). The “picker” with patterned hydrophilic sites can be positioned above, below or on the sides in relationship with the droplet of interest.

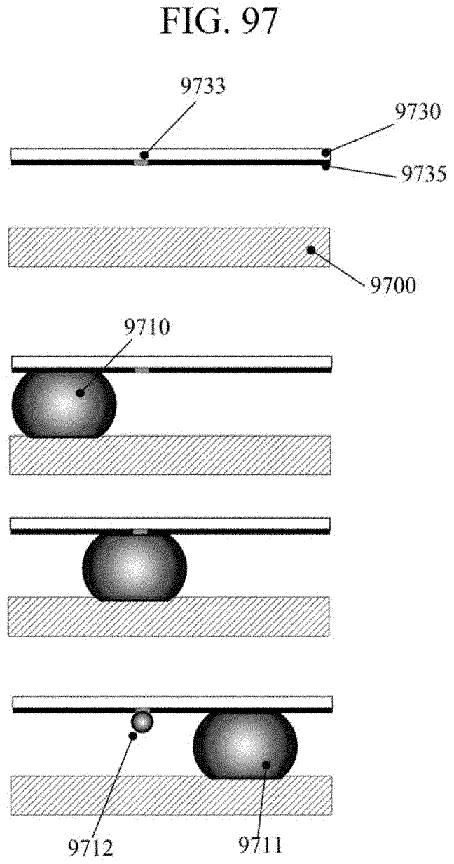

FIG. 97, depicts a picker 9720 is located above the droplet 9710, according to some embodiments. In some embodiments, a droplet 9710 is moved along an array 9700 having a two-plate configuration, wherein the bottom plate comprises the array 9700. The top plate 9730 may comprise a hydrophobic surface 9735 with a hydrophilic site or picker 9733 provide on the hydrophobic surface. While being shuttled, the one or more sides of the droplet surface make contact with the picker 9733. The surface 9735 the picker 9733 is provided may be largely hydrophobic, such that when the droplet 9710 comes in contact with the hydrophilic site 9733 a small and known portion of the droplet sticks (gets “picked”) to the hydrophilic site. This picking action is a way to split/aliquot a known amount of liquid 9712 from the large droplet 9711 which can then be used for many downstream applications.

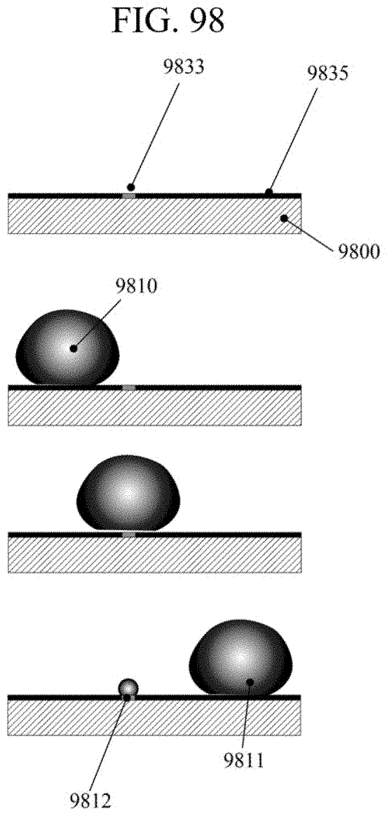

Alternatively, the removable film or the film frame or any component of the array device itself might be patterned to have a hydrophilic site to perform the picking operation. An example of this is show in FIG. 98. In some embodiments, as depicted in FIG. 98, a droplet 9810 is moved along an array 9800. The array may comprise a single sided configuration with only one plate comprising an EWOD or DEW array for droplet manipulation. In some embodiments, the array 9800 comprises a hydrophobic surface 9835. The hydrophobic surface 9835 may comprise a hydrophilic site 9833, such that when the droplet 9810 comes in contact with the hydrophilic site 9833 a small and known portion of the droplet sticks (gets “picked”) to the hydrophilic site. This picking action is a way to split/aliquot a known amount of liquid 9812 from the large droplet 9811 which can then be used for many downstream applications.

Alternatively, it may be possible to achieve the same functionality by making a slight modification to the “picker”. For example, the hydrophilic spots can be replaced with holes or capillaries of a known diameter. While a droplet makes contact with the “picker” surface, a small quantity of liquid is transferred from the larger droplet in to the holes or the capillary.

The droplets split using the mechanisms described herein can then be processed in various ways. For example, the aliquoted droplet may then be mixed with a fluorescent dye. This mixture can then be excited and read out using a flourometer (or an optical sensor) as described in other sections of the disclosure.

It may also be possible to mix the aliquoted droplet with a substantially larger droplet with the same solvent to dilute it. The process of splitting the droplet into tiny droplets and then diluting can be repeated over and over again to serially achieve various levels of dilutions as described herein.

### Computer-Vision

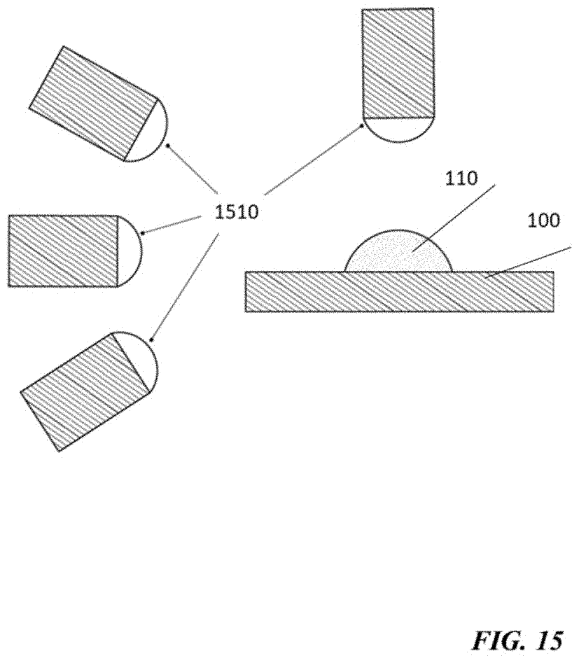

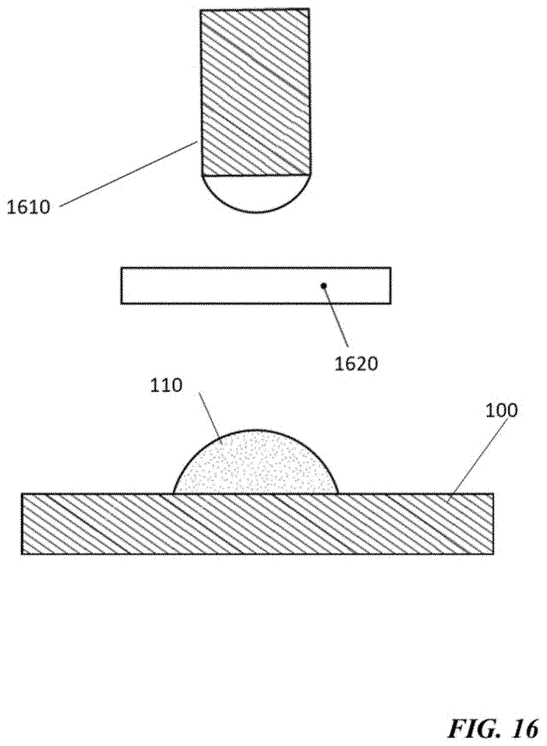

The configuration of a computer-vision system to monitor a droplet (110) on an array (100) described herein may include, for example, multiple light sources (1510) placed above the array, below the array, in the plane of the array, or any combination thereof (FIG. 15). The droplets may be sandwiched between two plates, with the top and bottom plates taking various configurations described herein. Droplet radius, height, volume, shape, absorbance, fluorescence, surface plasmon resonance, and other kinematic properties may be estimated from optical measurements correlated to lighting assessed from the computer-vision system.

Computer-vision based detection may be aided by the introduction of colored or fluorescent dyes into a droplet. Examples of dyes include, for example, cresol red in the visible (e.g., color marker and pH indicator) and ROX fluorescent in the infra-red (e.g., passive reference dye). Images of droplets may be taken at different optical wavelengths including, but not limited to, the infrared spectrum, visible spectrum, ultraviolet spectrum, or any combination thereof. The images can be taken using cameras designed to image the array at a wavelength range. An optical filter (1620) may be used to change optical properties (e.g., remove wavelengths) of a droplet (110) or an array (100) imaged by the camera (1610) (FIG. 16).

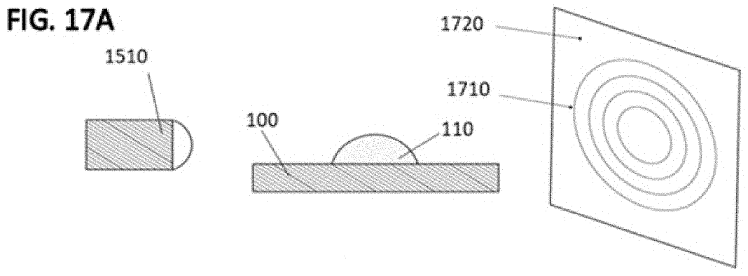

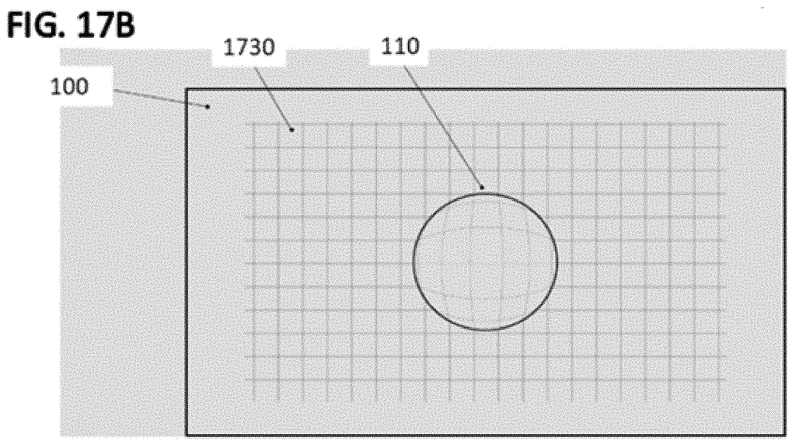

The volume of a droplet (110) of an array (100) may be estimated by, for example, employing a computer-vision based system imaging the interference pattern of light (1710) cast to the droplet by a light source (1510) onto an image sensor (1720) (FIG. 17A) or by imaging the deformation of a projected light pattern (1730) by the droplet (110) that is projected onto the array (100) (FIG. 17B).

The information derived from a computer-vision system may be processed in real-time. This processed information may be used to command introduction of a fluid to an array to, for example, compensate for evaporative loss. The positional information may be used to navigate droplets on the array using electromotive force-based actuation (e.g., EWOD or DEW).

The information may also be recorded (e.g., for later processing). Recorded information may be used to determine paths for droplet motion via electromotive force-based actuation (e.g., EWOD or DEW). This includes, for example, the movement of multiple droplets that may not cross paths or droplets that may have coordinated positions. Recorded information may also be used to determine evaporative properties of droplets with different physical and chemical properties. Information gathered on evaporative properties may be used to create timed fluid introduction routines. Timed fluid introduction routines may include, for example, a timer that, at a set or variable interval, may command the dispensing of a volume of liquid near or directly into existing fluid on the array. A sensor may measure the real-time change in volume and may introduce replenishing liquid into existing liquid on the array (e.g., using techniques described herein). Recorded information may be used to create a training dataset that may be employed in the creation of a machine learning model. A machine learning model may, for example, be used to detect the physical characteristics of a droplet within the array.

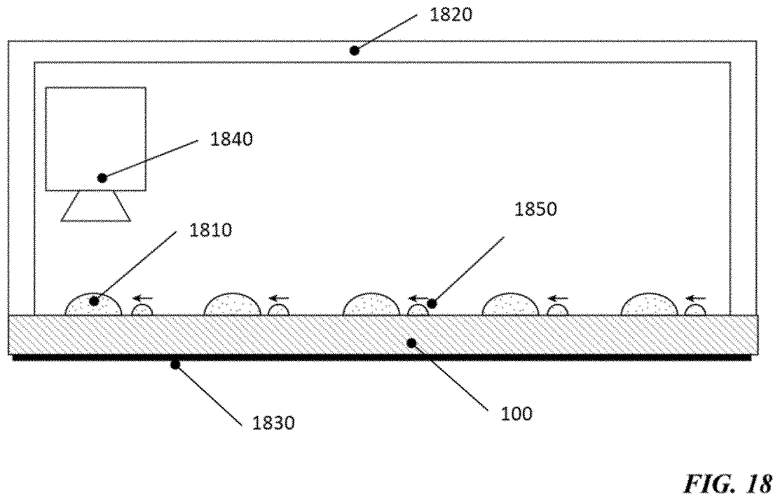

FIG. 18 shows one or more droplets (1810) being processed (e.g., moved, mixed, heated, cooled, etc.) simultaneously on an open array (100). The array may be placed in an enclosed chamber (1820) with uniform temperature and humidity (e.g., using a heater 1830). Uniform temperature and humidity in the enclosed chamber may provide similar processing conditions across all the droplets of an array or a plurality of arrays. One or more droplets may be designated using one or more sensors (1840, e.g., a camera) in an observation zone. For example, the sensor may detect change in volume of the sensed droplet (e.g., FIG. 18). In response to the detected volume change of a first droplet, the system may add a second droplet (1850, e.g., a replenishing droplet) to unmonitored droplets to correct for the volume change of the first droplet.

### EWOD-Actuated Mixing

EWOD-actuated mixing may be performed in open-plate, two-plate, or multi-plate systems described herein. EWOD-actuation may be used to mix droplets of the array. Some of the liquid of the droplet may be introduced to the array through a liquid handler, reservoir, tube/nozzle, or any combination thereof while the mixing regime is executed. The composition of the liquids can be homogeneous or heterogeneous. The droplet may contain at least one microbead. The microbead, or plurality thereof, may be magnetic. These beads may have affinity for biological or chemical material. A range of mixing patterns may be used to resuspend microbeads into the solution. A range of mixing patterns may be used to resuspend microbeads to enhance the reaction kinetics of a heterogeneous reaction. The mixing can be used to solubilize reagents. The mixing can be used to enhance reaction kinetics. In addition to mixing, heat, magnetic fields, or a combination thereof may be applied to accelerate the reaction. In some embodiments, DNA adapter ligation, which can proceed for hours, can be accelerated by incorporating electromotive force based (e.g., EWOD-based) mixing. In addition to EWOD-actuated mixing, one more of following example mixing modalities may be combined to mix droplets of different volumes (1 pL to 1 mL) and viscosities: acoustically induced mixing, liquid handling robot or robotic arm augmented mixing, and mechanical vibration induced mixing.

The frequency of the potential switches between electrodes in the EWOD array can impact the mixing efficiency. A range of the mixing frequencies (e.g., at most 10 kHz) and mixing patterns may be used for mixing. The actuation may create high Reynold's number (>4000) flows, resulting in eddies in order to achieve improved mixing efficiencies. Mixing efficiency may be assessed and monitored using a computer-vision based algorithm, which can employ metrics, such as, for example, dye intensity. The system may be used to provide feedback to the liquid handler, a controller attached to the array, or a combination thereof to compensate for any inefficiencies in mixing by altering parameters, including, but not limited to, mixing frequency, reaction time, and mixing pattern.

### Controlling Evaporation

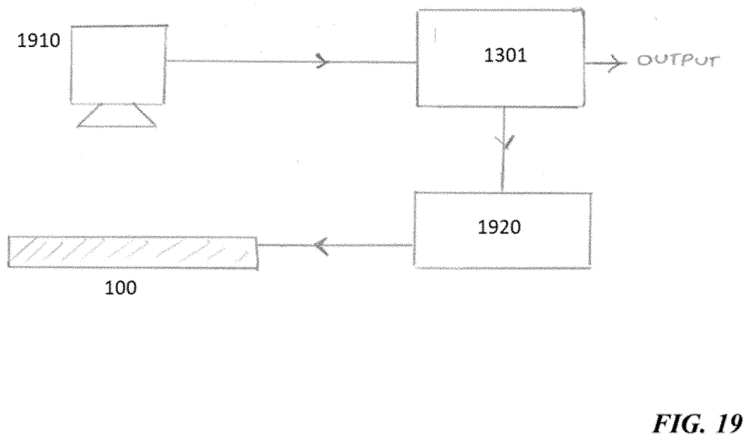

Evaporation of a liquid of said array can be controlled (e.g., reduced) in an open or closed array. The methods described herein may be used individually or in combination to compensate for evaporative loss. Provided systems and methods described herein, evaporative loss can be prevented at temperatures between −100° C. to 250° C. A system (FIG. 19) of one of more visualizing units (e.g., cameras, 1910) from all view angles of the array (100) may feed images of the array into the processing unit (1301). The processing unit may collect and process the images to generate data that can be used in real time or for post processing. The output data of the processing unit may include, but is not limited to: location tracking, droplet volume, presence of a single cell, presence of multiple cells, cellular activity, velocity and kinematic information, radius, shape, height, color, surface area, contact angle, reaction state, emittance, absorbance, or any combination thereof. The output data may be saved for post processing, or the processing unit may give commands to actuate by an actuator (1920) an input, an output, or any combination thereof adjacent to the array or on the array in real time. The processing unit may provide error correction instructions to the array. The processing unit may generate instructions regarding state for execution by either a human or an automated mechanism related to the array. The processing unit for the visualizing unit may be integrated with other software and hardware systems.

A computer-vision based system may be used for droplet detection, droplet volume estimation, or a combination thereof. Droplet volume may be estimated from geometric parameters of the droplet derived from processed images. Such parameters may include, but are not limited to, characteristic lengths, such as, for example, radius, height, contact angle, and projected surface area of the droplet. Liquid required to compensate for evaporative loss may be introduced into the droplet by, for example, manual pipetting, tubes, nozzles, inkjets, liquid handling robots, electromotive force-based actuation (e.g., EWOD), or any combination thereof of droplets from a reservoir.

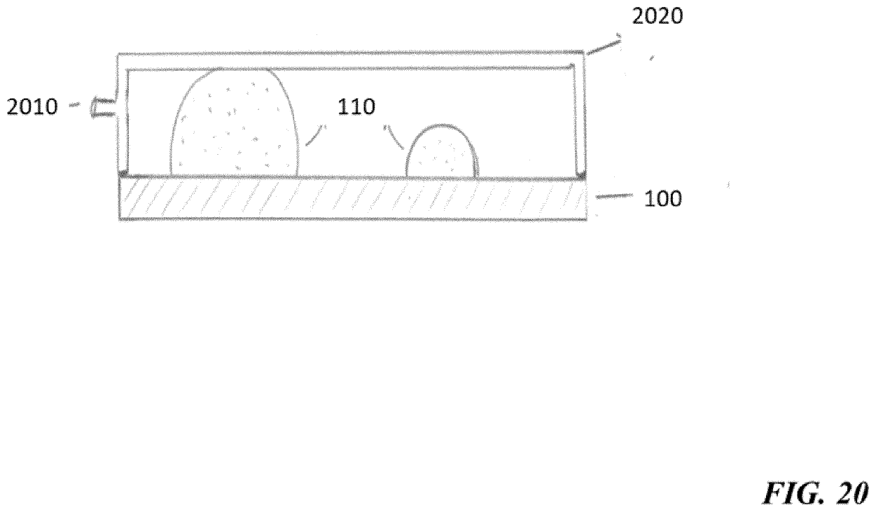

A top plate (2020) may be added to an array (100) to create a humidity chamber encasing the droplets (110) to prevent evaporative loss. This plate may be in contact with the droplets (FIG. 20). The chamber may contain an inlet (2010) for the introduction of humid air. This chamber may be pressurized, heated, or a combination thereof in order to prevent condensation of moisture on the internal surfaces. The top plate or parts of the chamber may be removed when direct and open access to the droplets is required.

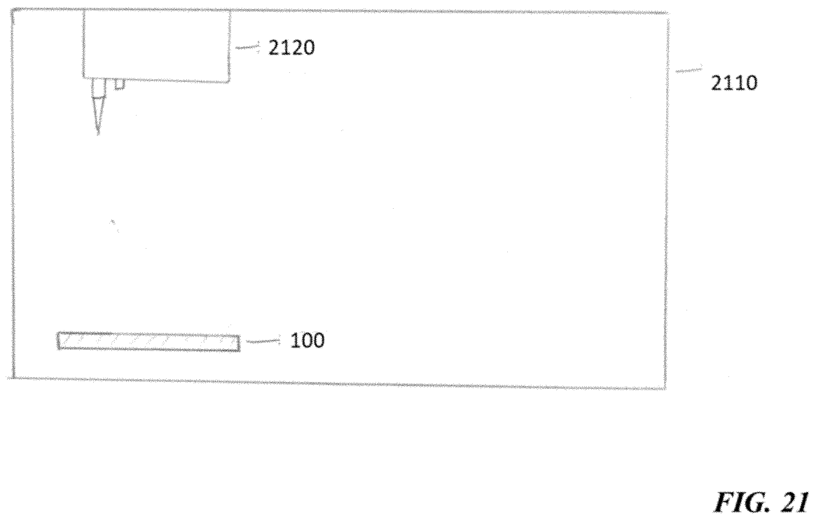

The array (100), or plurality thereof, may be housed in a chamber (2110), wherein the humidity can be controlled. This chamber may house, for example, the liquid handling robotic arm (2120), reagent reservoirs, and/or other components of the array (FIG. 21). The chamber may contain a pressure sensor. By measuring changes in vapor pressure inside the chamber, change in the volume of water can be computed. Any change in volume of a droplet can be detected and can be kept at a constant volume by feeding additional liquid to the droplet.

A top plate may be made of glass with a layer of indium tin oxide (ITO) or resistive material such as, for example, nichrome/platinum heaters. The top plate may have an array of electrodes or active electronic components. The top plate may have a layer of hydrophobic coating to enable smooth actuation of the droplet. The droplet may be covered on its sides by an immiscible oil or wax to further reduce evaporative losses.

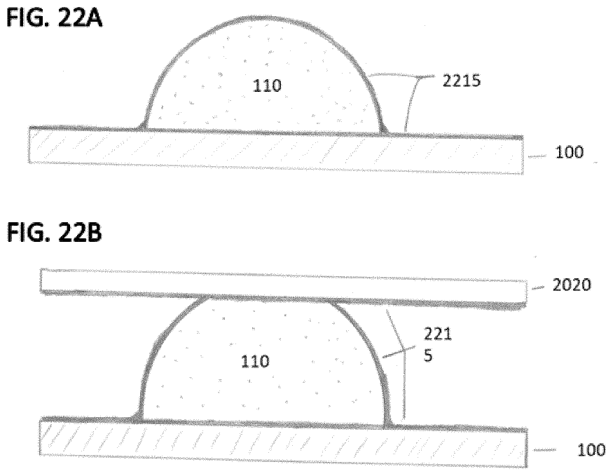

The droplet (110) of an array (100) may be covered or “cloaked” with a thin layer (e.g., a monolayer) of an immiscible low surface-energy liquid (2215). The immiscible low-surface energy liquid can minimize direct exposure of the droplet to the air, reducing evaporation (FIGS. 22A-22C). The immiscible low-surface energy liquid may be used in the one-plate (FIG. 22A) well as two-plate (2020, FIG. 22B) configurations of the array.

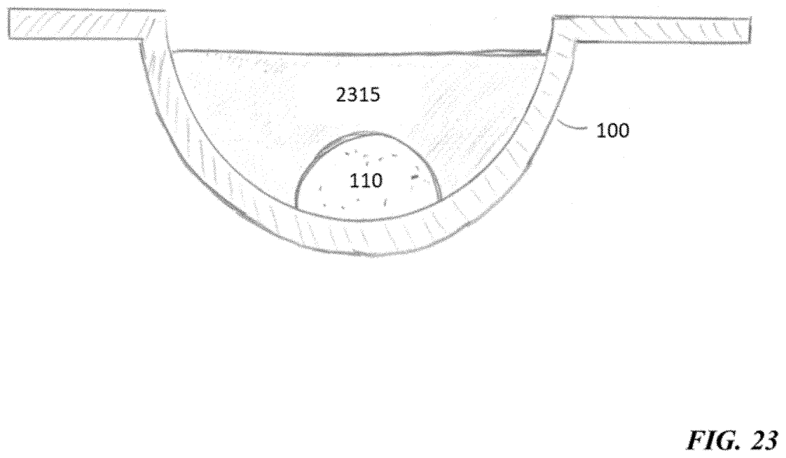

The droplet (110) of an array (100) may be immersed in an immiscible high vapor-pressure fluid (2315), which can result in no contact with air, mitigating evaporation (FIG. 23). This immiscible fluid may include, but is not limited to, mineral oil, silicone oil, fluorinated aliphatic compounds (e.g., FC-40), or any combination thereof.

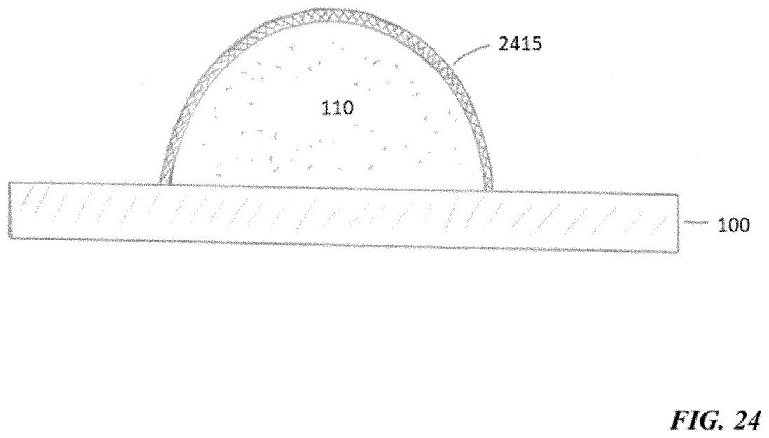

A droplet (110) of an array (100) may be enveloped in a thin three-dimensional (3D) polymeric film (2415) or membrane that can prevent exposure of the droplet to air, resulting in lower evaporation rates (FIG. 24). The film or membrane may be directly formed on the droplet or pre-formed before introduction to the droplet. The polymeric film may be removed (e.g., physically or with heat). The droplet can be transported and mixed with other droplets through electromotive-force based actuation.

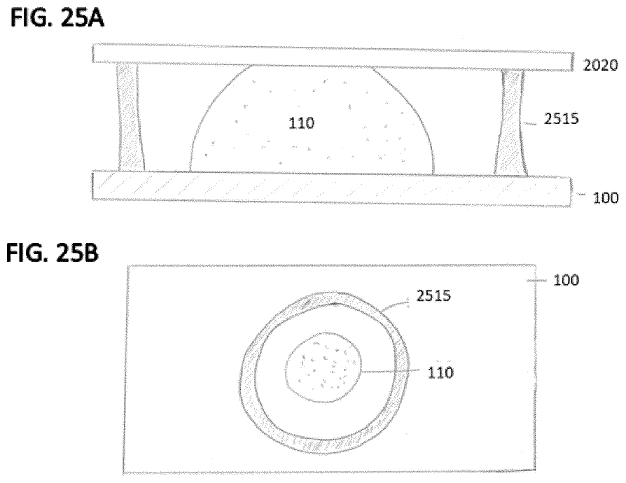

A droplet (110) of an array (100) can be enveloped by a seal (2515). The seal can be singly or repeatedly sealed and unsealed. The sealing and unsealing can be achieved, for example, by melting paraffin wax using a heated top plate (2020) or a rubber or silicone gasket may be used for the seal (FIG. 25A (side) FIG. 25B (top)).

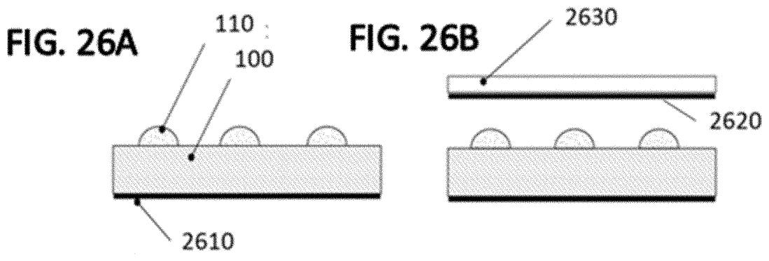

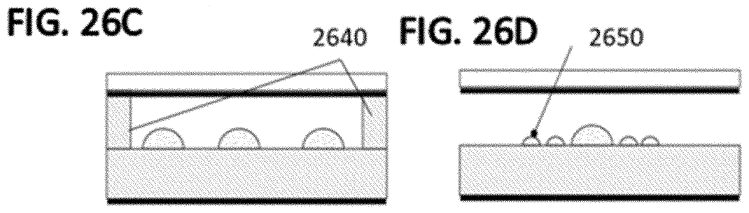

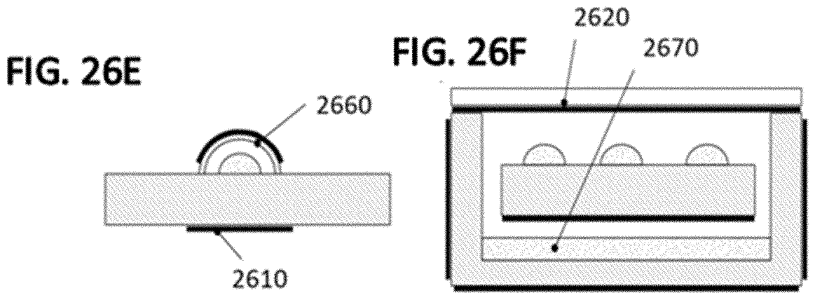

The rate of evaporation of a volume of liquid can be controlled as depicted in FIG. 26A-F. A droplet (110) of an open-surfaced array (100) may evaporate rapidly at elevated temperatures (FIG. 26A). For example, when a thermodynamic reaction occurs within or adjacent to the droplet on the array. Heating the droplet can be achieved using a heater below the array surface (2610). Heating the air around the droplet can reduce the rate of evaporation. The heating can be accomplished with, for example, the use of a heater below the array surface (2610) or a heated top plate (2620) of a transparent top plate (2630) (FIG. 26B). Enclosing the chamber (2640) around the array can further reduce the rate of evaporation (e.g., trapping humidity, FIG. 26C). The use of sacrificial droplets (2650) can increase the humidity in a local environment and slow evaporation (FIG. 26D). A small cap (2660), which, for example, can be larger than the evaporating droplet, may be used to contain humidity and control evaporation (FIG. 26E). Also, the entire array can include a water reservoir (2670) to control the humidity and rate of evaporation of a droplet (FIG. 26F). Uniform heating of a chamber of the array can prevent condensation, reaching near 100% relative humidity levels. Configurations depicted in FIG. 26A-F may also be combined to control the rate of evaporation.

In some embodiments, the relative humidity level achieved is about 50% to about 100%, about 60% to about 100%, about 70% to about 100%, about 80% to about 100%, or about 90% to about 100%. In some embodiments, the relative humidity level achieved is about 89% to about 100%. In some embodiments, the relative humidity level achieved is about 89% to about 90%, about 89% to about 91%, about 89% to about 92%, about 89% to about 93%, about 89% to about 94%, about 89% to about 95%, about 89% to about 96%, about 89% to about 97%, about 89% to about 98%, about 89% to about 99%, about 89% to about 100%, about 90% to about 91%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 91% to about 92%, about 91% to about 93%, about 91% to about 94%, about 91% to about 95%, about 91% to about 96%, about 91% to about 97%, about 91% to about 98%, about 91% to about 99%, about 91% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. In some embodiments, the relative humidity level achieved is about 89%, about 90%, about 91%, about 92%, about 93%, about 94%, about 95%, about 96%, about 97%, about 98%, about 99%, or about 100%. In some embodiments, the relative humidity level achieved is at least about 89%, about 90%, about 91%, about 92%, about 93%, about 94%, about 95%, about 96%, about 97%, about 98%, or about 99%. In some embodiments, the relative humidity level achieved is at most about 90%, about 91%, about 92%, about 93%, about 94%, about 95%, about 96%, about 97%, about 98%, about 99%, or about 100%.

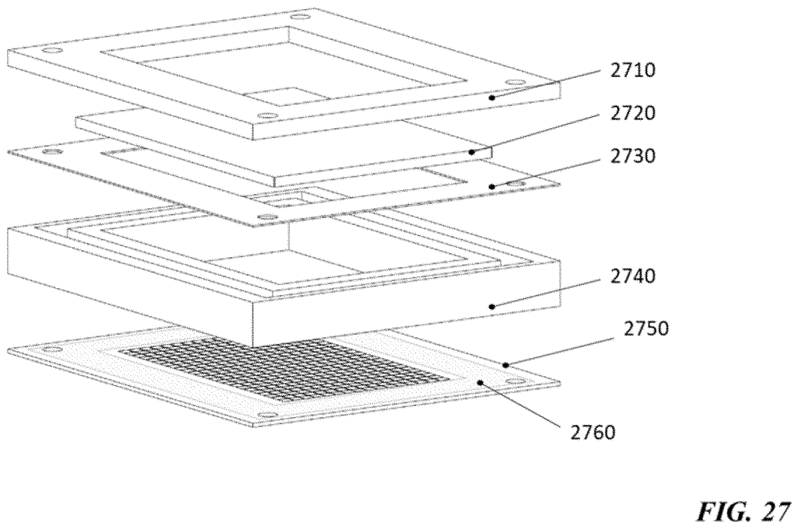

The arrays may be passively heated by the surrounding environment or may include active temperature control circuitry to maintain particular environmental conditions within the array. Active temperature control may be accomplished using, for example, an array depicted in FIG. 27, which array may be composed of a sealing top plate (2710), heated top plate (2720), a gasket (2730), sidewalls (2740), an array tile (2750), a resistive trace heater (2760), or any combination thereof. The top plate can be transparent, and heating methods may include, for example, the use of transparent electrodes, such as, for example, Indium Tin Oxide (ITO) or Aluminum Zinc Oxide (AZO). The sidewalls (2740) of the enclosure may be heated, for example, by an embedded conductor such as, for example, nichrome, thin-gauge copper wire, or by an embedded flexible circuit board with serpentine traces. Furthermore, the sidewalls may be heated passively from the top plate (2720), the bottom substrate (2760), or a combination thereof.

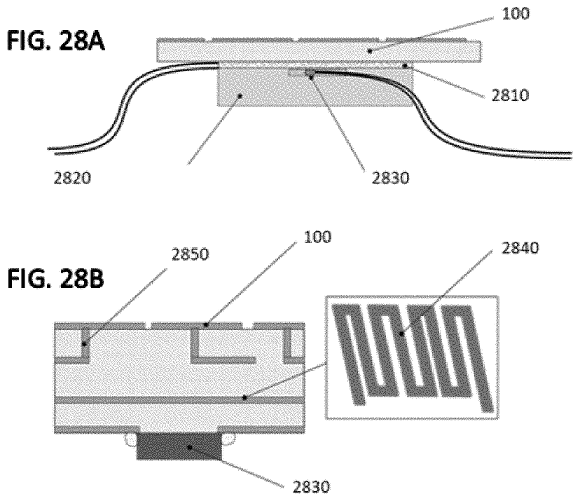

The array (100) may comprise a resistive film heater (2810), thermal insulation (2820), a temperature sensor (2830), or any combination thereof (FIG. 28A). Serpentine traces (2840) within an array substrate may be used to heat the sidewalls of the enclosure through, for example, a gap-filling thermally conductive seal. Heating an array substrate may be accomplished using, for example, a resistive film heater (2810) or by directly embedding serpentine copper traces (2840) within the substrate (FIG. 28A and FIG. 28B). A surface mount temperature sensor (2830) may be attached to the back side of an array substrate to sense and control the temperature (FIG. 28B). The array described herein may comprise via-in-pads (2850).

Beyond controlling evaporation, heated arrays may also be used for precise control of the droplet temperature. Droplets may be heated on an open surface with heaters embedded on or below the array substrate. Without some form of environmental control, these substrate heaters may experience large temperature differences between the internal droplet temperature and the temperature on the surface of the heater. These large temperature differences may lead to imprecise droplet temperature control and may be subject to large temperature fluctuations based on, for example, surrounding air currents. Furthermore, without environmental temperature control, the difference between the heater temperature and the droplet temperature may be a function of parameters, including, for example, droplet surface area to volume ratio, droplet size, and temperature setpoint. These enclosures may be completely sealed to prevent the escape of heated humid air, but they may also be left partially open. For example, this design may allow control of condensation within a cooling temperature environment.

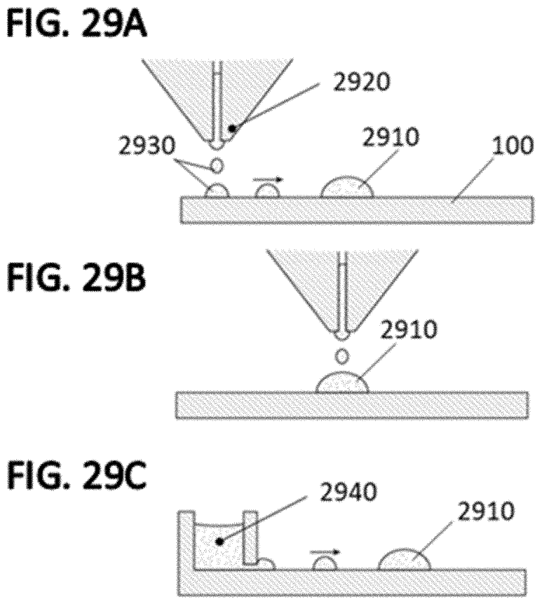

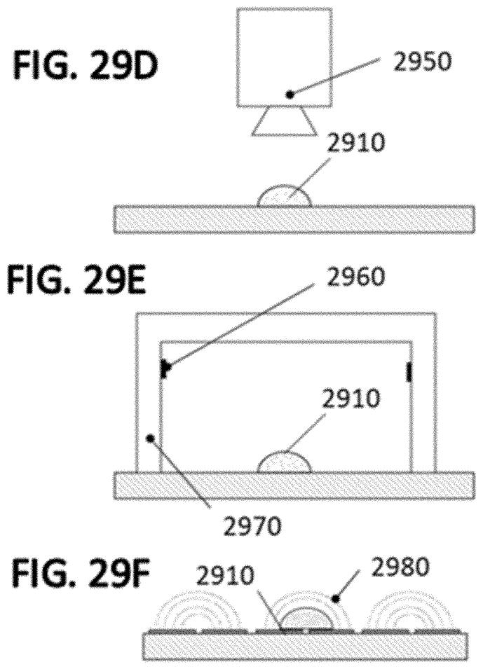

While an enclosed chamber, a heated chamber, or a combination thereof can help control the rate of evaporation, the rate of evaporation can also be controlled by actively replenishing the volume of a droplet (2910) of an array (100). This replenishment can be accomplished using a number of different dispensing methods that include, for example, syringe pumps, piezoelectric and solenoid dispensers, electrowetting-based droplet generators, microfluidic channels, pipettors, diaphragm pumps, or any combination thereof (2920, FIG. 29A). Each of these methods may be capable of creating droplets (2930) at a scale and resolution that is sufficient to maintain the volume of an evaporating droplet within at least a 30%, 20%, 10%, 5%, 1%, 0.1%, 0.01%, or less error margin. For example, to maintain a 40 μL droplet reaction volume, the droplet generator (#) can create 4 μL droplets with a resolution of at least 100 nL, 50 nL, 10 nL, 1 nL, 0.1 nL, 0.01 nL, or less.

The generated replenishing droplets (2930) can be introduced to the evaporating droplet directly (FIG. 29B) or through electrowetting motion (FIG. 29A and FIG. 29C). Introducing the replenishing droplets by electrowetting can, for example, provide a pre-heated replenishing droplets from, for example, a reservoir (2940), providing a way to maintain a precise and well-controlled temperature within the reaction volume of the replenished droplet.

A rate of compensation (e.g., droplet replenishment) can be determined through experimental data collection or it can be measured and actively controlled using a variety of sensors. For example, computer-vision techniques (e.g., using a camera (2950)) can be used to estimate droplet volume and are discussed further herein (FIG. 29D). Another sensing method may include, for example, humidity sensors (2960) within enclosed (2970) or semi-enclosed environments (FIG. 29E). With an enclosed volume at a temperature range, the mass of evaporated water in the atmosphere of the system can be estimated using saturation vapor pressure tables and the measured relative humidity. Capacitive sensing may also be used to detect a change in droplet volume (e.g., since the degree of capacitive coupling (2980, e.g., electric field) between neighboring electrodes will be significantly altered by changing droplet volumes (FIG. 29F)). Change in volume of the droplet (2910) can also be estimated by measuring the change in weight of the array (100) comprising the droplet.

The replenishing droplets may replenish at most about 1%, 5%, 10%, 15%, 20%, 30%, 40%, 50%, or more of the total volume of the replenished droplet. The replenishing droplets may replenish at least about 50%, 40%, 30%, 20%, 15%, 10%, 5%, 1%, or less of the total volume of the replenished droplet. The replenishing droplets may replenish from about 1% to about 50%, about 1% to about 20%, about 1% to about 10% of the total volume of the replenished droplet. The mixing of samples on the array (e.g., with concurrent heating) may result in, for example, more efficient reaction kinetics within a droplet and shorter reaction times (e.g., ligation reactions). The mixing within a droplet may induce, enhance, accelerate, or any combination thereof fragmentation of the nucleic acids (e.g., DNA).

### Magnetic Field Generation on an Array

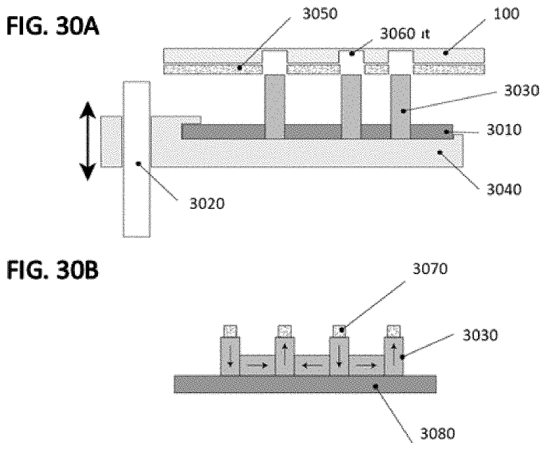

Biological workflows may function utilizing the ability to turn a magnetic source of an array “on” and “off”. This may be accomplished using a linear stage and actuator (3020) to lift or lower a set of magnets (3030) on a platform (3040) (FIG. 30A). The array substrate can be equipped with ferromagnetic shields (3050). The magnets can be, for example, electromagnets (e.g., solenoids). A ferromagnetic shield (3050) may also be used to increase the difference in magnetic field strength between “up” and “down” positions of an array of magnets. For example, when the magnets are “up” they may poke through the magnetic shield such that the shield acts as a flux guide to help concentrate the return flux. Also, when the magnets are below the ferromagnetic shield, the shield may act to diminish the field permeating through the array. Cutouts (3060) may also be made to allow the magnets to be positioned closer to the active array surface (100). The dimensions of the cutout may be at most about 0.001 nm, 0.01 nm, 0.1 nm, 1 nm, 10 nm, 100 nm, 1,000 nm, 10,000 nm, 100,000 nm, 1,000,000 nm, or more. The dimensions of the cutout may be at least about 1,000,000 nm, 100,000 nm, 10,000 nm, 1,000 nm, 100 nm, 10 nm, 1 nm, 0.1 nm, 0.01 nm, 0.001 nm, or less. The dimensions of the cutout may be from about 0.001 nm to about 1,000,000 nm, about 1 nm to about 10,000 nm, about 10 nm to about 1,000 nm.

The magnet array (3010) can be interchangeable such that different workflows can be accomplished by changing the position of an array of magnets to other arrangements or configurations (e.g., a Halbach array, FIG. 30B). For example, a Halbach array can be arranged with magnets (3030) such that the magnetic field pointing towards the array can be significantly stronger than the field on the underside of the magnets. Control of the magnetic field can be improved with ferromagnet flux focusers (3070), ferromagnetic back iron (3080), or a combination thereof.

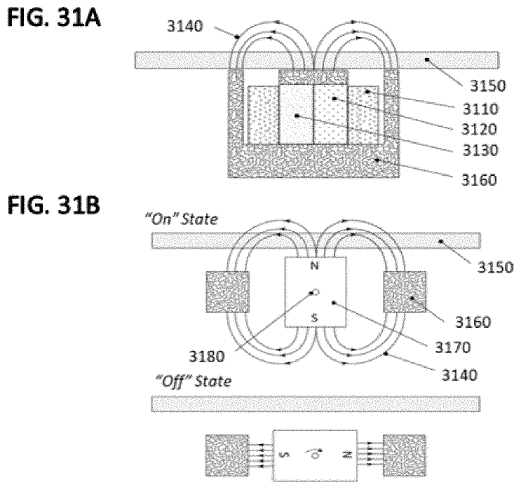

Switching a magnetic field on and off can also be accomplished using, for example, electro-permanent magnets and rotary switchable magnets (FIGS. 31A-31B). Electro-permanent magnets may be constructed from a coil (3110) wrapped around a magnetically-hard (3120, e.g. neodymium) and magnetically-soft (3130, e.g. alnico) magnets (FIG. 31A). A pulse of current may switch the polarity of the magnetically-soft magnet. For example, when the poles align, the electro-permanent magnet can generate a magnetic field (3140) that passes through the array surface (3150). Also, when the poles are antiparallel, the magnetic field can be confined within the flux guide (3160) of the electro-permanent magnet, producing relatively little magnetic field at the surface of the array substrate. A rotary switchable magnet works in a similar way by physically rotating a permanent magnet (3170) 90-degrees on an axis of rotation (3180) (FIG. 31B). In the on-state, the magnet can generate a magnetic field (3140) on the array surface (3150). When rotated 90-degrees, the ferromagnetic flux guides (3160) may confine the field and relatively little to none of the field may be produced at the surface of the array substrate.

### Reference Electrode Design and Placement

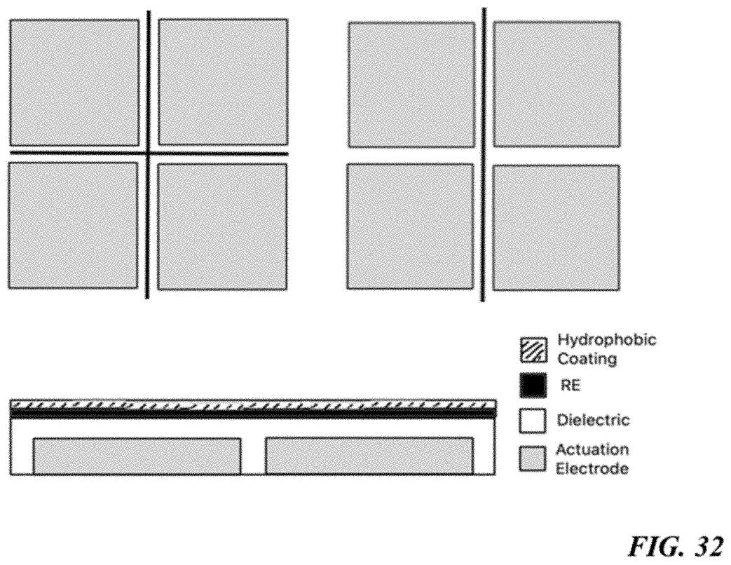

Electrode arrays may be used to create reference electrodes (REs) as described herein. The design and placement of the REs with respect to the actuation electrodes may be important for the methods and systems described herein. A single RE or a set of REs may be placed around an actuation electrode, or a plurality thereof, (in XY plane) or in between such actuation electrodes (in XY plane) as illustrated in FIG. 32. The REs may be located in a different plane along a Z-axis. In a non-coplanar arrangement, a layer of dielectric material may separate the REs and the actuation electrodes. The RE may be of an arbitrary shape and need not be a straight line as shown in FIG. 32. There may be one more REs organized as regularly spaced grids or irregular arrays. The configuration may include, but is not limited to, a top plate with a hydrophobic coating and a distance from the array with EWOD capabilities can be adjusted manually or through robotic actuation.

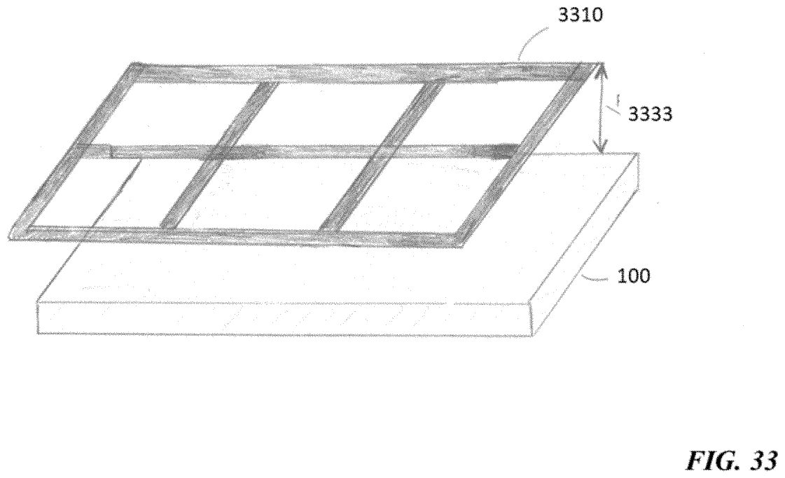

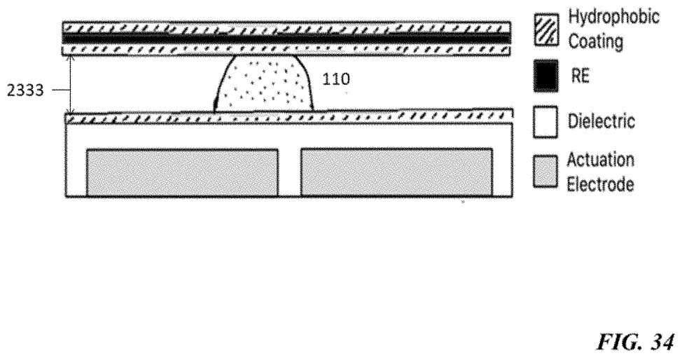

The RE may be a wire mesh or individual wires (3310, FIG. 33) placed above the actuation electrode plane with a space (3333) that can accommodate a droplet (FIG. 34). The RE may have a hydrophobic coating that enables smooth motion of the droplet. The height of the mesh/network of wires may be fixed or adjusted manually or by robotic actuation. A RE may be temporarily positioned to introduce electrowetting forces and subsequently removed for continuous droplet actuation. A temporary contact with a droplet can be sufficient to actuate the droplet for a finite amount of time. The reference electrode may be introduced again if the droplet stops responding to the electric field on the electrowetting array.

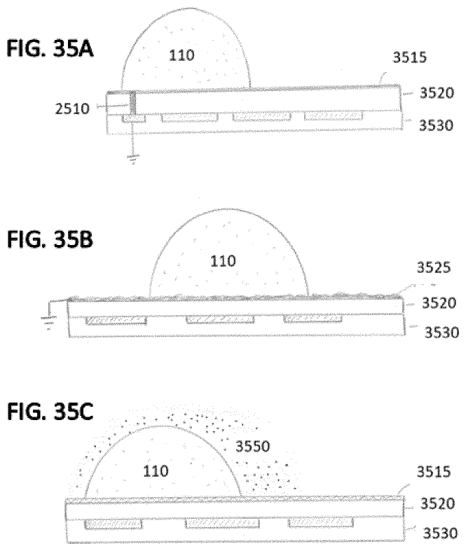

Regions of the topmost dielectric layer (3520) may be modified to become electrically conductive (FIG. 35A). The modified region may establish contact (3510) with a ground electrode and provide a reference for electrowetting operation. The ground electrode may be a dedicated ground electrode or it may be an actuation electrode that is temporarily grounded on the electrode array (3530). The system may operate with a hydrophobic coating (3515) that may surround the droplet (110). A region of the topmost dielectric layer may be modified through methods including, but not limited to: introducing defects in the layer through UV-treatment, treatment with plasma, inducing dielectric breakdown, applying physical force or pressure, using a material that is known to have a porous structure, or any combination thereof.

A layer of oil (3525), such as silicone oil, can serve as a hydrophobic coating as well as a reference electrode when grounded (FIG. 35B). The layer of oil may be slightly conductive or polar. The dielectric surface may contain microstructures introduced by the methods including, but not limited to, the methods described herein. These microstructures can wick oil and can be connected to a ground potential by, for example: a temporarily grounded actuation electrode, a dedicated ground electrode, a dedicated connection elsewhere on the array, or any combination thereof.

Ionized air (3550) may surround droplets in the array (FIG. 35C). Ionized air may be used for the array as a reference electrode for electrowetting actuation. Ionized air may be introduced through an ionized air blower and directed towards the droplet. A droplet may be permanently stuck to a location due to charging of the surface or the droplet (i. g., pinning) Droplet pinning may be mitigated by neutralizing the droplet with ions introduced through the blower.

### Dried/Lyophilized Reagents On-Chip

Chemical reagents, biological reagents, or a combination thereof may be lyophilized/dried/spotted on the surface of the array. The reagents may be spotted on the surface of a disposable cartridge that is compatible with the array. The reagent may include, but are not limited to, buffers, salts, surfactants, nucleic acids, proteins, stabilizing agents, microbeads, enzymes, antibiotics or any combination thereof. The reagents may be solubilized or resuspended in the appropriate solution by liquid handling systems, EWOD actuation, manual pipetting, or any combination thereof. Kits, in part or in whole, for myriad molecular biology workflows/processes, may be produced using dried reagents. The kit may comprise refrigerated conditions for storage. Molecular biology processes may include, but are not limited to, preparation of nucleic acid libraries for next generation sequencing and microbial analysis workflows, (e.g., antibiotic-resistant strain detection).

### EWOD-Enabled Magnetic Bead Wash

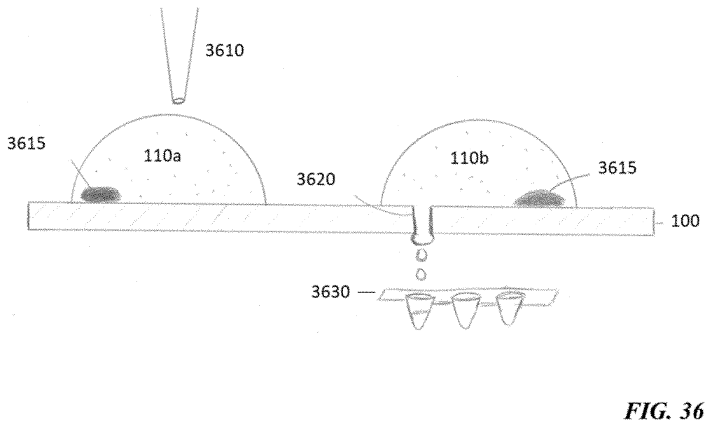

Magnetic particles (3615) may be manipulated on the surface of the chip by a controllable, localized magnetic field (FIG. 36). The magnetic particles may be made of, for example, microspheres. Controlling the localized magnetic field may be achieved by, for example, placing a solenoid, a magnet, a pair of magnets, or any combination thereof in the vicinity of the particles or by generating a magnetic field within the EWOD chip. Magnetic bead-based separations and washes may be performed on an EWOD-enabled array (100). The droplet (110) may be manipulated using the actuating electrodes which may also allow positioning of the droplet. The magnetic particles may be concentrated in a small region using the magnetic field. Liquids may be separated from the magnetic particles by EWOD-based, dielectrophoresis-based, or other electromotive force based actuation. Separation is possible in the open-plate and two-plate systems. Since the droplet can be positioned using EWOD actuation, the fluid (110a) may also be aspirated from the chip using a liquid handling robot (3610), leaving the magnetic particles on the chip surface. Removal of liquid (110b) may be achieved through a hole (3620), or a plurality thereof, in the array by employing capillary forces, pneumatic forces, electromotive forces, such as EWOD or dielectrowetting, or any combination thereof. This waste fluid may be collected in a reservoir positioned under the array (3630). A computer-vision-based algorithm may be used to inform and provide feedback to the liquid handler and/or array for the processes involving magnetic beads. The processes may include, for example, aspiration of the supernatant, resuspension of beads, preventing aspiration of magnetic beads along with the supernatant during removal of supernatant, or any combination thereof.

### Disposable Cartridge

Various methods through which the EWOD platform may be used along with a replaceable cartridge are described herein. A replaceable, flexible, or a combination thereof construct, such as, for example, a film or membrane, allows for reuse of the actuation and/or reference electrodes. The replaceable cartridge may also eliminate cross-contamination between samples in separate experiments or the same experiments. The disposable cartridge construct may contain combinations of dielectric, hydrophobic layers, reference electrodes, inlets, outlets, or any combination thereof for the introduction and removal of fluids. The replaceable construct may be permanently bonded to the array. The construct can be bonded to the actuation electrodes using adhesives, heat, application of vacuum, strong static electric field, or any combination thereof.

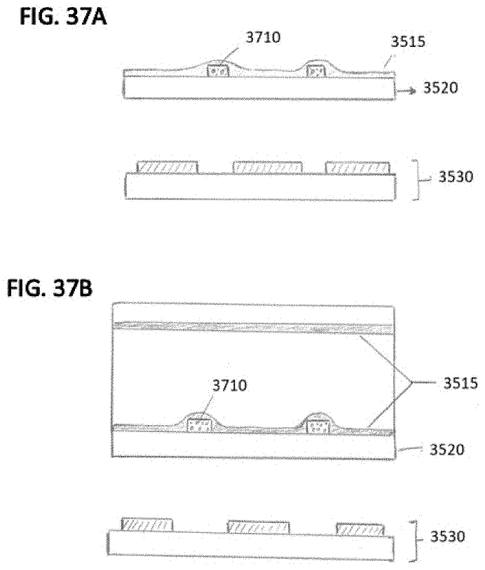

The disposable/replaceable cartridge may have an open surface for manipulating biological samples, chemical samples, or a combination thereof (FIG. 37A). The cartridge may comprise multiple layers, starting from a layer of dielectric (3520). A layer of conductive material (3710) may sit above the dielectric. The conductive layer may be grounded and used as a reference electrode for electrowetting or for sensing a parameter, or a plurality thereof, of the biological sample, chemical sample, or a combination thereof. The conductive layer and regions of dielectric with no conductive layer together may have a layer of hydrophobic coating (3515). Alternatively, the conductive layer and the dielectric layer can be coated with a slippery liquid coating, such as, for example, SLIPS coating. A second plate may be placed above a configuration similar to FIG. 37A (dielectric, conductive layer, hydrophobic coating) with a small gap in between (FIG. 37B). This small gap may be air or may be filled with a filler fluid. The bottom side of the second plate may be coated with a hydrophobic coating (3515). The bottom side of the second plate may be coated with a liquid layer using coatings, such as, for example, SLIPS.

The cartridges described herein may be temporarily positioned on an electrowetting array such that the actuation electrodes are located below the dielectric layer. The cartridges described herein may comprise an array of actuation electrodes attached permanently, below the dielectric layer. The entire stack (electrode array, dielectric, conductive layer, hydrophobic coating, air gap, (second plate if any)) may be disposable as a single unit. The cartridge may contain only a layer of dielectric that sits directly on to the actuation electrodes. The dielectric may be permanently bonded on to the actuation electrodes. The dielectric may have a layer of slippery coating (hydrophobic coating or SLIPS). Alternatively, the cartridge containing the dielectric and hydrophobic layers may be bonded to the actuation electrodes plate while the reference electrode is housed on a top plate. The reference electrode plate may be replaced or rinsed in order to avoid cross-contamination of droplets. The disposable cartridge may be placed on and removed from an electrowetting array by an automated robotic handler. The disposable cartridge may contain droplet sample while being handled by the robotic handler. The disposable cartridge can be on a conveyor belt and placed on an electrowetting array. The cartridge may be bound to the electrode array using vacuum or other pressure-based systems. After a single experimental run, the conveyor belt might remove the used part of the cartridge and introduce a new layer to the cartridge. The cartridge may comprise a mesh/network of conductive wires (acting as reference electrodes) that are held directly above the surface and not in contact with the surface. The wire mesh may be replaced or rinsed in order to avoid cross-contamination of droplets. The wire mesh may also be permanently fixed onto the cartridge and be disposed of with the rest of the cartridge. The disposable cartridge may have active electronics embedded below the dielectric or below the actuation electrodes to perform, for example, electrowetting operations, manipulate samples using other electromotive forces, measure analytes in the biological sample, or any combination thereof.

### Packaging and Driver Electronics

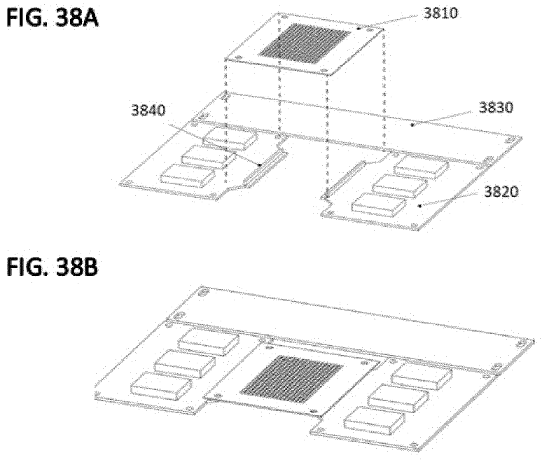

An array tile (3810) may be constructed such that it is separate from the electronics (3820) that control and drive the electrodes (FIGS. 38A-B). This may be beneficial, for example, to enable application-specific electrode geometries that are tailored to specific workflows or processes. The tile may be connected to the drive (3820) and control (3830) electronics through interfaces (3840), including, for example, fine-pitched elastomeric connectors, board-to-board connectors, pogo-pins, or any combination thereof.

It may also be beneficial to package the tile and drive electronics together and have a generic modular connection mechanism between the control electronics and the drive electronics. This can provide application specific electrode arrays that each have a varying number of electrodes and corresponding drive circuits. A benefit of this approach can be the reduction in numbers of connectors needed for, as an example, multiplexing drive circuits (e.g., ˜10 control signal connections can drive ˜100's of electrodes).

### Array-Loading Module

The systems and devices described herein can comprise a module configured to receive the array tiles (or multiple array tiles) described herein. The module configured to receive the array tiles can comprise electrical connectors that are in communication with the systems and devices described herein. In some embodiments, the module configured to receive the array tiles is aligned with the electrical connectors of the systems and devices described herein. The module configured to receive the array tiles can comprise a cover (e.g. a lid). The cover can be transparent or opaque. The cover can be hinged. In some embodiments, the hinged cover is used to contact the array tiles with the electrical connectors. In some embodiments, the hinged cover aligns the array tiles with the electrical connectors. In some embodiments, the module configured to receive the array tiles facilitates and/or maintains electric communication between the array tiles and the systems and devices described herein. The hinged cover can be coupled to the module by spring-loaded latches, thumb screws, magnets, or various other mechanical connectors. The array tiles may take any combination of the electrode layers, dielectric layers, slippery coatings and plate configuration described herein.

In some embodiments, the methods described herein further comprise disposing the array tile (or multiple array tiles) described herein into the module configured to receive the array tiles.

In some embodiments, the cover of the module comprises at least one light source. In some embodiments, the light source emits light. In some embodiments, the light source emits diffused light. In some embodiments, the cover is configured to facilitate observations of the array tiles by emitting light onto the array tiles. In some embodiments, the cover is configured to facilitate imaging of the array tiles as described herein. In some embodiments, the cover is configured to facilitate improvement of the systems and methods described herein by machine learning by emitting light onto the array tiles. Illumination of the droplets disposed onto the array tiles can increase contrast between biological samples of interest and a background. In some embodiments. In some embodiments, illumination of the array tile facilitates machine observation of the biological samples disposed onto the array tiles. In some embodiments, illumination of the array tile facilitates machine observation of the reactions undergone on the array tiles.

The cover may further comprise various environmental sensors and actuators to monitor and control the environment over the electrode array (e.g. the temperature and humidity controls described herein). In some embodiments, the various sensors and actuators comprised in the cover comprise optically transparent heaters, sponges or reservoirs for passive humidity control, and temperature and humidity sensors.

The cover can be configured to communicate with the array tiles. In some embodiments, the cover may be in electrical communication with the array tiles. In some embodiments, the cover may be in electrical contact with one or more reference electrodes comprised on the array tiles. In some embodiments, the cover is configured to electrically contact the array tiles by a spring connector. In some embodiments, the cover is configured to electrically contact the array tiles by a conductive paste. In some embodiments, the cover is configured to electrically contact the array tiles by another conductive means.

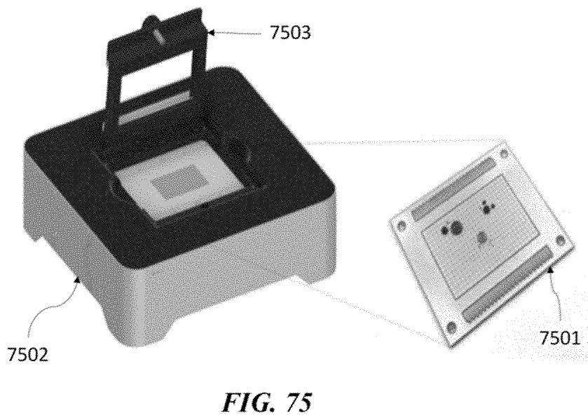

In some embodiments of the systems and devices described herein, the module is removable from the systems and devices described herein. In some embodiments of the systems and devices described herein, the cover is removable from the module. FIG. 75 exemplifies an embodiment of the module and cover described herein. The array tiles (7501) can be loaded into the embodiment of the systems and devices described herein (7502) by displacement of the array tiles in the module (7503) manually or with an automated plate-handling robot.

### Projection Apparatus

In some embodiments, the system is provided with a projector to manipulate and emit light onto an array. In some embodiments, a projector is mounted above the electrode array to project visual information onto the array. In some examples, the projection emits light incident on the location where a user should manually add new reagent droplets for the specified reaction. The projector may also be used as a general purpose indicator and display information, such as, the percentage of the reaction that has been complete, the time remaining, whether heating is currently active, whether magnetic manipulation is being performed, or other information regarding a state of the system. The projector may receive display information for what to display from an on-board or off-board computer either through a physical display connection (such as HDMI, USB, etc) or through a wireless display connection (such as bluetooth or wifi).

In some embodiments, the projector provides a source of illumination for various on-chip measurement processes that are enabled by a camera (or other optical sensors) mounted above, below, or on the sides of the electrode array. This may include, for example, projecting a pattern of light (such as stripes, spherical harmonics, or pseudo-random dot pattern) in order to image the droplets' topography and determine their three-dimensional shape and volume (via structured light scanning) The optical sensor and the projectors system may perform any of the optical measurements described herein (UV-visible spectrophotometry for absorption spectroscopy, Surface-Plasmon Resonance, NIR spectroscopy, Transmission spectroscopy, Fluorometric readouts, colorimetric readouts.

In some embodiments, the projector comprises a diffused light source, such as one or more light emitting diodes. In some embodiments, the projector comprises one or more digital micromirror devices or liquid crystal displays to produce the patterns of light. In some embodiments, the project comprises one or more optics to collimate the light source onto the digital micromirror devices or liquid crystal displays. In some embodiments, the projector comprises one or more optics to focus light patterns from the digital micromirror devices or liquid crystal displays onto the electrode array.

In some embodiments, the projector comprises a collimated light source. The collimated light source may be a laser. The projector may further comprise one or more scanning mirrors or galvanometers to direct the laser beam onto the electrode array.

The instrument may also include various forms of lighting indicators to help indicate the state of the machine. LED indicators that cast colored light below the machine, which may be used to display the state of the system. In some embodiments, the lighting indicators may display that the machine is idle, currently running, or waiting for user input.

In some embodiments, array tile contains photosensitive elements such as photoconductor as described in the photo-electrowetting and optoelectrowetting sections of the description herein. Light emitted from the projector system may induce surface energy change on the surface of the chip. The surface energy change may be used for manipulating droplets using electrowetting effect or manipulating microscopic objects (beads, single cell, droplets etc.) using attractive or repulsive forces.

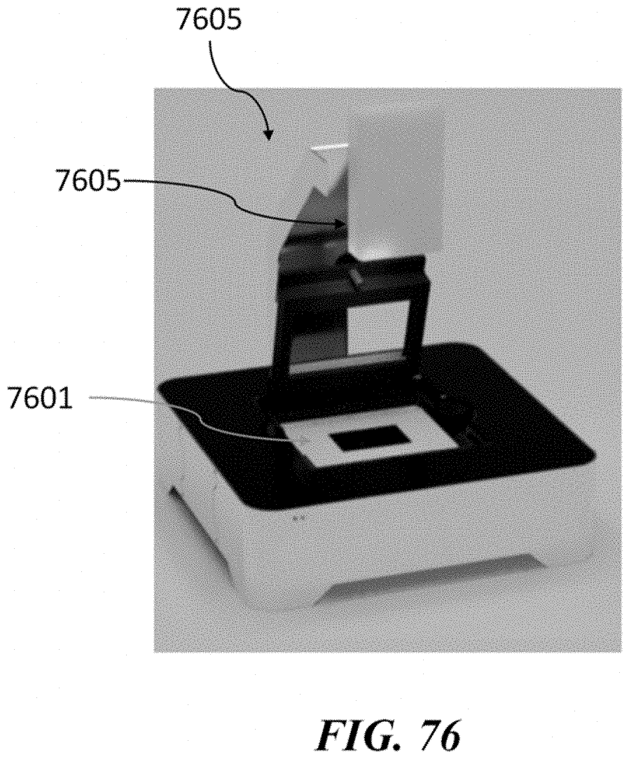

FIG. 76 exemplifies an embodiment of the module and projector described herein. In some embodiments, the system (7603) comprise a projector (7605) configured to emit a patterned incident on a array tile (7601).

In some embodiments, the methods described herein further comprise emitting a light pattern to project visual information onto the array. In some embodiments, the methods described herein further comprise manipulating light from a light source and projecting light patterns onto the array. In some embodiments, the methods described herein further comprise illuminating the array with and performing one or more optical measurements using optical sensors. In some embodiments, the methods described herein further comprise projecting a sequence of light patterns to manipulate droplets on the array.

### Large Volume Sample Processing

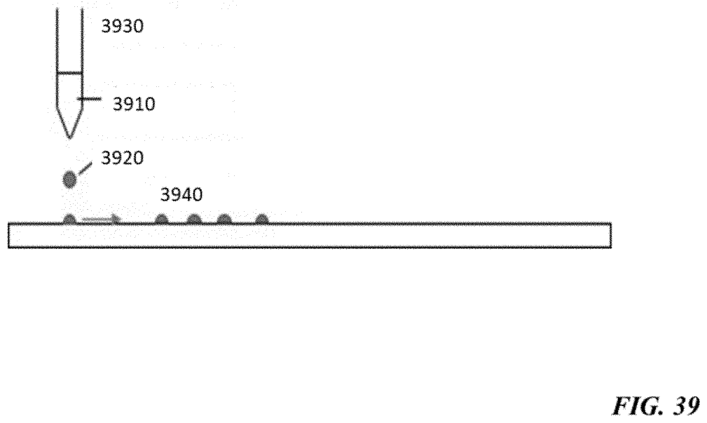

Processing large volume samples (e.g., microliter-, centiliter-, or milliliter-scale) may be carried out by segmenting or fractionating the starting material (3910, e.g., biological samples) into aliquots (3920) using a dispenser (3930), and then introducing the aliquots to a processing area of the array (3940) (FIG. 39). Input material can be processed on the array as droplets in parallel or sequentially. The input material may be, for example, biological samples (e.g., blood, tissue, or plasma) or environmental samples (e.g., water or soil). Sample processing on the array may involve, for example, extraction of nucleic acids (e.g., DNA, RNA), isolation of specific cell types (e g, immune cell subtypes, circulating tumor cells, or cells isolated from tissue biopsies), or isolation of extracellular vesicles (e.g. exosomes).

### Array Scaling

### Multiplexing

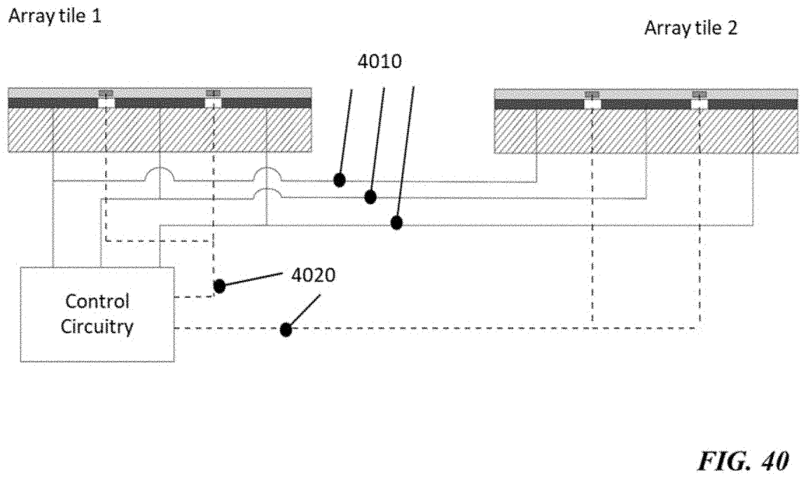

The number of drive signals can be reduced for scaling from a single array tile to a large number of array tiles (e.g., 10, 20, 30, 40, 50, 100, 500, or more array tiles) for parallel processing of samples (e.g. 96 samples processed simultaneously on 96 individual tiles). For example, a common drive signal (4010) can be used to actuate electrodes on multiple tiles simultaneously. Furthermore, the reference electrode(s) on each tile may be driven by separate signals (FIG. 40). At any given time, activating the reference electrodes (4020) on particular tile may enable droplet mobility on that tile, while the droplets on other (e.g., inactive) tiles may not experience an electromotive forces.

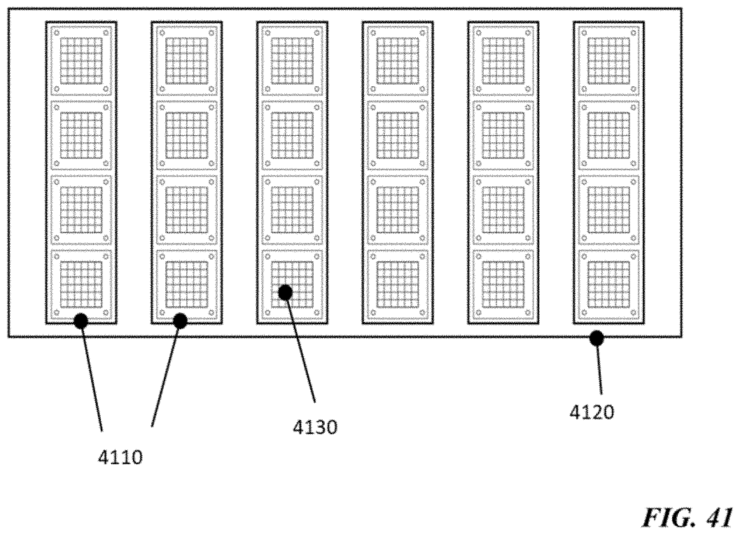

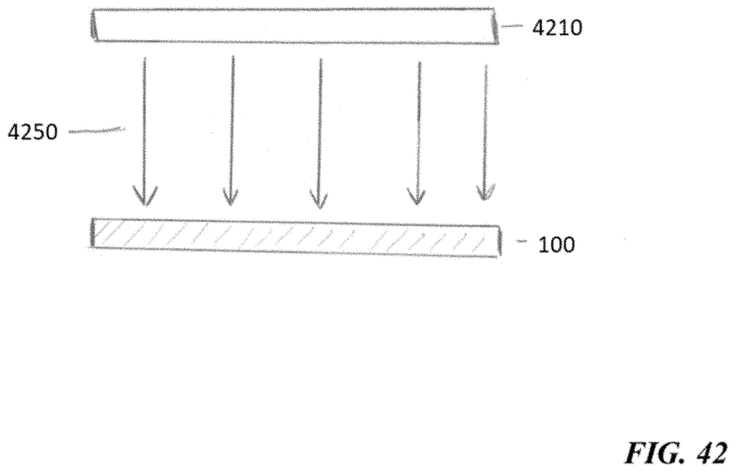

FIG. 41 shows the top view of a number of reconfigurable array tiles (4110) stacked next to each other in a reconfigurable bay (4120). Such an architecture may provide customization for the number of tiles to be activated for a run. The assembly may allow for loading a single tile (4130) or a column of tiles in a reconfigurable tray. The reconfigurable bay, trays, and tiles can be of any arbitrary shape. Multiple trays can be loaded on to a reconfigurable bay to process, for example, 8, 96, 384, 1,536, 6,144, 24,576, or more samples in parallel. The bays, trays, and tiles can be stacked vertically, horizontally, or a combination thereof.

### Individual Control Vs Global Control of Evaporation

Regulating evaporation of one or more droplets (samples) on the arrays can be accomplished by processing multiple samples on an array or a plurality of arrays. Enclosing a single droplet on an array tile using methods described herein may accomplish large-scale processing. An entire array tile or a plurality of array tiles may be covered to enclose one or more droplets simultaneously. Enclosures can be lowered on to the array before, during, and/or after droplet processing.

### Common Reagent Dispenser

The same set of reagents (e.g., biological samples, chemical reagents, solutions, nucleic acids (e.g., DNA, RNA, PNA, etc.), optical reagents, etc.) may be introduced to one or more tiles of an array (e.g., as in FIG. 41) while processing samples. A shared dispenser that distributes reagents across tiles may accomplish the introduction of such reagents. These dispensers may include dispensing mechanisms described herein. The dispensers can comprise one or more distinct channels. Each channel of the distinct channels may be utilized to dispense a single reagent throughout a given process. The dispensers may only comprise a single channel. The single channel may be used to dispense various reagents in a single process. A washing solution may be used to wash a single channel between dispensing different reagents to prevent any possible cross-contamination. The dispensers described herein can also be used to aspirate samples/reagents from the array surface. Wash steps can be performed between consecutive aspiration steps.

An array or a plurality of arrays may be positioned inside a liquid handling automation instrument as described herein. Samples and reagents may be dispensed on to the array by the liquid handler. The array, or plurality thereof, can be removed from the liquid handler (e.g., manually or autonomously) and located adjacent to the liquid handler.

### Single Sample to Multiple Samples

A two-step approach to developing and deploying biological and chemical automation workflows on the arrays may be performed using methods and systems described herein. The workflow may be developed on a single array element and the reactions may be iterated (e.g., manually or autonomously). An optimized workflow can be deployed across multiple arrays. For example, next generation sequencing (NGS) sample preparation workflows on a single sample processing unit can be developed. The developed single NGS sample preparation workflow can then be deployed on an array capable of processing 96 samples in parallel, each of these 96 samples being processed according to the developed single NGS sample preparation workflow.

### Bonding Films/Cartridges

Films, which may be disposable cartridge, (4210) may be coupled to the surface of the array (100) using adhesives. Such adhesives include, but are not limited to silicone, acrylic, epoxy, pressure sensitive adhesives and/or thermal glue (4250). In some cases, the films may be held firmly on the chip by using vacuum suction (4250) to eliminate any air gaps between the film and the surface of the chip (FIGS. 4A-4C). The film, which may be disposable, may be rigid or inflexible. The film may be a thin wafer of glass serving as the dielectric, coated with layers of hydrophobic coating, reference electrodes, or any combination thereof. This rigid film may be bonded to the array with EWOD capability using processes described herein. The methods described herein may be employed for bonding films to the chip surface in the one-plate as well as two-plate systems.

### Surface Coating

### Stacking Dielectric and Slippery Coatings Using Polymer Films

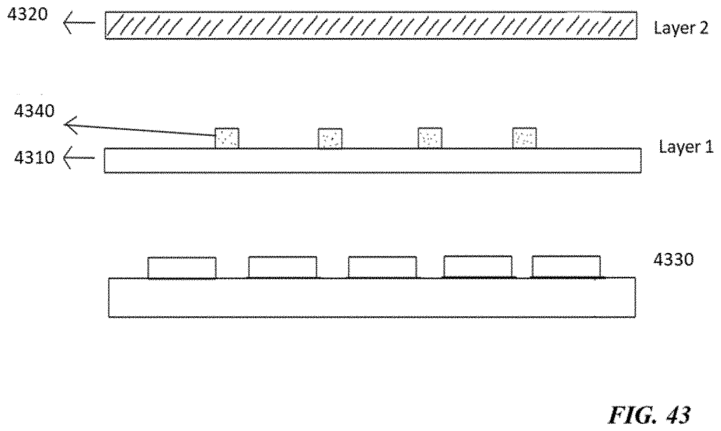

Stacking dielectric (4310) and slippery (4320) coatings on actuation electrodes (4330) of an EWOD array FIG. 43 may comprise at least two or more layers: for example, layer 1 may be a dielectric or a polymer film (4310) and layer 2 may be a slippery surface (4320, e.g., a porous polymer filled with oil or other hydrophobic material). Layer 1 may be the base layer. Layer 1 may comprise a polymer film (e.g. FEP, PFA, polyimide) with a thickness of at most 0.001 μm, 0.01 μm, 0.1 μm, 1 μm, 10 μm, 50 μm, 100 μm, 500 μm, or more. Layer 1 may comprise a polymer film (e.g. FEP, PFA, polyimide) with a thickness of at least 500 μm, 100 μm, 50 μm, 10 μm, 1 μm, 0.1 μm, 0.01 μm, 0.001 μm, or less. Layer 1 may comprise a polymer film (e.g. polyimide) with a thickness from about 0.001 μm to about 500 μm, about 1 μm to about 100 μm, or about 1 μm to about 50 μm. The top portion of layer 1 may comprise a layer of a conductive electrode array (4340). The thickness of the conductive electrode array may be at most 0.001 μm, 0.01 μm, 0.1 μm, 1 μm, 10 μm, 50 μm, 100 μm, 500 μm, or more. The thickness of the conductive electrode array may be at least 500 μm, 100 μm, 50 μm, 10 μm, 1 μm, 0.1 μm, 0.01 μm, 0.001 μm, or less. The thickness of the conductive electrode array may be from about 0.001 μm to about 500 μm, about 1 μm to about 100 μm, or about 1 μm to about 10 μm. The conductive electrode array may be, for example, a screen printed, a digitally printed, or electroplated (e.g., with a photolithographically patterned precursor) onto the film-based dielectric. Layer 2 may provide a slippery surface for droplet transport. Layer 2 may comprise a textured polymer material. The textured polymer may be a porous polymer film (e.g. ePTFE) filled with a lubricating oil (e.g. silicone oil). Layer 2 may also be made of other hydrophobic materials such as, for example, Teflon AF, CYTOP, fluoropolymers, silanes, or a combination thereof. Layer 2 may be made of any other slippery material described herein. Layer 2 may contain partially or fully conductive paths to the electrodes contained in Layer 1. Also, the top surface of layer 1 may not comprise an electrode array. For example, the electrode array may be embedded within or coupled to the top surface of Layer 2.

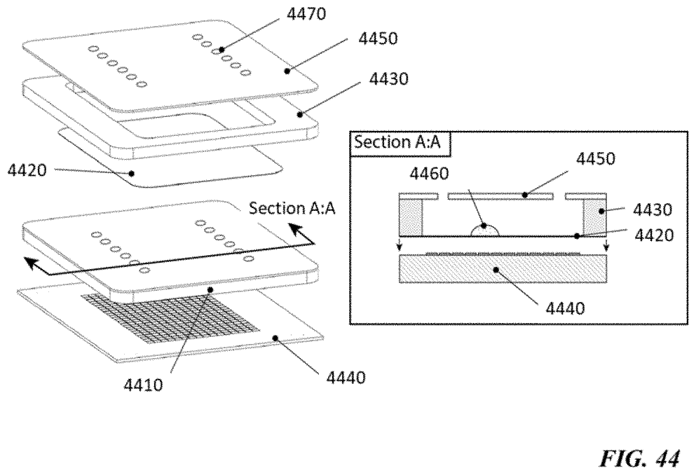

The entire construct of dielectric and slippery material (e.g. combination of polymer films) may be removable/disposable/replaceable in part or entirely (4410). The individual layers (4420, e.g., layer 1, layer 2, or a combination thereof) may be attached to a frame (4430) that fits onto the actuation electrodes to form the electrowetting array (4440, FIG. 44). This frame may contain a top plate (4450) to mitigate evaporation of a droplet (4460). The top plate may be heated. The top plate may have a layer of an electrode array. The top plate may have holes (4470) for introducing droplets or connecting to an external device to control, for example, pressure, humidity and temperature. This frame-and-layer construct, layer 1, layer 2, or any combination thereof may be removable/disposable/replaceable to avoid cross-contamination of samples being manipulated on an array surface.

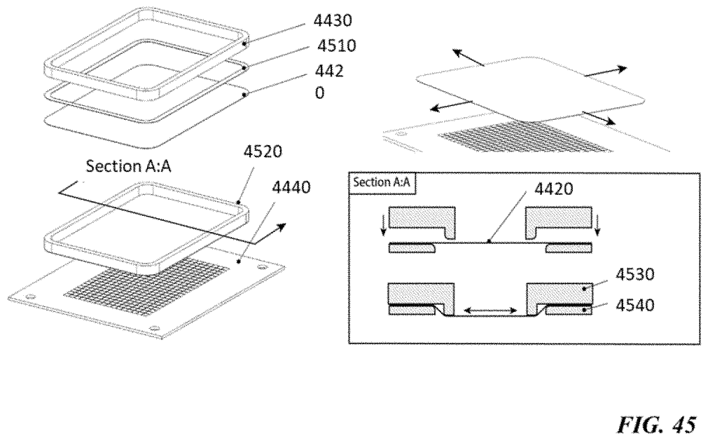

Layer 2 (e.g., textured solid filled with oil) may be directly applied to the electrode array. The electrode array may be coated with a protective conformal coating and, for example, a removable layer 2 (e.g., textured solid filled with oil) can then be applied. An adhesive (4510, FIG. 45) that is pressure sensitive, heat sensitive, or a combination thereof may be used to bond layer 1 to the array, layer 1 to layer 2, or a combination thereof. A film cartridge (4520) may be used to hold the frame configuration. A vacuum may be used to improve contact between the stacked layers and the array. Individual layers or their combinations can be attached to a frame.

### Conductive Layer Through Hydrophobic/Slippery Coating

Holes may be introduced to layer 2 of the stacked layers depicted in in FIG. 43. Conductive materials (e.g., carbon paste or silver nanoparticles) may be introduced into these holes. Alternatively, porous films filled with conductive material can be used for layer 2. Also, oil may be applied to the dielectric, which may yield a partial or fully conductive hydrophobic slippery layer. The porosity of the dielectric film may be sufficient and may eliminate the introduction of physical defects (e.g., holes). The physical defects alone may suffice in establishing a conductive pathway, eliminating the introduction of additive materials such as, for example, conductive materials (e.g., carbon paste or nanoparticles).

### Film Tension

Layer 1, layer 2, or a combination thereof may be kept under tension by individually using a tensioner (e.g., a spring-loaded tensioner). The tensioners may allow the layer stack to expand and contract during thermocycling to avoid formation of wrinkles or other defects on the EWOD array surface. The film under tension may be provided to a configuration depicted in FIG. 44. The film can undergo further tension by applying a top frame (4530) to a bottom frame (4540) while assembling the array (Section A:A, FIG. 45). Various methods of tensioning the film can be used including, for example, “frame-in-frame” tensioners (e.g., which sandwich the film between two frames and apply tension in all directions as the frames are brought together FIG. 45).

### Film Application Methods and Systems

A squeegee tool may traverse across the surface of the stacked films against the array to apply a film. The squeegee traversal process may also remove of wrinkles in the stacked films.

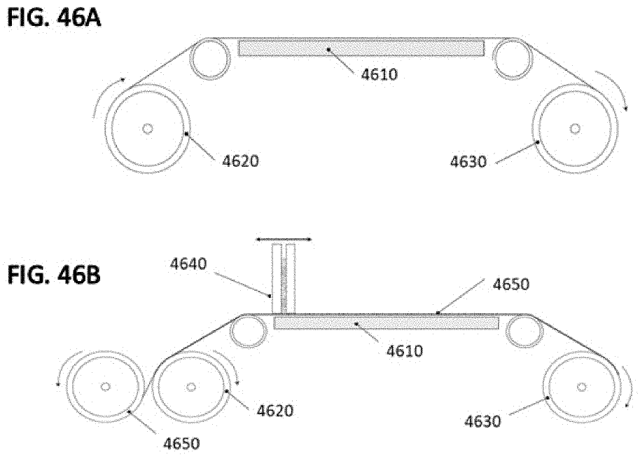

The film (e.g., layer 1, layer 2, or a combination thereof) may be applied onto the array (4610) using a roll-to-roll film conveyor system, for example, as shown in FIG. 46A. The film feed (4620) and used film (4630) rollers may be used to maintain tension in the film in order to avoid wrinkling/deformation of the film during thermocycling operations, sample manipulation, or a combination thereof. For example, the layers may be i) pre-assembled and placed on the same roller (FIG. 46A) or ii) located on separate rollers via a dispenser (4640) (FIG. 46B) and assembled prior to application on the array. Additionally, a porous membrane feed roller (4650) may be coupled with the film feed roller (4620) to provide a porous membraned film.

Layer 2 may require oiling before, in between, or during sample manipulation on the EWOD array. This may be accomplished using an oil dispenser (4640) (e.g., FIG. 46B). The oil dispenser may apply oil (4650) onto layer 2 by, for example, spraying, jetting, brushing, or any combination thereof. The oil dispenser may be integrated into the front roller to apply oil to layer 2 prior to its application onto the array surface.

### Consumables

### Preloading Frames+Filmframes

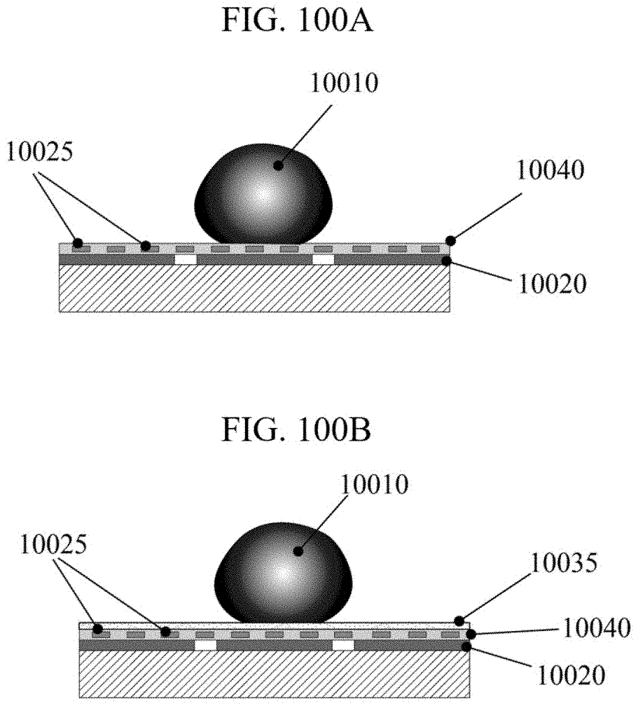

In one embodiment of an electrowetting device (or array device described herein), the reference electrode 10025 (electrode array connected to ground other potentials) may be placed on the same side of the droplet as the actuation electrodes as shown in FIGS. 100A and 100B. In some embodiments, the reference electrodes 10025 are embedded within a dielectric layer 10040. The dielectric layer 10040 comprising the reference electrodes 10025 may be disposed on top of a layer comprising electrodes 10020 which are facilitate movement or actuation of the droplet 10010. The top most surface may or may not have a slippery/hydrophobic coating 10035. It is advantageous to make a good DC electrical contact with the reference electrode in order to facilitate discharging any accumulated charge in the droplet.

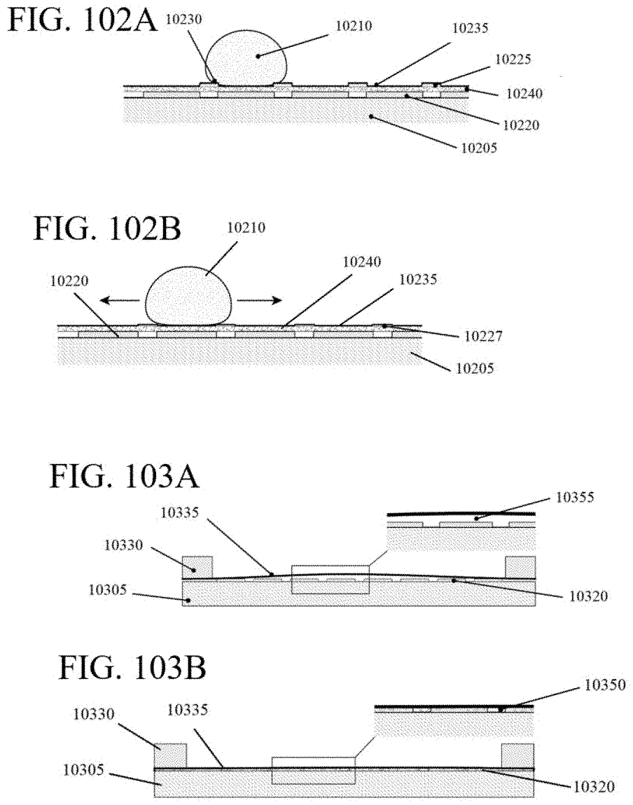

The reference electrode may connect to a known potential (for example to ground potential) via a frame 10150. This frame 10150 may be the same film to which the dielectric and the slippery layer are attached, as depicted in FIGS. 102A-102B. In some embodiments, the film frame 10150 is placed within and alignment frame 10155 and on top of substrate 10140. The frame 10150 may be held in position by one or more spring clips 10160. Within this film-frame construction, the reference electrode array may make electrical contact with the film-frame with a variety of different means. If the hydrophobic (or slippery) coating on the top surface of the reference electrode is thin (<5 um), sufficient conductivity may be established by simply adhering the film-frame to the reference electrode with a thin adhesive (e.g. cyanoacrylate). If, however, the hydrophobic coating is thick, electrical contact between the film-frame and the reference electrode may be established through the selective removal of the hydrophobic coating in regions followed by the use of cold solders such as metal-filled adhesives.

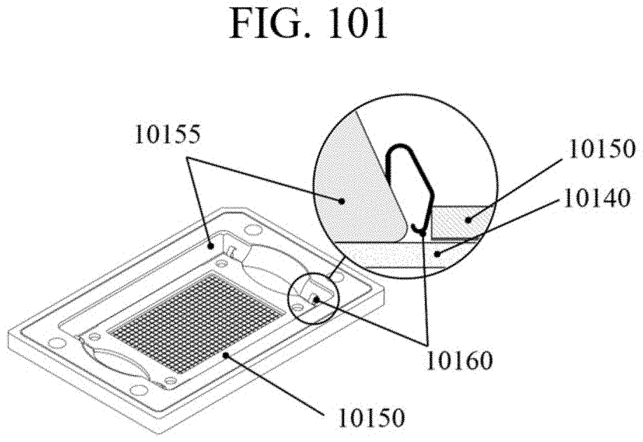

An electrical connection between the film-frame and the instrument may be established using conductive spring-loaded connectors (10160 as depicted in FIG. 101), soldered wires bolted-connections, or other means known to those skilled in the art.

In some embodiments, the configurations described herein can be applied to the cartridges described several sections above.

Film Composites

In order to prevent charge accumulation in a droplet, a patterned electrode may be used on the droplet-facing surface of the dielectric substrate. This patterned electrode may be made using a number of different fabrication methods including screen printing, flexographic printing, gravure printing, inkjet printing, sputtering, and vapor phase deposition techniques. The metallic inks used in the printing processes play an important role in determining the properties of the printed electrode. Silver-particle inks can regularly produce features sizes down to approximately 100 um and have a typical minimum thickness of deposition of approximately 1 um.

If a thin (typically <1 um) conformal hydrophobic coating is used to produce the hydrophobic layer of the coating stack, the thickness of the printed electrode is important in determining whether droplets will be able to move freely on the surface or be pinned in place. It is typically desirable for the trace-height of the printed features to be substantially smaller than the droplet itself. For 100 uL droplets or smaller, 1 um thick traces with a thin hydrophobic coating can greatly impede motion.

It is therefore desirable to pattern electrodes that are substantially thinner than lum, when using thin conformal hydrophobic coatings, as depicted in FIGS. 102A and 102B. Particle free ink formulations that exploit a chemical reaction that precipitate metallic particles are able to reach much smaller feature sizes (˜5 um) and produce much thinner traces (<100 nm). These inks can be patterned using conventional printing processes and are compatible with a variety of substrates including PET and PI dielectrics. FIG. 102A depicts a droplet 10210 to be transported across an array. In some embodiments, the array comprises a first layer of electrodes 10220 adjacent to a substrate 10205. A dielectric layer 10240 may be provided above the first layer of electrodes 10220. A second layer of electrodes 10225 may be provided above the dielectric layer 10240. A conformal coating 10235 may be provided on top of the second layer of electrodes 10225. In some embodiments, the conformal coating is hydrophobic. If the electrodes are too thick (e.g. produced by some screen-printing methods), they may create pinning features 10230 which impede movement of a droplet. Therefore, electrodes may be printed by methods disclosed herein to produce a layer of particle free electrodes 10227 which will not impede movement of a droplet, as depicted in FIG. 102B.

In some embodiments, the configurations described herein can be applied to the cartridges described several sections above.

Film Frame to Tile Application

In one embodiment of an electrowetting device, a thin (<5 um) porous film may be used to create a liquid infused surface on which droplets can freely move. This porous film may be attached to the dielectric film with the use of a film-frame that adheres the three layers (dielectric, porous, frame) at the periphery of the frame. This adhesion can be accomplished with a wet adhesive, dry adhesive, or through thermal lamination. These adhesive strategies can be selectively implemented in regions (e.g. along the periphery of the frame) or across the entire surface of the films.

Thermal lamination is possible when using certain combinations of materials. The dielectric film may be composed of PET, FEP, or PFA to allow for thermal lamination to a textured and porous membrane (Ex: PTFE porous membrane). This thermal lamination process results in a robust film that maintains a porous top surface that can be infused with a liquid to create a liquid infused surface, which enables high performance droplet mobility.

To achieve consistent droplet mobility across the entire electrode array, it is necessary for the film or coating stack-up to be in consistent and close contact with the electrode array and substrate. A variety of methods can be used to achieve this close contact. Tensioning devices can be used to stretch a film-based coating to ensure tight contact with the substrate. Alternatively, vacuum pressure can be used to pull the film tightly against the substrate through small holes or porous features in the substrate.

As depicted in FIGS. 103A and 103B, a substrate 10305 may be provided having comprising an electrodes 10320. In some embodiments, a film 10335 may be provided on over electrodes 10320 and held in place by a film frame 10330. Air bubbles 10355 may be trapped between the film 10335 and electrode array 10320 when the film is attached. These can be easily pushed to the edge of the film with the use of a squeegee or brush. In some embodiments, as depicted in FIG. 103Bb a filler-fluid 10350 is used to ensure good adhesion between the film-layer and the substrate. A thin layer of filler fluid 10350 may be placed between the electrode array 10320 and the bottom film-layer 10335 to smooth any wrinkling of the film and, through surface tension, removes any air gaps. Filler fluids may include a variety of insulating materials including silicone oil or fluorinated oils.

In some embodiments, the configurations described herein can be applied to the cartridges described several sections above.

Reservoir for Waste Disposal

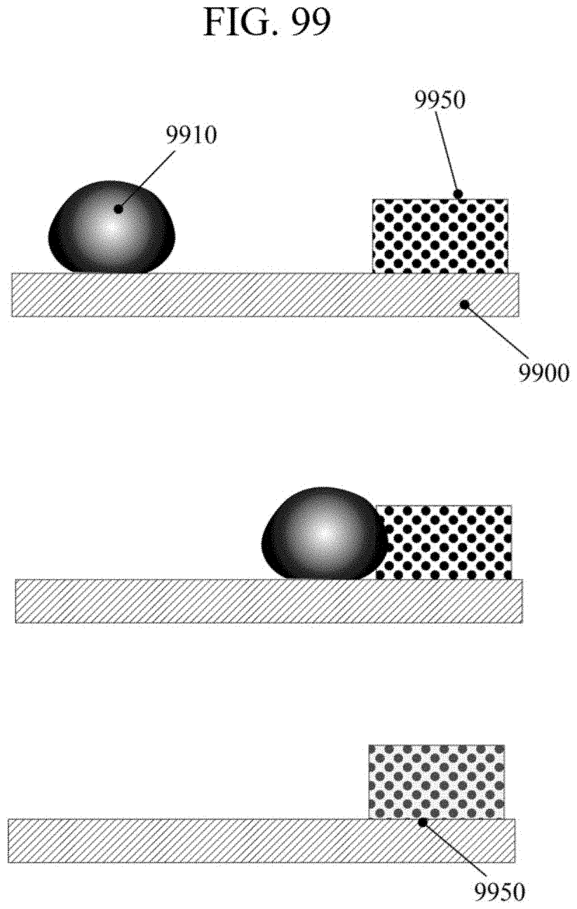

It may be possible to attach an absorptive material or sponge to the array device as shown in FIG. 99. A droplet 9910 can be transported using electromotive forces on the array device 9900 and contacted with the sponge 9950. Upon establishing contact the droplet may be absorbed by the sponge 9950. This may be one method to dispose of or store unusable liquids during, before or after running the biological workflow. It may also be possible to remove or dispose of unnecessary or wash buffers using any of the dispensers (capillary, automated pipettor, piezo actuator) described herein. It may also be possible to dispose of the waste liquid by sucking it through a hole on the array device (as described in other section of this disclosure).

### Improving Liquid Recovery

Volume error by liquid handling robots, which may be due to, for example, poor fluid recovery from a well-plate can be an issue. With EWOD-enabled aspirations, the volume errors may be significantly reduced by accurate positioning of the droplets through EWOD-actuation. This aspect may be coupled with a computer-vision based algorithm, which may feed back to the liquid handler. The system described herein may ensure <5% volume errors due to transfers. In some embodiments, activating EWOD-enabled mixing during aspirations may improve liquid recovery. The transfer efficiencies may be further improved by including mechanical, acoustic, or a combination thereof vibrations on the array. A potential may be applied to the pipette, resulting in electrowetting of the droplet into the pipette, further resulting in high transfer efficiency.

### Single Cell Isolation, Cell Barcoding, Tracking

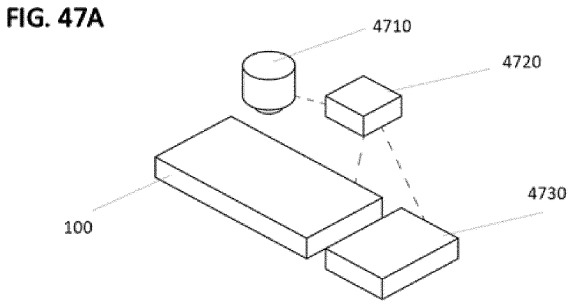

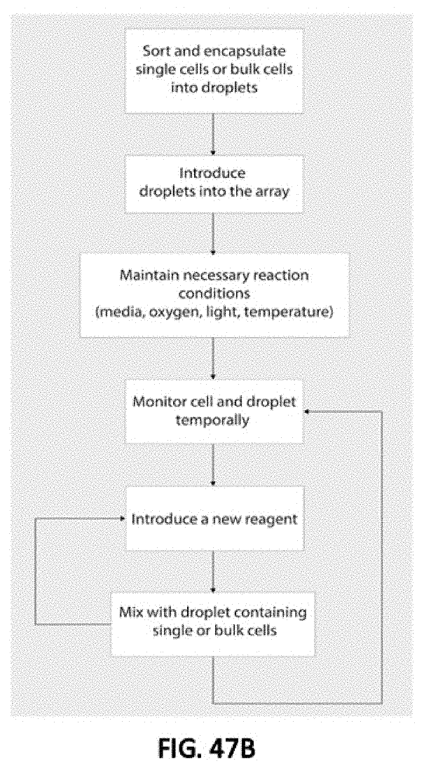

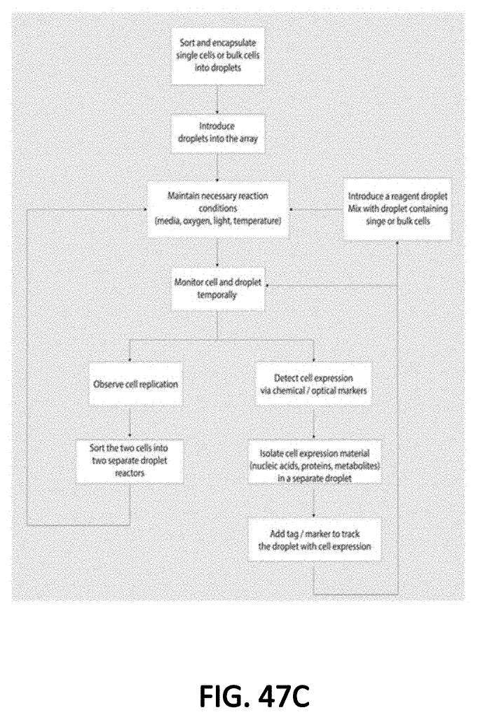

A single cell contained in a discrete droplet may be manipulated using electromotive forces, or other forces described herein, on the array (100, FIG. 47A). A single cell may be generated using a single cell sorter coupled to the array (4730). The cell may be, for example, a cancerous cell or a normal cell. The cell may be, for example, a mammalian cell, a plant cell, an insect cell, a bacterial cell, or a yeast cell. The array may temporally and spatially monitor the kinetics of cellular functions, such as, for example, cell growth, cell expression, cell division, or any combination thereof. A detector (4710, e.g., an optical microscope or a camera) may be coupled with the array for monitoring. A control module (4720) may be coupled with the array to control the processes described herein. Cellular expression may comprise, for example, nucleic acids, proteins, metabolites, ions, molecules expressed on a cellular surface, or any combination thereof. The system may introduce one or more reagents (or markers, such as, for example, antibodies) to a droplet containing a single cell (FIG. 47B). The system may introduce reagents for a cell's expression. The system may transport a droplet to various reaction conditions (e.g., temperature, oxygen, nutrients, etc.). The system may temporally monitor how the addition of a reagent (or reagents), or introduction to new reaction conditions, may affect the kinetics of cellular functions (e.g., growth, expression, replication, etc.).

The cellular functions discussed herein can be controlled for a group of cells (e.g., bacterial cells) contained in a droplet. A central reservoir of cells in relevant media may be contained on, adjacent to, or a combination thereof the array. Droplets from the reservoir of cells can be generated through electromotive force enabled actuation. Antibiotics or other cellular toxins may be introduced into the discrete droplets containing cells at any concentration. The cell growth in each droplet may be monitored. The cytotoxicity for each antibiotic/toxin (e.g., IC₅₀, LD₅₀, EC₅₀, ED₅₀, GI₅₀, MIC, etc.) can be tested. The array can, for example, aid the identification of antibiotic resistant strains in microbial analysis. The antibiotic/toxin may be lyophilized on the surface of the array or disposable cartridge. The antibiotic/toxin can be solubilized in a buffer or media prior to or during the introduction of droplets containing cells.

While the cell is contained in a droplet, expressed material derived from that cell may be isolated in a separate droplet (FIG. 47C). The droplet containing the expressed material may be tagged with a unique identifier (e.g., nucleic acid, peptide, antibody, etc.). The expressed material may be tagged with a unique identifier. The system may continuously monitor and measure the tagged droplet for further analysis. The droplet with tagged material may be combined with another droplet containing a single cell. Genomic material, such as, for example, DNA or RNA may be extracted from a cell, or a plurality thereof, in a droplet after lysing the cell, or plurality thereof. Nucleic acids from a cell, or a plurality thereof, may be tagged with a unique molecular identifier. The tagged or untagged genomic material may be isolated and used in library preparation for sequencing.

A cell may replicate to two, three, four, five, six, 10, 50, 100, 1,000, or more cells in a droplet. The system may view the droplet in real-time, and it may respond to the process by dividing the droplet to at least two droplets, compartmentalizing at least two cells into the at least two droplets. The system may process the single cell, or plurality thereof, in the droplets as described herein. The array may contain a 1, 2, 5, 10, 20, 50, 100, 1,000, or more droplets as compartments enclosing a single cell. All droplets on an array, or plurality thereof, may be monitored simultaneously. At least two droplets, each containing at least one cell, may be combined. The combined cells may be monitored to, for example, observe how they interact with each other. The compartmentalization of cells to a droplet may be done on an external device (4730, e.g., microfluidic, FACS, optical tweezers, manual picking, micromanipulators, etc.). The compartmentalized cells of a droplet may be introduced to an array (e.g., electrowetting array) for droplet manipulation after compartmentalization on an external device. The array may contain structures to isolate and store the cells. The compartmentalization of cells into individual droplets may be integrated, for example, on the array (e.g., electrowetting array) using, for example: an electrowetting or a dielectrowetting device, a dielectrophoretic device integrated with the array, optical tweezers integrated with the array, microfluidics devices integrated with the array.

### Multi-Story Chip

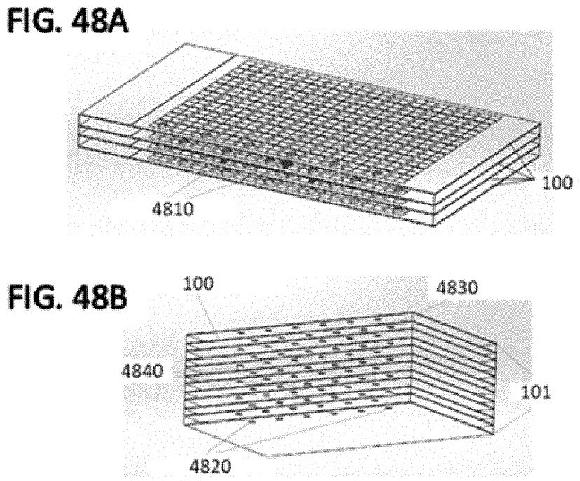

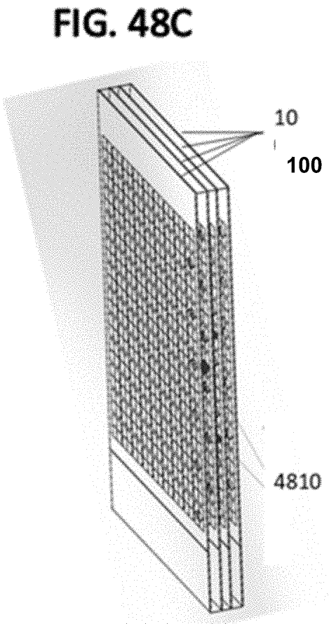

The systems and methods described herein can be in three-dimensional (3D) space. The device (101) may comprise multiple plates (100, FIGS. 48A-48C). The device may contain gaps between any plates of the array where liquids (4810) can be manipulated. The gap between any two plates may contain a filler fluid. The array may resemble a multi-story building (e.g., FIGS. 48A-48C). Any plate may contain arrays of electrodes for, for example, electrowetting, electrophoresis, dielectrowetting or other electro-motive force based actuation described herein. Alternately, the array may contain one or more piezoelectric actuators. Alternately, the array may comprise plates with gaps, holes, channels, or any combination thereof, where liquids can be flown by applying forces described herein (e.g., pressure, vacuum, elecro-motive).

The multistory device may be constructed using the methods and systems described herein. The device may include sensors for monitoring samples as described herein. Liquid input and liquid output devices coupled with arrays described herein can be coupled with a multi-story array. The multi-story chip may be positioned vertically (FIG. 48C), horizontally (FIG. 48B), diagonally, or any combination thereof. Liquids can be maneuvered (e.g., opposing forces of gravity) in the space vertically, horizontally, diagonally, or any combination thereof.

A multi-story array may actuate (e.g., move, mix, split, heat, cool, oscillate, bead-based wash) liquids (e.g., droplets) located between or on any two layers of the multi-story array. Liquid between two plates may touch both plates or it may touch only one plate. The plates may contain sensors to identify, for example, the position, size, composition, or any combination thereof of a liquid (e.g., a droplet). A liquid (e.g., a droplet) may be transferred from one plate (100) to another through a hole (4830), a channel (4840), or a combination thereof of a plate, or a plurality thereof (FIG. 48B). Any two sides of a plate, or plurality thereof, may be connected using, for example, pipes, tubes, microfluidic channels, electrowetting arrays, or a combination thereof. The holes may separate the sides of a plate with a semi-permeable membrane, a permeable membrane, a porous membrane, or any combination thereof. Liquids on multiple plates may be maneuvered simultaneously or separately and positioned such that they interact through the holes, pipes, membranes, tubes, or a combination thereof. The multi-layer array may be integrated with other arrays to flow liquids in and out of the multi-layer array through inlets/outlets (4820). External devices to flow liquid in and out may be, but are not limited to: tubes, microfluidic devices, liquid handling robots, liquid dispensers described herein, or any combination thereof.

The multi-story array may be used to process at least 1, 5, 10, 50, 100, 500, 1,000, 10,000, 50,000, 100,000, 500,000, 1 million, or more components described herein in parallel. The multi-story device may take the shape of a SBS plate in an XY plane. Any two layers of the device may be connected by holes. These holes may be used to transfer one or more samples from one plate to another. On each level of the array, the sample can undergo one or more process steps of biological workflow. Substantially all arrays of the multi-story array can act synergistically within a system.

### DNA Data Storage

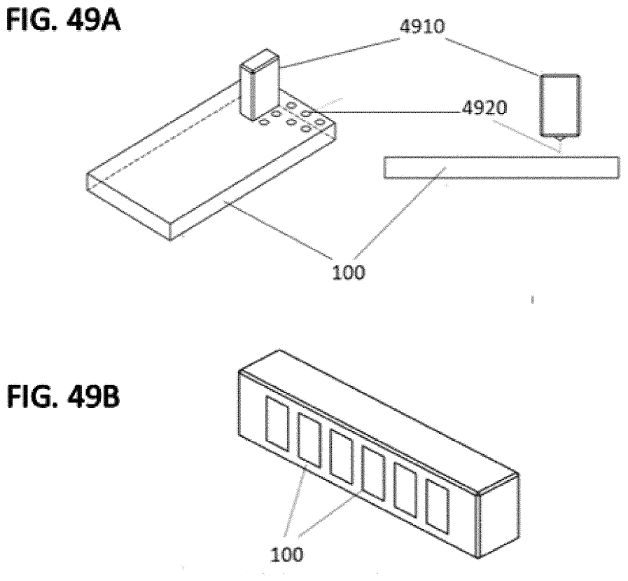

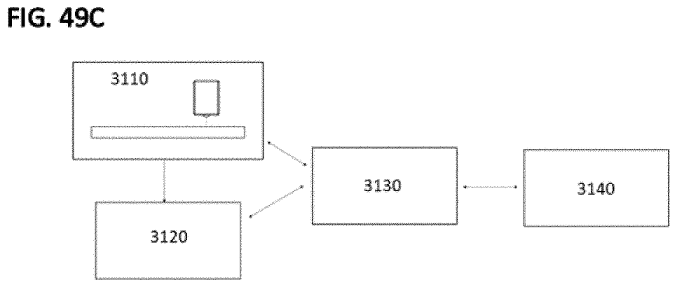

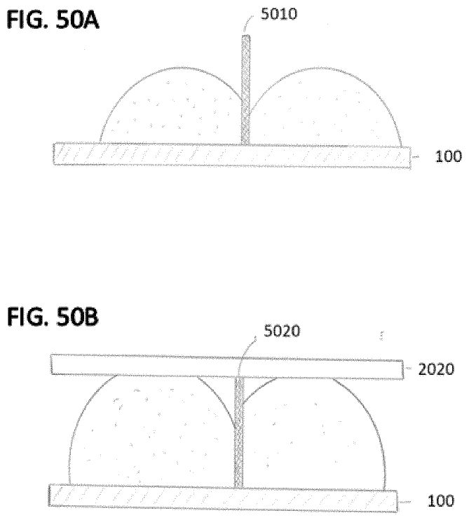

Polymeric material (4920, e.g., nucleic acids, peptides, or polymers described herein) may be deposited from an external device (4910) on the surface of an array (100) described herein (FIG. 49A). An external device may be, for example, a pipetting robot, inkjet nozzle, electro-fluidic pump, microfluidic dispensers, liquid dispensers described herein, or any combination thereof. The array can be a combination of the arrays described herein. The polymer material may be deposited onto a location of arrays described herein and retrievable in an addressable way. The addressing scheme may be 1:1 (i.e., every spot with a unique polymer has a unique address). Each polymer location may encode information. The array may contain encoded information as a data storage device. The data storage device may be used for archival (e.g., cold storage) purposes (FIG. 49B). The material on the array may be accessed (e.g., by shuttling droplets with reagents) and solubilizing the material into a droplet (FIG. 49C). The contents of the droplet may be sequenced (e.g., on a DNA sequencer) to retrieve information about the material. PCR amplification may be performed on a location of the array and subsequently transferred to a sequencer (4920). A controller (4940, e.g., CPU, microcontroller, Field Programmable Gate Array (FPGA)) may access data from the storage array by a sequencer (4920, e.g., nanopore) and use dispensing methods and systems described herein to perform data write operations (4910). The address of the components described herein may be performed by an address encoder or address decoder (4930). The polymer material may be deposited on a disposable cartridge as described herein or on a polymer film. The disposable cartridge or film may be placed on an electrowetting array. Information may be retrieved using appropriate reagents as described herein. Information may be “erased” from the array at any location programmatically by shuttling droplets with reagents.

### Droplets Separated by a Membrane on an Electrowetting Device

A membrane (5010, e.g., porous, permeable, semi-permeable) may be permanently or temporarily attached to an array (FIGS. 50A & 50B). Droplets may be actuated on the array and positioned to establish contact with the membrane simultaneously for a desirable amount of time. The droplets may exchange materials (biological and/or chemical) from one droplet to another. The system may be an open (FIG. 50A) or closed (FIG. 50B) system.

### Customizable EWOD Chip

A customizable EWOD chip may comprise a base layer of micro-sized actuation electrodes (less than about 1 mm) that may be coupled with a cartridge containing components described herein (e.g., additional electrodes, dielectric material, conductive material as reference electrode, hydrophobic coating, etc.). The microelectrodes, which may be contiguous, may be grouped together to form electrodes that can actuate droplets. The electrodes may be arbitrary in shape and size. The customizability in the sizing of the electrodes as well as the patterns of the array of electrodes may be used to process a variety of biological and chemical workflows. The micro-sized electrode array may be used for generation of actuation potential. The micro-sized electrode array may not be used for droplet manipulation. In some embodiments, another layer of electrodes can be coupled with the array for droplet actuation.

### Capacitive Sensors for Droplet Detection

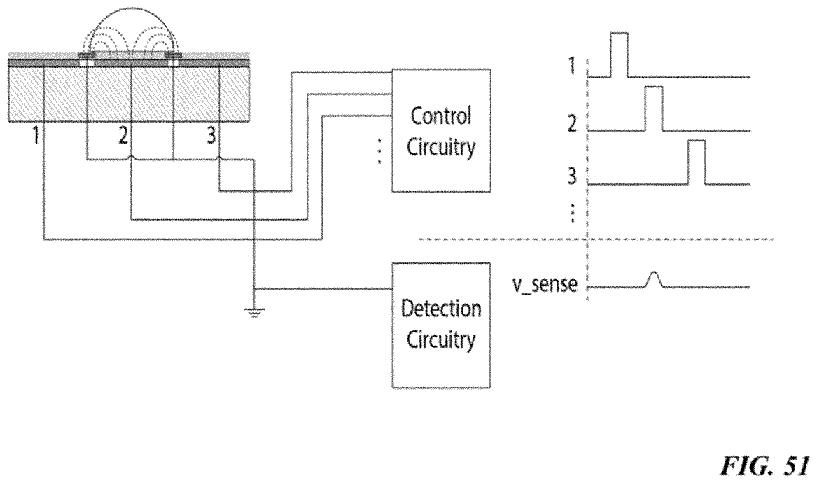

Detecting the position of droplets can be accomplished with systems beyond computer-vision. Capacitive sensing may enable the detection of droplets on an array surface. This can be accomplished by actuating an electrode and detecting a change in voltage on a neighboring electrode (FIG. 51). To detect the position of droplets of an array and their approximate sizes, electrodes in the array can be activated sequentially while circuitry monitors of a reference electrode that spans the entire array can be detected. For example, when an electrode is activated where a droplet is present, a change in voltage can be sensed on the reference electrode. The detection circuitry may comprise a feedback amplifier that buffers and scales voltage changes such that, for example, they can be read by digital circuitry on a microprocessor or other control circuitry.

The reference electrode can be coplanar with the actuation electrodes, can be separated from the actuation electrodes by a dielectric film layer, or can be opposite the actuation electrodes on top of the droplet. This reference electrode may be in conductive contact with the droplet but may also be insulated from the electrode array. The electrode may be porous or grid-shaped so as to not electrically shield the droplets from electrode array. The position detection technique may also be used without an array-spanning reference electrode, for example, detecting changes in the mutual capacitance between array electrodes. This technique may activate electrodes within the array and monitor neighboring electrodes through signal conditioning circuitry.

Electroporation with Second Electrode Layer

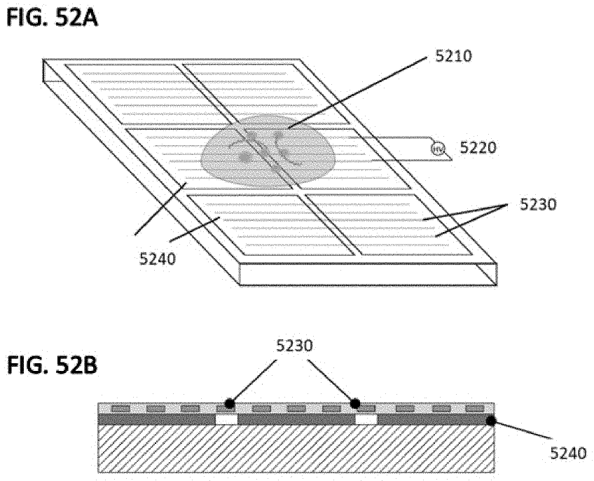

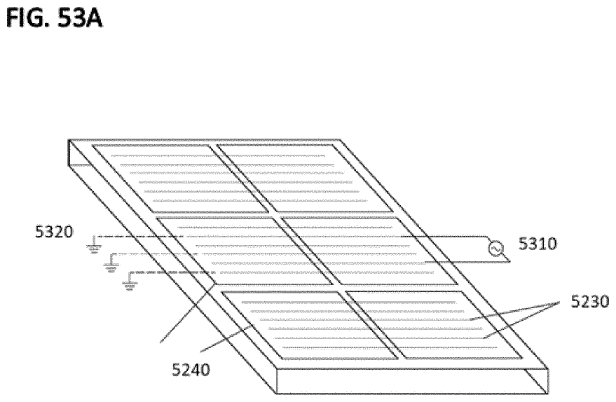

FIGS. 52A-52B shows a droplet (5210) on an open array encompassing cells and biomolecules (e.g., nucleic acids). The droplet sits above two layers of electrodes and may be separated from these electrodes by a dielectric layer. For example, the droplet is closer to the second layer (5230) of electrode than it is to the first layer (5240) of electrodes (FIGS. 52A and 52B). Alternating electrodes in the second layer of electrodes may be pulsed with high voltages (5220) to perform electroporation in the cells suspended in the droplets. The voltage of the electrodes can be at most about 1 volt (V), 100 V, 500 V, 1,000 V, 5,000 V, 10,000 V, 50,000 V, 100,000 V, or more. The voltage of the electrodes can be at least about 100,000 volts (V), 50,000 V, 10,000 V, 5,000 V, 1,000 V, 500 V, 100 V, 1 V, or less. The voltage of the electrodes can be from about 1 V to about 100,000 V, 100 V to about 5,000 V, or 500 V to about 1,000 V. The pulse width of the voltages can be at most about 0.00001 milliseconds (ms), 0.0001 ms, 0.001 ms, 0.01 ms, 0.1 ms, 1 ms, 10 ms, 100 ms, 1,000 ms, 10,000 ms, 100,000 ms, or more. The pulse width of the voltages can be at least about 100,000 ms, 10,000 ms, 1,000 ms, 100 ms, 10 ms, 1 ms, 0.1 ms, 0.01 ms, 0.001 ms, 0.0001 ms, 0.00001 ms, or less. The pulse width of the voltages can be from about 0.00001 ms to about 100,000 ms, about 0.001 ms to about 1,000 ms, or about 0.1 ms to about 100 ms. The electrodes can be an arbitrary shape. For example, with the first layer of electrodes as actuation electrodes, the second layer of electrode can be used as a reference electrode to generate electrowetting forces. Therefore, on the same surface of the array, droplet manipulation using EWOD and electroporation of cells may be accomplished.

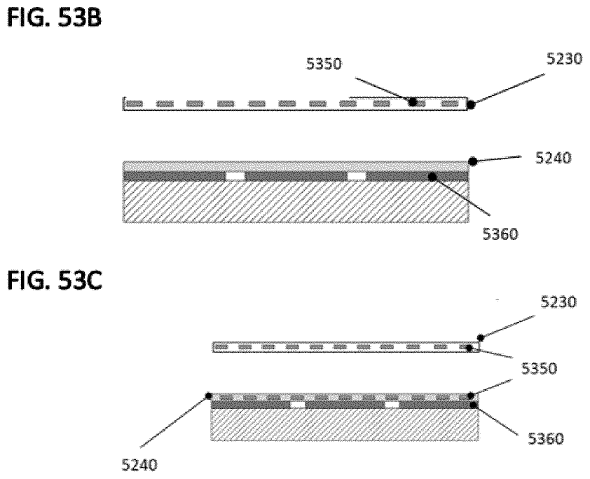

In another configuration, the array may consist of a top plate with another electrode array. A voltage can be applied between the electrode of the top plate and the electrode array of the bottom plate, which is closest to the droplet. The array in FIGS. 53A-53C can be used in an alternate configuration to transport, mix, and split droplets using dielectrowetting. For this, the second layer of electrode array may be used to affect droplet movement. The electrodes can be configured in a serpentine shape or they can be interdigitated. By applying alternating electric field (5310, e.g., AC) coupled to a grounded electrode between any two adjacent electrodes, dielectrowetting forces can be generated. FIG. 52B shows a side view of the arrays depicted in FIG. 52A and FIGS. 53A-53C. Additionally, the first layer of electrodes and the second layer can be used cooperatively for droplet transport using electrowetting (EWOD).

Similarly, in a two-plate system (e.g., FIG. 53B and FIG. 53C), the electrode (5350) on the top plate (with or without a slippery surface) (5230) can be used as a reference electrode or for electroporation (FIG. 53B). The electrode on the bottom plate (with or without a slippery surface) (5240) can comprise actuation electrodes (5360) for EWOD/DEW operations. Alternatively, a two-plate system may comprise both top plate (5230), bottom plate (5240), or a combination thereof (with or without slippery surfaces) may comprise electrodes (5350) that may be used as reference electrodes or for electroporation (FIG. 53C).

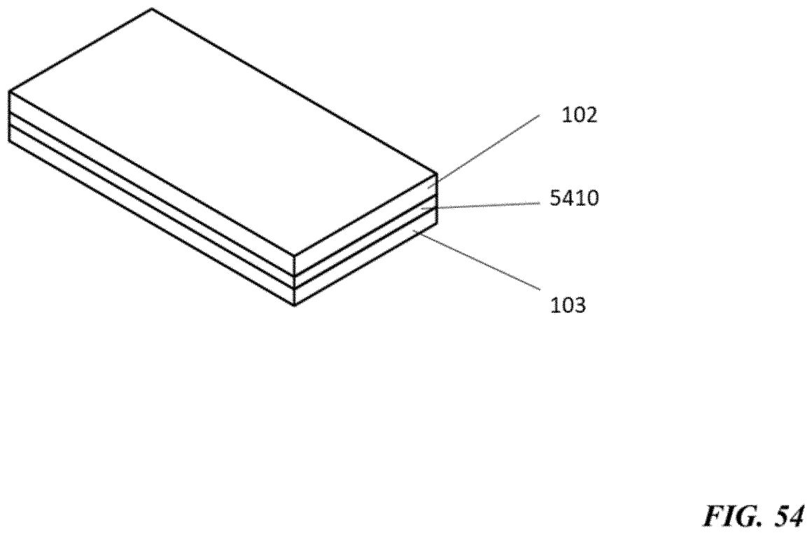

In a two plate electrowetting array, a standard capacitive sensing device (102, e.g., a touchscreen device) may be used as a second plate (FIG. 54). The capacitive device (102) may be used for measuring droplet characteristics described herein. The feedback from the capacitive device may be used for manipulating droplet characteristics described herein. The underlying array (103) can manipulate droplets in the space (5410) between the two plates. Alternately, a grid of transparent or conductive electrodes on the top surface can be used to perform the same functions. The second layer may have an additional layer of hydrophobic coating or a slippery coating (5410, e.g., SLIPS).

Polymerase Chain Reaction (PCR), Clean-Up, and Quantitative PCR (qPCR)

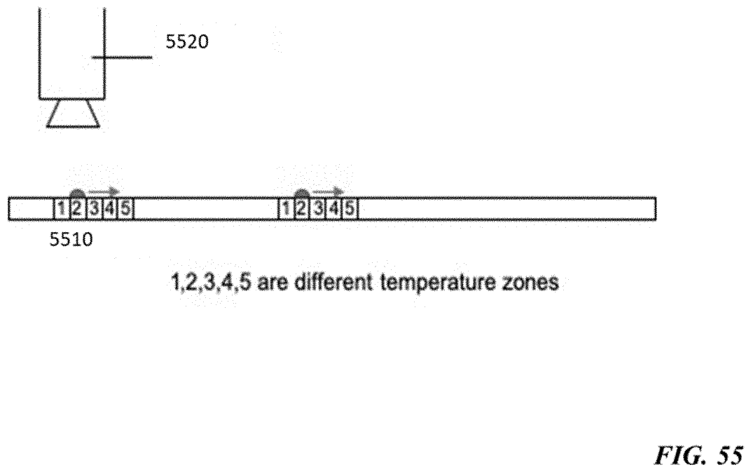

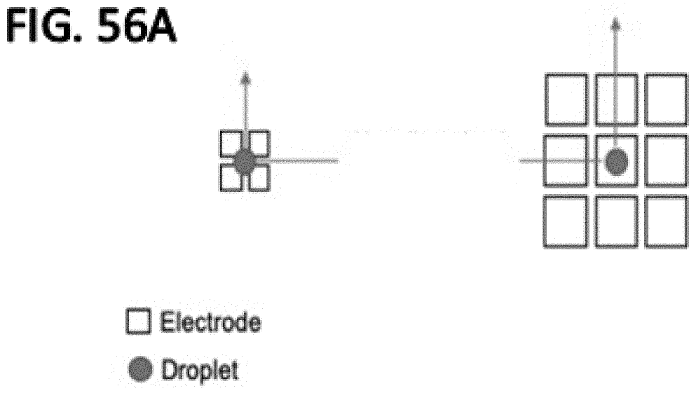

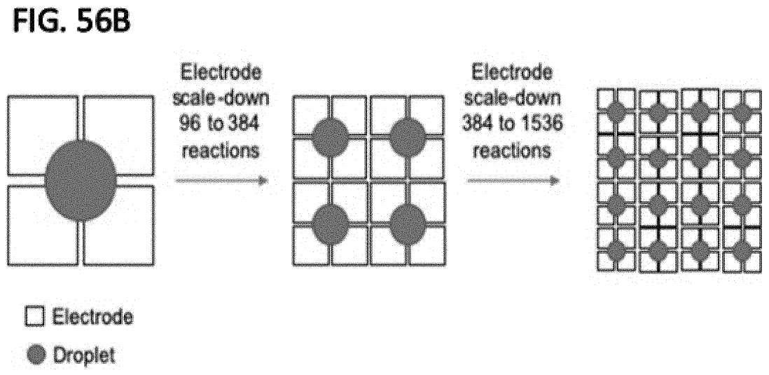

Nucleic acid molecules may be amplified by thermocycling-based Polymerase Chain Reaction (PCR) on an array described herein. A fixed region of the array may be heated or cooled. Alternatively, different regions on the array can be heated or cooled to different temperatures or temperature ranges (see zone 1, 2, 3, 4, 5 in FIG. 55). For example, one or more droplets containing PCR reagents and samples, can be shuttled back and forth between different zones (5510) of the array to perform PCR. A sensor (5520, e.g., a fluorescent camera) can be used to illuminate and record a signal (e.g., fluorescence) of the droplet on the array (FIG. 55). The detection may be carried out in real-time, providing qPCR functionality. For example, during qPCR operation, the signal may be read by monitoring a dsDNA binding dye (e.g., SYBR) or a fluorogenic probe (e.g., TaqMan). The signal may increase with accumulation of newly generated PCR products during each PCR cycle. To perform qPCR, an aliquot from a droplet (e.g., droplet volume can be on the pL-mL scale) can be used. By monitoring qPCR in this aliquot in real-time, the performance of the main sample can be inferred, and the amount of amplification required can be adjusted in response. PCR and qPCR operations on an array or a plurality of arrays may be multiplexed to track various amplicons in parallel (e.g., genes, markers of interest, NGS libraries, etc.). PCR and qPCR may be used for quantification of, for example, NGS libraries, gene expression, or target detection (e.g. diagnostics).

### Next-Generation Sequencing (NGS) Library Preparation and Evaporation Compensation

The systems and methods described herein may accomplish fully digital for high-throughput automation of NGS sample preparation. Whole genome sequencing (WSG) libraries can be prepared starting from purified DNA using systems and methods described herein. For example, DNA can be fragmented enzymatically on an array described herein, end-repaired, and A-overhangs added. Dual indexed barcodes can be ligated onto the DNA fragments and the final ligation product can be purified and size-selected by magnetic-bead based purification. The method may be performed on a single device described herein.

Evaporation compensation techniques described herein may not affect the reaction kinetics of NGS library preparation, providing applicability to a broad range of biological and chemical workflows described herein. Furthermore, a number of experiments can be run, and datasets can be built, from the same array for the evaporative loss for each of such chemical/biological reaction. For example, the datasets can be used to calculate compensation volumes required to keep a reaction volumes within a margin of error of, for example, 20%, 10%, 5%, 1%, or less. In reactions where there is volume loss, the compensation volume can be introduced in a timed fashion (e.g., in an open loop with no sensing and feedback). Alternatively, the dataset can be fed through a machine learning model to develop algorithms to learn how to estimate compensation volumes based on characteristics of the reactions. The datasets for feeding into the machine learning model can be generated from sensors adjacent to the arrays or can be generated from sensors external to the arrays. Similarly, datasets for improved ligation from simultaneous mixing and heating or improved fragmentation in response to active mixing on the array can be used to optimize performance of NGS sample preparation workflows using machine learning algorithms.

### Nanoliter NGS

Input material and reagent quantity can be scaled-down to nanoliter-size or picoliter-size reaction volumes (e.g., droplets) on the arrays described herein. Reagent concentration may remain constant (e.g., for accurate reaction stoichiometry). Reagent starting and final concentration may not remain constant (e.g., increased or decreased), for example, optimizing reaction efficiency in a nanoliter- or picoliter-sized reaction volume.

Nanoliter- or picoliter-sized droplets on the open surface of an array (e.g., EWOD array or DEP array) or solid support (e.g., glass) may contact a much smaller area of an array compared to droplets sandwiched between two plates. The smaller areal occupancy may allow a large number of droplets (e.g., thousands of nanoliter droplets and millions of picoliter droplets) to be packed in a small footprint of the array (e.g., size of a standard SBS well plate). On an open array, for example, with a smooth slippery surface and with no interfacial forces from a second surface (e.g., from a second plate), nanoliter-sized droplets can be transported and mixed with forces (e.g., electromotive force from EWOD). Furthermore, the droplets can be, for example, heated, cooled, subjected to magnetic fields, or any combination thereof. Actuation of nanoliter- or picoliter-sized droplets may be accomplished on electrodes of dimensions comparable to the droplet contact area (e.g., 0.00001 millimeters (mm), 0.0001 mm, 0.001 mm, 0.01 mm, 0.1 mm, 1 mm, 10 mm, 100 mm, 1,000 mm, or more). Alternatively, a continuous set of electrodes surrounding the nanoliter- or picoliter-sized droplet can be activated simultaneously to generate sufficient electromotive force for transportation of the droplet(s) (FIG. 56A). Reaction volumes and electrode sizes at this scale may provide at least about 1, 10, 100, 1,000, 10,000, 100,000, 1,000,000, or more reactions to proceed in parallel (e.g., making possible high-throughput applications in a the nanoliter- or picoliter-level (FIG. 56B). The process of scaling-down reactions, electrodes, input material, reagents, or any combination thereof may be automated using software simulation.

### High Molecular Weight (HMW) Nucleic Acid Isolation and Transfer

Intact genomic DNA can be greater than about 100 megabases (Mb) in length, but isolation protocols may fragment the genomic DNA to fragments of 10-200 kilobases (Kb) in length. However, as sequencing technologies are capable of processing longer read lengths (e.g., greater than about 1 Mb), the low yield of intact genomic DNA molecules (e.g., >100 kb) is an unresolved limitation of DNA isolation technologies.

Described herein are systems and methods that minimize mechanical fragmentation (e.g., due to shear forces of air-displacement pipetting) of nucleic acids (e.g., DNA). Described herein are systems and methods that reduce sample loss due to, for example, dead volumes of traditional handling devices. The systems and methods described herein may be capable of automating high throughput and high molecular weight (HMW) DNA isolation, wherein the median DNA fragment size is at least about 1 Kb, 10 Kb, 100 Kb, 1,000 Kb, 10,000 Kb, 100,000 Kb, 1,000,000 Kb, or more. The systems and methods described herein may be capable of automating high throughput and high molecular weight DNA isolation, wherein the median DNA fragment size is at most about 1,000,000 Kb, 100,000 Kb, 10,000 Kb, 1,000 Kb, 100 Kb, 10 Kb, 1 Kb, or less. The systems and methods described herein may be capable of automating high throughput and high molecular weight DNA isolation, wherein the median DNA fragment size is from about 1 Kb to about 1,000,000 Kb, 100 Kb to about 500,000 Kb, or about 1,000 Kb to about 100,000 Kb.

Described herein are universal open electrowetting-on-dielectric (EWOD) systems and methods that can manipulate reaction volumes suitable for HMW DNA isolation. By integrating capabilities such as, for example, magnetic bead separation and heater/cooler in the same system, the system and methods described herein may not comprise custom instrumentation. The systems and methods described herein can provide straightforward reprogramming to expand the number of executable workflows, enabling new recipes with, for example, variable input, reagents, incubations, wash steps, and thousands of droplets controlled in a programmable manner on a single device.

The system described herein can manipulate droplets on a 2D or 3D grid of electrodes in at least two configurations (e.g., droplets sandwiched between two plates separated by a small gap or on an open surface). For example, on a two plate PDM system (e.g., with electrodes dimension of 25 μm) a droplet of 5 pL can be aliquoted, transported, and mix with another droplet. On an open surface, EWOD device (e.g., with electrodes dimension of 2 mm) droplets (e.g., about 200 μL) can be manipulated. The systems described herein can handle volumes suitable for, for example, bulk DNA extraction (e.g., 100 μl to 1 ml) as well as droplets small enough to encapsulate single cells and individual nuclei (e.g., 50 nL).

Additionally, in order to increase the yield of HMW DNA from cell samples, enhanced agitation techniques may be performed on the array. Agitation techniques may include methods such as, for example, mechanical buzzers, shakers, vortexers, sonication, or any combination thereof. Magnetic micro-stirrers may be introduced into the samples to enhance mixing. These stirrers may be coupled with different magnet configurations described herein. Magnets of different shapes may be used to alter the shape and spread of the magnetic beads on the array. Magnets with tunable strength may be used to accommodate the magnetic beads being manipulated on the array.

DNA extracted from cells in a stabilization buffer can produce intact HMW DNA. For example, alginate hydrogels can be used as a scaffold material to stabilize HMW DNA. Alginate can form stable gels in the presence of cations, gelling conditions may be mild, and the gelation process can be reversed by, for example, extracting calcium ions (e.g., by adding citrate or EDTA). Extracted DNA can be stabilized in high viscosity/low-shear solutions (e.g., alginate droplets) formed on-chip. This stabilization method may allow the transfer (e.g. within a lab or by shipping between sites) of HMW genomic DNA without substantial degradation. HMW DNA can be stored in reagents to prevent shearing (e.g. alginate hydrogels). Extracted HMW DNA can be, for example, transferred to a tube after extraction or stored on the EWOD array. To prevent DNA shearing prior to sequencing, sequencing libraries can be assembled on the same device used for HMW DNA extraction. Similarly, nanopores may be integrated to the array for direct sequencing without sample transfer.

### Enzymatic Biopolymer Synthesis

Biopolymers (e.g., polynucleotides and polypeptides) may be synthesized on an array by dispensing and moving reagents sequentially, in parallel, or a combination thereof. The reagents may include, for example, nucleoside triphosphates, nucleotides, enzymes, buffers, beads, deblocking agents, water, salts, or any combination thereof. For example, polynucleotide (e.g. DNA) synthesis may occur directly on the surface of the array by functionalizing specific locations on the array. For example, the functionalized locations may act as reaction sites. DNA synthesis may also be performed on beads contained in droplets manipulated by the array (e.g., EWOD). DNA synthesis may be performed on the array at volumes on the scale of milliliters, microliters, nanoliters, picoliters, or femtoliters. DNA fragments may be assembled into longer fragments directly on the array by processes such as, for example, Gibson assembly. Combinatorial merging of droplets may be used to, for example, create a diversity of DNA fragments. The quality of the assembled DNA fragments can be assessed by sequencing library preparation on the array for downstream sequencing, such as, for example, Illumina- or Oxford Nanopore Technologies-based sequencing.

The reservoirs for storing reagents (e.g., nucleoside triphosphates, magnetic beads, enzymes, salts, water, cleaving agents, or deblocking reagents) may be integrated on the surface of the array, integrated above the array, or dispensed from an external reservoir using a dispensing method described herein.

1, 10, 100, 1,000, 10,000, 100,000, 1,000,000, or more reactions may be performed in parallel on a single array or on multiple arrays. Droplets with polynucleotide (e.g., DNA) sequences can be purified and size-selected using magnetic bead. The purified DNA sequences can be merged and assembled in a combinatorial fashion using DNA assembly techniques, such as, for example, Gibson assembly. The assembled polynucleotides (e.g., DNA) may contain errors. To correct for the errors, the assembled polynucleotide (e.g., DNA) may be treated with mismatch binding or mismatch cleaving proteins (e.g., MutS, T4 endonuclease VII, or T7 endonuclease I).

The arrays for synthesizing DNA using enzymatic processes can be stacked vertically or horizontally as described herein. The stacks may be connected to a cloud server infrastructure. For example, when a user procures a sequence of, for example, DNA, gene pools, RNA, guide RNA, or other biopolymers, the user can interact with a dashboard on a computer that connects directly to the cloud infrastructure. Upon submitting an input sequence for synthesis, a finite set of arrays may be instantiated on demand. The number of arrays can be from, for example, one to several billion. Once the arrays are instantiated, the entire synthesis process may be run autonomously.

### Sample Quantification

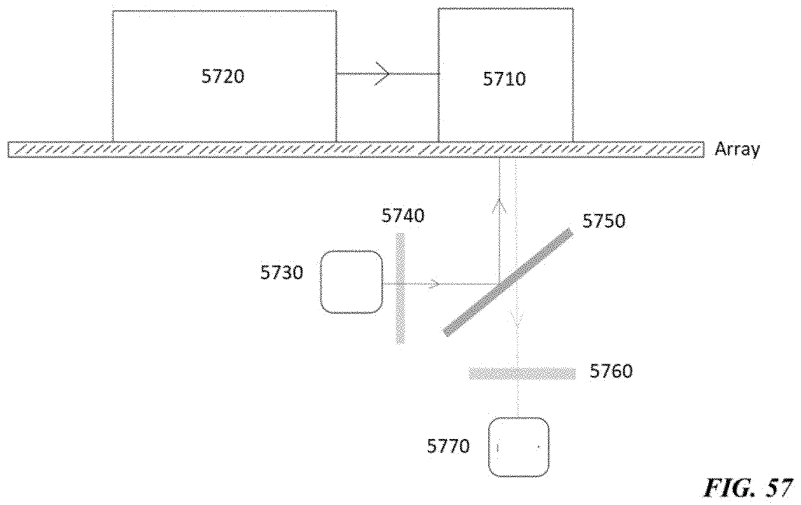

Optical-based (e.g., fluorescence) detection of nucleic acids (e.g., DNA) on the array may be implemented by using, for example, intercalating fluorescent dyes (e.g., SYBR Green) (e.g., a fluorescence detection mechanism is depicted in FIG. 57). For making fluorescence-based measurements, a sample can be positioned in the sample detection zone (5710) from another portion of the array (5720). The sample detection zone may be an optically clear path (e.g., transparent or a hole in the surface). The excitation source (5730), an excitation filter (5740), a mirror (5750), an emission filter (5760), the detection sensor (5770), or any combination thereof may be positioned below the sample to allow light to excite and travel back through the optically clear path.

For example, A size selection unit (5520) may precede the fluorescence-based detection zone. The size-based separation unit may employ electrophoresis or capillary electrophoresis to separate nucleic acid fragments based on their size. The size-separated sample can be passed through a detection zone where the fluorescence signal distribution of the sample may be indicative of the sample's size distribution. The total fluorescence of the sample may be used to quantify the concentration of total nucleic acid in the sample.

### Methods and Systems for Droplet Manipulation

In an aspect, the present disclosure provides a method for processing a plurality of biological samples. The method may comprise receiving, adjacent to an array, a plurality of droplets that may comprise the plurality of biological samples, and using at least the array to process the plurality of biological samples in the plurality of droplets or derivatives thereof at a coefficient of variation (CV) of at least one parameter of the plurality of droplets or derivatives thereof, or the array, of less than 20% at cross-talk between the plurality of droplets at less than 5%. This may be used to process the plurality of biological samples. The array may be an electrowetting device, as described elsewhere herein.

In another aspect, the present disclosure provides a system for processing a plurality of biological samples. The system may comprise receiving, adjacent to an array, a plurality of droplets that may comprise the plurality of biological samples, and using at least the array to process the plurality of biological samples in the plurality of droplets or derivatives thereof at a coefficient of variation (CV) of at least one parameter of the plurality of droplets or derivatives thereof, or the array, of less than 20% at cross-talk between the plurality of droplets at less than 5%. The system may be used to process the plurality of biological samples.

In another aspect, the present disclosure provides a system for biological sample processing, comprising: a housing configured to contain a plurality of arrays, wherein an array of the plurality of arrays is configured to (i) receive, adjacent to the array, a plurality of droplets comprising the plurality of biological samples, and (ii) use at least the array to process said plurality of biological samples in the plurality of droplets or derivatives thereof at a coefficient of variation (CV) of at least one parameter of the plurality of droplets or derivatives thereof, or the array, of less than 20% at cross-talk between the plurality of droplets at less than 5%. The plurality of arrays may be removable from the housing. The housing may be configured to couple to a nucleic acid sequencing platform. The housing may be a nucleic acid sequencing platform.

In another aspect, the present disclosure provides a method for customizing an array system for processing a plurality of biological samples. The method may comprise receiving a request for a configured array system from a user, which request may comprise one or more specifications, and using the one or more specifications to configure the array system to yield the configured array system, which configured array system may be configured to receive a plurality of droplets that may comprise the plurality of biological samples and may process the plurality of droplets or derivatives thereof at a coefficient of variation (CV) of at least one parameter of the plurality of droplets or derivatives thereof, or the array, of less than 20% at cross-talk between the plurality of droplets may be at less than 5%.

In another aspect, the present disclosure provides a method for processing a biological sample. The method may comprise providing, adjacent to an open array, a droplet that may comprise the biological sample, and that may use the open array to process the biological sample in the droplet or derivative thereof. During processing, a position of the static (or sessile) droplet may vary by at most 5% over a time period of at least 10 seconds.

In another aspect, the present disclosure provides a method for processing a biological sample. The method may comprise receiving, adjacent to an array, a droplet comprising the biological sample, and may use at least the array to process the biological sample in the plurality of droplets or derivatives thereof at a coefficient of variation (CV) of at least one parameter of the droplet or derivative thereof, or the array, of less than 20% at cross-talk between the droplet at less than 5%.

The at least one parameter may comprise one or more members selected from the group consisting of droplet size, droplet volume, droplet position, droplet speed, droplet wetting, droplet temperature, droplet pH, beads in droplets, number of cells in droplets, droplet color, concentration of chemical material, concentration of biological substance, or any combination thereof. The at least one parameter may be at least 1, 2, 3, 4, 5, 6, 7, 8, 9, 10, or more parameters. The at least one parameter may be a measurable property of a droplet.

In some embodiments, the concentration of a chemical material or biological substance within a droplet is monitored such that it does not exceed or fall below a predetermine threshold. In some embodiments, the predetermined threshold of a concentration of a chemical material or biological substance is 5%, 10%, 15%, 20%, 25%, 30%, 35%, 40%, 45%, 50%, 55%, 60%, 65%, 70% 75%, 80%, 85%, 90%, or 95%.

The configuration of the array may be selected from the group consisting of an open configuration with an electrode array, open configuration with no electrode array, open configuration with non-coplanar set of electrodes, two plates with an array of electrodes on one plate and no electrodes on the other plate, two plates with non-coplanar set of electrodes on one plate and no electrodes on the other plate, two plates with an array of electrodes on one plate and single electrode on the other plate, two plates with an non-coplanar set of electrodes on one plate and single electrode on other plate, two plates with electrodes arrays on both plates, two plates with non-coplanar set of electrodes on both plates, or any combination thereof. An open configuration may include an array with a set of electrodes and no opposing electrodes. An electrode array may be one or more electrodes. An electrode array may be embedded within another material. An array may be an electrowetting device. The array may be accessed at any time. The biological sample or droplet may be accessed at any time. The array, biological sample, droplet, or any combination thereof may be accessed by user or by a component of the array. The array, biological sample, droplet, or any combination thereof may be accessed by user or by a component of the array at any time. An open electrode array may allow access of samples from any angle without requiring the removal of a top plate. An open electrode array may have less friction than a closed array. An open array may allow for faster and more complete mixing due to the three dimensional nature of sample mixing.

In an open configuration, the array may include a plurality of electrodes in a substrate. The plurality of electrodes may be coplanar. Alternatively, subsets of the plurality of electrodes may not be coplanar. The array may not include any opposing electrode (i.e., a surface of the array may be open and not include an opposing plate). Alternatively, at least a portion of the array may include an opposing plate. The opposing plate may include one or more electrodes.

The plurality of biological samples may be processed using an electric force. The plurality of biological samples may be processed using an electric field. The plurality of biological samples may be processed using a force field. The plurality of biological samples may be processed by combining a force field with an electric field. The force field may be generated by fluid flow on the array or vibration of the array in which the force field or force may be selected from the group consisting of acoustic waves, vibrations, air pressure, light field (or electromagnetic field), magnetic field, gravitational field, centrifugal force, hydrodynamic force, electrophoretic force, dielectrowetting force, and capillary force. The force field may be a combination of two or more of the members of the group.

The plurality of biological samples may be processed with no more than 4, 3, 2, or 1 pipetting operation(s). For example, for two pipetting operations, the plurality of droplets may be deposited adjacent to the array using a pipette, processed on the array, and processed droplets may be removed from the array using another pipetting operation.

The array may comprise a plurality of sensors. The plurality of sensors may be used to measure signals from the plurality of droplets or the derivatives thereof before, during, or subsequent to the processing of the plurality of biological samples. The plurality of sensors may comprise an impedance sensor, a capacitance sensor (e.g., touchscreen), a pH sensor, a temperature sensor, an optical sensor, a camera (e.g., charged-coupled device (CCD) camera), a current measurement sensor, an electronic sensor for biomolecular detection, an x-ray sensor, electrochemical sensors, electrochemiluminescent sensors, piezoelectric sensors, or any combination thereof. The plurality of sensors may be used to detect contamination. The plurality of sensors may be used to detect biological materials (e.g., cells, tissue, nucleic acids, proteins, peptides), chemical materials (e.g., nanoparticles, beads, small-molecules), or a combination thereof.

The array may use the plurality of sensors in a feedback loop to regulate one or more parameters of the array while processing the plurality of biological samples. The plurality of sensors and feedback loop may be used to discover and optimize reaction conditions autonomously (e.g., without any user input). The plurality of sensors may be directly coupled to at least one droplet, such as directly in contact with the droplet or in contact with the droplet through one or more intervening layers (e.g., a dielectric layer).

The array may have the intake of at least one sensor directed towards at least one droplet. For example, for an optical sensor, a fiber optic may be directed towards at least one droplet. The fiber optic may then be coupled to a monochrometer with an attached CCD camera. This example may be used to determine the absorption or fluorescence spectrum of at least one droplet for the duration of the processing.

The temperature sensor may be a thermocouple. As an alternative, the temperature sensor may be an infrared (IR) temperature sensor.

The optical sensor may be a CCD camera or a photomultiplier tube. The optical sensor may have attached optics such as a monochrometer, one or more filters, or a series of lenses. The camera may be a camera with a fast refresh rate that may be used to capture the contact angle of one or more droplets. The camera may be a monochrome camera or a color camera. The current measurement sensor may have electrodes that may be used to interface with at least one droplet. The current sensor may be contactless. The electronic sensor for biomolecular detection may be based on enzymes or graphene. The x-ray sensor may be an x-ray diffraction instrument. The x-ray sensor may be an x-ray fluorescence detector.

The biological material detected using a sensor of the plurality of sensors may be, for example, a fluorescent protein, an antibody, an enzyme, a nucleic acid pair, or a combination of two or more biological materials. The cells detected using a sensor of the plurality of sensors may be, for example, prokaryotic cells, eukaryotic cells, or cells for the detection of toxins. The tissues detected using a sensor of the plurality of sensors may be, for example, any tissue (e.g., brain, skin, muscle, heart, lung, etc.) isolated from a subject or patient. The chemical materials detected using a sensor of the plurality of sensors may be, for example, fluorescent chemicals, chemicals with strong binding towards metals, or chemicals that undergo a transformation in the presence of an object of interest (e.g., a bicarbonate salt that is converted to CO₂ gas in the presence of an acid).

The biological materials as a sensor may be a fluorescent protein, an antibody, an enzyme, a nucleic acid pair, or a combination of two or more biological materials. The cells as sensors may be prokaryotic cells, eukaryotic cells, or cells for the detection of toxins. The tissues as sensors may be muscle fibers. The chemical materials as sensors may be fluorescent chemicals, chemicals with strong binding towards metals, or chemicals that undergo a transformation in the presence of an object of interest (e.g., a bicarbonate salt that is converted to CO₂ gas in the presence of an acid). In some embodiments, biomolecules (e.g., proteins/nucleic acids) are used as sensing elements in a sensor in order to detect, for example, target biomolecules or relevant analytes.

The electrochemical sensor may be an electrochemical gas sensor. The electrochemiluminescent sensor may be tris(bipyridine)ruthenium (II) chloride, a quantum dot, or a nanoparticle. The piezoelectric sensor may be used to detect pressure, acceleration, temperature, strain, force, or any combination thereof. The piezoelectric sensor may be made of a piezoelectric ceramic or a single crystal. The nucleic acid as a sensor may utilize a complimentary set of base pairs to a nucleic acid of interest. The nucleic acid as a sensor may be DNA or RNA. The nucleic acid as a sensor may be free in solution or associated with a substrate. The protein as a sensor may be an enzyme. The protein as a sensor may be fluorescent. The protein as a sensor may be free in solution or associated with a substrate. The nanoparticle sensors may be fluorescent, magnetic, or any combination of the two. The small molecule sensor may detect metals. The small molecule sensors may be fluorescent. The metals may be zinc, copper, iron, cobalt, mercury, silver, gold, manganese, chromium, nickel, or a combination thereof.

The at least one sensor of the plurality of sensors can measure location, droplet volume, presence of biological material, activity of biological material, droplet velocity, kinematics, droplet radius, droplet shape, droplet height, color, surface area, contact angle, reaction state, emittance, absorbance, or any combination thereof. A measurement of at least one sensor of the plurality of sensors may be used to further process at least one droplet, biological sample, or a combination thereof of the plurality of droplets, the plurality of biological samples, or a combination thereof. The further processing may comprise giving a command to actuate inputs, outputs, or a combination thereof adjacent to or on, or a combination thereof, the array in real time. The command may provide instructions to correct an error of the array. The error may be an error in location, droplet volume, presence of biological material, activity of biological material, droplet velocity, droplet kinetics, droplet radius, droplet shape, droplet height, color, surface area, contact angle, reaction state, emittance, absorbance, or any combination thereof.

The location may be of a droplet, a reagent, a biological sample, a component of the array, a position of the array, an area of the array, an area adjacent to the array, a point of the array, or any combination thereof. The location may be corrected by at least 0.001%, 0.01%, 0.1%, 1%, 5%, 10%, 15%, 20%, 30%, 40%, 50%, 60%, 70%, 80%, 90%, 95%, 99%, or more. The location may be corrected by at most 99%, 95%, 90%, 80%, 70%, 60%, 50%, 40%, 30%, 20%, 15%, 10%, 5%, 1%, 0.1%, 0.01%, 0.001%, or less. The location may be corrected from 0.001% to 20%, 0.01% to 10%, 0.01% to 5%, or 0.1% to 1%.

The droplet volume may comprise a volume of at least 1 picoliter (pL), 10 pL, 100 pL, 1 nanoliter (nL), 10 nL, 100 nL, 1 μL, 10 μL, 100 μL, 1 milliliter (mL), 10 mL or more. The droplet volume may comprise a volume of at most 10 mL, 1 mL, 100 μL, 10 μL, 1 μL, 100 nL, 10 nL, 1 nL, 100 pL, 10 pL, 1 pL, or less. The droplet volume may be corrected by at least 0.001%, 0.01%, 0.1%, 1%, 5%, 10%, 15%, 20%, 30%, 40%, 50%, 60%, 70%, 80%, 90%, 95%, 99%, or more. The droplet volume may be corrected by at most 99%, 95%, 90%, 80%, 70%, 60%, 50%, 40%, 30%, 20%, 15%, 10%, 5%, 1%, 0.1%, 0.01%, 0.001%, or less. The droplet volume may be corrected from 0.001% to 20%, 0.01% to 10%, 0.01% to 5%, or 0.1% to 1%. In some embodiments, a droplet is replenished if the volume of the droplet falls below a predetermined threshold. In some embodiments, the predetermined threshold may be a volume of at least 1 picoliter (pL), 10 pL, 100 pL, 1 nanoliter (nL), 10 nL, 100 nL, 1 μL, 10 μL, 100 μL, 1 milliliter (mL), 10 mL or more. In some embodiments, the predetermined threshold may be a volume at most 10 mL, 1 mL, 100 μL, 10 μL, 1 μL, 100 nL, 10 nL, 1 nL, 100 pL, 10 pL, 1 pL, or less. In some embodiments, a droplet is reduced if the volume of the droplet exceeds a predetermined threshold. In some embodiments, the predetermined threshold may be a volume of at least 1 picoliter (pL), 10 pL, 100 pL, 1 nanoliter (nL), 10 nL, 100 nL, 1 μL, 10 μL, 100 μL, 1 milliliter (mL), 10 mL or more. In some embodiments, the predetermined threshold may be a volume at most 10 mL, 1 mL, 100 μL, 10 μL, 1 μL, 100 nL, 10 nL, 1 nL, 100 pL, 10 pL, 1 pL, or less.

A biological sample may comprise a nucleic acid, protein, cell, salt, buffer, or enzyme, wherein said droplet comprises one or more reagents for nucleic acid isolation, cell isolation, protein isolation, peptide purification, isolation or purification of a biopolymer, immunoprecipitation, in vitro diagnostics, exosome isolation, cell activation, cell expansion, or isolation of a specific biomolecule, and wherein said droplet is manipulated by said reagents to perform said nucleic acid isolation, cell isolation, protein isolation, peptide purification, isolation or purification of a biopolymer, immunoprecipitation, in vitro diagnostics, exosome isolation, cell activation, cell expansion, or isolation of a specific biomolecule. The presence of the biological sample may be corrected by an amount of at least 0.001%, 0.01%, 0.1%, 1%, 5%, 10%, 15%, 20%, 30%, 40%, 50%, 60%, 70%, 80%, 90%, 95%, 99%, or more. The presence of the biological sample may be corrected by an amount of at most 99%, 95%, 90%, 80%, 70%, 60%, 50%, 40%, 30%, 20%, 15%, 10%, 5%, 1%, 0.1%, 0.01%, 0.001%, or less. The presence of the biological sample may be corrected by an amount from 0.001% to 20%, 0.01% to 10%, 0.01% to 5%, or 0.1% to 1%.

The activity of biological material may comprise enzymatic activity, cellular activity, small-molecule activity, reagent activity, wherein the activity may be a measure of affinity, specificity, reactivity, rate, inhibition, toxicity (e.g., IC₅₀, LD₅₀, EC₅₀, ED₅₀, GI₅₀, etc.), or any combination thereof. The activity of the biological sample may be corrected by an amount of at least 0.001%, 0.01%, 0.1%, 1%, 5%, 10%, 15%, 20%, 30%, 40%, 50%, 60%, 70%, 80%, 90%, 95%, 99%, or more. The activity of the biological sample may be corrected by an amount of at most 99%, 95%, 90%, 80%, 70%, 60%, 50%, 40%, 30%, 20%, 15%, 10%, 5%, 1%, 0.1%, 0.01%, 0.001%, or less. The activity of the biological sample may be corrected by an amount from 0.001% to 20%, 0.01% to 10%, 0.01% to 5%, or 0.1% to 1%.

In some embodiments, the droplet has a viscosity of about 0% glycerol to about 60% glycerol at room temperature (˜25° C.). In some embodiments, the droplet has a viscosity of about 0% glycerol to about 10% glycerol, about 0% glycerol to about 15% glycerol, about 0% glycerol to about 20% glycerol, about 0% glycerol to about 25% glycerol, about 0% glycerol to about 30% glycerol, about 0% glycerol to about 35% glycerol, about 0% glycerol to about 40% glycerol, about 0% glycerol to about 45% glycerol, about 0% glycerol to about 50% glycerol, about 0% glycerol to about 55% glycerol, about 0% glycerol to about 60% glycerol, about 10% glycerol to about 15% glycerol, about 10% glycerol to about 20% glycerol, about 10% glycerol to about 25% glycerol, about 10% glycerol to about 30% glycerol, about 10% glycerol to about 35% glycerol, about 10% glycerol to about 40% glycerol, about 10% glycerol to about 45% glycerol, about 10% glycerol to about 50% glycerol, about 10% glycerol to about 55% glycerol, about 10% glycerol to about 60% glycerol, about 15% glycerol to about 20% glycerol, about 15% glycerol to about 25% glycerol, about 15% glycerol to about 30% glycerol, about 15% glycerol to about 35% glycerol, about 15% glycerol to about 40% glycerol, about 15% glycerol to about 45% glycerol, about 15% glycerol to about 50% glycerol, about 15% glycerol to about 55% glycerol, about 15% glycerol to about 60% glycerol, about 20% glycerol to about 25% glycerol, about 20% glycerol to about 30% glycerol, about 20% glycerol to about 35% glycerol, about 20% glycerol to about 40% glycerol, about 20% glycerol to about 45% glycerol, about 20% glycerol to about 50% glycerol, about 20% glycerol to about 55% glycerol, about 20% glycerol to about 60% glycerol, about 25% glycerol to about 30% glycerol, about 25% glycerol to about 35% glycerol, about 25% glycerol to about 40% glycerol, about 25% glycerol to about 45% glycerol, about 25% glycerol to about 50% glycerol, about 25% glycerol to about 55% glycerol, about 25% glycerol to about 60% glycerol, about 30% glycerol to about 35% glycerol, about 30% glycerol to about 40% glycerol, about 30% glycerol to about 45% glycerol, about 30% glycerol to about 50% glycerol, about 30% glycerol to about 55% glycerol, about 30% glycerol to about 60% glycerol, about 35% glycerol to about 40% glycerol, about 35% glycerol to about 45% glycerol, about 35% glycerol to about 50% glycerol, about 35% glycerol to about 55% glycerol, about 35% glycerol to about 60% glycerol, about 40% glycerol to about 45% glycerol, about 40% glycerol to about 50% glycerol, about 40% glycerol to about 55% glycerol, about 40% glycerol to about 60% glycerol, about 45% glycerol to about 50% glycerol, about 45% glycerol to about 55% glycerol, about 45% glycerol to about 60% glycerol, about 50% glycerol to about 55% glycerol, about 50% glycerol to about 60% glycerol, or about 55% glycerol to about 60% glycerol at room temperature (˜25° C.). In some embodiments, the droplet has a viscosity of about 0% glycerol, about 10% glycerol, about 15% glycerol, about 20% glycerol, about 25% glycerol, about 30% glycerol, about 35% glycerol, about 40% glycerol, about 45% glycerol, about 50% glycerol, about 55% glycerol, or about 60% glycerol. In some embodiments, the droplet has a viscosity of at least about 0% glycerol, about 10% glycerol, about 15% glycerol, about 20% glycerol, about 25% glycerol, about 30% glycerol, about 35% glycerol, about 40% glycerol, about 45% glycerol, about 50% glycerol, or about 55% glycerol at room temperature (˜25° C.). In some embodiments, the droplet has a viscosity of at most about 10% glycerol, about 15% glycerol, about 20% glycerol, about 25% glycerol, about 30% glycerol, about 35% glycerol, about 40% glycerol, about 45% glycerol, about 50% glycerol, about 55% glycerol, or about 60% glycerol. In some embodiments, the droplet has a viscosity of about 40% glycerol at room temperature (˜25° C.).

In some embodiments, the droplet has a viscosity of about 0.1 centipoise (cP) to about 200 cP at room temperature (˜25° C.). In some embodiments, the droplet has a viscosity of about 0.1 cP to about 1 cP, about 0.1 cP to about 2 cP, about 0.1 cP to about 5 cP, about 0.1 cP to about 10 cP, about 0.1 cP to about 30 cP, about 0.1 cP to about 50 cP, about 0.1 cP to about 70 cP, about 0.1 cP to about 100 cP, about 0.1 cP to about 150 cP, about 0.1 cP to about 200 cP, about 1 cP to about 2 cP, about 1 cP to about 5 cP, about 1 cP to about 10 cP, about 1 cP to about 30 cP, about 1 cP to about 50 cP, about 1 cP to about 70 cP, about 1 cP to about 100 cP, about 1 cP to about 150 cP, about 1 cP to about 200 cP, about 2 cP to about 5 cP, about 2 cP to about 10 cP, about 2 cP to about 30 cP, about 2 cP to about 50 cP, about 2 cP to about 70 cP, about 2 cP to about 100 cP, about 2 cP to about 150 cP, about 2 cP to about 200 cP, about 5 cP to about 10 cP, about 5 cP to about 30 cP, about 5 cP to about 50 cP, about 5 cP to about 70 cP, about 5 cP to about 100 cP, about 5 cP to about 150 cP, about 5 cP to about 200 cP, about 10 cP to about 30 cP, about 10 cP to about 50 cP, about 10 cP to about 70 cP, about 10 cP to about 100 cP, about 10 cP to about 150 cP, about 10 cP to about 200 cP, about 30 cP to about 50 cP, about 30 cP to about 70 cP, about 30 cP to about 100 cP, about 30 cP to about 150 cP, about 30 cP to about 200 cP, about 50 cP to about 70 cP, about 50 cP to about 100 cP, about 50 cP to about 150 cP, about 50 cP to about 200 cP, about 70 cP to about 100 cP, about 70 cP to about 150 cP, about 70 cP to about 200 cP, about 100 cP to about 150 cP, about 100 cP to about 200 cP, or about 150 cP to about 200 cP at room temperature (˜25° C.). In some embodiments, the droplet has a viscosity of about 0.1 cP, about 1 cP, about 2 cP, about 5 cP, about 10 cP, about 30 cP, about 50 cP, about 70 cP, about 100 cP, about 150 cP, or about 200 cP at room temperature (˜25° C.). In some embodiments, the droplet has a viscosity of at least about 0.1 cP, about 1 cP, about 2 cP, about 5 cP, about 10 cP, about 30 cP, about 50 cP, about 70 cP, about 100 cP, or about 150 cP at room temperature (˜25° C.). In some embodiments, the droplet has a viscosity of at most about 1 cP, about 2 cP, about 5 cP, about 10 cP, about 30 cP, about 50 cP, about 70 cP, about 100 cP, about 150 cP, or about 200 cP at room temperature (˜25° C.).

In some embodiments, the droplet has a viscosity of about 0% glycerol to about 30% glycerol at room temperature (˜25° C.). In some embodiments, the droplet has a viscosity of about 0% glycerol to about 5% glycerol, about 0% glycerol to about 7.5% glycerol, about 0% glycerol to about 10% glycerol, about 0% glycerol to about 12.5% glycerol, about 0% glycerol to about 15% glycerol, about 0% glycerol to about 17.5% glycerol, about 0% glycerol to about 20% glycerol, about 0% glycerol to about 22.5% glycerol, about 0% glycerol to about 25% glycerol, about 0% glycerol to about 27.5% glycerol, about 0% glycerol to about 30% glycerol, about 5% glycerol to about 7.5% glycerol, about 5% glycerol to about 10% glycerol, about 5% glycerol to about 12.5% glycerol, about 5% glycerol to about 15% glycerol, about 5% glycerol to about 17.5% glycerol, about 5% glycerol to about 20% glycerol, about 5% glycerol to about 22.5% glycerol, about 5% glycerol to about 25% glycerol, about 5% glycerol to about 27.5% glycerol, about 5% glycerol to about 30% glycerol, about 7.5% glycerol to about 10% glycerol, about 7.5% glycerol to about 12.5% glycerol, about 7.5% glycerol to about 15% glycerol, about 7.5% glycerol to about 17.5% glycerol, about 7.5% glycerol to about 20% glycerol, about 7.5% glycerol to about 22.5% glycerol, about 7.5% glycerol to about 25% glycerol, about 7.5% glycerol to about 27.5% glycerol, about 7.5% glycerol to about 30% glycerol, about 10% glycerol to about 12.5% glycerol, about 10% glycerol to about 15% glycerol, about 10% glycerol to about 17.5% glycerol, about 10% glycerol to about 20% glycerol, about 10% glycerol to about 22.5% glycerol, about 10% glycerol to about 25% glycerol, about 10% glycerol to about 27.5% glycerol, about 10% glycerol to about 30% glycerol, about 12.5% glycerol to about 15% glycerol, about 12.5% glycerol to about 17.5% glycerol, about 12.5% glycerol to about 20% glycerol, about 12.5% glycerol to about 22.5% glycerol, about 12.5% glycerol to about 25% glycerol, about 12.5% glycerol to about 27.5% glycerol, about 12.5% glycerol to about 30% glycerol, about 15% glycerol to about 17.5% glycerol, about 15% glycerol to about 20% glycerol, about 15% glycerol to about 22.5% glycerol, about 15% glycerol to about 25% glycerol, about 15% glycerol to about 27.5% glycerol, about 15% glycerol to about 30% glycerol, about 17.5% glycerol to about 20% glycerol, about 17.5% glycerol to about 22.5% glycerol, about 17.5% glycerol to about 25% glycerol, about 17.5% glycerol to about 27.5% glycerol, about 17.5% glycerol to about 30% glycerol, about 20% glycerol to about 22.5% glycerol, about 20% glycerol to about 25% glycerol, about 20% glycerol to about 27.5% glycerol, about 20% glycerol to about 30% glycerol, about 22.5% glycerol to about 25% glycerol, about 22.5% glycerol to about 27.5% glycerol, about 22.5% glycerol to about 30% glycerol, about 25% glycerol to about 27.5% glycerol, about 25% glycerol to about 30% glycerol, or about 27.5% glycerol to about 30% glycerol at room temperature (˜25° C.). In some embodiments, the droplet has a viscosity of about 0% glycerol, about 5% glycerol, about 7.5% glycerol, about 10% glycerol, about 12.5% glycerol, about 15% glycerol, about 17.5% glycerol, about 20% glycerol, about 22.5% glycerol, about 25% glycerol, about 27.5% glycerol, or about 30% glycerol at room temperature (˜25° C.). In some embodiments, the droplet has a viscosity of at least about 0% glycerol, about 5% glycerol, about 7.5% glycerol, about 10% glycerol, about 12.5% glycerol, about 15% glycerol, about 17.5% glycerol, about 20% glycerol, about 22.5% glycerol, about 25% glycerol, or about 27.5% glycerol at room temperature (˜25° C.). In some embodiments, the droplet has a viscosity of at most about 5% glycerol, about 7.5% glycerol, about 10% glycerol, about 12.5% glycerol, about 15% glycerol, about 17.5% glycerol, about 20% glycerol, about 22.5% glycerol, about 25% glycerol, about 27.5% glycerol, or about 30% glycerol at room temperature (˜25° C.).

In some embodiments, the droplet has a viscosity of about 0.5 cP to about 15 cP at room temperature (˜25° C.). In some embodiments, the droplet has a viscosity of about 0.5 cP to about 1 cP, about 0.5 cP to about 2 cP, about 0.5 cP to about 3 cP, about 0.5 cP to about 4 cP, about 0.5 cP to about 5 cP, about 0.5 cP to about 7 cP, about 0.5 cP to about 9 cP, about 0.5 cP to about 11 cP, about 0.5 cP to about 13 cP, about 0.5 cP to about 15 cP, about 1 cP to about 2 cP, about 1 cP to about 3 cP, about 1 cP to about 4 cP, about 1 cP to about 5 cP, about 1 cP to about 7 cP, about 1 cP to about 9 cP, about 1 cP to about 11 cP, about 1 cP to about 13 cP, about 1 cP to about 15 cP, about 2 cP to about 3 cP, about 2 cP to about 4 cP, about 2 cP to about 5 cP, about 2 cP to about 7 cP, about 2 cP to about 9 cP, about 2 cP to about 11 cP, about 2 cP to about 13 cP, about 2 cP to about 15 cP, about 3 cP to about 4 cP, about 3 cP to about 5 cP, about 3 cP to about 7 cP, about 3 cP to about 9 cP, about 3 cP to about 11 cP, about 3 cP to about 13 cP, about 3 cP to about 15 cP, about 4 cP to about 5 cP, about 4 cP to about 7 cP, about 4 cP to about 9 cP, about 4 cP to about 11 cP, about 4 cP to about 13 cP, about 4 cP to about 15 cP, about 5 cP to about 7 cP, about 5 cP to about 9 cP, about 5 cP to about 11 cP, about 5 cP to about 13 cP, about 5 cP to about 15 cP, about 7 cP to about 9 cP, about 7 cP to about 11 cP, about 7 cP to about 13 cP, about 7 cP to about 15 cP, about 9 cP to about 11 cP, about 9 cP to about 13 cP, about 9 cP to about 15 cP, about 11 cP to about 13 cP, about 11 cP to about 15 cP, or about 13 cP to about 15 cP at room temperature (˜25° C.). In some embodiments, the droplet has a viscosity of about 0.5 cP, about 1 cP, about 2 cP, about 3 cP, about 4 cP, about 5 cP, about 7 cP, about 9 cP, about 11 cP, about 13 cP, or about 15 cP at room temperature (˜25° C.). In some embodiments, the droplet has a viscosity of at least about 0.5 cP, about 1 cP, about 2 cP, about 3 cP, about 4 cP, about 5 cP, about 7 cP, about 9 cP, about 11 cP, or about 13 cP at room temperature (˜25° C.). In some embodiments, the droplet has a viscosity of at most about 1 cP, about 2 cP, about 3 cP, about 4 cP, about 5 cP, about 7 cP, about 9 cP, about 11 cP, about 13 cP, or about 15 cP at room temperature (˜25° C.).

The droplet velocity may be at least 0.0001 centimeters/second (cm/s), 0.001 cm/s, 0.01 cm/s, 0.1 cm/s, 1 cm/s, 10 cm/s, 20 cm/s, 30 cm/s, 40 cm/s, 50 cm/s, 60 cm/s, 70 cm/s, 80 cm/s, 90 cm/s, 100 cm/s, or more. The droplet velocity may be at most 100 cm/s, 90 cm/s, 80 cm/s, 70 cm/s, 60 cm/s, 50 cm/s, 40 cm/s, 30 cm/s, 20 cm/s, 10 cm/s, 1 cm/s, 0.1 cm/s, 0.01 cm/s, 0.001 cm/s, 0.0001 cm/s, or less. The droplet velocity may be from 0.0001 cm/s to 100 cm/s, 0.001 cm/s to 70 cm/s, 0.01 cm/s to 50 cm/s, 0.1 cm/s to 40 cm/s, 1 cm/s to 25 cm/s, or 1 cm/s to 10 cm/s. The droplet velocity may be corrected by an amount of at least 0.001%, 0.01%, 0.1%, 1%, 5%, 10%, 15%, 20%, 30%, 40%, 50%, 60%, 70%, 80%, 90%, 95%, 99%, or more. The droplet velocity may be corrected by an amount of at most 99%, 95%, 90%, 80%, 70%, 60%, 50%, 40%, 30%, 20%, 15%, 10%, 5%, 1%, 0.1%, 0.01%, 0.001%, or less. The droplet velocity may be corrected by an amount from 0.001% to 20%, 0.01% to 10%, 0.01% to 5%, or 0.1% to 1%.

The kinematics may comprise the motion of points of the array, the motion of objects of the array, and the motion of systems of the array. The kinematics may be of a droplet, a reagent, a liquid, a solid, a gas, or any combination thereof. The kinematics may be corrected by an amount of at least 0.001%, 0.01%, 0.1%, 1%, 5%, 10%, 15%, 20%, 30%, 40%, 50%, 60%, 70%, 80%, 90%, 95%, 99%, or more. The kinematics may be corrected by an amount of at most 99%, 95%, 90%, 80%, 70%, 60%, 50%, 40%, 30%, 20%, 15%, 10%, 5%, 1%, 0.1%, 0.01%, 0.001%, or less. The kinematics may be corrected by an amount from 0.001% to 20%, 0.01% to 10%, 0.01% to 5%, or 0.1% to 1%.

The droplet radius may be at least 0.0001 μm, 0.001 μm, 0.01 μm, 0.1 μm, 1 μm, 5 μm, 10 μm, 20 μm, 30 μm, 40 μm, 50 μm, 60 μm, 70 μm, 80 μm, 90 μm, 100 μm, 500 μm, 1000 μm, 5000 μm, 10,000 μm, 50,000 μm, 100,000 μm, or more. The droplet radius may be at most 100,000 μm, 50,000 μm, 10,000 μm, 5000 μm, 1000 μm, 500 μm, 100 μm, 90 μm, 80 μm, 70 μm, 60 μm, 50 μm, 40 μm, 30 μm, 20 μm, 10 μm, 5 μm, 1 μm, 0.1 μm, 0.01 μm, 0.001 μm, 0.0001 μm, or less. The droplet radius may be from 1000 μm to 0.0001 μm, 500 μm to 0.01 μm, or 100 μm to 1 μm. The droplet radius may be corrected by an amount of at least 0.001%, 0.01%, 0.1%, 1%, 5%, 10%, 15%, 20%, 30%, 40%, 50%, 60%, 70%, 80%, 90%, 95%, 99%, or more. The droplet radius may be corrected by an amount of at most 99%, 95%, 90%, 80%, 70%, 60%, 50%, 40%, 30%, 20%, 15%, 10%, 5%, 1%, 0.1%, 0.01%, 0.001%, or less. The droplet radius may be corrected by an amount from 0.001% to 20%, 0.01% to 10%, 0.01% to 5%, or 0.1% to 1%.

In some embodiments, a droplet is replenished if the size of the droplet falls below a predetermined threshold. In some embodiments, a droplet is reduced if the size of the droplet exceeds a predetermined threshold. In some embodiments, the predetermined threshold may be a radius of at least 0.0001 μm, 0.001 μm, 0.01 μm, 0.1 μm, 1 μm, 5 μm, 10 μm, 20 μm, 30 μm, 40 μm, 50 μm, 60 μm, 70 μm, 80 μm, 90 μm, 100 μm, 500 μm, 1000 μm, 5000 μm, 10,000 μm, 50,000 μm, 100,000 μm, or more. In some embodiments, the predetermined threshold may be a volume at most 100,000 μm, 50,000 μm, 10,000 μm, 5000 μm, 1000 μm, 500 μm, 100 μm, 90 μm, 80 μm, 70 μm, 60 μm, 50 μm, 40 μm, 30 μm, 20 μm, 10 μm, 5 μm, 1 μm, 0.1 μm, 0.01 μm, 0.001 μm, 0.0001 μm, or less.

The droplet shape may be flat, round, spherical, oblong, oval, circular, or any combination thereof. The droplet shape may be corrected to be any shape. The droplet may be corrected to be flat, round, spherical, oblong, oval, circular, or any combination thereof.

The droplet height may be at least 0.0001 μm, 0.001 μm, 0.01 μm, 0.1 μm, 1 μm, 5 μm, 10 μm, 20 μm, 30 μm, 40 μm, 50 μm, 60 μm, 70 μm, 80 μm, 90 μm, 100 μm, 500 μm, 1000 μm, 5000 μm, 10,000 μm, 50,000 μm, 100,000 μm, or more. The droplet height may be at most 100,000 μm, 50,000 μm, 10,000 μm, 5,000 μm, 1000 μm, 500 μm, 100 μm, 90 μm, 80 μm, 70 μm, 60 μm, 50 μm, 40 μm, 30 μm, 20 μm, 10 μm, 5 μm, 1 μm, 0.1 μm, 0.01 μm, 0.001 μm, 0.0001 μm, or less. The droplet height may be from 1000 μm to 0.0001 μm, 500 μm to 0.01 μm, or 100 μm to 1 μm. The droplet height may be corrected by an amount of at least 0.001%, 0.01%, 0.1%, 1%, 5%, 10%, 15%, 20%, 30%, 40%, 50%, 60%, 70%, 80%, 90%, 95%, 99%, or more. The droplet height may be corrected by an amount of at most 99%, 95%, 90%, 80%, 70%, 60%, 50%, 40%, 30%, 20%, 15%, 10%, 5%, 1%, 0.1%, 0.01%, 0.001%, or less. The droplet height may be corrected by an amount from 0.001% to 20%, 0.01% to 10%, 0.01% to 5%, or 0.1% to 1%.

The surface area may be of a component of the array. The component of the array, for example, may be a droplet, a reagent, biological material, a film, a dielectric fluid, an hydrophobic liquid, or any combination thereof. The surface area may be at least 0.0001 μm², 0.001 μm², 0.01 μm², 0.1 μm², 1 μm², 5 μm², 10 μm², 20 μm², 30 μm², 40 μm², 50 μm², 60 μm², 70 μm², 80 μm², 90 μm², 100 μm², 500 μm², 1000 μm², 10,000 μm², 50,000 μm², 100,000 μm², 1,000,000 μm², 10,000,000 μm², 100,000,000 μm², or more. The surface area may be at most 100,000,000 μm², 10,000,000 μm², 1,000,000 μm², 100,000 μm², 50,000 μm², 10,000 μm² 1000 μm², 500 μm², 100 μm², 90 μm², 80 μm², 70 μm², 60 μm², 50 μm², 40 μm², 30 μm², 20 μm², 10 μm², 5 μm², 1 μm², 0.1 μm², 0.01 μm², 0.001 μm², 0.0001 μm², or less. The surface area may be from 100,000,000 μm², 10,000 μm² to 0.0001 μm², 500 μm² to 0.01 μm², or 100 μm² to 1 μm². The surface area may be corrected by an amount of at least 0.001%, 0.01%, 0.1%, 1%, 5%, 10%, 15%, 20%, 30%, 40%, 50%, 60%, 70%, 80%, 90%, 95%, 99%, or more. The surface area may be corrected by an amount of at most 99%, 95%, 90%, 80%, 70%, 60%, 50%, 40%, 30%, 20%, 15%, 10%, 5%, 1%, 0.1%, 0.01%, 0.001%, or less. The surface area may be corrected by an amount from 0.001% to 20%, 0.01% to 10%, 0.01% to 5%, or 0.1% to 1%.

The contact angle may be the contact angle of a droplet and a surface or any liquid surrounding the droplet and a surface. The contact angle may be at least 1°, 5°, 10°, 15°, 20°, 30°, 40°, 50°, 60°, 70°, 80°, 90°, 100°, 110°, 120°, 130°, 140°, 150°, 160°, 170°, or more. The contact angle may be at most 170°, 160°, 150°, 140°, 130°, 120°, 110°, 100°, 90°, 80°, 70°, 60°, 50°, 40°, 30°, 20°, 15°, 10°, 5°, 1°, or less. The contact angle may be from 170° to 1°, 150° to 5°, 120° to 5°, 120° to 90°, 90° to 5°, 90° to 60°, 60° to 5°, or 30° to 5°. The contact angle may be corrected by an amount of at least 0.001%, 0.01%, 0.1%, 1%, 5%, 10%, 15%, 20%, 30%, 40%, 50%, 60%, 70%, 80%, 90%, 95%, 99%, or more. The contact angle may be corrected by an amount of at most 99%, 95%, 90%, 80%, 70%, 60%, 50%, 40%, 30%, 20%, 15%, 10%, 5%, 1%, 0.1%, 0.01%, 0.001%, or less. The contact angle may be corrected by an amount from 0.001% to 20%, 0.01% to 10%, 0.01% to 5%, or 0.1% to 1%.

In some embodiments, a pH of a droplet is monitored by one or more methods as disclosed herein. In some embodiments, a pH of a droplet is maintained within a predetermined threshold. In some embodiments, pH of a droplet is maintained to not exceed 2, 3, 4, 5, 6, 7, 8, 9, 10, 11, 12, or 13. In some embodiments, a pH of droplet is maintained not to drop below 2, 3, 4, 5, 6, 7, 8, 9, 10, 11, 12, or 13.

The reaction state may be of a chemical reaction, a biochemical reaction, a biological reaction, or any combination thereof. The reaction state may be of a solid, a liquid, a gas, or any combination thereof. The reaction state may be altered by adding or removing constituents to or from the reaction, respectively. The constituent, or plurality thereof, may be a solid, liquid, gas, or any combination thereof. The addition or subtraction of constituents may be as a result of droplet motion. The addition or subtraction of constituents may be corrective. The addition or subtraction of constituents may be planned. The addition or subtraction of constituents may be planned according to a pre-programmed method.

The array may comprise a plurality of elements which may comprise: a plurality of heaters, a plurality of coolers, a plurality of magnetic field generators, a plurality of electroporation units, a plurality of light sources, a plurality of radiation sources, a plurality of nucleic acids sequencers, a plurality of biological protein channels, a plurality of solid state nanopores, a plurality of protein sequencers, a plurality of acoustic transducers, a plurality of microelectromechanical system (MEMS) transducers, a plurality of capillary tubes as liquid dispensers, a plurality of holes for dispensing or transferring liquids using gravity, a plurality of electrodes in a hole to dispense or transfer liquids using electric field, a plurality of holes for optical inspection, a plurality of holes for liquids to interact through membranes, or any combination thereof. The plurality of elements may comprise less than or equal to about 100, 90, 80, 70, 60, 50, 40, 30, 20, 10, 5, 4, 3, 2 or less of each element. The plurality of elements may comprise greater than or equal to about 2, 3, 4, 5, 6, 7, 8, 9, 10, 20, 30, 40, 50, 60, 70, 80, 90, 100 or more of each element.

The heater may have a maximum temperature less than or equal to about 150° C., 125° C., 100° C., 75° C., 50° C., 25° C., or less. The heater may be thermoelectric, resistive, or heated by a heat transfer medium (e.g., a recirculated hot water loop). The cooler may have a minimum temperature greater than or equal to about −50° C., −25° C., −10° C., −5° C., 0° C., 10° C., or more. The cooler may be thermoelectric, evaporative, or cooled by a heat transfer medium (e.g., a water chiller).

The magnetic field generator may be for magnetic bead based operations or for other operations requiring magnetic field. The magnetic field generator may be electromagnets.

The electroporation unit may be two or more electrodes on either side of the droplet.

The light source may be broadband, monochromatic, or a combination thereof. The light source may be an incandescent source, a light emitting diode (LED), a laser, or a combination thereof. The light source may emit polarized light, collimated light, or a combination thereof. The plurality of radiation sources may emit ultraviolet light (light of a wavelength from 10 nm to 400 nm), x-rays, gamma rays, alpha particles, beta particles, or a combination thereof. The radiation source may be collimated.

The nucleic acid sequencer may be a Maxam-Gilbert sequencer or a Sanger sequencer. The biological protein channel may be a biological nanopore. The biological protein channel may be a hemolysin or an MspA porin. The solid state nanopore may be silicon nitride or graphene. The protein sequencer may be a mass spectrometer, a single molecule sequencer, or an Edman degradation sequencer. The nucleic acid sequencing may comprise sequencing by synthesis, pyrosequencing, sequencing by hybridization, sequencing by ligation, sequencing by detection of ions released during polymerization of DNA, single-molecule sequencing, or any combination thereof. The single molecule sequencing may be nanopore sequencing. The single molecule sequencing may be single molecule real time (SMRT) sequencing.

The acoustic transducer may be subsonic, ultrasonic, or a combination thereof. The acoustic transducer may be coupled to the array by an acoustic coupling medium. The acoustic coupling medium may be a solid or a liquid. The MEMS transducer may measure force, pressure, or temperature. The capillary tubes as liquid dispensers may be about 2 millimeters (mm) in diameter, 1.5 mm in diameter, 1 mm in diameter, 0.5 mm in diameter, 0.25 mm in diameter, or smaller. There may be 1, 2, 3, 4, 5, 10, 50, 100, or more capillary tubes in the array. The holes for dispensing or transferring liquids using gravity may be treated with different materials to increase or decrease the hydrophobicity of the hole. There may be 1, 2, 3, 4, 5, 10, 50, 100, or more holes in the array. The holes may be at least about 100 μm, 200 μm, 300 μm, 400 μm, 500 μm, 600 μm, 700 μm, 800 μm, 900 μm, 1,000 μm, 1,100 μm, 1,200 μm, 1,300 μm, 1,400 μm, 1,500 μm, 1,600 μm, 1,700 μm, 1,800 μm, 1,900 μm, 2,000 μm, or more in diameter. The holes may be at most about 2,000 μm, 1,900 μm, 1,800 μm, 1,700 μm, 1,600 μm, 1,500 μm, 1,400 μm, 1,300 μm, 1,200 μm, 1,000 μm, 900 μm, 800 μm, 700 μm, 600 μm, 500 μm, 400 μm, 300 μm, 200 μm, 100 μm, or less in diameter. The holes may be from 100 μm to 500 μm in diameter. The electrode in a hole to dispense or transfer liquid may use the electrowetting effect. The holes may be for optical inspection. The holes may be of a size described herein. The holes for liquids to interact through a membrane may have a membrane of a material described herein. The holes may be used for any combination of dispensing or transferring liquids using electric field, pneumatic forces, optical inspection, allowing liquids to interact through membranes.

The array may interface with a liquid handling unit, which the liquid handling unit may direct the plurality of droplets adjacent to the array. The liquid handling unit may be selected from the group consisting of robotic liquid handling systems, acoustic liquid dispensers, syringe pumps, inkjet nozzles, microfluidic devices, needles, diaphragm based pump dispensers, piezoelectric pumps, and other liquid dispensers. The robotic liquid handling systems may be stationary liquid dispensing platforms or be motorized for mapped liquid dispensing. The robotic liquid handling systems may have one or more tips for dispensing liquid. The acoustic liquid dispensers may dispense liquid volumes from less than 1 nanoliter (nL). The acoustic liquid dispensers may have from about 1 to 1600 wells for liquid storage. The syringe pumps may be configured to handle from 1 to 10 or more syringes in parallel. The syringe pumps may use syringes from less than 1 mL in volume to 50 mL or more. The inkjet nozzles may be fixed head or disposable head nozzles. The inkjet nozzles may comprise an array of nozzles from about 1 nozzle to 10 nozzles or more. The inkjet nozzles may be driven by piezoelectric actuators or by thermal drop creation. The microfluidic devices may comprise arrays of microfluidic channels ranging from 1 channel to 1000 or more. The microfluidic devices may be used to start a reaction before the liquid is dispensed into the droplet. The needles may range in size from less than 7 gauge to 24 gauge or more. The needles may comprise an array with a number of needles from 1 needle to 100 needles or more. The diaphragm pump may have a diaphragm made from rubber, thermoplastic, fluorinated polymer, another plastic, or any combination thereof.

The array may be coupled to a reagent storage unit, a sample storage unit, a plurality of reagent storage units, a plurality of sample storage units, or any combination thereof. The reagent storage unit, sample storage unit, plurality of reagent storage units, plurality of sample storage units, or any combination thereof may comprise at least one multi-well plate, tubes, bottles, reservoirs, inkjet cartridges, plates, petri dishes, or any combination thereof. A multi-well plate may include at least about 2, 6, 12, 24, 48, 96, 384, 1536, 3456, 9600, or more wells. The tubes may be selected from Eppendorf tubes or falcon tubes. The bottles may be made of glass, polycarbonate, polyethylene, or another material compatible with what may be stored in the bottle. The bottles may have a capacity of greater than about 10 mL, 20 mL, 30 mL, 40 mL, 50 mL, 60 mL, 70 mL, 80 mL, 90 mL, 100 mL, 200 mL, 300 mL, 400 mL, 500 mL, 600 mL, 700 mL, 800 mL, 900 mL, 1 L, 2 L, 3 L, 4 L, 5 L, or more. The bottles may be replicable. The reservoir may be a high-performance liquid chromatography (HPLC) solvent reservoir. The reservoir may be made of glass, polycarbonate, polyethylene, or another material compatible with what may be stored in the reservoir. The reservoir may have a capacity of greater than about 10 mL, 20 mL, 30 mL, 40 mL, 50 mL, 60 mL, 70 mL, 80 mL, 90 mL, 100 mL, 200 mL, 300 mL, 400 mL, 500 mL, 600 mL, 700 mL, 800 mL, 900 mL, 1 L, 2 L, 3 L, 4 L, 5 L, 6 L, 7 L, 8 L, 9 L, 10 L, 15 L, 20 L, 25 L, 30 L, 35 L, 40 L, 45 L, 50 L, or more. The inkjet cartridge may be commercially available, made specifically for the array, or a combination thereof. The inkjet cartridge may dispense liquid by thermal methods, piezoelectric methods, or a combination thereof. The inkjet cartridge may be refillable, disposable, or have both refillable and disposable components. The inkjet cartridge may contain at least 1, 2, 3, 4, 5, 6, 7, 8, 9, 10, or more different liquids. The plate may be a medium for cell growth. The medium for cell growth may be agar. The agar may have nutrients for the promotion of cell growth. The nutrients for the promotion of cell growth may be blood, derived from blood, sugars, other essential nutrients, or any combination thereof. The petri dishes may incorporate plates. The petri dishes may be bare. The petri dishes may be made of glass, plastic, or a combination thereof. The petri dish may be a replicate organism detection and counting (RODAC) plate. The plurality of wells of the multi-well plate may be thermally conductive, electronically receptive, or a combination thereof. The reagent or sample may be manipulated in or out of the well by an electric field, a magnetic field, an acoustic wave, heat, pressure, vibration, a liquid handling unit, or a combination thereof.

The array may comprise a coating. The coating may be a hydrophobic coating. The coating may be a hydrophilic coating. The coating may comprise both hydrophobic and hydrophilic coatings. The coating may be cleaned by washing. The coating may reduce evaporation. The coating may reduce evaporation by 10% to 100%. The coating may reduce evaporation by 50% to 100%. The coating may reduce biofouling. The coating may reduce biofouling by 10% to 100%. The coating may be resistant to biofouling. The coating may be antibiofouling. The hydrophobic coating may be a fluoropolymer, a polyethylene, or a polystyrene. The hydrophobic coating may also be a modification of the surface with molecules, such as fatty acids, polyaromatic compounds, or the like. For example, oleic acid may be bound to the surface, presenting a carbon chain that would increase the hydrophobicity of the surface. The hydrophilic coating may be a hydrophilic polymer such as poly-vinyl alcohol, poly-ethylene glycol, or the like. The coating comprising both hydrophobic and hydrophilic coatings may be combination of the hydrophilic and hydrophobic polymers above, or it may be a polymer that has both hydrophilic and hydrophobic properties, for example, a copolymer.

The coating may be easily cleaned by washing. Such a coating can be slippery to the samples placed on it to facilitate easy removal of those samples. The droplet may include a coating to prevent or reduce evaporation of material from within the droplet to an environment external to the droplet, from the environment to within the droplet, or any combination thereof. Such coating may reduce evaporation of content from within the droplet by at least 10%, 20%, 30%, 40%, 50%, 60%, 70%, 80%, 90% or more. The coating may be a polymeric coating (e.g., polyethylene glycol). The coating may be formed as a skin around the droplet. The coating may be generated, for example, by bringing the droplet in contact with a fluid comprising a polymeric material (e.g., a polymer or polymer precursor). When the polymeric material comes in contact with the water droplet, diffusion of the fluid into water induces polymerization or cross-linking.

The coating may reduce biofouling, or the accumulation of undesired biological species, by being biocidal or non-toxic. Examples of biocidal coating may be coatings containing a moiety toxic to biological systems, such as tributyl tin or other biocides. Examples of non-toxic coating may include coating with decreased attachment of biological species, such as fluoropolymers or polydimethylsiloxane. Such a coating may be antibiofouling.

The coefficient of variation may be less than 15%, 10%, 5%, or 1%. For example, a coefficient of variation of 1% in droplet size means that for the same series of processes performed on a number of droplets, the standard deviation of the change in droplet size divided by the mean decrease in droplet size would be 1%.

The processing of the plurality of biological samples may comprise nucleic acid sequencing. The nucleic acid sequencing may comprise polymerase chain reaction (PCR). The PCR may comprise highly multiplexed PCR, quantitative PCR, droplet digital PCR, reverse transcriptase PCR, or any combination thereof. The highly multiplexed PCR may be a single or multiple template PCR reaction. The quantitative PCR may use a variety of makers to show the PCR products in real time, such as Sybr green or the TaqMan probe. The droplet digital PCR may use initial droplets from less than 1 microliter to more than 50 microliters, and may separate those droplets into more than 10,000 droplets via an oil water emulsion technique. The reverse transcriptase PCR may be one step or two steps, (i.e., it may require only one droplet or multiple droplets to be completed). The reverse transcriptase PCR may utilize endpoint or real time quantification of the products, which can be done using fluorescence measurements.

The processing of the plurality of biological samples may comprise sample preparation for genomic sequencing. The preparation for genomic sequencing may involve removing DNA from a host cell, cell-free DNA, or any combination thereof. The preparation for genomic sequencing may involve amplification to provide enough DNA for sequencing. The preparation for genomic sequencing may utilize enzymatic fragmentation of the DNA, mechanical fragmentation of the DNA, or any combination thereof.

The processing of the plurality of biological samples may comprise a combinatorial assembly of genes. The combinatorial assembly of genes may comprise a Gibson Assembly, restriction enzyme cloning, gBlocks fragments assembly (IDT), BioBricks assembly, NEBuilder HiFi DNA assembly, Golden Gate assembly, site-directed mutagenesis, sequence and ligase independent cloning (SLIC), circular polymerase extension cloning (CPEC), and seamless ligation cloning extract (SLiCE), topoisomerase mediated ligation, homologous recombination, Gateway cloning, GeneArt gene synthesis, or any combination thereof.

The processing of the plurality of biological samples may comprise cell-free protein expression. The cell-free protein expression may be used to express toxic proteins. The cell-free protein expression may be used to incorporate non-natural amino acids. The cell-free protein expression may utilize phosphoenol pyruvate, acetyl phosphate, creatine phosphate, or any combination thereof as an energy source. The cell-free protein expression may be done at ambient temperatures, temperatures below ambient temperature (e.g., 0° C.), temperatures above ambient temperature (e.g., 60° C.), or any combination thereof.

The processing of the plurality of biological samples may comprise preparation for plasmid DNA extraction. The preparation for plasmid DNA extraction may comprise precipitating the DNA from a lysed cell solution. The preparation for plasmid DNA extraction may comprise using a spin-column based separation technique. The preparation for plasmid DNA extraction may comprise a phenol-chloroform extraction.

The processing of the plurality of biological samples may comprise extracting ribosomes, mitochondria, endoplasmic reticulum, golgi apparatus, lysosomes, peroxisomes, centrioles, or any combination thereof. The ribosomes, mitochondria, endoplasmic reticulum, golgi apparatus, lysosomes, peroxisomes, centrioles, or any combination thereof may remain intact.

The processing of the plurality of biological samples may comprise extraction of nucleic acids from cells. The extraction of nucleic acids from cells may further comprise extracting long strands of nucleic acid, where the long strands of nucleic acid remain completely intact. The long strands of nucleic acid may also be at least 10, 100, 1,000, 10,000, 100,000, 1,000,000, or more base pairs long. The extraction of nucleic acid may involve the lysing of cells via the addition of surfactants and detergents such as octyl glucoside, sodium dodecyl sulfate, or octyl phenol ethoxylate. The extraction of nucleic acids may involve centrifugation, including ultracentrifugation.

The processing of the plurality of biological samples may comprise sample preparation for mass spectrometry. Sample preparation for mass spectrometry may involve cell lysis, digestion, protein amplification, DNA amplification, or other standard sample preparations. Sample preparation for mass spectrometry may include application of a sample to an electrospray ionization (ESI) substrate, incorporation into a matrix-assisted laser desorption ionization (MALDI) matrix, or other preparation for ionization. Mass spectrometry may include ion trap, quadrupole, and other detection methods. The inlet of the mass spectrometer may be directly coupled to at least one droplet. The inlet of the mass spectrometer may be adjacent to one or more droplets. The sample for mass spectrometry may be transferred to the inlet of the mass spectrometer by pipetting.

The processing of the plurality of biological samples may comprise sample extraction and library preparation for nucleic acid sequencing. The nucleic acid sequencing may comprise sequencing by synthesis, pyrosequencing, sequencing by hybridization, sequencing by ligation, sequencing by detection of ions released during polymerization of DNA, single-molecule sequencing, or any combination thereof. The single molecule sequencing may be nanopore sequencing. The single molecule sequencing may be single molecule real time (SMRT) sequencing.

The processing of the plurality of biological samples may comprise DNA synthesis using oligonucleotide synthesis, enzymatic synthesis, or any combination thereof. The oligonucleotide synthesis may be solid state, liquid phase, performed in solution, or any combination thereof. The oligonucleotide synthesis may produce oligonucleotides that may be at least 2, 5, 10, 20, 30, 40, 50, 100, 200, 300, 400, 500, or more nucleotides. The enzymatic synthesis may use polymerases, transferases, other enzymes, or any combination thereof.

The processing of the plurality of biological samples may comprise DNA data storage, random-access of stored DNA and DNA data retrieval through DNA sequencing. DNA data storage may utilize strands of DNA having greater than about 10, 50, 100, 150, 200, 250, 500, 1,000, 5,000, 10,000, 100,000, 1,000,000, or more base pairs. DNA sequencing may include at least one PCR reaction, a Maxam-Gilbert sequencer, a Sanger sequencer, or any combination thereof. The nucleic acid sequencing may comprise sequencing by synthesis, pyrosequencing, sequencing by hybridization, sequencing by ligation, sequencing by detection of ions released during polymerization of DNA, single-molecule sequencing, or any combination thereof. The single molecule sequencing may be nanopore sequencing. The single molecule sequencing may be single molecule real time (SMRT) sequencing.

The processing of the plurality of biological samples may comprise nucleic acid extraction and sample preparation integrated directly into a sequencer. The nucleic acid extraction and sample preparation may be performed directly on the array. The nucleic acid extraction and sample preparation may be performed adjacent to the array. The sequencer may be adjacent to the array. The sequencer may be coupled to the array. The sequencer may be directly on the array.

The processing of the plurality of biological samples may comprise CRISPR genome editing. The editing may comprise Cas9 protein, Cpf1 endonuclease, crRNA, tracrRNA, or any combination thereof. A repair DNA template may be used during the editing process. The repair DNA template may be a single-stranded oligonucleotide, double-stranded oligonucleotide, or a double-stranded DNA plasmid.

The processing of the plurality of biological samples may comprise transcription activator-like effector nucleases (TALENs) genome editing. The processing of the plurality of biological samples may comprise zinc fingers nuclease gene editing.

The processing of the plurality of biological samples may comprise at least one high-throughput process. The high-throughput process may be automated to not require input. The high-throughput process may comprise at least one of the assays or characterization methods applied to at least one of the sample types that are described herein.

The processing of the plurality of biological samples may comprise the screening of a plurality of chemical compounds against a plurality of cells. The chemical compound may be one or more chemical compounds. The chemical compound may show a biological effect. A biological effect may be the promotion or inhibition of cellular growth, the signaling of a cellular process to begin or end, the induction of cell division, or the like.

The chemical compounds may be antibacterial. Antibacterial chemicals may inhibit the growth of bacteria from at least 5% to greater than 99%. Antibacterial chemicals may kill bacteria.

The chemical compound may be screened for biological activity. The chemical compound may use the sensors of the array to determine biological activity. For example, an array of fluorescence detectors may be used to determine the relative amount of a fluorescent protein in a biological sample exposed to a chemical compound of interest. Similarly, for example, a microscope may be used to assay the total number of a cell species after exposure to a chemical compound. The chemical compound may be isolated. The isolation may involve centrifugation, transfer via pipetting or another liquid transfer technique, precipitation, a chromatographic technique (e.g., column chromatography, thin layer chromatography, etc.), distillation, lyophilization, or recrystallization. The screen for biological activity may involve mixing at least one biological sample in at least one droplet with at least one chemical.

The cells may be bacterial cells. The bacterial cells may be disease causing. The bacterial cells may be resistant to antibiotics. The bacterial cells may be genetically modified.

The cells may be eukaryotic cells. The eukaryotic cells may be single celled organisms (e.g. protozoans, algae), diatoms, fungal cells, insect cells, animal cells, mammalian cells, or human cells. The eukaryotic cells may be derived from single celled organisms (e.g. protozoans, algae), diatoms, fungi, insects, animals, mammalians, or humans. The eukaryotic cells may be derived from a larger tissue or organ. The eukaryotic cells may be genetically modified. The eukaryotic cells may be suspected of having or carrying a disease.

The cells may be prokaryotic cells. The prokaryotic cells may be genetically modified.

The processing of the plurality of biological samples may comprise culturing cells, thereby producing cultured cells. The culturing of the cells may occur in discrete droplets. The culturing of the cells may occur in discrete physical compartments. The culturing of cells may be done autonomously (with no input required). The culturing of cells may be performed on solid, liquid or semi-solid media. The culturing of cells may occur in 2 or 3 dimensions. The culturing of cells may be done under ambient or non-ambient conditions (e.g., elevated temperature, low pressure, etc.). The discrete physical compartments may be discrete electrowetting chips.

The interactions between the cultured cells or between cultured cells and at least one biological sample may be determined. The interaction of two or more samples of cultured cells may be determined by mixing. The interaction of at least one biological sample and the cultured cells may be determined by mixing, applying the cultured cells directly onto the biological sample, or applying the biological sample directly onto the cultured cells. Applying the cultured cells may involve transferring liquid cell culture or placing a solid cell culture onto the sample of interest.

The cultured cells may be assayed on the array, or the plurality of arrays as described herein.

The cultured cells may be isolated from culture. The isolation may involve centrifugation, transfer via pipetting or another liquid transfer technique, precipitation, scraping the cells off of the culture, or a chromatographic technique (e.g., cellular chromatography). The isolated cells may be transferred to an external container. The external container may be a society for biomolecular screening (SBS) format plate, a petri dish, a bottle, a box, another culture medium, or the like.

The isolated cells may be prepared for nucleic acid sequencing.

The isolated cells may be prepared for protein analysis. The protein analysis may be an amino acid analysis, size analysis, absorption analysis, the Kjeldahl method, the Dumas method, western blot analysis, high-performance liquid chromatography (HPLC) analysis, liquid chromatography—mass spectrometry (LC/MS) analysis, or enzyme-linked immunosorbent assay (ELISA) analysis.

The isolated cells may be prepared for metabolomic analysis. The metabolomic analysis may be aqueous metabolite profiling, lipid metabolite profiling, nuclear magnetic resonance spectroscopy (NMR) analysis, or a mass spectrometry analysis.

The array may comprise a plurality of lyophilized reagents, dry reagents, stored beads, or any combination thereof. The plurality of lyophilized reagents, dry reagents, stored beads, or any combination thereof may be reconstituted. The lyophilized reagents may include proteins, bacteria, microorganisms, vaccines, pharmaceuticals, molecular barcodes, oligonucleotides, primers, DNA sequences for hybridization, enzymes (e.g., glucosidase, alcohol dehydrogenase, a DNA polymerase, etc.) and dehydrated chemicals. The dry reagents may include chemical powders (e.g. salts, metal oxides, etc.), biologically derived chemicals, dry buffer chemicals, other bioactive chemicals, and the like. The stored beads may be magnetic beads, beads for the storage of bacteria, enzymes, oligonucleotides, or molecular sieves. The molecular barcodes may be DNA fragments with at least 5, 10, 20, 30, 40, 50, 60, or more base pairs. The oligonucleotides may be at least 2, 5, 10, 20, 30, 40, 50, 100, 200, 300, 400, 500, or more nucleotides. The primer may be DNA or RNA. The DNA sequences for hybridization may be used to detect small differences in nucleotide order. The DNA sequence may be used in conjunction with mismatch detection proteins.

The droplet, a plurality of droplets, derivatives thereof, or any combination thereof may be used to reconstitute the lyophilized reagents, dry reagents, stored beads, or any combination thereof. The reconstitution may solubilize, suspend, or form colloids of the lyophilized reagents, dry reagents, stored beads, or any combination thereof. The reagents may be prefabricated into a component of the array.

The array may store a plurality of reagents as a solid, liquid, gas, or any combination thereof. The array may condense, sublime, thaw, evaporate, or any combination thereof, the stored reagent. The reagent may be a compressed gas (e.g., air, argon, nitrogen, oxygen, carbon dioxide, etc.), a solvent (e.g., water, dimethyl sulfoxide, acetone, ethanol, etc.), a cleaner (e.g., ethanol, SDS, liquid soap, etc.), or a solution (e.g., a buffer, a chemical dissolved in a liquid, etc.). For an example of the array performing a physical state transformation of a stored reagent, solid carbon dioxide (dry ice) may be sublimed to provide cold carbon dioxide gas to a droplet. Another example may be for the array to boil water to introduce steam into a droplet or to clean the array.

The array may dispense a plurality of liquids. The array may use a variety of methods to dispense the plurality of liquids, such as, for example by pipetting, condensing, decanting, or any combination thereof, employing devices such as: microfluidic device, diaphragm pump, nozzle, piezoelectric pump, needle, tube, acoustic dispenser, capillary, or any combination thereof. The plurality of liquids may be from at least 2, 3, 4, 5, 6, 7, 8, 9, 10, 20, 30, 40, 50, 60, 70, 80, 90, 100, 500, 1,000, or more liquids.

The array may mix a plurality of liquids. The mixing may be performed by stirring, sonication, vibration, gas flow, bubbling, shaking, swirling, and electrowetting forces. The plurality of liquids may be from at least 2, 3, 4, 5, 6, 7, 8, 9, 10, 20, 30, 40, 50, 60, 70, 80, 90, 100, 500, 1,000, or more liquids. The liquids may be in the form of at least one droplet. The at least one droplet may be on an electrowetting array.

The processing of the plurality of biological samples may be automated (e.g., made able to be run without user input). The automation may use a program to run. The program may be a machine learning algorithm. The program may utilize a neural network. The automation may be controlled by a device. The device may be a computer, a tablet, a smartphone, or any other device capable of executing the code. The automation may interface with one or more components of the array (e.g., sensors, liquid handling devices, etc.) to perform the processing. In some embodiments, the automation may use a camera that tracks the size of a droplet on the array. When the droplet has lost sufficient volume due to evaporation, as determined by a computer vision program, the automation would instruct the liquid handling unit to dispense a precise amount of liquid to the droplet to maintain a pre-programmed volume. In this embodiment, an open configuration may allow for easier observation of the droplets.

The array may be reusable. The array may have a replaceable surface. The array may have a replaceable film. The array may have a replaceable cartridge. The replaceable cartridge may comprise a film. The film may be attached to the array. The film may be fastened to the array using vacuum. The film may be coupled to the array using an adhesive. The adhesive may be non-reactive, pressure-sensitive, contact reactive, heat reactive (e.g., anaerobic, multi-part (e.g., polyester, polyols, acrylic, etc.), pre-mixed, frozen, one-part), natural, synthetic, or any combination thereof. The adhesive may be applied by spraying, brushing, rolling, or by a film or applicator. The adhesive may be, but is not limited to, silicone, acrylic, epoxy, polyurethane, starch, cyanoacrylate, polyimide, or any combination thereof. The array may be reused from at least 2, 3, 4, 5, 6, 7, 8, 9, 10, 20, 30, 40, 50, 60, 70, 80, 90, 100, 500, 1,000, or more times. The replaceable surface may be easy to remove and reattach to the array. The replicable surface may be a layer of a liquid. The liquid may be an oil. The replaceable film may be a polymer (e.g., polyethylene, polytetrafluoroethylene, polydimethylsiloxane, etc.). The replaceable film may be from 1 nanometer to 1 millimeter thick. The replaceable cartridge may comprise a new electrowetting chip. The replaceable cartridge may comprise a new surface to be placed on the electrodes of an electrowetting chip.

The array may be washed. The array may be washed entirely. The array may be washed partially. The array may be washed using a material stored in the reagent dispensing array. The array may be washed using a solid cleaner (e.g., powdered soap, a solid antimicrobial, etc.), a liquid cleaner (e.g., liquid soap, ethanol, etc.), or a gaseous cleaner (e.g., steam). About 1% to 100% of the array may be washable.

The array may be disposable. The disposable array may comprise the entire sample assembly. The disposable array may comprise the surface of an electrowetting chip. The disposable array may be easily removed.

The volume of biomolecules of the array may be manipulated as a mixture. The volume of biomolecules may comprise a plurality of nucleic acids, protein sequences, or a combination thereof. The plurality of nucleic acid, protein sequences, or a combination thereof may be manipulated by modulation of local surface charge without physical contact on the mixture by another component of the array. For example, an electrowetting chip may be used to move a droplet containing a number of nucleic acids by changing the surface wetting properties of the droplet. This would allow the droplet to move without contact from another component of the array. The mixture may be within a droplet. The droplet may comprise a volume of at least 1 picoliter (pL), 10 pL, 100 pL, 1 nanoliter (nL), 10 nL, 100 nL, 1 μL, 10 μL, 100 μL, 1 milliliter (mL), 10 mL or more. The mixture may comprise a protein with DNA ligase activity. The mixture may comprise a protein with DNA transposase activity. The protein with DNA ligase activity may be derived from a virus (e.g., T4), a bacteria (e.g., E. coli), or a mammal (e g, human DNA ligase 1). The protein with DNA transposase activity may be derived from a bacteria (e.g., Tn5) or a mammal (e g, sleeping beauty (SB) transposase). The volume of biomolecules of the assay may be manipulated with lateral geospatial movement of the mixture of at least 1 mm. The volume of biomolecules of the assay may be manipulated by a predetermined or preprogrammed set of commands. The commands may be associated with a particular location of the array.

The array may comprise reagents for conducting a strand displacement amplification reaction, a self-sustained sequence replication and amplification reaction or a Q3 replicase amplification reaction. The reagent for conducting a strand displacement amplification reaction may be Bst DNA polymerase, cas9, or another hemiphosphorothioate form nicking protein. A self-sustained sequence replication and amplification reaction reagents may be avian myeloblastosis virus (AMV) reverse transcriptase (RT), Escherichia coli RNase H, T7 RNA polymerase, or any combination thereof. The reagents for the Q3 replicase amplification reaction may be derived from the Q3 bacteriophage, E. coli, or any combination thereof.

The array may comprise reagents including a DNA ligase, a nuclease or a restriction endonuclease. The DNA ligase may be derived from a virus (e.g., T4), a bacteria (e.g., E. coli), or a mammal (e g, human DNA ligase 1). The nuclease may be an exonuclease (starting digestion from the end of a molecule) or an endonuclease (digesting from somewhere other than the end of a molecule). The nuclease may be a deoxyribonuclease (operating on DNA) or a ribonuclease (operating on RNA). The restriction endonuclease may be a type I, II, III, IV, or V restriction endonuclease. An example of a restriction endonuclease may be cas9 or a zinc finger nuclease.

The array may comprise reagents for the preparation of an amplified nucleic acid product. The reagents for the preparation of an amplified nucleic acid product may be Bst DNA polymerase, deoxyribonucleotide triphosphate, fragments of E. coli DNA polymerase 1, avian myeloblastosis virus reverse transcriptase, RNase H, T7 DNA dependent RNA polymerase, Taq polymerase, other DNA polymerases/transcriptases, or any combination thereof.

The array may be a component in the manufacture of a kit or system for the diagnosis or prognosis of a disease. The kit may process a biological sample. The biological sample may be a sample derived from a patient. In some embodiments, the array may be used to process a sample derived from a patient suspected of having a disease. The disease may be a disease classified by the Centers for Disease Control and Prevention (CDC). The array may mix the sample with a reagent. The array may mix the sample with a reagent for separating cells from serum. The array may process the cells, or derivatives thereof. The array may transfer cells, or derivatives thereof, to an optical device coupled to the array. The cells, or derivatives thereof, may be processed according to methods described herein.

The array may include a protein with nucleic acid cleavage activity. The array may include a biomolecule with RNA cleavage activity. The protein with nucleic acid cleavage activity may be a ribonuclease, a deoxyribonuclease, or any combination thereof. The biomolecule with RNA cleavage activity may be a small ribonucleolytic ribozyme, a large ribonucleolytic ribozyme, or any combination thereof.

An interchangeable set of reagents may be introduced by at least one solid phase support. The solid phase support may be a paper strip. The solid phase support may be a microbead. The solid phase support may be a pillar. The pillar may be attached to the base of the support or integral to the support. The solid phase support may be a strip of microwells. The solid phase support may be a glass slide, a scoop, or a plastic film. The solid phase support may be a bead. The bead may be magnetic. The interchangeable set of reagents may be chemical reagents (e.g., small molecules, metals, etc.), biological species (e.g., proteins, DNA, RNA, etc.), processing reagents (e.g., PCR reagents, etc.).

The interchangeable set of reagents may be introduced by at least one secondary support. The secondary support may be a strip of microwells. The secondary support may be a SBS plate, petri dish, bottle, slide, or another container. The interchangeable set of reagents may be chemical reagents (e.g., small molecules, metals, etc.), biological species (e.g., proteins, DNA, RNA, etc.), processing reagents (e.g., PCR reagents, etc.).

The array may contain a template independent polymerase. The template independent polymerase may be a terminal deoxynucleotidyl transferase (TdT). The array may include an enzyme that limits nucleic acid polymerization. The enzyme that limits nucleic acid polymerization may be an apyrase. The array may have sensors to detect the presence of at least one terminal ‘C’ tail in a nucleic acid molecule. The at least one terminal ‘C’ tail may be isolated. The apyrase may be derived from E. coli, S. tuberosum, or an arthropod.

The plurality of biological samples of the array may be stored by drying. The drying may be performed by heating, vacuum, flowing gas, lyophilization, or any combination thereof. The samples may be stored on the array or in another container. The other container may be a glass slide, petri dish, media bottle, tube, or (micro)well array.

The plurality of biological samples of the array may be retrieved by rehydration. The rehydration may be performed by adding liquid to or blowing a gas containing liquid over the dried plurality of biological samples. The rehydrated plurality of biological samples may be manipulated with any of the liquid handling mechanisms stated above.

The plurality of biological samples may be deposited onto the plurality of arrays in SBS format or on any random location of the plurality of arrays, thereby producing at least one deposited biological sample. The SBS format may be the dimensions of a 96 well plate. The deposited biological sample may be a solid or a liquid.

The plurality of biological samples may be deposited using commercial acoustic liquid handlers in preparation for manipulating samples on the chip. The acoustic liquid handlers may be an Echo® or an ATS Gen5®. The at least one deposited biological sample may be used for cell-free synthesis. The at least one deposited biological sample may be used for combinatorially assembling large DNA constructs. The combinatorially assembling large DNA constructs may be a Gibson assembly, circular polymerase extension cloning, and DNA Assembler method.

The processing of the plurality of biological samples may comprise at least one of the following assays, or any combination thereof: digital PCR, isothermal amplification of nucleic acids, antibody mediated detection, enzyme linked immunoassay (ELISA), electrochemical detection, colorimetric assay, fluorometric assay, and micronucleus assay.

The digital PCR assay may process droplets from at most about 1,000 microliters, 900 microliters, 800 microliters, 700 microliters, 600 microliters, 500 microliters, 400 microliters, 300 microliters, 200 microliters, 100 microliters, 50 microliters, 10 microliters, 1 microliter, 0.1 microliters, 0.01 microliters, 0.001 microliters, 0.0001 microliters, or less. The digital PCR may use initial droplets from at least about 0.0001 microliters, 0.001 microliters, 0.01 microliters, 0.1 microliters, 1 microliter, 10 microliters, 50 microliters, 100 microliters, 200 microliters, 300 microliters, 400 microliters, 500 microliters, 600 microliter, 700 microliters, 800 microliters, 900 microliters, 1,000 microliters, or more. The digital PCR may use initial droplets from about 100 microliters to about 1 microliter. The digital PCR may use initial droplets from about 50 microliters to about 1 microliter. In some embodiments, the digital PCR assay may separate a droplet, or a plurality thereof, into at least about 1, 2, 5, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more, droplets. The droplet, or plurality thereof, may be separated by an oil water emulsion technique.

The isothermal amplification of nucleic acids may be PCR, strand-displacement amplification (SDA), rolling circle amplification (RCA), loop-mediated isothermal amplification (LAMP), nucleic acid sequence based amplification (NASBA), helicase-dependent amplification (HDA), recombinase polymerase amplification (RPA), cross-priming amplification (CPA), or any combination thereof.

The antibody mediated detection may be used to detect cells, proteins, nucleic acid molecules (e.g., DNA, RNA, PNA, etc.), hormones, antibodies, small molecules, or any combination thereof. The antibody mediated detection may comprise antibodies that comprise antigen-binding sites specific to detect a cell, protein, nucleic acid, or any combination thereof. The antibody may be naturally-derived. The antibody may be a synthetic antibody. The synthetic antibody may be a recombinant antibody, a nucleic acid aptamer, a non-immunoglobulin protein scaffold, or any combination thereof.

The enzyme linked immunoassay (ELISA) may be direct, sandwich, competitive, reverse type, or any combination thereof. The ELISA may detect, quantify, or a combination thereof, substances, such as, for example, peptides, proteins, antibodies, hormones, small-molecules, or any combination thereof.

The electrochemical detection may be an oxidation- or reduction-based electrochemical detection. The oxidation- or reduction-based electrochemical detection may be conductometric, potentiometric, voltammetric, amperometric, coulometric, impedimetric, or any combination thereof. The electrochemical detection may be used to detect a cell, proteins, nucleic acids, hormones, small-molecules, antibodies, or any combination thereof. The electrochemical detection may detect electric currents generated from oxidative or reductive reactions of biological samples. The electrochemical detection may detect electric currents generated from oxidative or reductive reactions of biological samples.

The colorimetric assay may be used to detect cells, nucleic acids, proteins, small-molecules, antibodies, hormones, or any combination thereof. The colorimetric assay may be used to assay an absorption of a wavelength of at least 240 nm, 280 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, 1250 nm, 1500 nm, 1750 nm, 2000 nm, 2400 nm, or more. The colorimetric assay may be used to assay an absorption of a wavelength of at most 2400 nm, 2000 nm, 1750 nm, 1500 nm, 1250 nm, 1000 nm, 950 nm, 900 nm, 850 nm, 800 nm, 750 nm, 700 nm, 650 nm, 600 nm, 550 nm, 500 nm, 450 nm, 400 nm, 350 nm, 300 nm, 280 nm, 240 nm, or less. The colorimetric assay may be used to assay an absorption of a wavelength from about 2400 nm to about 240 nm. The colorimetric assay may be used to assay an absorption of a wavelength from about 1000 nm to about 100 nm. The colorimetric assay may be used to assay an absorption of a wavelength from about 900 nm to about 400 nm. The colorimetric assay may be performed on solid, liquid, or gaseous samples. The colorimetric assay may use a broadband light source (e.g., an incandescent source, an LED, etc.), a laser source, or a combination thereof. The light source may be passed through a variety of optical elements (e.g., lenses, filters, mirrors, etc.) before and after it interacts with the sample. The transmitted or reflected light may be detected (e.g., by a mirror, a fiber optic, etc.) via a charge-coupled device (CCD), a photomultiplier tube, an avalanche photodiode, or any combination thereof. The detector may be coupled to a wavelength selecting device, such as, for example, a monochrometer or a filter or set of filters.

The fluorometric assay may be used to detect cells, nucleic acids, proteins, small-molecules, antibodies, hormones, or any combination thereof. The fluorometric assay may be used to assay an absorption of a wavelength of at least 240 nm, 280 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, 1250 nm, 1500 nm, 1750 nm, 2000 nm, 2400 nm, or more. The fluorometric assay may be used to assay an absorption of a wavelength of at most 2400 nm, 2000 nm, 1750 nm, 1500 nm, 1250 nm, 1000 nm, 950 nm, 900 nm, 850 nm, 800 nm, 750 nm, 700 nm, 650 nm, 600 nm, 550 nm, 500 nm, 450 nm, 400 nm, 350 nm, 300 nm, 280 nm, 240 nm, or less. The fluorometric assay may be used to assay an emission of a wavelength from about 2400 nm to about 240 nm. The fluorometric assay may be used to assay an emission of a wavelength from about 1000 nm to about 100 nm. The fluorometric assay may be used to assay an emission of a wavelength from about 900 nm to about 400 nm. The fluorometric assay may use a broadband light source (e.g., an incandescent source, an LED, etc.), a laser source, or a combination thereof. The light source may be passed through a variety of optical elements (e.g., lenses, filters, mirrors, etc.) before and after it interacts with the sample. The fluoresced light may be detected via a CCD, a photomultiplier tube, an avalanche photodiode, or any combination thereof. The detector may be coupled to a wavelength selecting device, such as a monochrometer or a filter or set of filters. For example, a fluorometric assay may be used to determine the concentration of reduced NADPH, as it fluoresces in its reduced form but not in its oxidized form. In this example, the intensity of the observed fluorescence over time would correspond linearly with the amount of reduced NADPH in the sample.

The micronucleus assay may evaluate the presence of micronuclei in a biological sample. The micronuclei may contain chromosome fragments produced from DNA breakage (clastogens) or whole chromosomes produced by disruption of the mitotic apparatus (aneugens). The micronucleus assay may be used to identify genotoxic compound. The genotoxic compound may be a carcinogen. The micronucleus assay may be performed in vivo or in vitro. The in vivo micronucleus assay may utilize bone marrow or peripheral blood from a biological sample. The in vitro micronucleus assay may utilize cells or tissues derived from a plurality of biological samples.

The processing of the plurality of biological samples may comprise isothermal amplification of at least one selected nucleic acid, which may comprise: providing at least one sample that may comprise at least one nucleic acid by merging droplets containing a plurality of reagents effective to permit at least one isothermal amplification reaction of the sample without mechanical manipulation; and conducting at least one isothermal amplification reaction to amplify the nucleic acid.

The at least one isothermal amplification of at least one selected nucleic acid may be PCR, strand-displacement amplification (SDA), rolling circle amplification (RCA), loop-mediated isothermal amplification (LAMP), nucleic acid sequence based amplification (NASBA), helicase-dependent amplification (HDA), recombinase polymerase amplification (RPA), cross-priming amplification (CPA), or any combination thereof. The at least one isothermal amplification may be at least 2, 3, 4, 5, 6, 7, 8, 9, 10 or more isothermal amplifications.

The at least one nucleic acid may be at least 10, 100, 1,000, 10,000, 100,000, 1,000,000, or more base pairs long. The merging droplets may be at least 2, 3, 4, 5, 6, 7, 8, 9, 10 or more droplets. The plurality of reagents may be any of the isothermal amplification reagents described herein.

The processing of the plurality of biological samples may comprise a device to detect a polymerase chain reaction (PCR) product on at least one droplet. The droplet may be an aqueous droplet. The device may: create at least one droplet containing a plurality of nucleic acid and protein molecules on an electrowetting array; perform the PCR reaction while the aqueous droplets are present on the array surface; and interrogate the droplet with a detector. The PCR product may be DNA or RNA. The protein molecules may be enzymes, utilized in the PCR reaction, or used to report the progress of a reaction (e.g., luminescent). The performance of the PCR reaction may include agitating the sample (e.g., stirring, vibration, electrowetting based movement, etc.), heating or cooling the sample (using the aforementioned heater and cooler arrays), and controlling the droplet size. The detector may be any detector described herein.

The device may comprise a plurality of reporter molecules. The reporter molecules may be fluorescent reporter molecules. The plurality of fluorescent reporter molecules may be separated by at least one enzyme from at least one quencher molecule during the PCR reaction. The at least one enzyme may comprise a polymerase, oxidoreductase, transferase, hydrolase, lyase, isomerase, or ligase. The plurality of fluorescent reporter molecules may be a protein, a luminescent small molecule, a luminescent nucleic acid, or a nanoparticle.

The nucleic acid may be detected by a sensor. The sensor may detect a radiolabel. The sensor may detect a fluorescent label. The sensor may detect a chromophore. The sensor may detect a redox label. The sensor may be a p-n-type diffusion diode. The nucleic acid may be detected by a smartphone.

The processing of the plurality of biological samples may include binding at least one biomolecule on the array. The at least one biomolecule may be immobilized on a surface. The at least one biomolecule may be immobilized on a diffusible matrix. The at least one biomolecule may be immobilized on a diffusible bead. The at least one biomolecule may be a protein, a compound derived from a biological system (e.g., a signaling molecule, a cofactor, etc.), a pharmaceutical, a molecule exhibiting or suspected of exhibiting biological activity, a carbohydrate, lipid, a nucleic acid, a natural product, or a nutrient. The immobilization may be by adsorption, ionic interaction, covalent bonding, or intercalation. The surface may be an electrowetting chip, a polymer, a dielectric, a metal, a fiber based sheet (e.g., a paper strip), or a stationary phase (e.g., silica gel). The diffusible matrix may be a polymer, a tissue (e.g., collegian), or an aerogel. The diffusible bead may be a polymer bead, a molecular sieve, or a bead formed of biological materials (e.g., a beaded protein or nucleic acid). The location of the biomolecule may be identified by a coding scheme. The coding scheme may be a preprogrammed method to determine the location of the biomolecule. The coding scheme may be based on a moiety to which it is immobilized.

In some embodiments, detectable labels may fluorescent labels for emitting a specific wavelength. In some embodiments, the fluorescent labels emit light upon excitation by a light source. In some embodiments, the detectable labels emit light at a wavelength of 380-450 nm. In some embodiments, the detectable labels emit light at a wavelength of 450-495 nm. In some embodiments, the detectable labels emit light at a wavelength of 495-570 nm. In some embodiments, the detectable labels emit light at a wavelength of 570-590 nm. In some embodiments, the detectable labels emit light at a wavelength of 590-620 nm. In some embodiments, the detectable labels emit light at a wavelength of 620-750 nm. In some embodiments, interchangeable optical filters are utilized by a computer-vision system. In some embodiments, optical filters are used in combination with one or more optical sensors or image sensors of the computer-vision system. In some embodiments, the optical filters are provided to filter wavelengths produced by detectable labels, such that only one or more labels corresponding to samples of a particular type are to be detected or monitored by the system. In some embodiments, the system may comprise one or more optical sensors, wherein each optical sensor is provided with a specific filter to monitor a specified label corresponding to samples of a particular type, as described herein.

In some embodiments, the array may induce an interaction of the plurality of biomolecules from two or more non-continuous liquid volumes without mechanical manipulations. The interaction may be mixing, a chemical reaction, adsorption, or an enzymatic reaction. Without mechanical manipulations may mean that the moving part of the interaction may be the two or more non-continuous liquid volumes. The plurality of biomolecules may be at least one of a protein, a compound derived from a biological system (e.g., a signaling molecule, a cofactor, etc.), a pharmaceutical, a molecule exhibiting or suspected of exhibiting biological activity, a carbohydrate, lipid, a nucleic acid, a natural product, or a nutrient.

The array may prepare an amplified nucleic acid product without mechanical manipulations. The array may conduct a diagnostic test on a nucleic acid sample without mechanical manipulations. The array may conduct a diagnostic or prognostic test on a biological sample without mechanical manipulations. The plurality of biological samples may be suspected of containing a nucleic acid biomarker.

The array may comprise a gas source that contacts and may be absorbed by at least one droplet. The at least one droplet may be manipulated on the device. The gas may be air, nitrogen, argon, carbon dioxide, hydrogen, or water vapor. The at least one droplet may absorb at least 0%, 10%, 20%, 30%, 40%, 50%, 60%, 70%, 80%, 90%, 99%, or more of the gas. The manipulation may be due to the pressure the gas exerts on the at least one droplet.

The plurality of biological samples may include reagents for conducting a strand displacement amplification reaction, a self-sustained sequence replication, an amplification reaction, or a Q3 replicase amplification reaction. The reagent for conducting a strand displacement amplification reaction may be Bst DNA polymerase, cas9, or another hemiphosphorothioate form nicking protein. A self-sustained sequence replication and amplification reaction reagents may be avian myeloblastosis virus (AMV) reverse transcriptase (RT), Escherichia coli RNase H, T7 RNA polymerase, or any combination thereof. The reagents for the Q3 replicase amplification reaction may be derived from the Q3 bacteriophage, E. coli, or any combination thereof i.

The array may receive at least one instruction from a remote computer to process the array of biological samples. The at least one instruction may be at least 1, 2, 3, 4, 5, 6, 7, 8, 9, 10, 20, 30, 40, 50, 60, 70, 80, 90, 100, 200, 300, 400, 500, 600, 700, 800, 900, 1,000 or more instructions. The remote computer may be any system capable of sending instructions (e.g., a desktop computer, a laptop computer, a tablet, a smartphone, an application-specific integrated circuit, etc.). The remote computer may not require user input to send the at least one instruction.

The array may be preprogrammed to perform the process on the array of biological samples. The preprogramming may be for at least 1, 2, 3, 4, 5, 6, 7, 8, 9, 10, 20, 30, 40, 50, 60, 70, 80, 90, 100, 200, 300, 400, 500, 600, 700, 800, 900, 1,000 or more steps of the process. The preprogramming may be stored in the array (e.g., on a hard drive, on a flash memory unit, on erasable programmable read-only memory (EPROM), on a tape cassette, etc.) or stored on an attached system capable of sending instructions (e.g., a desktop computer, a laptop computer, a tablet, a smartphone, an application-specific integrated circuit, etc.).

The array may receive information related to a DNA sequence. The information related to a DNA sequence may include the length of the DNA sequence, the composition of the DNA sequence (e.g., the total number of a given base, the sequence of the bases, etc.), or the presence of a particular DNA sequence. The DNA sequence may trigger an automated process. The information related to the DNA sequence may trigger an automated process. The automated process may include conversion of the DNA sequence into at least one constituent oligonucleotide sequence. The at least one constituent oligonucleotide sequence may be assembled, error corrected, reassembled, or any combination thereof, into DNA amplicons. The DNA amplicons may direct production of RNA, proteins, biological particles, or any combination thereof. The biological particles may be derived from a virus.

The array may produce at least one peptide or antibody from a DNA template. The array may produce using in vivo methods (e.g., using cells to produce) or cell-free production (e.g., not requiring a living organism to produce). The peptide may be at least 1, 2, 3, 4, 5, 6, 7, 8, 9, 10, 15, 20, 25, 30, 35, 40, 45, 50, or more amino acids. The amino acids may be naturally occurring or non-naturally occurring. The antibody may be surface bound or free. The antibody may be derived from any of the plurality of biological samples.

The array may partition at least one droplet into a plurality of droplets by: electromotive force, electrowetting force, dielectrowetting force, dielectrophoretic effect, acoustic force, hydrophobic knife, or any combination thereof. The electrowetting force may be induced by a configuration of the array mentioned above. The dielectrophoretic effect may be photoinduced (electromagnetic radiation may be used to induce the effect). The dielectrophoretic effect may be induced by wires, sheets, electrodes, or any combination thereof created by photolithography, laser ablation, electron beam patterning, or any combination thereof. The wires, sheets, and electrodes may be made of metals (e.g., gold, copper, silver, titanium, etc.), alloys of metals, semiconductors (e.g., silicon, gallium nitride), or conductive oxides (e.g., indium tin oxide). The acoustic force may be ultrasonic. The acoustic force may be generated by a transducer. The hydrophobic knife may be a hydrophobic microtome or a hydrophobic razor blade.

The partitioning may dispense reagents. The reagents may be any of the reagents as described herein.

The partitioning may dispense samples. The samples may be a plurality of biological samples. The samples may be non-biological samples (e.g., chemicals).

The partitioned droplets may be mixed to execute a reaction. The reaction may be an amplification reaction, a chemical transformation, a binding reaction, the reaction of an antimicrobial agent with a microbe, or a reaction mentioned above.

The partitioned droplets may be analyzed using the sensors. The sensors may be any of the sensors from the array of sensors mentioned above.

The partitioned droplets may be mixed with at least one target droplet to maintain a constant volume on the at least one target droplet. The constant volume may be determined by computer vision (coupled cameras and an algorithm), mass, or optical spectroscopy (e.g., absorption spectroscopy).

The array may process multiphase fluids. The fluids may have at least 2, 3, 4, 5, 6, or more phases. For example, a droplet of water containing a colloid that is itself surrounded by a droplet of oil would have 3 phases.

The array may use dielectrophoretic forces (DEP) for cell sorting, cell separation, manipulating at least one bead, or any combination thereof. The DEP may be photoinduced (electromagnetic radiation may be used to induce the effect). The DEP may be induced by wires, sheets, electrodes, or any combination thereof created by photolithography, laser ablation, electron beam patterning, or any combination thereof. The wires, sheets, and electrodes may be made of metals (e.g., gold, copper, silver, titanium, etc.), alloys of metals, semiconductors (e.g., silicon, gallium nitride), or conductive oxides (e.g., indium tin oxide). The bead may comprise a magnetic bead, a bead for the storage of bacteria, an enzyme, an oligonucleotide, a nucleic acid, an antibody, a PCR primer, a ligand, a molecular sieve, or any combination thereof. The sorting and separation may be used for pre-concentrating at least one cell in raw clinical samples. The raw clinical samples may be derived from the plurality of biological samples. The raw clinical samples may be from a subject having or suspected of having a disease.

A biological sample, or a plurality thereof, may be deposited on an array or a plurality of arrays. The plurality of array may comprise at least two arrays. An array of the plurality of arrays may comprise a surface. The surface may comprise glass, a polymer, ceramic, metal, or any combination thereof. The surface may comprise a EWOD array, a DEW array, a DEP array, a microfluidic array, or any combination thereof. The plurality of arrays may comprise at least 2, 3, 4, 5, 6, 7, 8, 9, 10, 20, 30, 40, 50, 60, 70, 80, 90, 100, 200, 300, 400, 500, 600, 700, 800, 900, 1,000, or more arrays. The plurality of arrays may comprise most 1,000, 900, 800, 700, 600, 500, 400, 300, 200, 100, 90, 80, 70, 60, 50, 40, 30, 20, 10, 9, 8, 7, 6, 5, 4, 3, or 2 arrays. The plurality of arrays may comprise from 1,000 to 2 arrays, 500 to 2 arrays, 500 to 100 arrays, 100 to 2 arrays, 100 to 50 arrays, 50 to 2 arrays, 50 to 10 arrays, or 10 to 2 arrays. An array of the at plurality of arrays may be adjacent to another array of the plurality of arrays. The arrays may be horizontally, vertically, or diagonally adjacent.

The surface may have a thickness of at most 1,000 μm, 500 μm, 100 μm, 90 μm, 80 μm, 70 μm, 60 μm, 50 μm, 40 μm, 30 μm, 20 μm, 10 μm, 5 μm, 1 μm, 0.1 μm, 0.01 μm, or less. The surface may have a thickness of at least 0.01 μm, 0.1 μm, 1 μm, 5 μm, 10 μm, 20 μm, 30 μm, 40 μm, 50 μm, 60 μm, 70 μm, 80 μm, 90 μm, 100 μm, 500 μm, 1,000 μm, or more. The surface may have a thickness from 1,000 μm to 0.01 μm, 500 μm to 1 μm, 100 μm to 1 μm, or 50 μm to 1 μm.

The surface may have a roughness of at most 1,000 μm, 500 μm, 100 μm, 90 μm, 80 μm, 70 μm, 60 μm, 50 μm, 40 μm, 30 μm, 20 μm, 10 μm, 5 μm, 1 μm, 0.1 μm, 0.01 μm, 0.001 μm, or less. The surface may have a roughness of at least 0.001 μm, 0.01 μm, 0.1 μm, 1 μm, 5 μm, 10 μm, 20 μm, 30 μm, 40 μm, 50 μm, 60 μm, 70 μm, 80 μm, 90 μm, 100 μm, 500 μm, 1,000 μm, or more. The surface may have a roughness from 1,000 μm to 0.001 μm, 500 μm to 0.01 μm, 100 μm to 0.1 μm, or 50 μm to 0.1 μm.

The surface may comprise a layer of a liquid that has a wetting affinity characteristic for the surface. The liquid may be immiscible with a droplet or a plurality thereof. The liquid may be dispensed on the surface. An upper surface of the liquid may reduce friction between a droplet, or a plurality thereof, and the surface as compared to the droplet directly contacting the surface.

The plurality of arrays may contain a channel, a hole, or any combination thereof. The plurality of arrays may contain a plurality of channels, a plurality of holes, or any combination thereof. The channel, or plurality thereof, may traverse between at least one surface. A gas, liquid, solid, or any combination thereof may be transferred through a channel or a hole. A gas, liquid, solid, or any combination thereof may be transferred through a plurality of channels or a plurality of holes. The gas, liquid, solid, or any combination thereof may be transferred from one array to another array. The arrays may be adjacent to each other. The gas, liquid, solid, or any combination thereof may be transferred from one array to at least one other array. The gas, liquid, solid, or any combination thereof may be transferred from one array to at least two, three, four, five, six, seven, eight, nine, ten, or more arrays.

At least two droplets of the plurality of droplets may be separated by at least one membrane. The membrane may comprise metal, ceramic (e.g., aluminum oxide, silicon carbide, zirconium oxide, etc.), homogeneous films (e.g., polymers (e.g., cellulose acetate, nitrocellulose, cellulose esters, polysulfone, polyether sulfone, polyacrilonitrile, polyamide, polyimide, polyethylene, polypropylene, polytetrafluoroethylene, polyvinylidene fluoride, polyvinylchloride, etc.)), heterogeneous solids (e.g., polymeric mixes, mixed glasses, etc.), a liquid (e.g., emulsion liquid membranes, immobilized (supported), liquid membranes, molten salts, hollow-fiber contained liquid membranes, etc.), or any combination thereof. The membrane may allow passage of molecules, ions, or a combination thereof from one side of the membrane to the other. The membrane may be impermeable, semi-permeable, permeable, or a combination thereof. The permeability may separate according to size, solubility, charge, affinity, or a combination thereof. The membrane may be porous or semi-porous. The membrane may be biological, synthetic, or a combination thereof. The membrane may facilitate exchange of constituents of one droplet to another droplet. The may membrane facilitate passive diffusion, active diffusion, passive transport, active transport, or any combination thereof. The membrane may be a cation exchange membrane, a charge mosaic membrane, a bipolar membrane, an anion exchange membrane, an alkali anion exchange membrane, a proton exchange membrane, or a combination thereof. The membrane may be permanently or temporarily attached to the array, or plurality thereof.

### EXAMPLES

### Example 1: Next-Generation Sequencing Library Preparation Platform

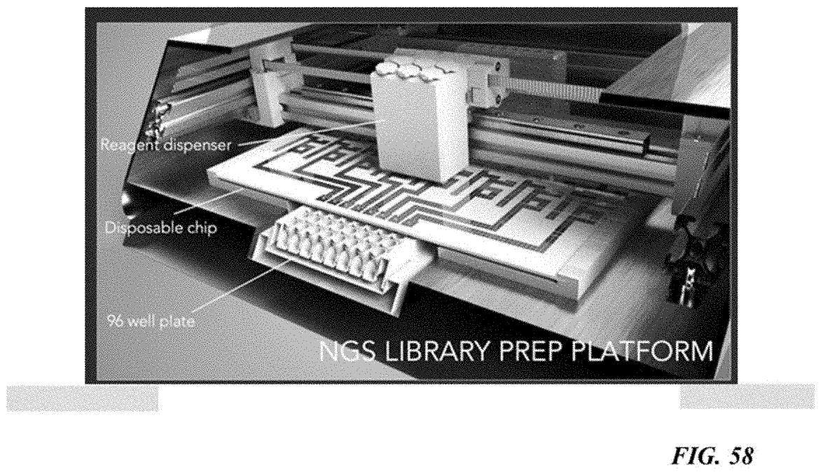

FIG. 58 represents an example of a next-generation sequencing library preparation platform described herein. The system is capable of processing biological samples, and comprises: a reagent dispenser, a plurality of 96 well plates, and a disposable chip. The reagent dispenser processes, for example, various biological samples (e.g., proteins, peptides, nucleic acids, polymers, monomers, cells, tissues, etc.), chemical reagents, solvents, liquids, gasses, solids, or any combination thereof. The disposable plate provides a surface for sample manipulation that is done directly on the surface, in an open environment, using, for example, acoustic waves, vibrations, air pressure, light field, magnetic field, gravitational field, centrifugal force, hydrodynamic forces, electrophoretic forces, dielectrowetting force, capillary forces, or any combination thereof. The samples are moved on the disposable chip to an assay, such as, for example, a 96 well plate, where attributes of a sample are measured. Reactions are done either directly on the surface or in the assay. Reagents are combined in the reagent dispenser, on the surface of the system, in the assay, or any combination thereof. Measurements of the biological sample, or plurality thereof, are performed in the reagent dispenser, on the surface of the system, in the assay, or any combination thereof. The system is pre-programmed, controlled in real time by a user, or any combination thereof. The system provides a way to manipulate biological samples for next generation sequencing library preparation with minimal sample manipulation.

### Example 2: Droplet Evaporation

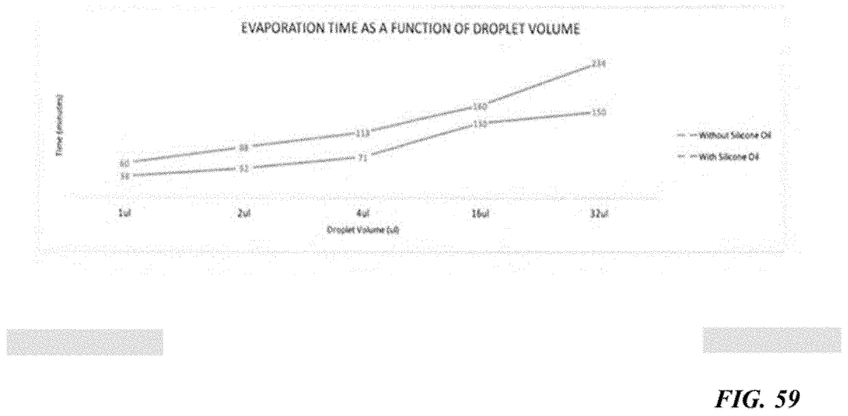

The liquid of a droplet, or plurality thereof, in an open or a closed system evaporates over time. This evaporation is controlled by the systems and methods described herein. The presence of a silicone-based oil on the surface of the systems described herein significantly reduces the rate of evaporation over time, as depicted in FIG. 59. The silicone-based oil reduces the rate of evaporation by forming a thin film around a droplet, or plurality thereof. The droplet, or plurality thereof, and the silicone-based oil are immiscible as a result of the difference in density between the liquid droplet and the silicone-based oil. The thin film has other beneficial effects, such as, for example, reduction in loss of sample due to splitting of the droplet at least as a result of the immiscibility of the droplet and the film, increased rate of motion of the droplet at least as a result of a reduction in friction between the droplet and the surface, reduction in contamination of the droplet at least as a result of the film acting as a barrier, and reduction in cross contamination at least as a result of the aforementioned factors.

### Example 3: Well Plates Stacked on the Bottom of Liquid Dispenser

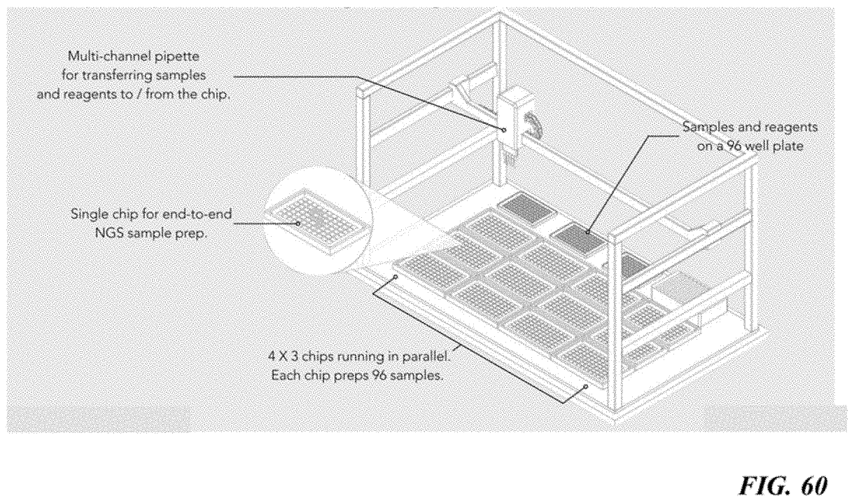

FIG. 60 shows an implementation of array based system for processing large number of samples in parallel. Arrays are stacked on a horizontal base platform (the deck) of a liquid handling instrument. Additionally, the deck may contain microwell plates for storing reagents and samples. A three axis motion platform freely moves adjacent to or above the arrays and reagent plates. The motion platform transfers samples and reagents between the well plates and the arrays. The arrays may include fully closed electrowetting devices, fully open electrowetting devices, partially open (partially closed) devices, or any combination of technologies described herein. The arrays are capable of performing heating and cooling operations for enzymatic reactions and PCR amplification. The arrays can also apply a magnetic field for magnetic-bead based wash. By combining the ability to mix liquids, heat, cool, and apply magnetic field, the arrays prepare libraries for next-generation sequencing. The same array setup can be programmatically repurposed to prepare libraries for PacBio instrument based on SMRT sequencing, nanopore sequencers, miniprep for plasmid extraction, enzymatic DNA synthesis, cell screening and many other applications. A single array can process 1 to 100 samples for sequencing. It is possible to enclose the entire liquid handling instrument and the array setup to maintain a certain level of humidity and prevent external contamination while conducting experiments.

### Example 4: Factory Scale Box

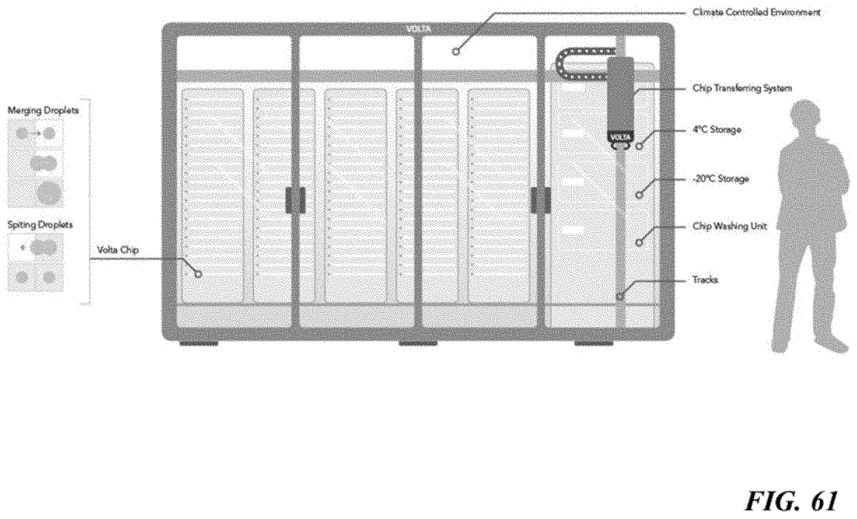

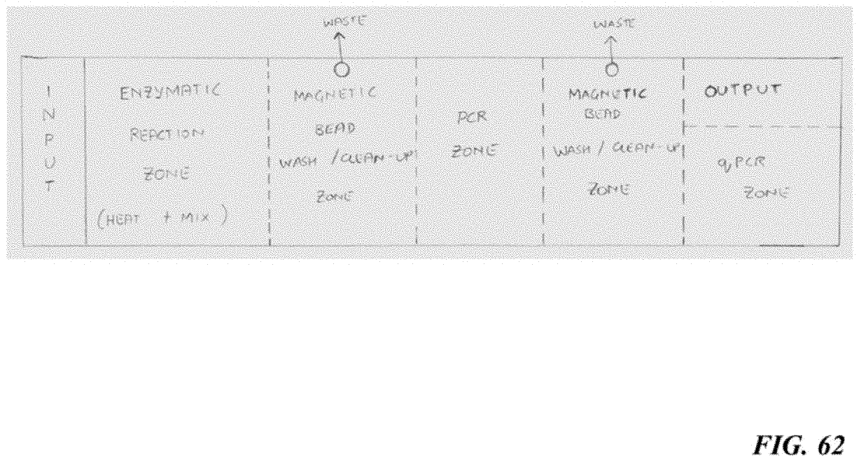

FIG. 61 shows an implementation of an automated system for genomic factories and synthetic biology factories. The system stacks arrays of liquid processing units (LPU) in vertical and horizontal fashion. Each LPU is capable of processing samples using arrays described herein. The LPUs can be inserted and removed from the front. The LPUs are “hot pluggable”, i.e., they can be plugged in and out while the rest of the system operates. The LPUs receive power and signal for processing samples through electronic connections located inside the system. The LPUs are accessible on the front for introduction of reagents and samples. Additionally, reagents can also be introduced onto the system using tubing that runs on the back and/or on the inside of the system. The system may contain optical systems and cameras for monitoring the progress of reactions on LPUs. The system contains one or more robotic transport system and hands (labeled chip transfer system in FIG. 61) for placing the LPUs in their respective locations. The robotic system dispenses liquids in and out of the processing units. The system can contain cold storage for storing reagents. Prior to transferring the samples/reagents on to the LPUs the system thaws them. The LPU then processes samples and reagents for genomics sequencing, synthesis of biopolymers such as DNA, cell screening, gene assembly or any other application. Furthermore, the system is capable of washing the LPUs. The entire system can

### Example 5: Single NGS Library Prep Chip: Potentially Part-Open Part-Closed; Pulling Out Samples for QC

FIG. 62 shows an array that runs library prep for next-generation sequencing preparation. The array may be an electrowetting array, DEW array, or DEP array. The array consists of an input zone where samples and reagents are deposited. The array consists of an output zone from which samples are transferred into a microwell plate. Between the input and the output port, samples and reagents in droplet form are transported, mixed, heated and cooled. A typical input to the array is a purified genomic along with reagents for library preparation. The array may contain specific zones for heating/cooling or the entire array may be heated/cooled. The heated parts of the array may be enclosed to reduce evaporation. The entire array may also be enclosed to reduce evaporation. Parts of the arrays contain actuators for generating magnetic field. These parts are used to perform magnetic bead based wash. One of the zones on the array can take an aliquot of sample and run quality control on the sample through fluorometric measurement. The array may take the sample through temperature cycles to perform library quantification through qPCR. Two or more arrays can be adjacent to each other and process multiple samples simultaneously and finally pool them.

### Example 6: NGS Library Preparation with Evaporation Compensation

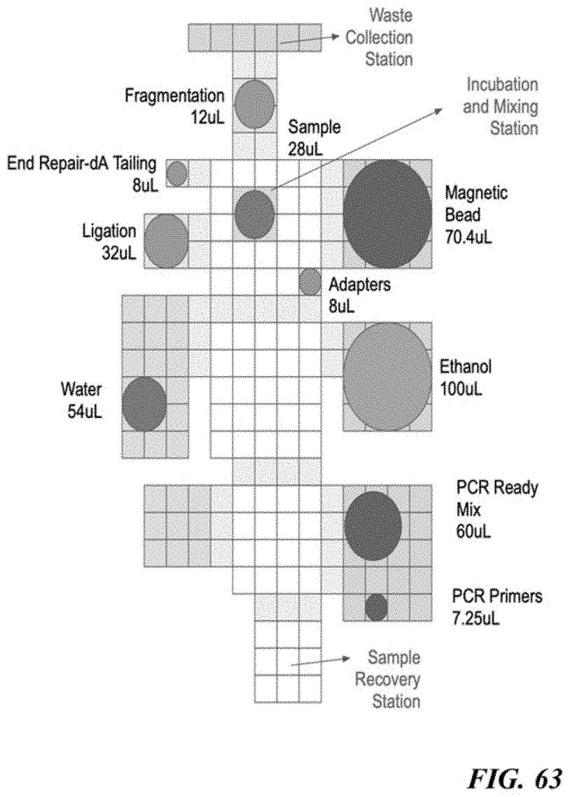

NGS library preparation was performed on the EWOD array (array “tile” substrate) using a commercially available kit. FIG. 63 shows the placement of reagents and a sample on the array. The reagents for this workflow include: fragmentation enzyme and fragmentation buffer mix, end-repair enzyme and A-tailing mix, Ligation enzyme and buffer mix, Ampure magnetic beads (e.g., micron sized beads), ethanol, unique DNA barcodes (e.g., with sequencing adaptors for Illumina sequencers), PCR master mix, and water. These reagents were introduced on the array tile either manually or with an automated system using dispensing mechanisms described herein. The reagents were mixed with the sample and incubated at various temperatures in a predetermined sequence. During heated reactions (e.g., fragmentation, end-repair/dA-tailing) and reactions carried out at room temperatures (e.g., ligation of adapter to the fragmented DNA), a combination of techniques were used to compensate for volume lost due to evaporation. The evaporation compensation techniques used are described herein and are depicted in FIGS. 29A-29F. Additionally, the techniques described in FIGS. 26A-26F, and other techniques described herein, can be combined with techniques in FIGS. 29A-29F to regulate volumes of the droplets. With these evaporation compensation techniques, the reaction volume for, for example, fragmentation, end-repair/A-tailing and ligation reactions was maintained at a steady volume (e.g., with less than 10% CV deviation) throughout the reaction. With this method, NGS libraries were produced on the array using fully automated or a semi-automated methods, maintaining the final reaction volume within a 10% margin of error. The final library was then mixed with PCR primers and PCR ready Mix on the array. This mixture (e.g., library, PCR primers and PCR mix) was then PCR-amplified using an external thermocycler. Alternately, the PCR amplification is performed on the array or another element of the array.

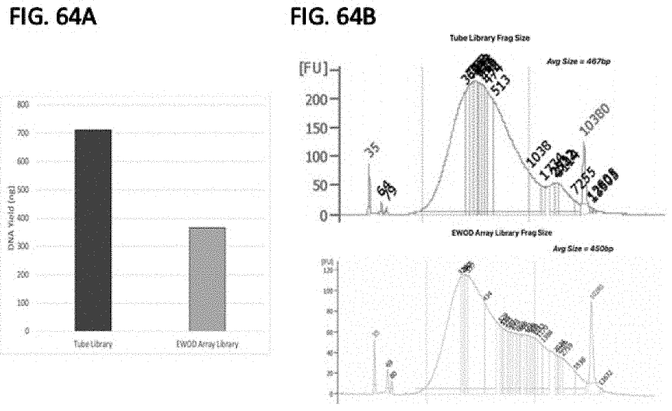

The final yield of DNA for libraries prepared on the array tile (e.g., using automated or semi-automated techniques described herein) is comparable to the yield obtained from libraries prepared traditionally (e.g., manually) in tubes as seen in FIG. 64A. Additionally, the average fragment size (e.g., 450 bp) of the DNA from libraries prepared on the array is also comparable to the fragment size (467 bp) of the library prepared manually (e.g., in tubes) as seen in FIG. 64B. This data supports that the evaporation control techniques described herein involving timed replenishing droplets, with real-time control computer-vision based volume estimation, or other techniques described herein are effective for NGS library preparation.

### Example 7: Extraction of High Molecular Weight (HMW) Nucleic Acid

Cells from various sources (e.g., mammalian, bacterial, plants) are lysed directly on the array by merging a droplet containing cells with another droplet containing lysis agent (e.g., detergent or enzymatic). This mixture is heated and mixed (e.g., separately or simultaneously) on the EWOD array to promote lysis of cells and, if applicable, lysis of the nucleus. Enzymatic digestion of proteins, RNA, or a combination thereof are performed to improve the purity of the sample. While cells are lysed, the progress of lysis reaction and lysis efficiency is monitored via DNA-specific fluorescent stains. DNA is purified directly on the array by solid phase (e.g., bead-based capture or by precipitation (e.g. salt and ethanol or phenol-chloroform extraction)). Recovered DNA is manipulated and transferred to different locations of the array by EWOD with minimal shearing. DNA purity, critical for high quality long-read sequencing, is improved by increasing the number of washing cycles performed on the array Small DNA fragments are removed using silica-nanostructured magnetic disks. The yield of recovered DNA is increased by performing additional successive elution in buffers.

After DNA extraction, samples are analyzed by Pulse Field Gel Electrophoresis (PFGE), quantifying size distribution for each sample relative to one another and analyzing commercially available ladders (BioRad) and ImageJ (NIH) profile analysis tools. For smaller inputs, (e.g., cell input) recovery/size distribution is measured by Femto Pulse (Agilent) and qPCR for lower input amounts. Genomic intactness is assessed by additional complementary methods, e.g. the BioNano Genomics Saphyr System, allowing rapid and cost-effective prototyping at a macro scale as well as independent comparability of data using the Saphyr System.

The passivation of the EWOD surface is determined by testing DNA deposition and retention in the presence of solution- and surface-deposited PEG200 or BlockAid (Invitrogen) passivated devices. Measurements are obtained i) by staining the surfaces after use with Hoechst 33342, ii) calculating surface retention of a commercial preparation of Lambda DNA (New England Biolabs, linearized 48.5 Kb), and/or iii) measuring % loss by qPCR of sample pre- and post-manipulation and input quantities from 10⁹ to 102 copies of DNA.

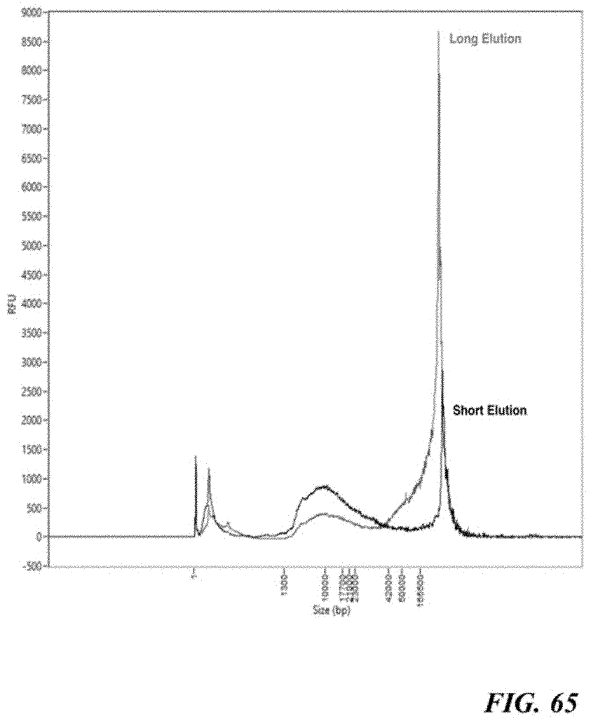

Mammalian cell lysis, RNA, and protein digestion followed by HMW DNA isolation was performed on the EWOD array. The distribution of the high molecular weight DNA fragments are seen in FIG. 65 with DNA fragments larger than 165,000 bp being isolated from the sample. The longer duration of elution recovers more DNA (e.g., indicated by the taller peak) and is a way to obtain higher DNA yield.

### Example 8: Stability Buffer for Nucleic Acid Transfer

A stabilization buffer is created using commercial preparations of long Lambda DNA and HMW DNA preparations from a validated protocol using the Bionano Genomics Saphyr and Femto Pulse platforms. Small (e.g., 5 ul) hydrogel droplets are created by mixing of 2×2.5 μl droplets containing an alginate solution and a calcium ion solution (e.g. CaCl₂)). Droplets are mobilized by addition of a larger volume (e.g., ˜15 μl) and made available for transport. Gel particles are released by citrate or EDTA solution, and percent recovery of intact fragments are measured by Femto Pulse and PFGE, internally, and Saphyr, as an external service. Other high viscosity buffers for retention/elution media for the EWOD device include, for example, sucrose, PEG, and polyvinylpyrrolidone (PVP) content buffers and Nanobind nanostructured silica disks (Circulomics).

Stability is measured by Femto Pulse and PFGE through freeze-thaw cycles and accelerated stability testing at elevated temperatures, simulated using Arrhenius kinetic parameters to a minimum 3 months at −20° C. Cell input and measure recovery/size distribution are titrated by Femto Pulse and qPCR for lower inputs. Sample loss is determined by qPCR at input quantities from 10⁹ to 10³ copies of DNA.

### Example 9: Whole Genome Sequencing on Cellular Nucleic Acids

Genomic intactness is demonstrated by long read sequencing through an Oxford Nanopore device. DNA can be extracted using protocols described herein alongside Qiagen HMW kit and Loman protocols. Libraries are prepared according to an optimized protocol for keeping strands in >1 Mb lengths. The repeatability of the extractions is evaluated by sequencing a minimum of 3 each of Qiagen and Loman libraries and 7 Flexomics libraries to ensure robustness of evaluation of size performance. Regular input and low input (e.g., 1000 cells) libraries are assessed. At low input, ˜24 subsets are barcoded of 1000 cells each to provide enough material for downstream sequencing (˜150 ng theoretical).

Cell HMW DNA input is titrated down, for example, i) by supplementation with carrier DNA, e.g. Lambda DNA, to ensure balanced library preparation or ii) dilution of an absolute number of cells and scaling of library preparation and analysis reagents for subsequent reactions. Lambda DNA is biotinylated (e.g. with Pierce 3′ biotinylation kit, Thermo Fisher) to allow depletion to concentrate on-target library prior to sequencing. Performance of the ONT transposase library preparation is assessed on-device, e.g., without moving the sample to a separate tube.

Maps generated from experiments described herein are compared to the literature reported GM12878 genomes to determine completeness of the sequencing libraries generated.

### Example 10: Enzymatic DNA Synthesis

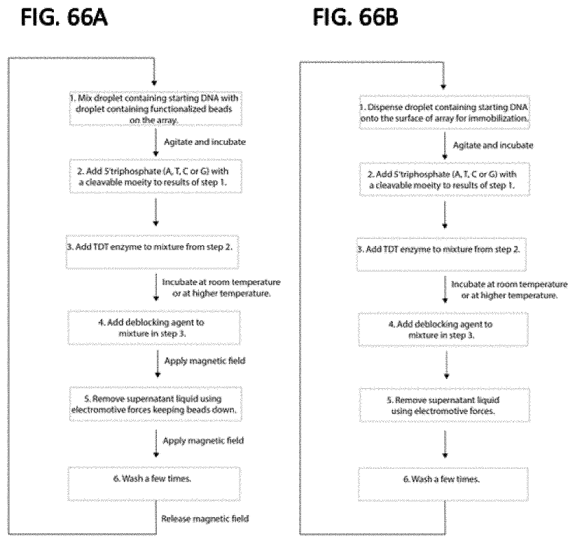

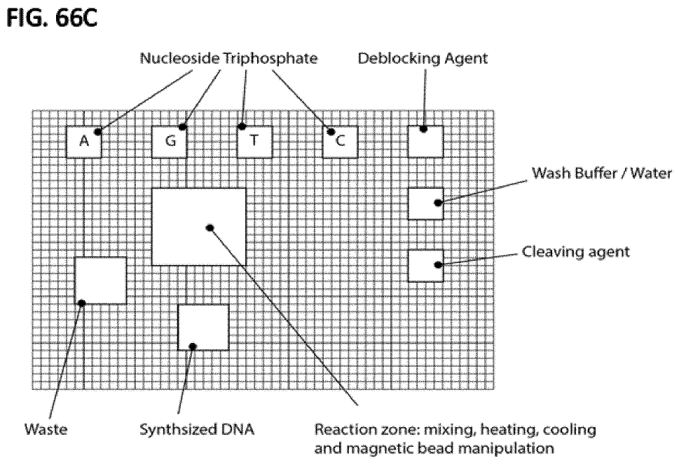

A method to synthesize polynucleotides (e.g., DNA) using an enzyme catalyzed process in an aqueous medium on arrays described herein was performed. Terminal Deoxynucleotidyl Transferase (TDT) is a template independent polymerase that catalyzes the formation of phosphodiester bonds between the 3′ and 5′ end of DNA. FIG. 66A and FIG. 66B show example workflows to afford the synthesis of DNA. FIG. 66C shows a schematic diagram for a single reaction site that performs step by step addition of nucleotides to synthesize a long molecule of DNA.

A droplet containing a starting DNA material with an unprotected 3′-hydroxyl group is mixed with a droplet containing functionalized magnetic beads. After a brief period of agitation, the DNA molecules are bound to the magnetic beads. Alternatively, a droplet containing starting DNA material is dispensed onto a location of the array that is functionalized to immobilize the DNA to a solid support. A droplet containing a nucleoside 5′-triphosphate with a cleavable/removable moiety is mixed with the droplet containing immobilized starting DNA. TDT enzyme, which catalyzes the 5′ to 3′ phosphodiester linkage between the unprotected 3′-hydroxyl end of the starting DNA and the 5′-phosphate end of the nucleoside triphosphate, in a droplet is then merged and mixed with the droplet containing immobilized DNA. The reaction is incubated for at room temperature or higher temperature for 5-30 minutes.

A droplet containing a deblocking agent is then mixed with the subsequent reaction mixture, producing the nucleotide with a free 3′-hydroxyl. In the case of using magnetic beads for immobilization, a magnetic field is then applied to pull the beads down to the surface of the array and the excess liquid is removed. The beads are then washed multiple times (e.g., 2-4) by flowing a washing buffer over the beads. The washed liquid is then discarded to the waste area of the array. Additional nucleotides are added to the DNA by repeating the method described above. During each addition of a nucleoside triphosphate, a controller instructs the array to dispense one of the nucleoside triphosphates from respective reservoirs. After multiple iterations, a polynucleotide of known sequence is produced, staying immobilized either to the beads or on the functional surface of the array. The final DNA product is cleaved and released from the surface (e.g., the beads or the surface of the array) by bringing a droplet containing a cleaving agent. The final product is then suspended in a droplet and recovered from the array.

Errors in DNA synthesis can be corrected with mismatch binding and mismatch cleaving proteins. A mismatch binding protein (e.g., MutS) is bound to a magnetic bead and mixed with a droplet containing assembled DNA comprising at least one error (e.g., identified as distortion in the double helix). For example, DNA molecules comprising an error are bound to the magnetic beads and the DNA without errors are not attached to the beads. The beads are then moved to another area of the array using a magnetic field, removing the DNA comprising at least one error. The excess liquid containing DNA with no errors is separated from the beads using electromotive force (e.g. EWOD).

Alternately, errors are corrected using mismatch cleaving enzymes, such as, for example, T4 endonuclease VII or T7 endonuclease I. A droplet comprising a cleaving enzyme is mixed with a droplet containing assembled DNA. The mismatch cleaving enzymes target the regions at or near the errors. The error-free fragments are then retrieved using magnetic bead-based separation. Alternately, exonucleases are used to remove additional errors on fragments left over by the mismatch cleaving enzymes. These trimmed fragments are assembled correctly using PCR assembly in a droplet.

The assembled and error corrected DNA is amplified using PCR in a droplet. The final product from PCR is then prepared into libraries for sequencing on the array using methods described herein. The libraries are sequenced using any of the sequencing techniques described herein for final sequence verification of the synthesized DNA.

### Example 11: Next-Generation Sequencing (NGS) Library Preparation

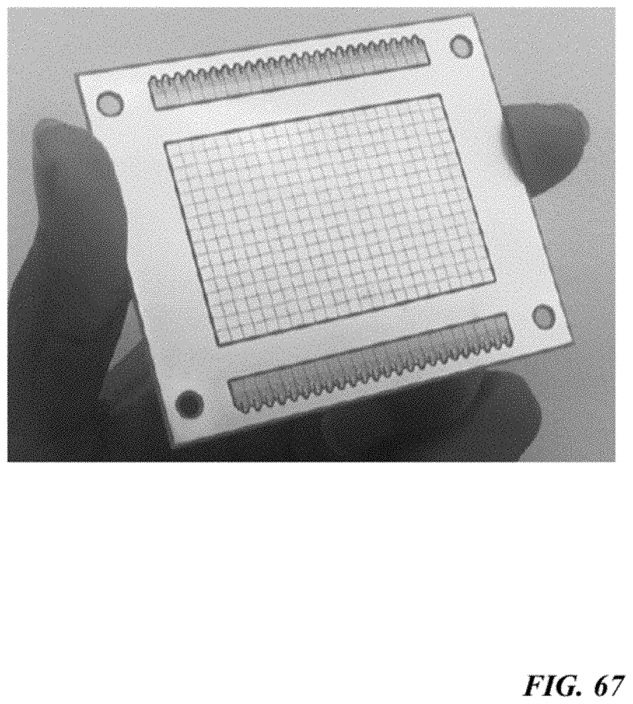

224 nanograms (ng) of purified genomic DNA was used as starting material and Genome In A Bottle NA12878 was used as the DNA source. All steps from DNA fragmentation, end-repair/A-tailing, ligation and DNA purification/size selection are performed on the device shown in FIG. 67. Final libraries were amplified by two cycles of PCR, which was performed on a thermal cycler in a separate post-PCR area. A control library was performed off-chip manually for data comparison. Libraries were quantified by Qubit and fragment size distribution was assessed by BioAnalyzer. Libraries were normalized accordingly and sequenced on a NextSeq500 (e.g., shallow sequencing with initial mid-output run at 2×75 cycles and 2×8 cycles for the indexes followed by additional coverage generation with a high-output 2×150 cycles run). Sequencing data was demultiplexed using Illumina's bcl2fastq v2.20 without adapter trimming. Bioinformatics analysis was performed using well-established algorithms (e.g., FASTQC, BWA-MEM, SAMtools, Picard and GATK).

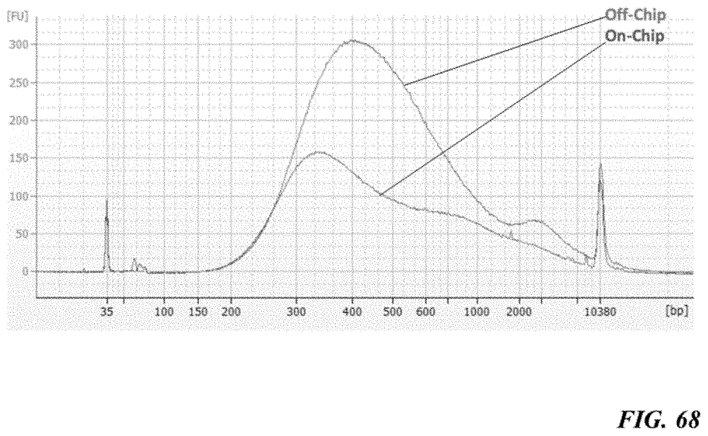

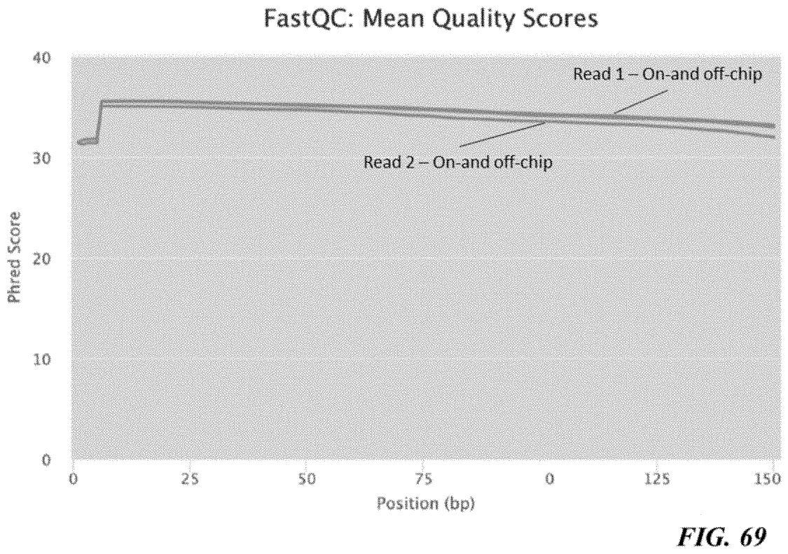

The library prepared on chip generated enough material for sequencing (Table 1). The off-chip control generated ˜2.3× more DNA material than the on-chip experiment; however, the average fragment size was higher than described previously for both on-chip and off-chip libraries (Table 1 & FIG. 68). All sequencing and mapping QC data demonstrated that high quality sequencing libraries were generated (Table 1), with Q30 >90% (FIG. 69), % PF reads >90%, using systems and methods described herein.

TABLE 1

| Metrics | Target Value | | On Chip | Manual - Off-chip |
|---|---|---|---|---|
| DNA Yield | 125-625 | ng | 294.32 | 712.4 |
| Average | 250 | bp | 450 | 467 |
| fragment Size | | | | |

| Number of | — | 158693266 | 106693500 |
|---|---|---|---|
| Reads | | | |
| Q30 | >75% | 93.35% | |
| PF Reads | >80% | 92.90% | |
| % Duplicates | — | 9.35% | 8% |
| % PF reads | >0.9 | 0.994232 | 0.993074 |
| aligned (hg19) | | | |
| Median | — | 9 | 7 |
| Coverage | | | |
| HET SNP | >0.8 | 0.836866 | 0.875379 |
| Sensitivity | | | |

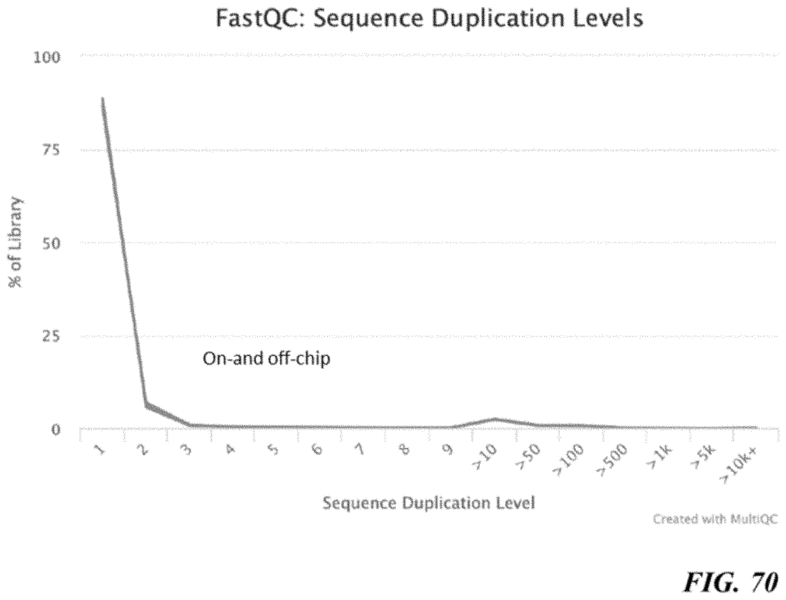

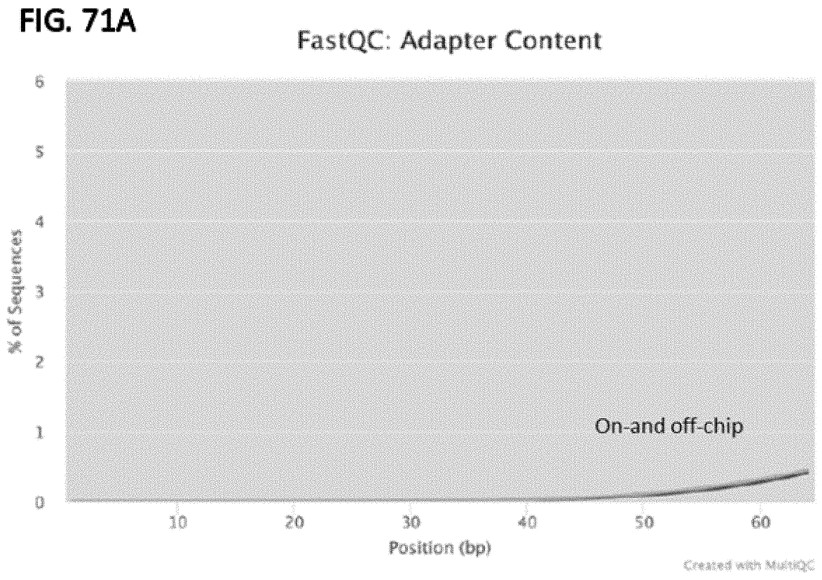

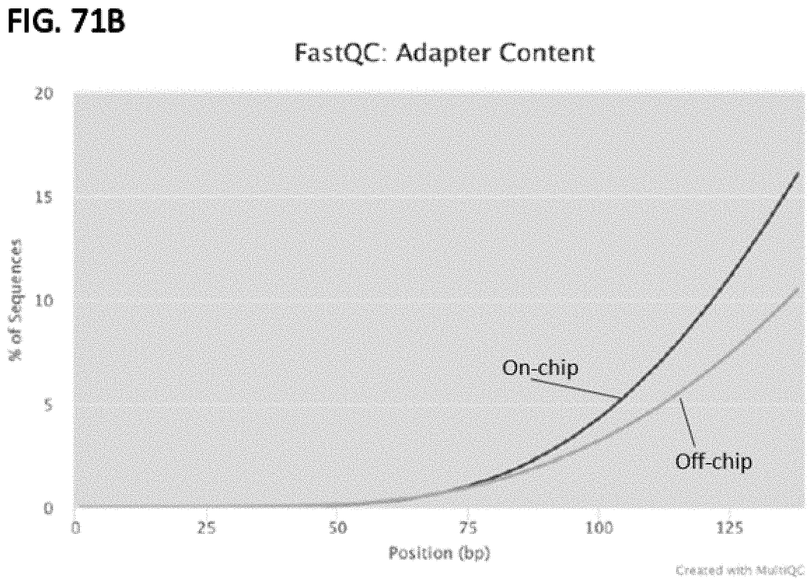

The level of duplicates for both on and off-chip libraries was low (FIG. 70) and <10% overall (Table 1). The low level of duplicated reads was also reflected in the limited content in adapters. Our initial shallow sequencing (2×75) indicated <1% adapter contamination (FIG. 71A) while up to 15% and 10% adapters for on-chip and off-chip libraries, respectively, were detected when increasing sequencing depth and read length to 2×150 (FIG. 71B). The difference between on and off-chip can be due to the higher number of reads generated for the on-chip library compared to off-chip control.

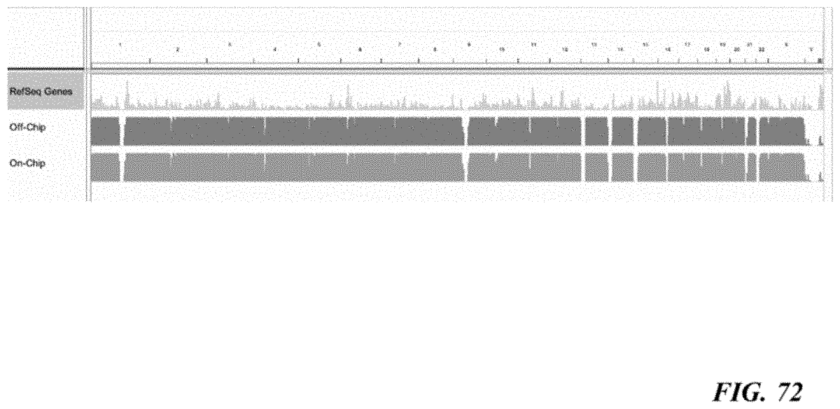

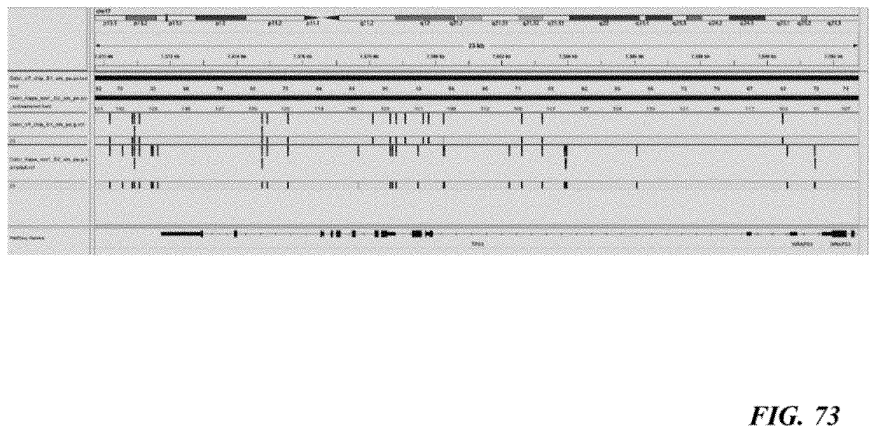

Mapping rate of passed-filter reads was high (>99%) and coverage across the genome was comparable between both libraries (FIG. 72), at a median coverage of 9× and 7× for the on-chip and off-chip libraries, respectively. Variants and the ability to call single nucleotide polymorphisms (SNPs) was determined. The heterozygous (HET) single nucleotide polymorphism (SNP) sensitivity was comparable at similar coverage between on- and off-chip (Table 1). This was confirmed by looking specifically at SNPs on the TP53 locus where identical genotypic variants were detected for both libraries in intergenic regions (FIG. 73).

### Example 12: Workflow Sample Preparation for DNA Sequencing on an Array

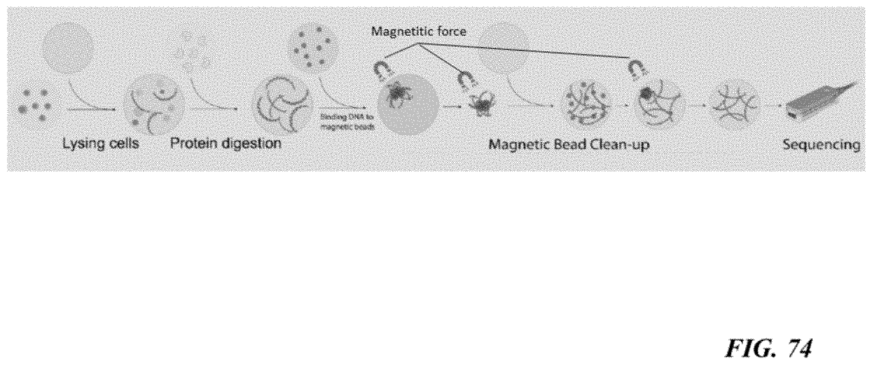

An example of a workflow for NGS on an array described herein is shown in FIG. 74. Cells in a droplet on an array are lysed on the array by introducing another droplet comprising chemical or enzymatic cellular lysis reagents. The proteins contained in the droplet are degraded by introducing degradation enzymes contained in another droplet of the array, and magnetic particles specific for DNA molecules are introduced to the droplet containing the DNA molecules. The magnetic beads are attached to the surface of the array or the magnetic beads are suspended in a droplet. The DNA molecules are separated and isolated from the cellular debris and degraded proteins using magnetic fields of the array. The isolated DNA, either attached to magnetic particles suspended in solution or magnetic particles coupled to the array, undergoes a magnetic bead washing process. The DNA is introduced to a DNA sequencer on, adjacent to, or separate from the array. The DNA is sequenced.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Example 13: Serial Dilution

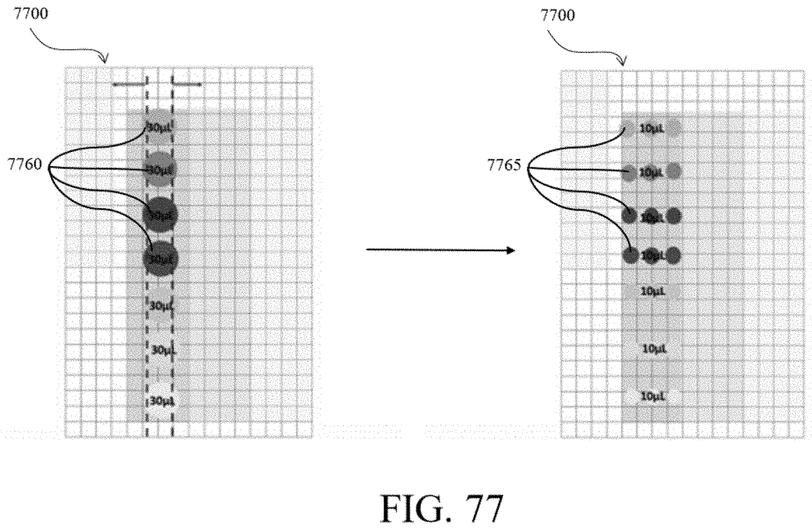

Serial dilution is an essential liquid handling technique broadly used in various kinds of assays. FIG. 77 depicts a serial dilution processes during library quantification on an array, according to some embodiments. This technique may be used to create various droplets containing different concentrations of given biological markers. Various readouts that may be derived from these droplets created by serial dilution may be used to generate standard curves and assess assay sensitivity. Serial dilution may be performed by successive droplet splitting and dilution by water or buffer addition. Droplet splitting may be achieved by electrowetting in a two plate system or by driving a droplet over a hydrophobic “slicer”, as described herein. The successive splitting and dilution steps may be performed manually or in a fully automated fashion (e.g. directed by a machine algorithm based on data points generated from previous assays). The serial dilution and droplet volume accuracy may be recorded by volume measurement using machine vision or other droplet sensing techniques described herein. These measurements occur at multiple time points (e.g. every second, every 1-4 seconds, etc.).

FIG. 77 depicts a plurality of samples disposed on an array 7700, as disclosed herein. In some embodiments, as depicted, larger sample 7760 droplets are separated into smaller sample droplets 7765. In some embodiments, concentrations of a chemical or biological substance contained by the droplet are maintained as after the droplets have been separated. In some embodiments, a heating system is turned off during separation of droplets.

### Example 14: DNA/RNA Amplification, Detection and Quantification

The array devices described here in and an instrument built using the array devices may be used in conjunction with various commercially available and newly developed kits that employ various types of nucleic acid amplification (e.g. PCR, RPA, RCA, linear amplification). DNA and/or RNA may be used as starting material for amplification. DNA and/or RNA may be directly extracted from cells on-chip prior to amplification and detection (e.g. by qPCR) as described in other sections and/or examples. In the case of RNA, a qPCR workflow may be set-up as either one-step (RT-qPCR, e.g. TaqMan™ RNA-to-CT™ 1-Step Kit) or two-steps (reverse transcription then PCR) reactions (e.g. ThermoFisher Maxima H Minus Reverse Transcriptase, SuperScript™ IV First-Strand Synthesis System). Several samples may be pooled in a single reaction to increase the number of tested samples (e.g. high-throughput diagnostic) and individual samples being retested in case the pool is positive. Multiple targets may be multiplexed and assessed in a given sample or pool of samples. Reagents may be provided separately and added to the surface of the device at the time of the experiment or may be part of the consumable itself (consumable can mean film frames, EWOD array, or EWOD tile described in previous applications), for example, some reagents might be lyophilized on the surface of the consumable and then resuspended in water on the device at the beginning of the experiment. Quantification experiments (e.g. gene expression, genotyping, library quantification, diagnostic) may be run in duplicates or triplicates in parallel on the array to increase accuracy. Replicates might be initiated from separate aliquots (droplets) of starting material introduced independently on the consumable or the initial sample may be aliquoted directly on-chip by droplet splitting (e.g. for simultaneous manipulation). Reagents and samples may be prepared and stored on chip (e.g primer mix, master mix) and brought up to the right concentration by droplet splitting based serial dilution. The chip may comprise a reagent reservoir adjacent to the array. The reagent reservoir can be in fluid communication with the surface of the array. Reagents and samples may be kept in a cold zone (4° C.) on the array tile until used. Fluorescent or colorimetric measurement on the array may be used for detection (e.g. positive vs negative diagnostic test) or for quantification (e.g. gene expression, sequencing library quantification). In the case of library quantification, quantification results may be used for downstream sample normalization and pooling prior to sequencing.

Nucleic acids may be amplified by isothermal amplification (e.g. LAMP, RPA, RCA, SDA), at a constant temperature (e.g. 37° C., 65° C. or room temperature). Nucleic acids from RNA samples may be reverse-transcribed on the array device prior to isothermal amplification. During amplification, the signal may be detected in real time by monitoring a dsDNA binding dye (e.g. SYBR), a fluorogenic probe/molecular beacon or turbidity. The amplification signal may be quantified at the end of the reaction or monitored over time with the UV-visible camera connected to the device. Sample of interest, positive and negative control may be run in parallel on the same array. Resulting amplicons may be extracted for downstream applications (e.g. library preparation for Next Generation Sequencing). Throughout these reactions, the evaporation and humidity control methods and systems can be applied to the array to maintain constant physical properties of the droplet(s), the array itself, and the area adjacent to the array and/or the droplet(s).

### Example 15: RT-LAMP to Detect SARS-nCoV-2 in Patient's Sample

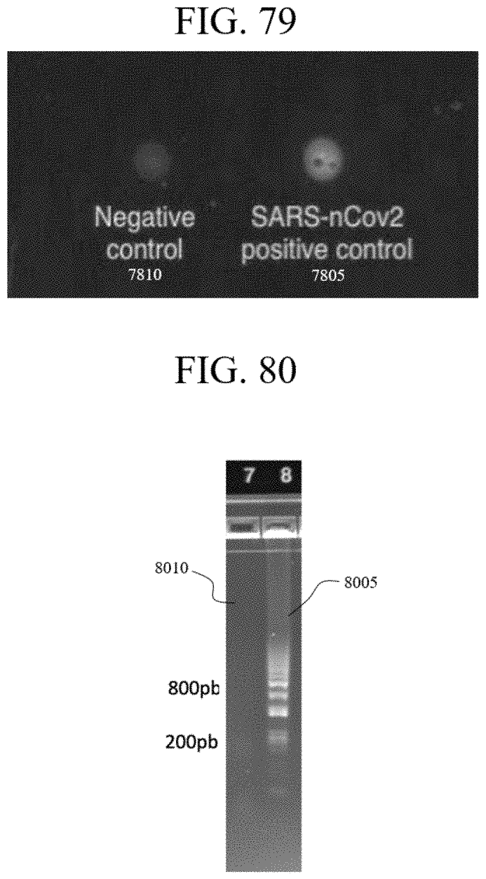

The assay enables RT-LAMP isothermal amplification of specific targets: certain regions of SARS-nCoV2 RNA and positive control. After the RT-LAMP amplification is performed, positive/negative readout may be detected directly and indicate sample/patient results. A fluorescent LAMP dye, binding only to double stranded DNA, may be added to the master mix. The intensity of the fluorescence is correlated to the quantity of DNA in the sample and measured by fluorescence. FIG. 79 shows a comparison between a negative viral RNA control 7910 and a positive viral RNA control 7905, extracted according to the embodiments described herein. If the sample contains the viral RNA, the droplet may appear fluorescent, as depicted in FIG. 79.

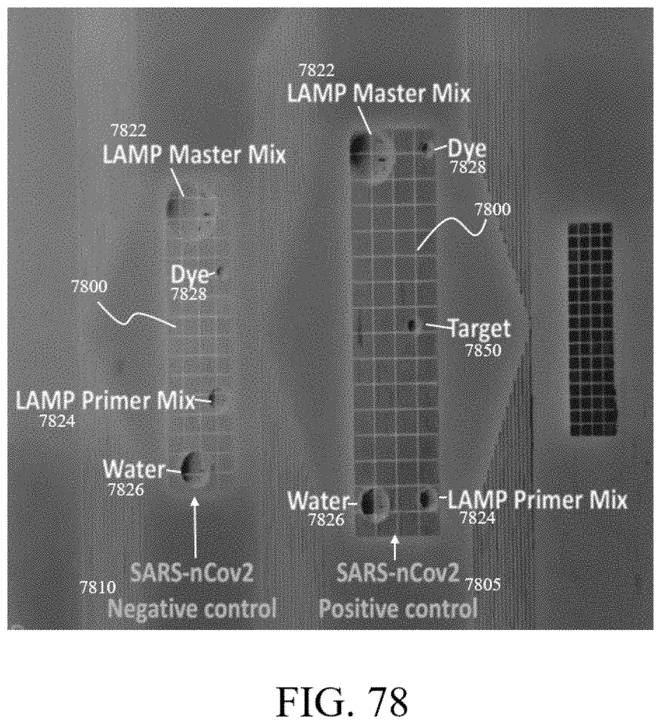

The reagents (WARMSTART® 2×Master Mix from NEB, dH2O) and targets 7850 (e.g. DNA or RNA) may be mixed on the device 7800 and heated at the required temperature (e.g. 60-65° C.), as depicted by FIG. 78. Positive controls 7805, negative controls 7810 and targets 7850 may be amplified either serially or in parallel on the array 7800. In some embodiments, reagents comprise a LAMP Master Mix 7822, a LAMP Primer Mix 7824, water 7826, a dye 7828, or any combinations thereof.

The RT-LAMP reaction uses pairs of inner primers (FIB, BIP), outer primers (F3, B3) and loop primers (LB, LF). Each of the inner primers possesses a sequence complementary to one chain of the amplification region. The reverse transcription and elongation reactions are sequentially repeated by Reverse transcriptase and DNA polymerase-mediated strand-displacement synthesis (present in WARMSTART® 2×Master Mix from NEB). This method operates on the fundamental principle of the production of a large quantity of DNA amplification products with a mutually complementary sequence and an alternating, repeated structure. LAMP may amplify a few copies of DNA to 10⁹ copies in less than an hour under isothermal conditions with specificity. Throughout these reactions, the evaporation and humidity control methods and systems can be applied to the array to maintain constant physical properties of the droplet(s), the array itself, and the area adjacent to the array and/or the droplet(s).

TABLE 2
Primer design for SARS-nCoV-2 detection & positive control

| Target | SEQ ID NO | Sequence 5′→3′ |
|---|---|---|
| SARS-nCov2 Ngene | SEQ ID NO: 1 | F3 AACACAAGCTTTCGGCAG |
| | SEQ ID NO: 2 | B3 GAAATTTGGATCTTTGTCATCC |
| | SEQ ID NO: 3 | FIP TGCGGCCAATGTTTGTAATCAGCCAAGGAAATTTTGGGGAC |
| | SEQ ID NO: 4 | BIP CGCATTGGCATGGAAGTCACTTTGATGGCACCTGTGTAG |
| | SEQ ID NO: 5 | LF TTCCTTGTCTGATTAGTTC |
| | SEQ ID NO: 6 | LB ACCTTCGGGAACGTGGTT |
| RNase P | SEQ ID NO: 7 | F3 TCCAGTTACGCTGGAGTCT |
| | SEQ ID NO: 8 | B3 AAGATCACGCGCCATCAG |
| | SEQ ID NO: 9 | FIP GCTCGCAGGTCCAAATCTGCACTGAATGATATGCGGCCTCG |
| | SEQ ID NO: 10 | BIP CTCTGCGCGGACTTGTGGAGCAGGGACATCCCAGAGACT |
| | SEQ ID NO: 11 | LF CACCGCCATGCTGAAGTC |
| | SEQ ID NO: 12 | LB CAGCCGCTCACCGTGAGTT |
| Synthetic COVID- | SEQ ID NO: 13 | F3 TGGCTACTACCGAAGAGCT |
| 19 RNA_Ngene | SEQ ID NO: 14 | B3 TGCAGCATTGTTAGCAGGAT |
| | SEQ ID NO: 15 | FIP TCTGGCCCAGTTCCTAGGTAGTCCAGACGAATTCGTGGTGG |
| | SEQ ID NO: 16 | BIP AGACGGCATCATATGGGTTGCA-CGGGTGCCAATGTGATCT |
| | SEQ ID NO: 17 | LF GGACTGAGATCTTTCATTTTACCGT |
| | SEQ ID NO: 18 | LB ACTGAGGGAGCCTTGAATACA |

In order to increase the specificity of the assay, a molecular beacon probe may be added to the reaction. The molecular probe is a LB primer modified at its extremity in order to have the shape of a circular probe. Each extremity is bound to a fluorophore and a quencher. When the molecular beacon probe is not linked to the target, the fluorescence is attenuated by the quencher. Once the LB finds its complementary sequence on the target, the circular structure is disturbed, triggering the emission of a fluorescent signal.

TABLE 3
Molecular Beacon probe design for SARS-nCoV-2 & positive control detection

| Name | SEQ ID NO | Sequence 5′→3′ |
|---|---|---|
| 2019- | SEQ ID NO: 19 | FAM-AGCGGCACCTTCGGGAACGTGGTTGCCGCT-BHQ1 |
| nCoV_N_LBP | | |
| Rnase P_LBP | SEQ ID NO: 20 | FAM-AGCGGCTCAGCCGCTCACCGTGAGTTAGCCGCT- |
| | | BHQ1 |
| Synthetic COVID- | SEQ ID NO: 22 | FAM- |
| 19 RNA_LBP | | AGCGGCTACTGAGGGAGCCTTGAATACAAGCCGCT- |
| | | BHQ1 |

After each amplification, the cDNA/DNA may be purified directly on the array by using paramagnetic SPRI beads (e.g. AMPure XP for PCR Purification). The amplicons washed and eluted in 20 μL may be analyzed by gel electrophoresis, as depicted in FIG. 80. FIG. 80 shows a comparison between a negative viral RNA control 8010 and a positive viral RNA control 8005, according to some embodiments.

### Example 16: Enzyme-Linked Immunosorbent Assay (ELISA)

Immunoassays, including ELISA are among the most utilized tools in research and diagnostics since they can be used for rapid testing and deployed as point-of-care (POC) tools.

Immunoassays are broadly used for diagnostics by detecting either macromolecule or small molecule in biological fluids (e.g. whole blood, serum, saliva) Immunoassays rely on the ability of an antibody or antigen to bind to a specific structure of a molecule. ELISA tests are broken into several types of tests based on how analytes, antigens, antibodies are bonded and used. For instance, direct ELISA, is based on the binding between an antigen and a specific antibody attached to an enzyme (e.g. HRP). A substrate (e.g. OPD, TMB, ABTS, PNPP), added into the reaction, changes color upon reaction with the enzyme, confirming the presence of the antigen in the sample. Sandwich ELISA, competitive ELISA, reverse ELISA are based on the same principle, the only difference is the order in which antibodies, analyte, and antigens are added into the reaction Immunoassays may be quantitative or qualitative. Qualitative immunoassay gives to the user, an answer about the presence or absence of the analyte (e.g. antigen, antibody). While, the quantitative immunoassay gives to the user the concentration of the analyte in the sample.

Various types of immunoassays, including sandwich ELISA, competitive ELISA, reverse ELISA, qualitative and quantitative ELISA may be performed on the array device, in a droplet-based format, for the detection of a broad range of analytes from a wide variety of biological fluids (e.g. whole blood, serum, saliva).

Samples and reagents may be introduced on the array tile either manually or in an automated fashion. In the case of whole blood, EDTA or citrate may be added to the whole blood droplet, in order to reduce coagulation. Serial dilutions of samples, positive and negative controls may be performed in parallel to allow for quantitative measurements. Qualitative measurement may also be performed by successive sample dilution and on-chip measurement until optimum concentration is reached for a proper readout. Sample (e.g. plasma) may be diluted on-chip to minimize backgrounds caused by non-specific binding. Throughout these reactions, the evaporation and humidity control methods and systems can be applied to the array to maintain constant physical properties of the droplet(s), the array itself, and the area adjacent to the array and/or the droplet(s).

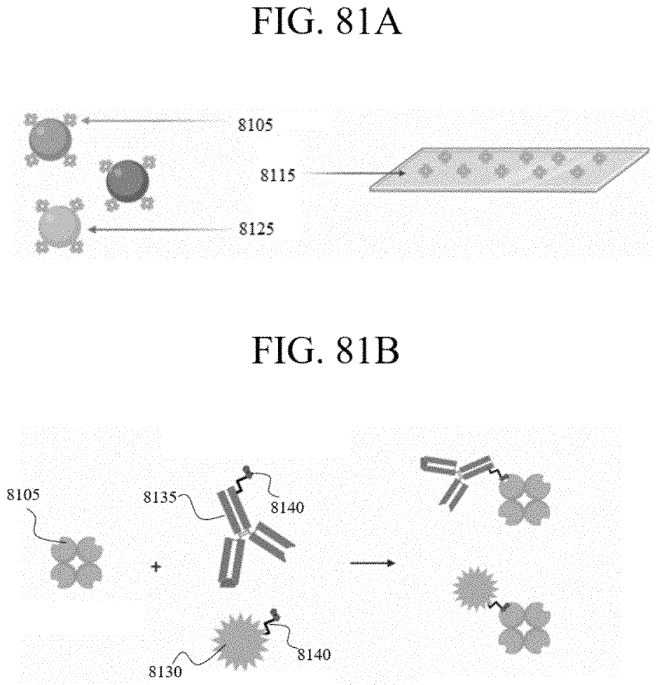

A primary antibody or an antigen is immobilized on a surface. The surface may be the surface of a film (or dielectric or hydrophobic surface) covering the array tile, beads such as magnetic beads, or particles (e.g. plasmonic magnetic nanoparticles). The surface, film, beads or particles may be coated with streptavidin, a protein with high affinity for biotin. A recombinant antibody or antigen with a biotinylated extremity may be attached to the surface, film, beads or particles by streptavidin-biotin binding, as depicted in FIG. 81A. Streptavidin may be saturated (e.g. with 0.1% BSA) in order to reduce non-specific binding. FIG. 81A depicts embodiments of beads 8125 and film 8115 coated with streptavidin 8105.

The immobilization of antibody or antigen surface, film, beads or particles may be performed on the array tile by mixing and incubating the antibody or antigen of interest either on the streptavidin surface or together with streptavidin coated beads/particles. A magnet may be used to concentrate or immobilize the coated magnetic beads in a specific location on the consumable.

The streptavidin 8105, or the streptavidin coated film 8115 as depicted in FIG. 81A, may be provided to the customer as a lyophilized or freeze-dried sample in order to preserve its activity. A rehydration step may then be performed on the array tile prior to the experiment.

FIG. 81B depicts bonding of streptavidin 8105 to antigens 8130 and antibodies 8135, according to the embodiments described herein. In some embodiments the antigens 8130 and/or antibodies 8135 are biotinylated. In some embodiments, the antigens and/or antibodies comprise a linker 8140. In some embodiments, bonding of streptavidin to a antigen or antibody forms a biotin streptavidin complex.

Simultaneous antigens or antibodies may be detected on the same device with the use of the functionalized surface, beads or particles as described herein. After the antigen or antibody capture, end-to-end workflow may be performed on the device, unbound material being washed off, detection by a conjugated tracer antibody with an enzyme and the use of enzymatic markers enabled by successive flushing of reagents and washing solutions on our surface, beads or particles. The readout may consist in the detection of a chromogenic product that develops from the substrate, the reaction may be monitored live and stopped by the addition of an acidic or basic solution to the reaction. The absorbance linked to the chromogenic reaction may be read directly on the device thanks to an absorbance reader, such as an integrated spectrophotometer system, as depicted in FIG. 11F. This system can be extended to one or more droplets, allowing the simultaneous measure of several samples or biomarkers, as depicted in FIG. 11H. Throughout these reactions, the evaporation and humidity control methods and systems can be applied to the array to maintain constant physical properties of the droplet(s), the array itself, and the area adjacent to the array and/or the droplet(s).

### Example 17: SARS-CoV2 IgG Detection

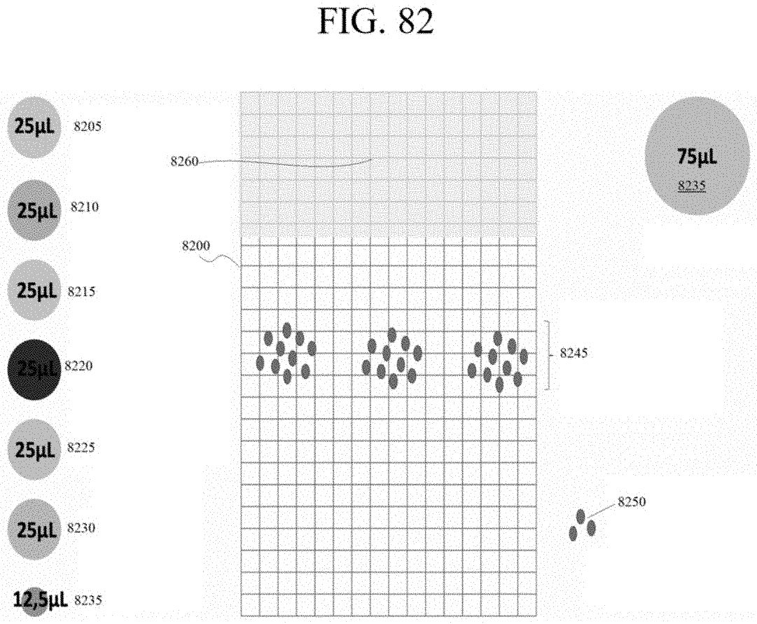

Such immunoassays described herein may enable SARS-CoV-2 IgG detection. The immunoassay may be performed in a droplet format on the arrays described herein. The FIG. 82 depicts an embodiment of droplet based SARS-CoV-2 IgG detection. Samples (e.g. plasma or serum) and reagents may be introduced on the array tile 8200 either manually or in an automated fashion. Precise droplet volumes are depicted in FIG. 82, according to some embodiments. Volume of droplets may be reduced to promote sensitivity and reduce cost. The surface used to detect IgG in samples may be magnetic beads coated with SARS-cov2 Nucleocapsid protein. Throughout these reactions, the evaporation and humidity control methods and systems can be applied to the array to maintain constant physical properties of the droplet(s), the array itself, and the area adjacent to the array and/or the droplet(s).

The immunoassay may comprise a positive control 8205, a negative control 8210, a sample 8215, biotinylated human IgG 8220, HRP-Streptavidin concentrate 8225, TMP substrate 8230, stop solution 8235, wash buffer 8240, streptavidin magnetic coated beads 8245, and magnetic beads coated with a viral RNA 8250 (e.g. SARS-CoV-2 nucleocapsid protein). In some embodiments, the precise volumes of the immunoassay comprise 25 μL of a positive control 8205, 25 μL of a negative control 8210, 25 μL of a sample 8215, 25 μL of a biotinylated human IgG 8220, 25 μL of a HRP-Streptavidin concentrate 8225, 25 μL of a TMP substrate 8230, 12.5 μL of a stop solution 8235, and 50 μL of a wash buffer 8240. In some embodiments, the array 8200 comprises a waste zone 8260.

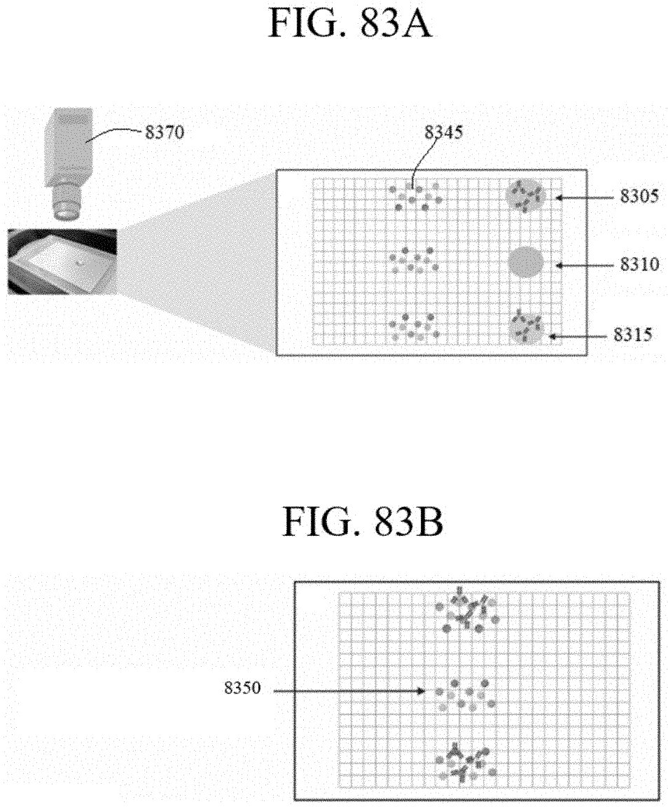

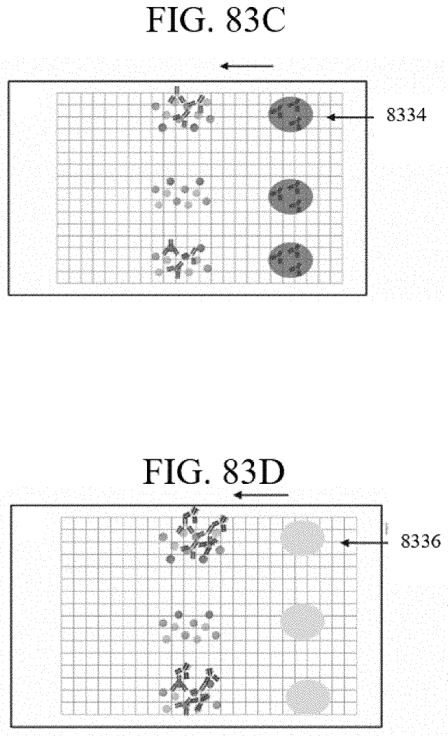

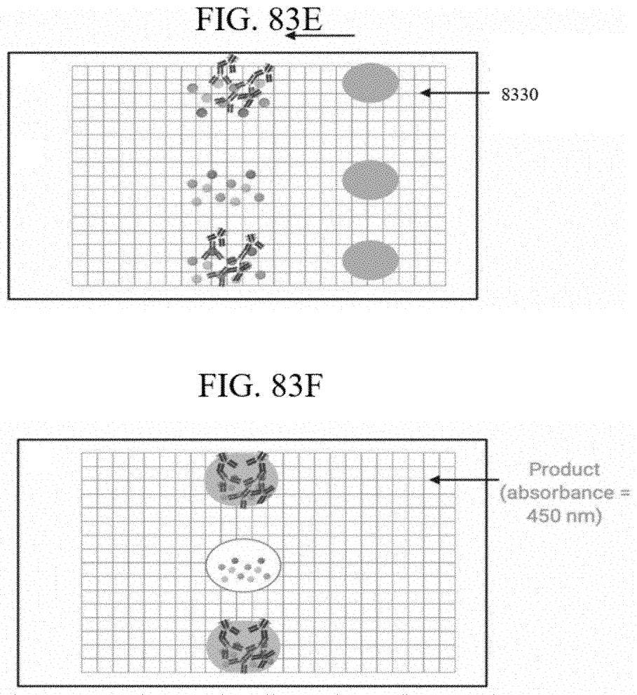

FIGS. 83A-83F depict a process of SARS-CoV2 IgG detection, according to some embodiments. As depicted in FIG. 83A, the coated magnetic beads 8345 may be concentrated on the array tile 8300 using a magnet located in proximity to the array device. These beads 8345 are coated with a biotinylated SARS-CoV-2 nucleocapsid protein (e.g. Acrobiosystems, Origene, Sydlabs, Sinobiological, . . . ). Once the magnetic beads are fixed, “positive droplet” containing IgG 8305, “negative droplet” which doesn't contain IgG 8310, and samples are moved on the magnetic beads, and mixed. If the sample 8315 comes from an immunized patient, it contains IgG. The SARS-CoV-2 nucleocapsid protein beads have a high affinity for IgG, and fix them. Since, in the negative control, there is no SARS-nCov2 IgG, the beads may remain unbounded. Throughout these reactions, the evaporation and humidity control methods and systems can be applied to the array to maintain constant physical properties of the droplet(s), the array itself, and the area adjacent to the array and/or the droplet(s).

A washing step is performed with a washing solution containing Tween and PBS (e.g. wash buffer concentrate 20×, Raybiotech). The washing step allows the removal of unfixed material, only SARS-nCov2 IgG remains fixed on the magnetic beads. On the positive control and sample site, in the case of if the sample is derived from an immunized patient, what remains is just the primary antibody bound to the beads, whereas on the negative control, beads remain naked.

As depicted in FIG. 83B, the beads may be pulled down on the array tile by applying a magnetic field. The excess liquid is may be transported to the waste zone using electrowetting. A droplet containing a solution of Tween and PBS (wash solution) may be moved over the beads. The beads may still be held down while moving this wash solution. The wash routine may be repeated about 4 times. Various concentrations of Tween and PBS may be used for the washing steps. The beads may be released for proper mixing. After this, the beads may then again be held in place while removing the supernatant using electrowetting. Throughout these reactions, the evaporation and humidity control methods and systems can be applied to the array to maintain constant physical properties of the droplet(s), the array itself, and the area adjacent to the array and/or the droplet(s).

As depicted in FIG. 83C, droplets containing tracer antibodies 8334 (e.g. RayBiotech, Abcam) conjugated with a biotin may be moved on the magnetic beads. Tracer antibodies have a high affinity for IgG (primary antibody) and bind it. A washing step may be performed in order to remove unbound material.

As depicted in FIG. 83D, droplets containing HRP-Streptavidin concentrate 8336 may be moved on the target zone and fix the tracer antibody on the streptavidin-biotin part, allowing the presence of a HRP extremity. A washing step may be performed in order to remove unbound material.

As depicted in FIG. 83E, droplets containing substrates 8330 such as TMB (e.g. RayBiotech, Promega, ThermoFisher) may be moved on the magnetic bead, resulting in a chromogenic product in contact with the complex IgG-Tracer antibody.

As depicted in FIG. 83F, in the case of a positive droplet containing IgG, the droplet will present an absorbance at 450 nm. On the contrary, in the case of the absence of IgG in a patient's sample, the droplet will not show the absorbance at around 450 nm. The reaction may be stopped by adding a droplet containing 2M Sulfuric Acid (e.g. RayBiotech, Fisher Scientific). The absorbance may be read directly on the array with an UV-Visible camera 8370 (as depicted in FIG. 83A) provided with the device. Throughout these reactions, the evaporation and humidity control methods and systems can be applied to the array to maintain constant physical properties of the droplet(s), the array itself, and the area adjacent to the array and/or the droplet(s).

### Example 18: Simultaneous Nucleic Acid Based Diagnostics and Serological Test

Simultaneous nucleic acid and protein analysis such as immunoassays may be performed in parallel on the same array. The array may be partitioned into different zones. For example, a first zone may comprise a nucleic acid based diagnostics zone and a second zone may comprise a serological tests zone.

In the case of a qPCR/qRT-PCR/PCR/iNAAT amplification and detection, an incubation zone with adjustable temperature may be dedicated to each specific type of assay. Nucleic acid based diagnostics and serological testing may be performed as described above. Fluorescent or colorimetric methods may be used for readout and signal detection, either in real time or at the end of the reaction for either a quantitative nucleic acid amplification or immunoassay testing.

### Example 19: Simultaneous DNA/RNA Extraction and Testing

DNA and RNA may be analyzed simultaneously on the device. DNA/RNA may be extracted from cells (mammalian, bacterial, plants), virus, biological fluids. DNA and RNA may be extracted and purified using detergent and enzymatic based lysis followed by magnetic bead based purification (e.g. Zymo, Qiagen, ThermoFisher kit). Once RNA/DNA extracted, an enzymatic digestion of proteins and/or RNA/DNA may be performed to improve the purity of the sample. In the case of a RNA extraction and testing, a reverse transcription may be performed on the same array with random primers, oligo-dT primers or gene specific primers in the presence of a reverse transcriptase (e.g. Maxima H Minus Reverse Transcriptase, SuperScript™ IV First-Strand Synthesis System). DNA or cDNA may then be amplified and detected as described above (c.f. qPCR, iNAAT). Throughout these reactions, the evaporation and humidity control methods and systems can be applied to the array to maintain constant physical properties of the droplet(s), the array itself, and the area adjacent to the array and/or the droplet(s).

### Example 20: Gene Assembly

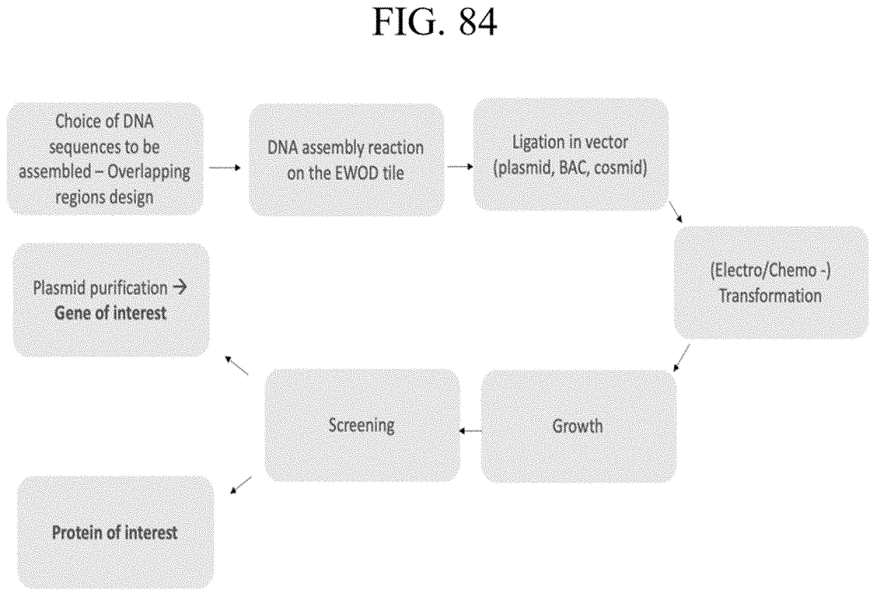

DNA assembly is becoming an essential tool in Synthetic Biology to create customized DNA fragments for various downstream applications (metabolic engineering, DNA library preparation, whole genome assembly, combinatorial assembly, data storage, novel natural product discovery..). The invention provides a unique way to overcome the 96/384 well plates limitation by implementing a high throughput combinatorial DNA assembly technique in a droplet on the array devices as described herein. The invention enables end-to-end fully-automated processes from DNA assembly to protein expression on the EWOD array. An illustrative, but non-limiting flow chart showing steps in one sequence for multipart DNA assembly is depicted by FIG. 84. Throughout these reactions, the evaporation and humidity control methods and systems can be applied to the array to maintain constant physical properties of the droplet(s), the array itself, and the area adjacent to the array and/or the droplet(s).

Various gene assembly methods based on homology, recombination, amplification and digestion/restriction may be implemented on the array. In certain embodiments, the assembly system may use Gibson assembly, Golden Gate, Gateway, Ligation Independent Cloning (LIC), GeneArt II or Overlap Extension PCR (OE-PCR) method. Approaches such as Gibson assembly may assemble up to 6 fragments in an “one-pot” reaction whereas other combinatorial and chain reactions may assemble up to hundreds different fragments. Many commercial kits such as NEBuilder HiFi assembly Mix™, the NEB® Golden Gate Assembly Kit (BsaI), the Golden GATEway Cloning Kit and The GeneArt™ Type IIs Assembly Kit may be translated to our platform.

A large range of DNA fragment lengths may be assembled, for example short genes such as GFP gene may be assembled from shorter fragments or longer DNA fragments (e.g. LacZ gene) may be assembled and cloned into larger constructs such as plasmids. Regions of DNA too large to be amplified by PCR may be divided into multiple overlapping PCR amplicons and then assembled into one piece.

Droplets containing reagents and gene fragments to be assembled may be moved, merged and mixed in either a predetermined automated fashion, a random automated fashion or in a manual fashion on the array device. Droplets may be heated to specific temperatures (e.g. 50° C.) in specific steps and areas of the array to enhance assembly efficiency. In certain embodiments, the device may integrate different types of heating pads at different temperatures enabling complementary reactions (e.g. PCR) on the same device thus enabling the amplification of assembled products. Droplets may be tracked by machine vision or sensing during each step of the workflow. The sensing and feedback from machine vision can be used to find optimal conditions for assembly of DNA fragments with high yield. Throughout these reactions, the evaporation and humidity control methods and systems can be applied to the array to maintain constant physical properties of the droplet(s), the array itself, and the area adjacent to the array and/or the droplet(s).

### Example 21: Gibson DNA Assembly

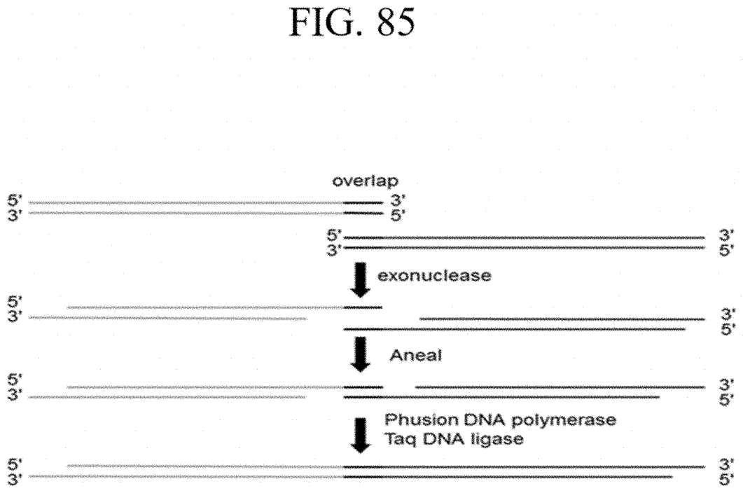

The Gibson DNA assembly method, as depicted FIG. 85 (see, e.g., Gibson et al. (2009) Nature Meth., 6: 343-345), is a one step isothermal in vitro recombination method for assembling very small (˜100 base pairs) to very large fragments of DNA (˜500-kb). The process uses T5 exonuclease*, Phusion polymerase* and Taq ligase* as the primary components. The steps involved in assembling two DNA fragments is as follows: The two fragments of DNA to be assembled are mixed with T5 exonuclease, Phusion polymerase and Taq Ligase. On an array device, each of these elements are in a droplet form and throughout these reactions, the evaporation and humidity control methods and systems can be applied to the array to maintain constant physical properties of the droplet(s), the array itself, and the area adjacent to the array and/or the droplet(s).

Upon incubation of the above mixture at 50° C. on the array device following sequence of events occur within the droplet. The T5 exonuclease starts chewing back the double stranded DNA from the 5′ end, leaving the terminal ends sequence overlaps exposed. The complementary single-stranded DNA overhangs are then annealed. Following the annealing of two strands, the Phusion polymerase fills the gaps and the ligase finally seals the nicks.

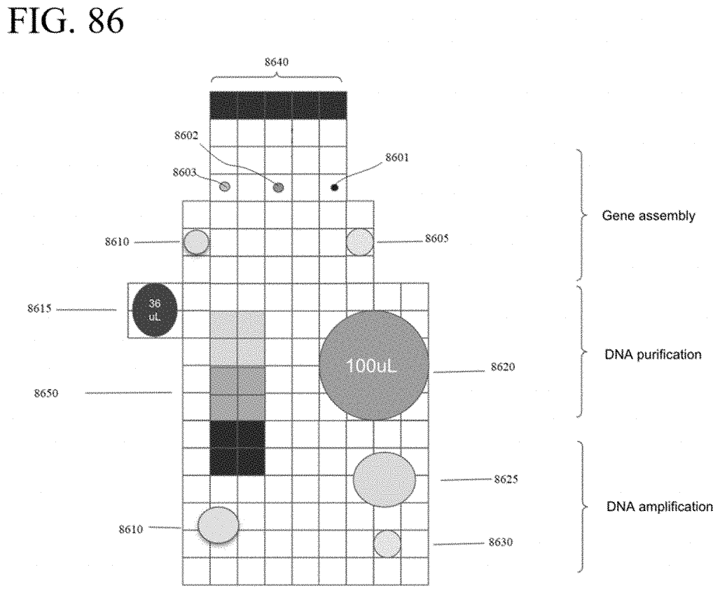

This method was used to assemble the GFP gene (720 bp) divided into 3 equal fragments of 240 bp. The Gibson assembly is a standardized and scar-less method. The process to be performed is a one-step reaction on the EWOD array 8600 (array device) using the NEBuilder HiFi assembly mix′ kit. The assembly reaction is followed, on the same array, by a DNA purification and amplification. FIG. 86 depicts the disposition of the droplets on the chip 8600 for the consecutive DNA assembly, purification and amplification. The reagents for this workflow include: the 3 fragments of interest (8601, 8602, 8603), the Assembly Mix 8605, Nuclease-free water 8610, AMPure magnetic beads 8615, Ethanol 8620, the PCR Master Mix 8625 and the primers 8630 (all in droplet form on the array device). Droplets may be actively mixed at station 8650 during the incubation to improve the yield and the efficiency of the DNA assembly. In embodiments, 5 μL of fragments of interest (8601, 8602, 8603), 10 μL of the Assembly Mix 8605, 36 μL of magnetic beads 8615, 100 μL of Ethanol 8620, and 5 μL of the PCR Master Mix 8625 are provided.

For successful assembly of short fragments, any two fragments may be designed with an overlapping region. These overlapping regions may be either manually designed to allow strong and complementary hybridization or done automatically (e.g. Gibson Assembly® primer design). The length of the overlapping region may vary from 20 to 60 pb but in certain cases may be up to 100 pb to enhance the annealing of the fragments to be assembled.

The reagents may be introduced on the array tile either manually or in an automated fashion. The reagents may be mixed with the three fragments, incubated at various temperatures in a predetermined sequence. The order of sequences, incubation temperature and time may be optimized by testing random configurations on the assay, first in a manual way then in a complete autonomous way. The droplet containing the final fragment assembled may be purified on the same array using magnetic beads. Purified assembled fragments may be mixed with PCR primers and PCR Mix on the array and amplified by PCR. PCR amplification can be performed on the array device by either cycling the temperature of a droplet locally or by shuttling the droplet back and forth on the array device with temperature gradient. Time of incubation, mixing during the assembly step, increasing the concentrations of initial fragments and adding poly ethylene glycol (PEG) may be optimized to improve the assembly yield and limit the number of undesired mutations in the final assembled fragments. Waste area 8640 may be provided to discard waste samples.

In some embodiments, the array 8600 comprises one or more regions to perform more than one process. Array 8600 may comprise a region designated for gene assembly. Array 8600 may comprise a region designated for DNA purification. Array 8600 may comprise a region designated for DNA amplification. Regions may be provided with specific reagents for carrying out the designated processes on the particular region. This partitioning of an array can be performed by the methods and systems described herein.

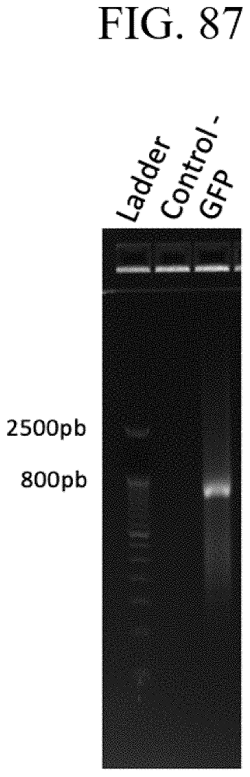

FIG. 87 shows 1-2% Gel electrophoresis of a PCR-amplified synthetic GFP gene. The GFP gene was assembled on the array device in a droplet.

### Example 22: Golden Gate Assembly

The Golden Gate Assembly is a restriction/digestion-based method commonly used for DNA assembly. This technique is highly recommended for site-directed mutagenesis, custom-specific TALEN in vitro construction and combinatorial libraries construction of diverse populations. This method presents two main advantages i) The ability to overcome the issue experienced by long repeated sequences ii) provides a scar-less assembled fragment. The process is a “one pot digestion-ligation” using a type IIS restriction endonuclease (i.e. BsaI, BsmBI) and a T4 DNA ligase as primary components. The two or more fragments to be assembled flanked by IIS restriction sites are cleaved outside the recognition site, therefore enabling a scar-less assembly. The overlapping regions of the digested fragments are then ligated by a ligase.

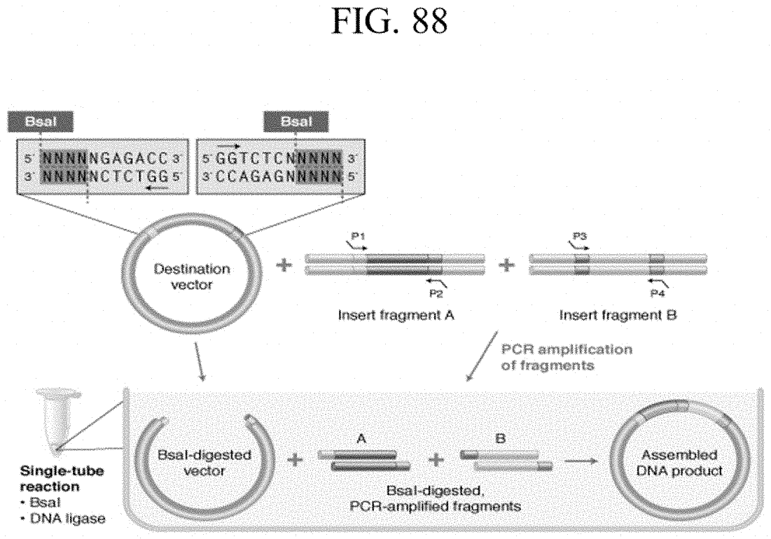

FIG. 88 depicts the process of a Golden Gate assembly method. Here the Golden Gate assembly was used to assemble the LacZ gene (3 075 bp) that were divided in 6 equivalent fragments of 520 bp flanked by BsaI recognition site on the EWOD array. The DNA assembly is performed using the NEB Golden Gate Assembly kit (BsaI). The assembly reaction may be followed, on the same array, by a DNA purification and amplification (purification and PCR amplification described in other sections). The reagents for this workflow include: the 6 fragments of interest, T4 DNA ligase buffer, T4 DNA ligase, BasI enzyme, Nuclease-free water, AMPure magnetic beads, Ethanol, PCR Master Mix, and primers. These reagents may be merged in a pre-defined order either manually or in an automated fashion on the array device. The final mix of all the components is performed on the array and followed by 30 cycles of thermal cycling between 37° C. for 5 mins and 16° C. for 5 mins. The final product is purified and amplified as described herein. It is possible to measure the amount of DNA being assembled in real time using fluorescence or other detection methods and stop the cycling process when an appropriate amount of DNA is produced. This is a unique feature about being able to assemble DNA on an array device that is monitored using optical sensors. Alternately, it might be possible to measure the DNA quantity, yield and assembly efficiency using other electrochemical sensing methods. Throughout these reactions, the evaporation and humidity control methods and systems can be applied to the array to maintain constant physical properties of the droplet(s), the array itself, and the area adjacent to the array and/or the droplet(s).

### Example 23: Quality Control of DNA Assembly Efficiency

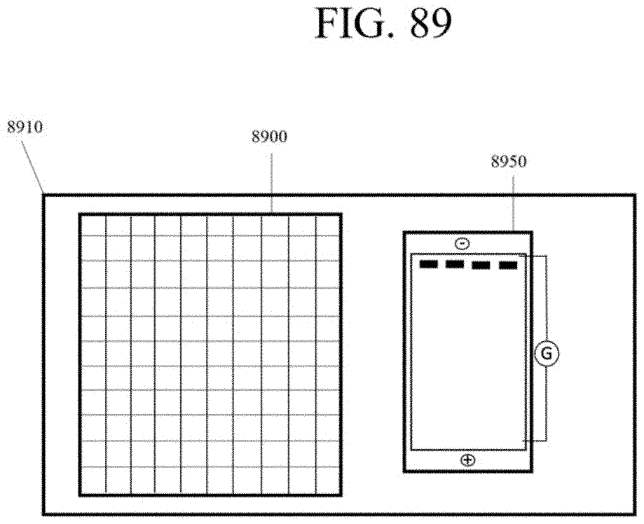

Many features such as purity, size, accuracy and quantification are assessed both on the array device and off-chip (outside the array device) as quality controls. Size and purity control may be performed using electrophoresis-based methods as shown in FIG. 89, sequencing techniques (Illumina, PacBio, Oxford Nanopore.) and DNA quantification techniques. The electrophoresis device 8950 integrated with the EWOD array 8900 as shown in FIG. 89 and provided on a tile 8910. The electrophoresis device may be replaced by other measurement techniques for measuring purity and size of DNA on the array device. DNA quantification may be done using the qPCR directly on the array as previously described herein (see previous writeup). As purity is a key element, removing the non-assembled fragments is an important step in the workflow. The removal of the non-assembled fragments can be done in many ways. One way to do this is using magnetic bead based size selection described in other sections (selecting DNA fragments of a known fragment size by carefully controlling the concentration of the DNA and magnetic beads). Another way to do this is to use magnetic beads as well, but instead of size selection you would use the beads as solid support to immobilize the fragments that need to be assembled. For instance, the 5′-end of the first fragment may be biotinylated prior to assembly and the surface (e.g. beads or tile) coated with Streptavidin. The Streptavidin-biotin interaction keeps the first fragment bound to the bead and the assembly process for DNA progresses on the bead. A droplet with these beads on which DNA is assembled may contain unassembled fragments. It is possible to wash away these unassembled fragments and any other impurity using the combination of electric field and magnetic field on the array device as described in other sections. These wash steps may occur between the serial assemblies of DNA fragments, therefore removing the non-assembled fragments. Gel migration-extraction and size-selection with SPRI may be also used in conjunction on the device. Throughout these reactions, the evaporation and humidity control methods and systems can be applied to the array to maintain constant physical properties of the droplet(s), the array itself, and the area adjacent to the array and/or the droplet(s).

### Example 24: Dilution to Single Molecule and Amplification

Single molecule amplification and detection is becoming a more and more common tool for genetic disease investigation (e.g. detection of rare genetic variants, analysis a copy variation number, NGS, calculation of the abundance of a specific loci, detection of methylation status), diagnostic (e.g. pathogen detection and quantification), pharmacogenomics and drug discovery (e.g. antibody screening). This revolution is also becoming a first-choice method to overcome the traditional cell-based cloning for generating novel, error-free DNA. A Microfluidic In vitro Cloning based on single molecule amplification is implemented on the EWOD array.

Partitioning is the key step of single molecule analysis. The main idea is to start with a pool of biomolecules (DNA/RNA/protein) and segregate to a unique molecule partition (e.g. pools). Isolation of a single DNA molecule is defined by a probabilistic model following a Poisson distribution. In order to perform on this array, we may start with a reservoir (e.g. a droplet disposed on an array) containing molecules of interest. The array device splits and creates smaller droplets to carry out serial dilution as described in other sections of the present disclosure. The splitting operation and the resulting newly created droplets contain single molecules that follow a probabilistic model (Poisson's distribution).

The reservoir droplet may start from different initial samples such as blood, RNA/DNA/protein extraction product. For example, with DNA as the starting material, each DNA molecule may be identified using for example barcodes enabling a specific detection of a single DNA template after partitioning. Biomolecules partitioning may be implemented on the array tile according to different methods. Partition may be obtained on the tile by serial dilution where droplets may be successively diluted to a final limiting dilution. Splitting droplets is essential to perform the dilution, therefore a hydrophobic slicer (or other splitting mechanism) may be used on the tile. On the other hand, applying precise and accurate opposite electrical force on the droplets may be an alternative to split it.

Another method that may be used for the partitioning is an emulsion-based technique. A continuous flow may pass through a channel (encapsulating bioreagents in nano-femtoliter droplet), beads emulsion-based technique (BEAMing). The channel and creation of the emulsion may be done directly on the array device or can be done externally first and then the resulting emulsion can be introduced onto the device. The small volumes involved and the massive parallel partitioning enable the implementation of multiplex assay for multiple targets in a single run. Polymerase Chain Reaction (PCR) amplification is the traditional amplification method for this application, however isothermal amplification such as RCA, LAMP and RPA might also be used. The amplified virtually error-free product can be retrieved on the tile for downstream applications (e.g NGS, protein expression, etc.)

### Example 25: Cloning

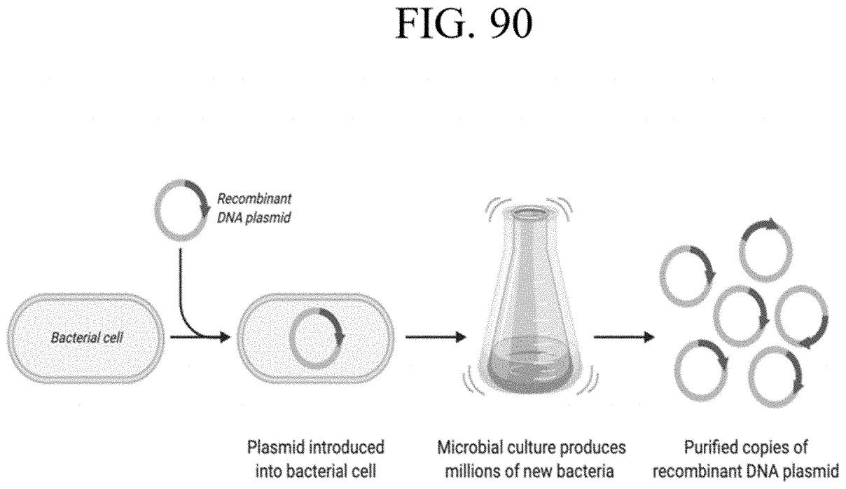

DNA cloning is the gold standard application to amplify and study a gene. FIG. 90 depicts the basic principle of cell-based DNA cloning. The invention provides a unique way for executing an end-to-end cloning application, from amplification to protein expression on the array device all carried out in droplet form.

Different types of starting material (e.g. PCR amplicon, synthetic genes, cDNA, extracted DNA) in droplets may be used on the array device. The nucleic acid template may be from various sources (e.g. whole organisms, tissues, cells, plants, organelles, synthetic). The gene of interest (GOI) may be introduced into an exogenous vector prior to cellular amplification. Various types of cells may be used on the array for the amplification (e.g. mammalian cells, bacteria, yeast, insects). Depending on the application and the length/complexity of the GOI, different vectors may be used on the array. Commonly, bacterial plasmid (e.g. pET, puC19, pGEM-T) are used as primary vector but others such as Bacterial Artificial Chromosome (BAC), viral vector (lentivirus, retrovirus) may also be used on the array. Various methods, such as Restriction/Digestion system, Ligation and Sequence Independent Cloning (LIC), Gibson cloning method, Golden Gate method, Gateway method, may be used on the array to ligate the GOI to the backbone vector. Transformation of the micro-organism by the complex GOI/backbone may be performed using either chemical or electrical strategies on the array. Parallel transformations and plating may be performed on the array enabling a high throughput screening of DNA fragments and produced proteins. Throughout these reactions, the evaporation and humidity control methods and systems can be applied to the array to maintain constant physical properties of the droplet(s), the array itself, and the area adjacent to the array and/or the droplet(s).

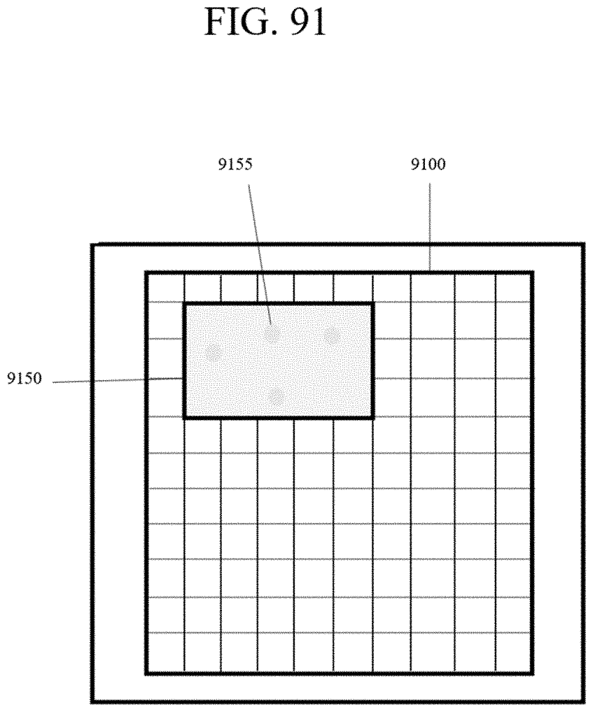

For example, both the synthetic GFP (sGFP) gene and pET vector 22b (+) (5 493 pb), described herein, may be digested using NcoI, phosphorylated and ligated on the tile in a fully- or semi-automated way. Prior to digestion, sGFP may be amplified by PCR on the array using primers flanked by NcoI recognition sites. Digestion may be performed on the array at room temperature and competent cells (Escherichia coli BL21 (DE3)) may be added on the array for chemical transformation with the ligated-product. The transformation is performed on heat pads of different temperatures for the heat-shock process (30 s at 42° C. then 5 min at RT). The transformed cells 9155 may be plated on the array 9100 using an agarose-coated surface 9150, as depicted in FIG. 91. Colonies may be screened by various molecular methods such as PCR colony or sequencing. Colonies transformed by the recombinant plasmid will express intracellular GFP and display a fluorescent phenotype when visualized under a fluorescent camera that may be provided with the device.

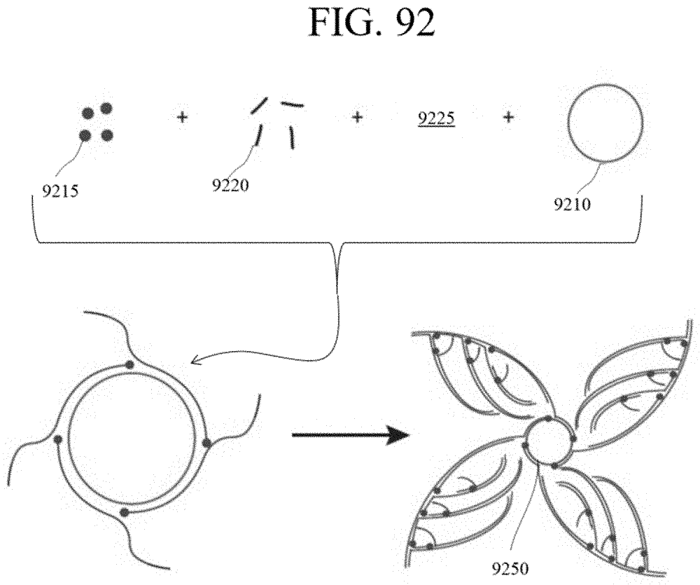

Although standard methods for cloning foreign DNA use biological hosts, cell-free methods are becoming increasingly popular to overcome limitations such as difficulties to clone high levels of sequence complexity (e.g. retro-viral long terminal repeats, gene editing vectors.) or potential cell toxicity. For DNA amplification various methods may be implemented on the array such as the isothermal Multi Primers Rolling Circle Amplification (MPRCA), as depicted by FIG. 92. For this technique, droplets 9210 containing template DNA, Φ29 DNA polymerase 9215, primers 9220, pyrophosphatase and dinucleotides 9225 may be loaded, mixed and incubated at 95°, 30° C. and 65° C. on the array. The final product 9250 may be purified on the array as described herein and analyzed by DNA quantification and gel electrophoresis. Said primers, may comprise random hexamer primers.

For protein expression, various sources and samples may be used to initiate cell-free cloning and expression on the array. Protein expression may be performed on the device using exogenous transcriptional/translational machinery. Such method may be translated on the array using methods such as the NEBExpress cell-free E. coli protein Synthesis system, the Expressway™ Mini Cell-Free Expression System and the Next Generation Cell Free Protein Expression Kit (Wheat Germ).

For the NEBExpress cell-free E. Coli synthesis system workflow, reagents (NEBExpress® S30 Synthesis Extract, buffer, RNA polymerase, RNase inhibitor and the plasmid template) may be dispensed on the array either manually or in an automated fashion. For example, the synthetic GFP gene, previously assembled may be ligated to the pET 22 (+) that contains all the regulatory elements needed for the protein expression. The droplets may be moved, merged and mixed for 3 hours at 37° C. in a fully or semi-automated fashion. Final product may then be analyzed on a SDS-PAGE or on a chromatography-based column.

### Example 26: Single Cell and Single Bead in Picoliter Droplets

Single cell analysis has become a critical tool to answer a broad range of biological questions. The capability for the arrays described herein to manipulate a broad range of volumes from picoliters (or as low as femtoliters) to microliters allows the capture and manipulation of individual cells. Successive droplet splitting (see the New Droplet Actuation Mechanisms section) may be used in conjunction with machine vision based cell detection for the isolation of individual cells. Isolated cells may be fluorescently labeled for the isolation of specific cell types (see cell enrichment section). Cell isolation may be performed in an automated fashion with automatic cell detection. Cells may be assayed directly on the array by combining the cell isolation to previously described sample processing (e.g. qPCR, sequencing library preparation). Cells may be lysed directly on the array to capture their genetic material (e.g. DNA, RNA) or content (e.g. proteins). Individual cells may be tested for specific features such as expression of a specific gene or presence of a particular DNA mutation. In a similar manner to encapsulation and isolation of a single cell in a droplet, functionalized beads may be individually isolated in droplets on the array device. Isolated beads may carry oligonucleotides for the capture of DNA/RNA, antibodies for the capture of specific cell types, peptides or proteins/enzymes to assay cell responses in droplets. In some embodiments, a cell and a bead or several cells and several beads may be isolated in a single droplet for further processing or analysis.

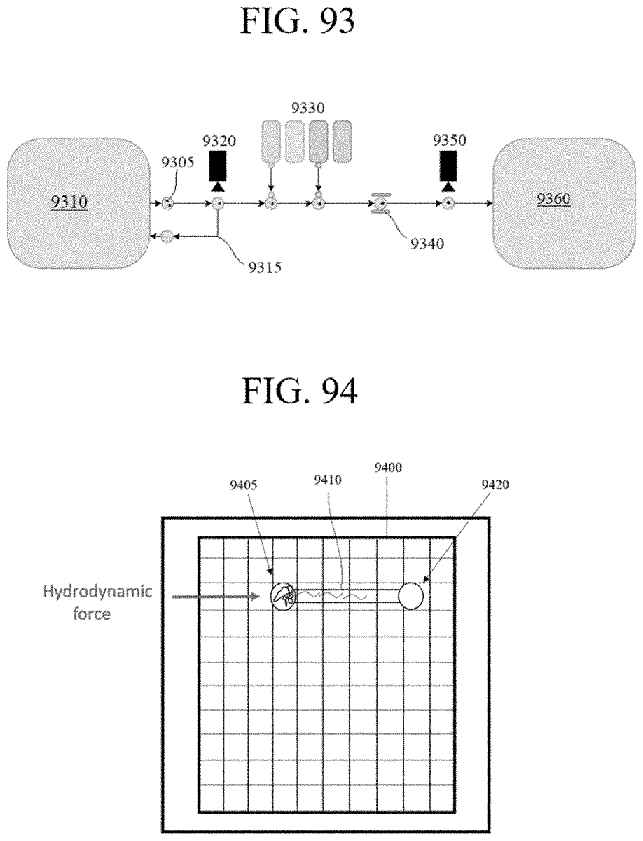

This entire process, according to some embodiments, is depicted in FIG. 93. In some embodiments, droplets 9305 are provided by a reservoir 9310. The droplets may be processed at a screening station 9320, wherein the screened to determine if they comprise only a single cell. If the droplets comprise more than a single cell, they may follow a return path 9315 and return to the reservoir. Droplets containing a single cell may be mixed with one or more reagents provided by one or more reagent reservoirs 9330. Droplets may then continue on to a heating station 9340. At the heating station 9340, samples may be heated to induce incubation. Sample droplets may then proceed to detection station 9350. After detection of the sample, droplets may proceed to a waste reservoir 9360. In these embodiments wherein the droplets are of femtoliter to microliter-sized volumes, the droplets are especially susceptible to evaporation. Throughout these reactions, the evaporation and humidity control methods and systems can be applied to the array to maintain constant physical properties of the droplet(s), the array itself, and the area adjacent to the array and/or the droplet(s).

### Example 27: Linked/Synthetic Long Reads

The barcoding and reading by sequencing of individual short fragments of a given DNA fragment is an alternative to long-read sequencing that takes advantage of the throughput and low cost of short read sequencing (e.g. Illumina sequencing). Long DNA fragments may be singly isolated in individual droplets by serial dilution and droplet splitting to achieve a state where most droplets contain no more than one single fragment. Alternatively, water in oil type emulsion may be prepared on the array to isolate these single fragments in individual droplets. The isolation, fragmentation and barcoding of short sub-fragments may be performed in each droplet by successive enzymatic digestion (e.g. fragmentase), end-repair and barcode ligation. The conversion of these long fragments into shorter barcoded fragments may be processed in parallel for a large number of fragments. This process may take place on the same array on which high molecular weight DNA was extracted (described in prior writeup). Barcode fragments may be purified and pooled on the array prior to sequencing.

### Example 28: Processing of High Molecular Weight DNA

Shearing or fragmentation of HMW DNA is a requirement of some long-read sequencing technologies (e.g. PacBio). Currently mechanical methods such as hydrodynamic shearing, nebulization, sonication (e.g.Bioruptor or g-Tube) are used to produce a narrow size range of DNA of 10-30 kb. IN some embodiments, as depicted in FIG. 94, on the array 9400, HMW DNA 9405 may be pushed into a cylindrical extra-modular tube 9410 to which a specific liquid flow may be applied in a specific hydrodynamic pattern in order to tightly shear the DNA, to provide sheared DNA at an output 9420.

Alternatively, the HMW DNA may be loaded on the array in a bubble of high surface tension. The hydrodynamic energy released during the bursting process would then fragment the DNA in smaller fragments. The HMW DNA may also be sheared on the array using bead-beating. The input DNA would be added to pre-loaded beads (e.g. Zymo Research WashingBeads) on the array. The HMW DNA—beads complex may be mixed on the array in either a predetermined automated fashion or in a manual fashion at a specific frequency and for a certain period of time to generate a narrow range of DNA fragments.

Specific DNA fragments size may be selected on the array by electrophoresis. The sheared DNA may be loaded in different agarose prefilled areas of the array and a specific voltage may be applied. Fragments of different molecular weights would migrate at specific speed and separate on the array. DNA fragments of desired length may then be extracted.

Various long-read library preparation workflows may be implemented on the array (i.e. Oxford Nanopore, Pacific Biosciences). For example, the SMRTbell HiFi library prep method may be translated to the array by performing all the different steps in an end-to-end fashion, from DNA damage repair, End-repair/A-tailing, Adapter ligation to digestion and purification. The bioreagents needed for this workflow may be dispensed either manually or in an automated fashion in an optimized spatial disposition on the array. The droplets may be merged, incubated and mixed in either a predetermined automated fashion or in a manual fashion.

### Example 29: Software Architecture

A user interface that allows a user to configure the actuation of droplets (actuation means to subject a droplet to motion, mixing, heating or other operations) on an array device can be applied to a computer processor configured to directed the methods and systems described herein. On the user interface, one can define a biological or chemical protocol to be performed on the array device. Through this interface, information about liquids (such as prescribing volumes) to be used in a protocol may be entered manually by a user or automatically populated using natural language processing algorithms. The prescribed volumes may be translated into compatible volumes for the array device (volumes that are appropriatec on the array device). This translation can be achieved by normalizing maximum and minimum values and then calculating the relative intermediate volumes. It may be possible that liquids with different chemical properties spread differently on the array device and hence occupy different number of actuation electrodes on the array device. These droplet volumes may be adjusted to improve mobility on the array device within the normalized range.

The software interface stores a set of values referred to as “droplet interaction properties”. These could include but are not limited to reagent compatibility (the ability for reagents to come into contact without affecting biological properties), history of its temperature over time, history of it's volume or reagent concentrations. Droplet interaction properties may be entered manually by the user or automatically recorded by the software using sensors such as temperature probes and optical sensors. These properties may be used to dictate which droplets may contact the same regions on the array device. These interaction properties may also be used to determine the ability and order for droplets to come into contact with each other (mixing or traversing same paths). Droplets may be grouped by common properties in software in order to generate a user interface and automated droplet pathways. Protocols may be generated by adding droplets to the array device. Droplet footprints on the grid area may be determined using the automatically calculated volumes. These footprints may be used to determine the area contaminated by a droplet. Contaminated areas may be stored and displayed to the user for the purpose of determining droplet placement and clean usable area on the array device. Throughout these reactions, the software can direct the evaporation and humidity control methods and systems to the array to maintain constant physical properties of the droplet(s), the array itself, and the area adjacent to the array and/or the droplet(s).

While a protocol is being executed on the device, “droplet interaction properties” may be recorded. These properties include but are not limited to constituent reagents, temperatures, presence of sample and errors during execution of a protocol. These properties may be displayed over a live video feed of droplets on an array device or accessed through a simulation of the protocol during execution. Areas previously covered by a selected droplet may be highlighted over the video feed, in the simulated grid area or projected (via a projector mounted above the array device) onto the physical grid area.

Data on the operation and performance of the device (array device or an instrument using the array device) may be collected by various sensors and software components. These sensors may include but are not limited to optical, capacitive, temperature and humidity sensors. The software components may include but are not limited to wireless communications, wired communications, device connections and user interactions. The data collected may be logged in order to diagnose device operations and malfunctions. This data may also be used to detect errors in real time. These detections may be used to notify users in real time when a user's intervention is required. This intervention may be administered locally by controls available on the device (for example a physical button or a software UI element) or remotely by the user or support team. The collected data may also be used to optimize the user interface.

A digital projector may be mounted over the grid area. This projector may be used to aid the user in manual pipetting of liquids onto the grid area. This may be accomplished by projecting lines or other patterns to guide the user to the desired position or region. Information about droplet positions, volumes and other droplet properties may be projected onto the grid area during operation to aid the user in monitoring of protocols. User aids such as progress to desired volume during pipetting may also be displayed when interacting with the device. Colors may be projected onto droplets in order to highlight positions, contaminated areas (areas already traversed by another droplet) on the array and future paths in order to correlate the physical grid area to the software simulation.

Neural networks may be trained to test for the presence or absence of droplets in an image. These machine learning models may be trained for various fields of view over the grid area of the arrays device. The models may then be used to determine which electrode areas are in contact with droplets. Using algorithms such as the sliding window method, confidence in droplet positions may be assigned and then correlated to expected droplet positions based on those prescribed by the planned protocol. This data may be used to adjust electrode states and to flag potential errors in operation. Neural networks may also be trained to correlate images of droplets to their volumes. These models may be created for various types of liquids in order to accurately predict droplet volumes with different properties. This data may be used to control feedback for use in applications such as droplet evaporation. These models may be used on live video feeds of droplets during device operation.

Biological protocol documents define the physical operations such as liquid mixing and heating as well as required reagents and liquid volumes. These protocols are broken down into step by step instructions which contain parameters to define these reagents and operations such as reagent concentrations and mixing speed. The feasibility of these operations and liquids on the array device can be determined by examining the parameters and comparing known limitations of the array device to deduce compatibility. The compatibility of these properties including but not limited to reagents, physical operations, droplet volumes and chemical reactions may be determined experimentally. These properties may then be used to develop a filter which is employed to determine whether a standard protocol is compatible or incompatible with the array device. A list of descriptors for these compatible and incompatible properties may then be compiled and used to create a natural language processing model. This model may be trained to extract the overall structure as well as the aforementioned compatibility properties from a standard protocol document. The extracted information may be passed through the filter to determine whether the standard protocol is compatible with the device. Once compatibility is determined, the key information may then be used to inform the translation of the standard protocol operations to device specific operations. These operations may be compiled and used to generate a device compatible protocol. Furthermore, web scraping algorithms may be developed which can locate biological protocol documents and compile them into a database. The data in the database may then be fed as inputs to the natural language processing model which determines compatibility and translates to device protocols. These protocols may then be collected and added to a library of protocols.

### Example 30: Reconfigurable Chips

Biological protocols on the array device define the area traversed by droplets. Some areas will be traversed more often by droplets, for example the area where a magnetic field is present or where a droplet may get heated. Likewise, some areas may not be traversed as often—for example locations where a droplet of certain composition is mixed. These high traffic and low traffic areas may be shared between biological protocols which share similar operations. These common areas may be correlated to find patterns in array device usage. As a library of protocols is generated, algorithms may be developed which can find large scale patterns. These patterns may consist of information such as grid space (grid space implies actuation electrode arrays of the array device) usage or required heater magnet, and/or electrode arrangements. The data extracted by these algorithms may be used to optimize the physical layout of the grid area (arrangement of actuation electrodes) based on the requirements of a protocol. The physical layout generated may then be translated to a chip design where circuit routing and physical assembly are automatically determined. As compatible protocols are generated or as users create custom protocols, optimized chips (chips imply array device, EWOD arrays) may be generated and made available to fabricate.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-124. (canceled)

125. A method for droplet processing, comprising:
a. providing an electrowetting array;
b. dispensing a droplet adjacent to said electrowetting array; and
c. subjecting said droplet to conditions sufficient to perform a chemical reaction, biological reaction, or biochemical reaction within said droplet while said droplet is adjacent to said electrowetting array, wherein during said chemical reaction, biological reaction, or biochemical reaction, a volume of said droplet is maintained to within about 20% of a threshold volume, wherein said threshold volume is based on said chemical reaction, biological reaction, or biochemical reaction.

126. The method of claim 125, wherein said electrowetting array comprises one or more electrodes.

127. The method of claim 126, wherein a temperature sensor is coupled to a side of said electrowetting array.

128. The method of claim 125, further comprising providing a temperature control element external to said electrowetting array.

129. The method of claim 125, wherein an original volume of said droplet and said threshold volume differ by at most about 10%.

130. The method of claim 125, wherein an original volume of said droplet and said threshold volume differ by at most about 5%.

131. The method of claim 125, wherein an original volume of said droplet and said threshold volume differ by at most about 1%.

132. The method of claim 125, wherein an original volume of said droplet and said threshold volume differ by at most about 0.01%.

133. The method of claim 125, wherein an original volume of said droplet and a threshold volume of said droplet differ by at most about 5% over a time period of at least 10 seconds.

134. The method of claim 125, wherein during said chemical reaction, biological reaction or biochemical reaction, a module in fluid communication with said electrowetting array is used to provide a liquid to said droplet to maintain said volume to within 20% of said threshold volume.

135. The method of claim 134, wherein said module dispenses a replenishing droplet when said volume and said threshold volume differ by greater than about 20%.

136. The method of claim 125, further comprising providing one or more humidity sensors adjacent to said electrowetting array.

137. The method of claim 125, further comprising using one or more sensors to detect a change in said volume of said droplet.

138. The method of claim 125, wherein said chemical reaction, said biological reaction, or said biochemical reaction is a nucleic acid isolation to generate a nucleic acid fragment, optionally wherein said fragment comprises at least 100,000 kilobases (kb).

139. The method of claim 135, wherein said replenishing droplet is dispensed,
a. directly onto said droplet; or
b. onto said electrowetting array, and said electrowetting array is used to merge said droplet with said replenishing droplet.
